(19) United States
(12) Patent Application Publication
(10) Pub. No.: US 2025/0043355 A1
(43) Pub. Date: Feb. 6, 2025

(54) PAN-CANCER TRANSCRIPTIONAL SIGNATURE

(71) Applicant: ONTARIO INSTITUTE FOR CANCER RESEARCH (OICR), Toronto (CA)

(72) Inventors: Philip AWADALLA, Toronto (CA); Fabien LAMAZE, Toronto (CA)

(21) Appl. No.: 17/612,695

(22) PCT Filed: May 20, 2020
(86) PCT No.: PCT/CA2020/050678

§ 371 Date: Nov. 19, 2021

Related U.S. Application Data
(60) Provisional application No. 62/850,903, filed on May 21, 2019.

(51) Int. Cl. C12Q 1/6886; G16B 25/10; G16B 40/20; G16H 50/20

(52) U.S. Cl. CPC C12Q 1/6886; G16B 25/10; G16B 40/20; G16H 50/20; C12Q 2600/158

(57) ABSTRACT

There is described herein a pan-cancer transcriptional signature. In one aspect, there is described a method of diagnosing cancerous cells in a patient, the method comprising: a) providing a sample containing genetic material from patient cells suspected of being cancerous; b) determining or measuring expression levels in the patient cells of at least 3 of the 1919 genes listed in Table B; c) computing a score using a classifier that takes said expression level values as input, the classifier having been previously trained on known cancerous and non-cancerous samples; wherein the score provides a likelihood of a cancerous cell.

### RELATED APPLICATIONS

This application is a national phase application under 35 U.S.C. § 371 of International Application No. PCT/CA2020/050678 filed May 20, 2020, which claims priority to U.S. Provisional Application No. 62/850,903 filed on May 21, 2019. The entire contents of each of the above-referenced disclosures is specifically incorporated herein by reference without disclaimer.

### FIELD OF THE INVENTION

The invention relates to the use of patient features for treating cancer and methods of informing the same.

### BACKGROUND OF THE INVENTION

Extensive phenotypic variations between cancer and normal tissues are likely attributable to clonal heterogeneity within a single tumor (1-9). This heterogeneity represents a major challenge for the discovery of diagnostic and monitoring biomarkers with respect to sampling and potential causes of error in interpretation. This reveals the importance of the combination of biomarkers to enhance the detection sensitivity for screening and diagnostic development (10). Furthermore, biomarker development is often restricted to individual diseases which may limit our ability to distinguish and capture relevant oncogenic processes across cancer types. Thus, the discovery of common biomarkers for early diagnosis, prognosis, and surveillance of tumors is essential to enable a tailored approach to therapy.

It is becoming increasingly critical of personalized cancer diagnostics to access ancillary molecular testing with minimally invasive procedures (11). Fine needle aspiration and core needle biopsy provide biosample of solid organ tumors for histology and immunohistochemistry investigation by expert pathologist. However, tumor heterogeneity represents a challenge and new liquid or solid molecular test must have a very high precision to overcome false positive rate and unnecessary follow-up to support expert diagnostic decisions (12). Gene expression levels have increasingly emerged as an attractive biomarker option to interrogate for broad cancer diagnostics and for tumoral sub classification and prognostication and drug resistance (13-15). Processes of aberrant transcriptomic regulation are commonly observed among cancerous tissues and contribute to high levels of phenotypic heterogeneity within cellular populations with respect to transcriptomic states and response to therapy (1, 8, 16). However, there is little insight into what is driving transcriptomic diversification origins and whether it is transient or stable over time and space, in and across, tumoral populations.

Quantitative assessments of current cancer biomarkers help to differentiate cancer cells from which the cancer originates. However, their usefulness may be altered by processes such as the loss of a tumor-specific marker expression during disease progression; for example, the loss of Nkx2-1 in non-small cell lung cancer leads to the differentiation into gut-like cells and contribute to tumoral plasticity (17). Furthermore, as we have recently demonstrated, gene expression signatures are modulated by both genetic ancestry and environmental exposures which directly impact inter-individual gene expression profiles (18).

### SUMMARY OF THE INVENTION

Applicant has integrated differential gene expression with machine learning modelling to identify and characterize the diversification and convergence of gene expression regulation processes during carcinogenesis in space and time. Applicant discovered 1,917 pan-cancer genes commonly deregulated between pairs of healthy and tumor tissue biopsies across 15 cancers. Applicant developed a predictive model, which identified 30 biomarkers and 150 orthologues to predict the carcinogenesis and tumor of origin. Applicant validated models on over 21,000 primary and metastatic human or non-human biopsies from 38 cancers, and achieved a F1-scores up to 99.4% regardless of tissue of origin or cancer stages. Applicant validated the functional evidence of an evolutionary convergence in mammalian carcinogenesis. Our findings could be used for diagnostic tests, monitoring, prognosis, treatment stratification, and improved management of patients with cancer.

Accordingly, in an aspect, there is provided a method of diagnosing cancerous cells in a patient, the method comprising: a) providing a sample containing genetic material from patient cells suspected of being cancerous; b) determining or measuring expression levels in the patient cells of at least 3 of the 1919 genes listed in Table B; c) computing a score using a classifier that takes said expression level values as input, the classifier having been previously trained on known cancerous and non-cancerous samples; wherein the score provides a likelihood of a cancerous cell.

In a further aspect, there is provided a computer-implemented method of diagnosing cancerous cells in a patient, the method comprising: a) receiving, at at least one processor, data reflecting expression levels of at least 3 genes of the 1919 genes listed in Table B in the patient cells; b) constructing, at at least one processor, a patient profile based on the expression levels; c) computing, at the at least one processor, a prediction score using a classifier that takes said expression level values as input, the classifier having been previously trained on known cancerous and non-cancerous samples; wherein the score provides a likelihood of a cancerous cell.

In a further aspect, there is provided a computer program product for use in conjunction with a general-purpose computer having a processor and a memory connected to the processor, the computer program product comprising a computer readable storage medium having a computer mechanism encoded thereon, wherein the computer program mechanism may be loaded into the memory of the computer and cause the computer to carry out the method described herein.

In a further aspect, there is provided a computer readable medium having stored thereon a data structure for storing the computer program product described herein.

In a further aspect, there is provided a device for diagnosing cancerous cells in a patient, the device comprising: at least one processor; and electronic memory in communication with the at least one processor, the electronic memory storing processor-executable code that, when executed at the at least one processor, causes the at least one processor to: a) receive data reflecting expression levels of at least 3 genes of the 1919 genes listed in Table B from the patient cells; and b) compute, at the at least one processor, a prediction score using a classifier that takes said expression level values as input, the classifier having been previously trained on known cancerous and non-cancerous samples; wherein the score provides a likelihood of a cancerous cell.

In a further aspect, there is provided a method of diagnosing cancerous cells in an animal, the method comprising: a) providing a sample containing genetic material from the animal's cells suspected of being cancerous; b) determining or measuring expression levels of at least 3 genes of the 150 genes listed in Table I in the animal cells; c) computing a score using a classifier that takes said expression level values as input, the classifier having been previously trained on known cancerous and non-cancerous samples; wherein the score provides a likelihood of a cancerous cell.

### BRIEF DESCRIPTION OF FIGURES

These and other features of the preferred embodiments of the invention will become more apparent in the following detailed description in which reference is made to the appended drawings wherein:

FIG. 1. Conserved pan-cancer gene expression profiles across human cancer types. (A) Volcano plot reveals the fold change of expression of 1,917 significantly differentially expressed (bonferroni corrected p-value<0.05 and log 2 fold-change >1) genes between tumour and normal biopsies (n=1,434). (B) Decreased expression of H/f in 47 additional match paired samples across 9 cancer types not included in the differential gene expression analysis. (C) Increased expression Fanci in 47 additional match paired samples across 9 cancer types not included in the differential gene expression analysis. (D) Gene Set Enrichment Analysis (GSEA), revealed an enrichment of cancer hallmarks and common druggable targets. (E) Differentially expressed genes involved in cancerous and precancerous conditions, and common druggable targets.

FIG. 2. Pan-cancer gene expression signatures predict the phenotypic status of a biopsy in human. (A) Cross-table of pan-cancer RF-RKFCV diagnostics of tumour and healthy samples in validation cohorts (training cohort n=15,507). RF-RKFCV model with 10 predictor genes. (B) RF-RKFCV model with 30 predictor genes. (C) RF-RKFCV model with 100 predictor genes. (D) Receiver operating curves for the RF-RKFCV 10 model. (E) Receiver operating curves for the RF-RKFCV 30 model.

FIG. 3. Prediction of tumour phenotypic signatures using common pan-cancer gene expression. (A) Statistics on the predictive performance of the Random Forest (RF-RKFCV) models to predict the tumour tissue of origin (TTO), on the independent validation cohorts (n=5,484), after training on 15,507 biopsies. The first model TTO-450 is based on 450 predictor genes and the second model TTO-30 is based on 30 predictor genes. Median estimates across cancers are reported. Cross-table of pan-cancer TTO diagnostics of tumours of origin samples in the independent validation cohorts (n=5,484). The modeling was done training cohort on 15,507 biopsies, using (B) 450 predictor genes (C) 30 predictor genes.

FIG. 4 Dedifferentiation and convergence across mammalian cancers. (A) Mammalian phylogeny. (B) Pan-mammalian prediction of tumour and healthy biopsies. The Random Forest model (RF-RKFCV) was trained on 15,507 human samples with 150 on-to-one orthologues, subset of the common pan-cancer dysregulated genes. Cross-table and statistics on the predictive performance of the Random Forest model are given for three mammalian species, including the Tasmanian Devil (n=48), the mouse (n=24), and dog (n=67). (C) Performance of the pan-mammalian RF-RKFCV model. Statistics on the predictive performance of the Random Forest model are given for three mammalian species with breast cancers

FIG. 5. Gene biotypes distribution for the multidimensional scaling and differential gene expression analyses.

FIG. 6. Schematic design of the gene expression analysis.

FIG. 7. Feature selection for the prediction of the healthy and tumor biopsy status.

FIG. 8. Deconvolution of the predicted status class emitted by the RF-RKFCV with 30 predictor genes.

FIG. 9. Feature selection for the prediction of tumor types.

FIG. 10. Metastatic sample assignation.

FIG. 11 shows a suitable configured computer device, and associated communications networks, devices, software and firmware to provide a platform for enabling one or more embodiments as described herein.

Table A. Samples descriptions by consortium and cancer types.

Table B. Differentially regulated genes between paired healthy and tumor tissue biopsies.

Table C. Recursive feature elimination (RFE) analysis for tumor status and carcinogenesis prediction.

Table D. Comparison of 8 different predictive models for cancer diagnosis with 30 genes.

Table E. 30 biomarkers and their importance in the RF-RKFCV.

Table F. 100 biomarkers and their importance in the RF-RKFCV.

Table G. Random Forest RKFCV model with 30 genes performance on external validation data sets.

Table H. 450 Biomarkers used for the cancer types modelling and their importance in the RF-RKFCV modelling.

Table I. 10 biomarkers and their importance in the RF-RKFCV.

Table J. 150 biomarkers from to 1:1 mammalian orthologous (Human, Mouse, Dog and Tasmanian Devil) and their importance in the RF-RKFCV.

Table K. Most stable genes across cancer and normal tissues.

### DETAILED DESCRIPTION

In the following description, numerous specific details are set forth to provide a thorough understanding of the invention. However, it is understood that the invention may be practiced without these specific details.

Cancers are characterized by extensive genetic and phenotypic variations which represent a critical challenge to the development of reliable diagnostic tools.

We characterised a pan-cancer carcinogenesis transcriptional signature by using a differential gene expression analysis on 1,434 paired healthy and tumor tissues, from 15 cancer types. For carcinogenesis and diagnostic modelisations in space and time, we used machine learning algorithms on RNA sequencing data of over 20,000 biopsies from 38 different cancer types and mammalian tissue. In addition, we performed RNA sequencing of 48 tumoral ovarian tissue samples as an external validation set.

We designed a conceptual and analytical framework for early and follow-up diagnostic tests with the potential to detect cancerous cells of any origin, grade or stage. We identify a common set of 1,917 genes between cancerous and normal tissues. Only 10 genes are sufficient to reliably discriminate cancerous from healthy tissues with F1-score of 98.7%, and of 99.4% when using 30 genes. Our model is robust to differences between cancers, tissues or stages, with F1-scores all above 99% of stages and 100% for 70% of the cancers. Our model also shows conserved molecular carcinogenic programming across mammals. Final, we develop a molecular taxonomic model of cancers based on gene expression profile, by achieving a balanced median accuracy of 97.7% and specificity of 99.9%.

Our study lay robust set classifiers to provide new venues for personalized medicine, with respect to enabling a tailored approach to therapy.

Accordingly, in an aspect, there is provided a method of diagnosing cancerous cells in a patient, the method comprising: a) providing a sample containing genetic material from patient cells suspected of being cancerous; b) determining or measuring expression levels in the patient cells of at least 3 of the 1919 genes listed in Table B; c) computing a score using a classifier that takes said expression level values as input, the classifier having been previously trained on known cancerous and non-cancerous samples; wherein the score provides a likelihood of a cancerous cell.

The term “level of expression” or “expression level” as used herein refers to a measurable level of expression of the products of biomarkers, such as, without limitation, the level of messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

As used herein, the term “control” refers to a specific value or dataset that can be used to prognose or classify the value e.g. expression level or reference expression profile obtained from the test sample associated with an outcome class. A person skilled in the art will appreciate that the comparison between the expression of the biomarkers in the test sample and the expression of the biomarkers in the control will depend on the control used.

The term “differentially expressed” or “differential expression” as used herein refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example as measured by the amount of messenger RNA transcript and/or the amount of protein in a sample as compared with the measurable expression level of a given biomarker in a control.

The term “subject” as used herein refers to any member of the animal kingdom, preferably a human being and most preferably a human being that has, has had, or is suspected of having cancer.

The term “sample” as used herein refers to any fluid, cell or tissue sample from a subject that can be assayed for biomarker expression products and/or a reference expression profile, e.g. genes differentially expressed in subjects.

In some embodiments, the at least 3 genes are genes found in at least one of Tables E, F, and I. Preferably, the at least 3 genes are genes found in at least two of Tables E, F, and I. More preferably, the at least 3 genes are genes found in all of Tables E, F, and I.

In some embodiments, the at least 3 genes is at least 10 genes.

In some embodiments, the at least 3 genes is at least 30 genes.

In some embodiments, the at least 3 genes is at least 100 genes.

In some embodiments, the at least 3 genes is at least 20, 40, 50, 60, 70, 80, 90, 150, 250, 300, 350, 400, 450, 500 or 1800 genes.

In one preferable embodiment, the at least 3 genes are the 10 genes in Table I. Further preferably, the at least 3 genes consists of the 10 genes in Table I.

In one preferable embodiment, the at least 3 genes are the 30 genes in Table E. Further preferably, the at least 3 genes consists of 30 the genes in Table E.

In one preferable embodiment, the at least 3 genes are the 100 genes in Table F. Further preferably, the at least 3 genes consists of the 100 genes in Table F.

In some embodiments, the method further comprises determining the tissue of origin of the patient cell by: d) determining or measuring expression levels in the patient cells of at least 3 genes of the 450 genes listed in Table H; e) computing a score using a classifier that takes said expression level values as input, the classifier having been previously trained on known cancerous and non-cancerous samples from known tissues of origin; wherein the score provides a likelihood of the patient cell's tissue of origin. Preferably, the at least 3 genes are the genes with the highest VarImp

In some embodiments, the at least 3 genes is at least 20, 30, 40, 50, 60, 70, 80, 90, 100, 150, 200, 250, 300, 350, 400, 450, 500 or 1800 genes.

In some embodiments, the cancer is selected from the cancers identified in Table A.

In some embodiments, if there is a low likelihood of cancer, further comprising managing the patient with active surveillance. Or, if there is a high likelihood of cancer, further comprising treating the patient with surgery, endocrine therapy, chemotherapy, radiotherapy, hormone therapy, gene therapy, thermal therapy, or ultrasound therapy.

The term “low risk” or “low likelihood” as used herein in respect of cancer refers to a statistically significant lower risk of cancer as compared to a general or control population. Correspondingly, “high risk” or “high likelihood” as used herein in respect of cancer refers to a statistically significant higher risk of cancer as compared to a general or control population.

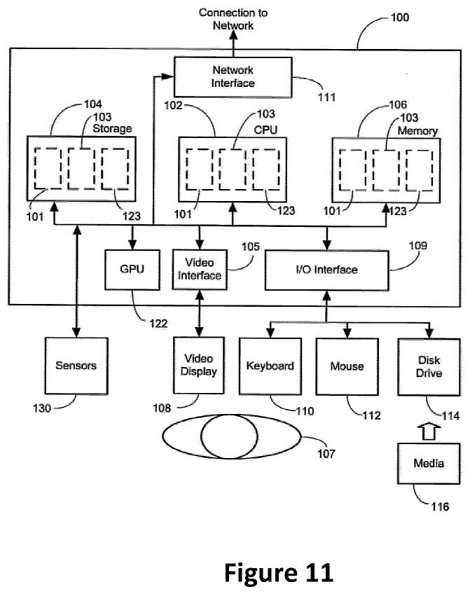

The present system and method may be practiced in various embodiments. A suitably configured computer device, and associated communications networks, devices, software and firmware may provide a platform for enabling one or more embodiments as described above. By way of example, FIG. 11 shows a generic computer device 100 that may include a central processing unit (“CPU”) 102 connected to a storage unit 104 and to a random access memory 106. The CPU 102 may process an operating system 101, application program 103, and data 123. The operating system 101, application program 103, and data 123 may be stored in storage unit 104 and loaded into memory 106, as may be required. Computer device 100 may further include a graphics processing unit (GPU) 122 which is operatively connected to CPU 102 and to memory 106 to offload intensive image processing calculations from CPU 102 and run these calculations in parallel with CPU 102. An operator 107 may interact with the computer device 100 using a video display 108 connected by a video interface 105, and various input/output devices such as a keyboard 115, mouse 112, and disk drive or solid state drive 114 connected by an I/O interface 109. In known manner, the mouse 112 may be configured to control movement of a cursor in the video display 108, and to operate various graphical user interface (GUI) controls appearing in the video display 108 with a mouse button. The disk drive or solid state drive 114 may be configured to accept computer readable media 116. The computer device 100 may form part of a network via a network interface 111, allowing the computer device 100 to communicate with other suitably configured data processing systems (not shown). One or more different types of sensors 135 may be used to receive input from various sources.

The present system and method may be practiced on virtually any manner of computer device including a desktop computer, laptop computer, tablet computer or wireless handheld. The present system and method may also be implemented as a computer-readable/useable medium that includes computer program code to enable one or more computer devices to implement each of the various process steps in a method in accordance with the present invention. In case of more than computer devices performing the entire operation, the computer devices are networked to distribute the various steps of the operation. It is understood that the terms computer-readable medium or computer useable medium comprises one or more of any type of physical embodiment of the program code. In particular, the computer-readable/useable medium can comprise program code embodied on one or more portable storage articles of manufacture (e.g. an optical disc, a magnetic disk, a tape, etc.), on one or more data storage portioned of a computing device, such as memory associated with a computer and/or a storage system.

In aspect, there is provided a computer-implemented method of diagnosing cancerous cells in a patient, the method comprising: a) receiving, at at least one processor, data reflecting expression levels of at least 3 genes of the 1919 genes listed in Table B in the patient cells; b) constructing, at at least one processor, a patient profile based on the expression levels; c) computing, at the at least one processor, a prediction score using a classifier that takes said expression level values as input, the classifier having been previously trained on known cancerous and non-cancerous samples; wherein the score provides a likelihood of a cancerous cell.

In aspect, there is provided a computer program product for use in conjunction with a general-purpose computer having a processor and a memory connected to the processor, the computer program product comprising a computer readable storage medium having a computer mechanism encoded thereon, wherein the computer program mechanism may be loaded into the memory of the computer and cause the computer to carry out the method described herein.

In aspect, there is provided a computer readable medium having stored thereon a data structure for storing the computer program product described herein.

In aspect, there is provided a device for diagnosing cancerous cells in a patient, the device comprising: at least one processor; and electronic memory in communication with the at least one processor, the electronic memory storing processor-executable code that, when executed at the at least one processor, causes the at least one processor to: a) receive data reflecting expression levels of at least 3 genes of the 1919 genes listed in Table B from the patient cells; and b) compute, at the at least one processor, a prediction score using a classifier that takes said expression level values as input, the classifier having been previously trained on known cancerous and non-cancerous samples; wherein the score provides a likelihood of a cancerous cell.

In aspect, there is provided a method of diagnosing cancerous cells in an animal, the method comprising: a) providing a sample containing genetic material from the animal's cells suspected of being cancerous; b) determining or measuring expression levels of at least 3 genes of the 150 genes listed in Table I in the animal cells; c) computing a score using a classifier that takes said expression level values as input, the classifier having been previously trained on known cancerous and non-cancerous samples; wherein the score provides a likelihood of a cancerous cell.

The advantages of the present invention are further illustrated by the following examples. The examples and their particular details set forth herein are presented for illustration only and should not be construed as a limitation on the claims of the present invention.

### Examples

### Methods and Materials

### I. RNA-Seq Data

I.1. Differential gene expression analysis: In total, we analyzed 1434 paired non-overlapping RNA seq samples from PCAWG (n=) and from TCGA (n=), from 15 cancer types. PCAWG RNA-seq data (SYNAPSE ID) was aligned with the human reference genome (GRCh37.p13) using the read aligner STAR (version 2.4.0i, 2-pass). Gencode (release 19) was used for the reference annotation (36). We processed non-overlapping healthy and tumor RNA-seq samples from TCGA with the same pipeline as for PCAWG.

I.2. Modeling analysis, we used the PCAWG data set (n=490 from 1,188, Freeze v1.4), TCGA (n=11,284) and GTEX (n=9,217, Release: V6p) for a total of 20,991 non-overlapping samples. We used reprocessed data by recount projects and accessible through the recount2 database for consistency to reduce batch effect due to different RNA sequencing pipelines in the TCGA and GTEx. All raw sequencing data were processed with Rail-RNA as described in Nellore et al. (40). The results used here are in whole or part based upon data generated by the TCGA Research Network: www.cancer.gov/tcga.

I.3 To test for a pan-cancer gene expression signature conserved across mammals, we downloaded mouse (Mus musculus) cancer models, p53−/− murine lung cancer model and WT control (GSE59831) as well as RNA-seq from 24 mammary gland samples of MMTV-PyMT mouse models (GSE76772) and healthy mouse tissue (GSE76772). Reads were aligned on the mm9 genome, and raw gene counts were computed with HTseq 0.6.1p1 (37).

I.4. External validation: We gathered RNA-seq datasets that have been deposited in GEO and reanalyzed by the recount2 database as well as from the expression atlas and array express, for a total of 34 external data set encompassing 32 normal and 675 cancer cells and breast, lung, liver and ovarian cancer tissues. We included cancer subtypes not found in the training set to test for robustness. In addition, we sequenced the transcriptome of 48 ovarian cancer biopsies, obtained from the Ontario Tumor Bank (ethics approval #35033, issued by the University of Toronto, the office of the vice-president, research and innovation). Total RNA was extracted from flash frozen tissue with RNeasy Mini Kit (Qiagen #74104) to a concentration of 250 ng and a Targeted RIN of 7 or above. Libraries were constructed with the NEBNext® Ultra™ II Directional RNA kit with a ribosomal RNA depletion step, according to the manufacturer's protocol. Samples were sequenced on an Illumina HiSeq 2500 platform with the sequencing kit HiSeq SBS Kit V4 (250 bp, 250 cycles) at a sequencing depth of 100 million reads. Quality control on the sequenced reads was done using FastQCr, and adaptors were trimmed down using TrimGalore (v.0.4.5). Reads were aligned on the human genome assembly GRCH38 with STAR (v.2.4.2). The outputted BAM files were cleaned according to the Broad's Best Practice pipeline for RNA-Seq data. Raw counts were computed with both HTSeq (37) and Salmon (ref).

### II. Differential Gene Expression Analyses

Pan-cancer analysis of paired healthy and tumor tissue biopsies: We selected 1,434 paired healthy and tumor samples from TCGA and PCAWG representing 15 different cancers types, each represented by at least 19 paired biopsies. Each paired healthy and primary tumor biopsy was sampled from the same tissue. This design increases the robustness of our analysis by controlling for potential confounding factors like genetic background and environment as well as various batch effects (eg. age, sex). We selected genes having at least one count per million (CPM) in at least 90% of samples, resulting in a set of 20,614 genes in order to remove lowly expressed genes that contributed to increase the signal-to-noise ratio across samples. To control for batch effects, we computed two surrogate variables, with the sva R package and the svaseq( ) function while controlling for gender, cancer types and donors (38). The surrogate variables were included in the DGE analysis along with other covariates: gender, cancer type and donor ids (for repeated measures). We set the design for the generalized linear model with donor ids being nested with cancer types to control for repetitive measures as described below:

$$model : {Y}_{ij} ∼ {β}_{j} SV {1}_{i} + {β}_{j} SV {2}_{i} + {β}_{j} {G}_{i} + {β}_{j} {C}_{i} + {β}_{j} {CD}_{i} + {β}_{j} {S}_{i} + {ϵ}_{ij} ,$$

Where SV1 and SV2 represent the two surrogate variables, the gender G, the cancer type C, the donor id D and the status of the biopsy S for the i^{th} biopsy and j^{th} gene and E represents the residual error. To avoid imbalance in our design that could drive the gene expression signal toward a cancer with most individuals, as we are interested in an overall status effect (healthy vs. tumor), we used a strategy of resampling without replacement 10 paired healthy and tumor biopsies from 15 different cancers. This resampling strategy was performed 1,000 times with the same model described above. We performed the DGE analyses with DESeq2, with a generalized linear model and a negative binomial distribution and computed the Wald statistic and coefficients. A Bonferroni correction was applied to the estimated p-values, a distribution was then built to select the top genes with a median Bonferroni value below 0.05 and log 2 fold change above 1.

### III. Gene Annotation and Pathway Enrichment

Pathway enrichment analysis following differential gene expression analysis was done with ReactomeFi and cytoscape with the genes ranked by median Bonferroni corrected p-values. We carried out a second pathway enrichment analysis using the g:Profiler R package (39) with the following settings: ranked input gene list, only GO biological processes and Reactome pathways considered, with a minimum of five and a maximum of 1000 genes per gene set. We set the g:Profiler multiple internal testing correction to FDR estimates, with a minimum of three genes shared with gene list and gene set, and electronic gene annotations (IEA) included.

### IV. Machine Learning Analyses

### IV.1. Pan-Cancer Signature for the Status Prediction of any Given Biopsies

We built a data set from the PCAWG, TCGA and GTEx RNA-seq data, representing 20991 unique biopsies. This dataset includes 38 different cancer types and is divided into 396 metastatic, 9941 healthy tissue, 10581 primary tumor, 11 additional primary, and 62 recurrent biopsies (Table A). This data set comprises tumor biopsies (liquid or solid) from stage 0 to stage four, with a median cellularity of 80% ranging from 0% to 100%. We divided the data set into a training set, representing 70% of the data, and a testing set (30% of the data). We took care that at least 10 healthy and tumor biopsies are represented in the testing set for robust predictive evaluation. Metastatic, additional primary and recurrent biopsies were excluded from the training set. Raw counts provided by PCAWG or the recount2 databases were used as input.

Selection of the predictors: We selected the top predictors from our set of top 1,000 genes differentially expressed among cancers, using a recursive feature selection with a random forest machine-learning classifier algorithm. The feature selection and training were only done on the training set. We trained a random forest algorithm to test for the best combination and number of features to predict the status of a biopsy (tumor or healthy). The classifier was trained with a repeated cross validation of 10 folds repeated 10 times. We tested independently for 10, 20, 30, 40, 50, 60, 70, 80, 90, 100, 150, 200, 250, 300, 350, 400, 450, 500 and 1000 genes as best predictors of the status.

Comparison of different machine learning models for algorithm selection: We compared 13 different models for cancer status prediction using 30 best predictive features as selected by the RFE algorithm: All models were trained with the same training set, and were set with the same seeds and the same repeated leave group out cross-validation parameters (K=10 with 10 repeats and 70% of training set used as training). We tested the following models: Bootstrap Aggregation of a CART algorithm (bagCART), Classification and Regression Tree (CART), deep boosting (deepboost), Gradient Boosting Machines (GBM), K-Nearest Neighbour (KNN), Linear Discriminant Analysis (LDA), Random Forest (RF), Support Vector Machine (SVM). For the comparison only, we took the basic parameters of each algorithm. We tested for the imbalance in number of samples within each category (healthy or tumor) during the sampling process and scaling parameters prior computation. As they did not impact the predictive outcome (data not shown) they are not used subsequently.

Final modeling: To test for the robustness of our model, we first used a K-fold repeated cross validation strategy with K=10 and 10 repeats of 70% of the dataset. Predictions were assessed only once in the testing set (30% of the data). We assessed model performance of our two classes classifier with a receiver operating characteristic (ROC) curve, Matthews's correlation coefficient, accuracy, specificity and sensitivity. For independent evaluation, we downloaded from the recount2 database additional independent RNA-seq datasets representative of the 38 cancer types as well as cancer types that have not been trained (Table A) to test for the robustness of our set of pan-cancer biomarkers for rare or unknown cancer diagnostics. Early detection, tumor heterogeneity and cancer types: We investigated the algorithm performances for early cancer detection and tumor heterogeneity and cancer types by grouping the classification output into a stage/grade, cellularity and cancer types categories.

Effect of ischemia time on status prediction: We tested if ischemia time impacted the predictive performances, thus affecting the generalization of our models, as it is known to impact the gene expression (35). We removed brain tissue samples having enzymatic degradation from the training set, as this tissue had the longest ischemia time, and samples with more than four hours of total ischemia time. We kept only blood samples prior two hours from death. These selective criteria led to a selection of 915 out of 9,115 samples from the GTEx, for a final training set of 3,306 samples (1653 tumor and healthy). We trained a new model using a RF-RKFCV algorithm with 30 predictor genes selected as previously described. On the independent validation set, the model reached a performance within 5% of the model including degraded tissue in the training set, with F1-score of 98.08% vs. 99.36%, recall of 98.18% vs. 99.4% and the precision of 97.98% vs. 99.33%. This demonstrates the robustness of our model to identify normal tissue and may be a valuable tool for non-optimal sampling and processing protocol where tissue may degrade.

### IV.2. Pan-Mammalian Transcriptional Signature of Cancer

From our set of best predictors of the biopsy status, we selected only single-family orthologous genes between human and mouse (Mus Musculus). This represents a set of 150 orthologous genes. We used the same strategy as for our final modeling with a random forest algorithm and train the model only on human RNA-seq data (n=14,693) for complete independence and test if the signature is conserved by the modeling and the classification of any mammalian biopsies.

### IV.3. Transcriptional Signature for Cancer of Origin Profiling

For this specific question, we used the exact same dataset (n=20,991) describe in the “Pan-cancer signature of the status of any given biopsies” section. For this question we still included non-tumor biopsies for safety checks and ensure that still the signature of healthy and any tumor types biopsies are different. We search for the best feature predictor with RFE and tested the same machine learning models as described before, with the same strategy. In the modeling we test if the transcriptional signature is solely able to classify 38 different types of cancers accurately and a non-cancer category.

### Results and Discussion

### Tumors Exhibit a Heterogeneous Transcriptional Signature

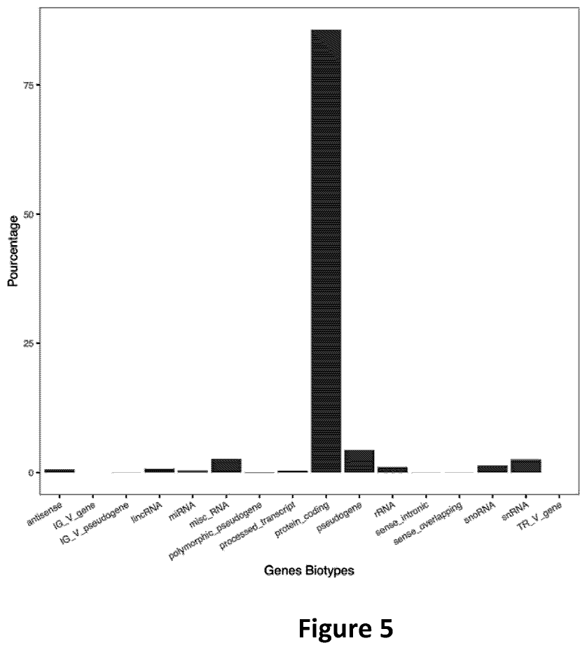

Transcriptional signatures can result from a combination of genetic variation across individuals, tissular gene expression, environmental exposure, tumor microenvironment, evolutionary processes and developmental plasticity (17, 19, 29). As expected, we observe a tissue-specific transcriptional signature in healthy tissue adjacent to tumor samples (FIG. 5 and data not shown). However, the transcriptomes of tumor samples show more heterogeneity and do not distinguish the tissue of origin as well as for the matching healthy samples, as observed by the reduced amount of variance (40.64% vs. 27.78%) and greater overall distance within tissue (data not shown). This result is concordant with previous observations of transcriptomic regulatory convergence in cancers (3, 19).

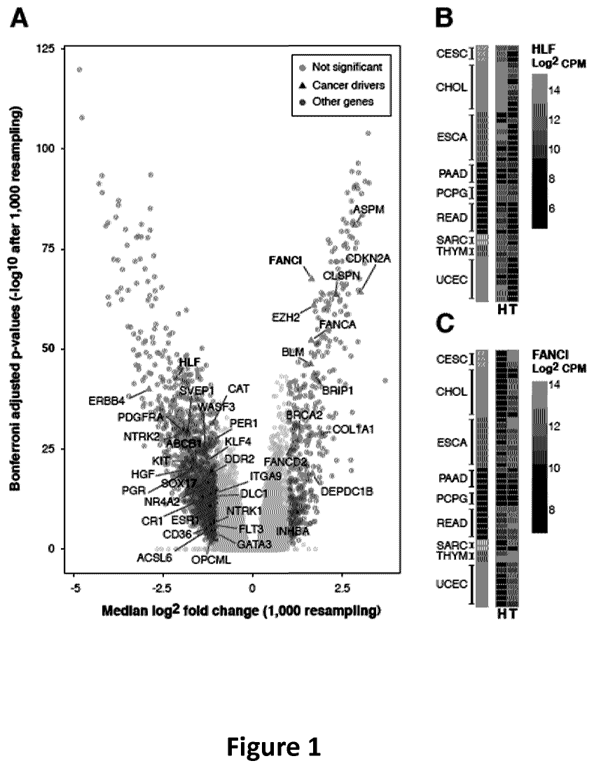

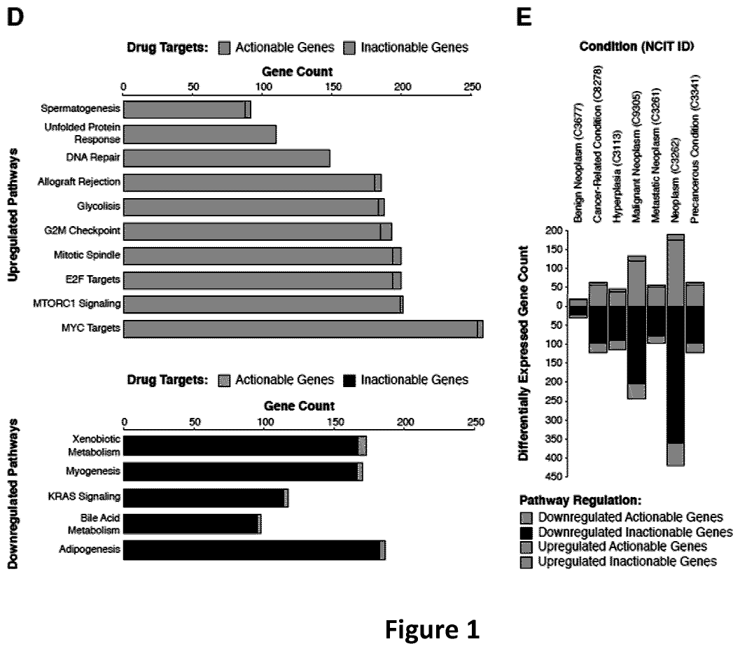

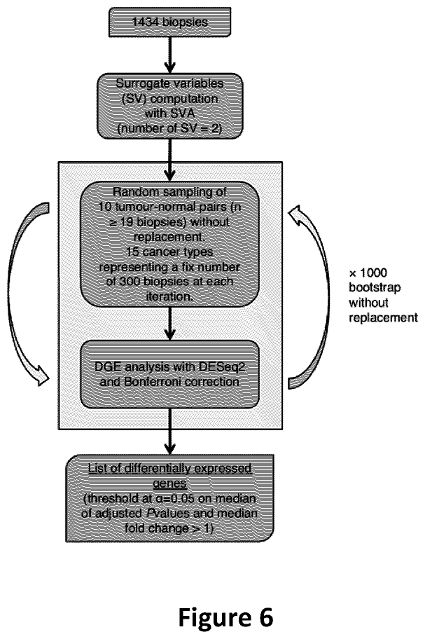

We therefore tested if a convergent cancer transcriptomic signature could be modeled from the increasingly heterogeneous transcriptome from tumoral tissue compared to healthy tissues (data not shown). We identified differentially expressed genes from TCGA and PCAWG RNA sequencing (RNA-seq) expression data between paired healthy and tumor tissue biopsies (n=1,434) from 15 different cancers types originating from 11 tissues having at least 19 donors (Table A and data not shown). We controlled for the imbalance in the design across cancers and genetic background with a bootstrapping strategy (FIG. 6). We identified a pan-cancer gene set of 1,919 differentially expressed genes between tumor and healthy tissue (FIG. 1A, Table B, and data not shown). Notably, the upregulation of DNA damage response repair genes (FIG. 1B-D) and pathways supports an increased genomic instability as a result of an elevated DNA replication rate and mRNA production (20, 21). The pan-cancer gene expression signature captures some of the major hallmarks of cancer biology functions, including cell cycle and division, DNA repair, as well as other signaling and recombination pathways or processes (FIG. 1B-D). These genes are also significantly targeted by 7 transcription factors: TWIST1, RSRFC4, MZF-1, KLF, GEMIN3, GKLF, BRN1 and a micro RNA has-miR-335-5p (corrected p-value<0.01), important in many cellular processes associated with cancer development. Our pan-cancer gene expression signature captures molecular information of cancer biology and its microenvironment (FIG. 1C-D), as well as tissular and a tumoral specificity, which can be used to model the pathological tumoral state and the origin of the biopsy (FIG. 1E).

### Pan-Cancer Transcriptomic Signature Predicts Tumoral Cell State

We then validate the discovery of a pan-cancer carcinogenic gene expression signature from 15 matched tumors and healthy tissues, using machine learning algorithms modeling on 20,991 biopsies.

We trained models on the raw count data normalized using seven constitutively expressed orthologous genes from 15,507 biopsies including only primary sites from 38 cancers gathered from TCGA, ICGC and PCAWG, and normal tissue samples from the GTEx (Table A). Paired biopsies used in the DGE analysis were included only in the training set to keep discovery and validation sets independent. Model selection was based on a random forest (RF), by tuning parameters for the number of trees and the number of features to grow the trees. To minimize overfitting, we used 10 folds cross validation (CV) on the training set (n=15,507, TCGA, ICGC and PCAWG) to optimize the choice of tuning parameters for classification with a training weight of 50/50 for each class. The 10-fold CV was repeated 10 times and tuning parameters were specified based on the average across the repeats. The final model accuracy is taken as the mean of the number of repeats. Further, we validated our final model with both an independent validation set of 5,484 biopsies (TCGA, ICGC and PCAWG) and an external validation set of 1,546 biopsies from the Gene Omnibus (GEO) and European Bioinformatics Institute (EBI).

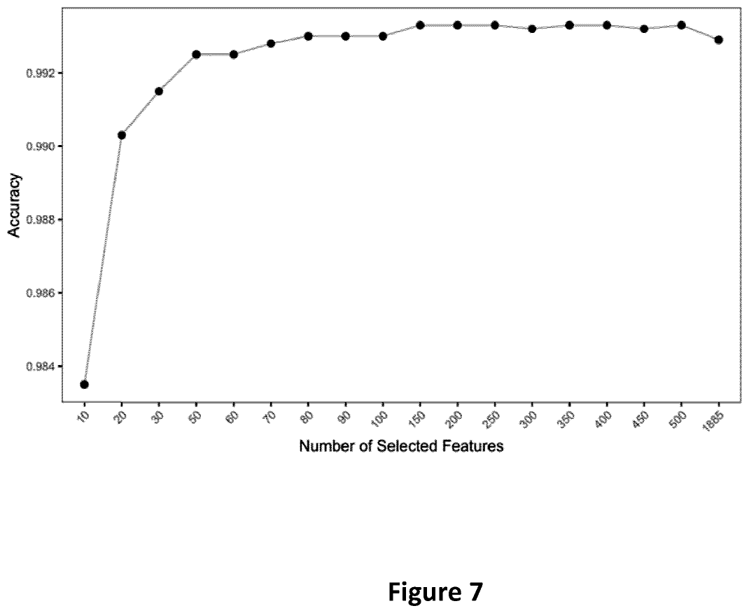

We selected the best performing predictor genes for higher generalizability by having the minimum variance in predictive performance during the internal validation process. We performed a Recursive Feature Elimination (RFE) based on a random forest classifier with both a repeated K-fold cross-validation (RKFCV) and a Boruta algorithm to iteratively remove genes less relevant than random probes (22). We kept the most parsimonious number of predictors provided by either algorithm. Only 10 genes are required for an internal validation accuracy performance above 98%, while 30 genes represent the best trade-off between performance and variance and 100 genes give the best performances for the classification of healthy and tumor biopsies (FIG. 7, Table C, D). These low numbers of biomarkers are suitable for gene panels in a clinical setting. Among the eight algorithms tested, the random forest, gradient boosting machines, and deepboost had the best validation performances with Area Under the Receiver Operating Characteristic curve above 0.99 (Table D).

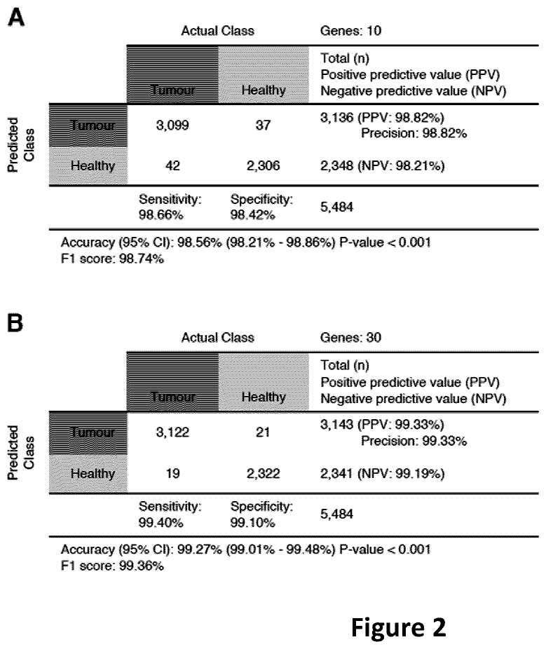

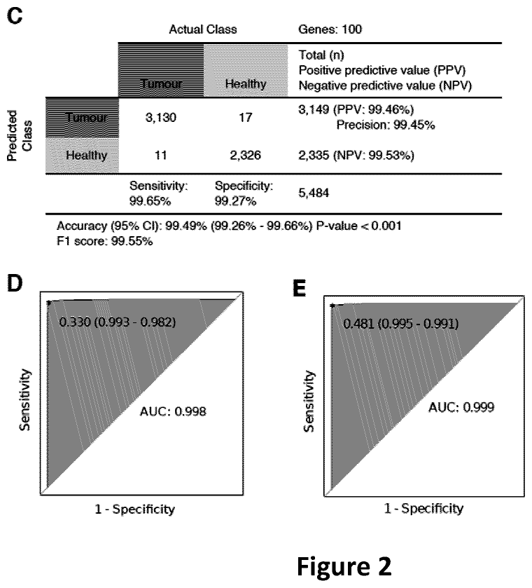

We trained a RF-RKFCV algorithm, one of the top performing algorithms. After fine-tuning the RF-RKFCV algorithm, the performance on an independent validation set (n=5,484), was high and very stable, regarding the number of genes we selected. We obtained F1-scores of 98.74%, 99.36% and 99.55% (FIG. 2 A-C) respectively for 10, 30 and 100 predictor genes (Table I, E, F), with 99.40% sensitivity and 99.33% precision using 30 genes (FIG. 2B). We achieved up to 100% F1-score, recall and precision on an external validation set (Table G), confirming the performance of our model when tested on different sequencing platforms, batch, and tissue preservation.

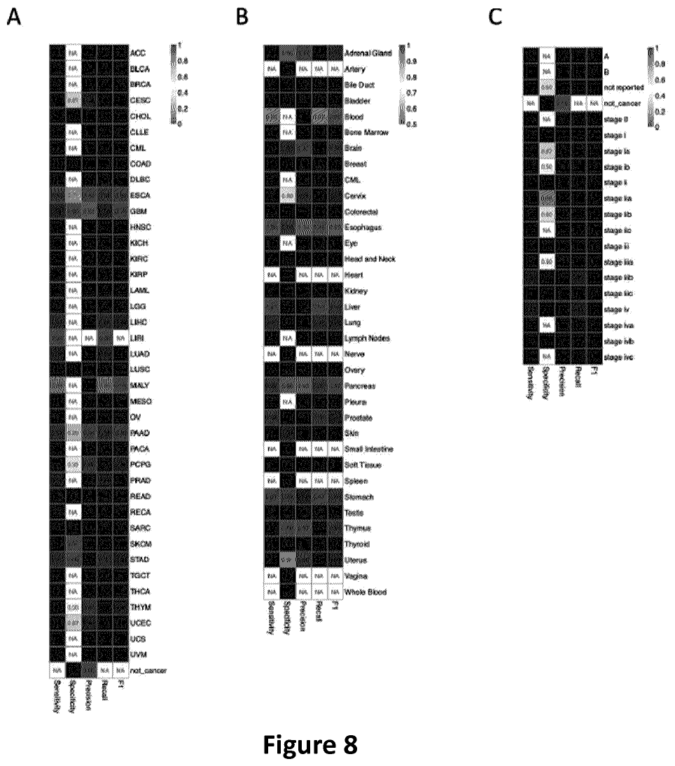

We then investigated the performance of the RF-RKFCV algorithm modeling the carcinogenic state of biopsies across cancers, tissues and stages in the independent validation set. The model was robust at classifying tumor and normal biopsies, achieving a F1-score of 100% for 26 out of 38 cancer types (FIG. 8A) and F1-scores above 95% among the 26 tissue types for which tumor biopsies were available (FIG. 8B). Also, the model achieved a performance of up to 100% F1-score for the ovarian tissue, with the lowest scores observed in the liver tissue with 90% (FIG. 8B). The algorithm correctly classified biopsies for both early and late-stage cancers with a F1-score, recall and precision were all above 99% (FIG. 8C).

### Conservation of a Carcinogenesis Signature in Eutherian.

We investigated other mammalian cancer types to further test for consistency, of our model, and for conservation and convergence in carcinogenesis in mammals (FIG. 5A). We investigated the transcriptomes from 24 mouse breast biopsies, ranging from hyperplasia to late carcinoma (23). We developed a model based on single gene orthologous families. Using a recursive feature elimination strategy on 1,167 genes, we discovered a set of best 150 orthologous gene predictors and trained a RF-KFCV model on our human training set (FIG. 5B)(Table J). We accurately classify early and late stage tumors as well as healthy mammary gland biopsies with a predictive recall of 100%, precision of 66.67% and F1-Score of 80% for mouse (FIG. 5C). This model is able to predict the tumoral state, of human, with highly predictive scores, with a recall of 99.42%, precision of 99.58% and F1-Score of 99.50%. Our model was highly predictive of the carcinogenesis state of non-human mammals when trained exclusively on human cancers biopsies. This result gives evidence of an evolutionary convergence of mammalian tumor cells through the rewiring of the same targeted pathways.

### Pan-Cancer Gene Expression Signature Predicts the Tumor Primary Site

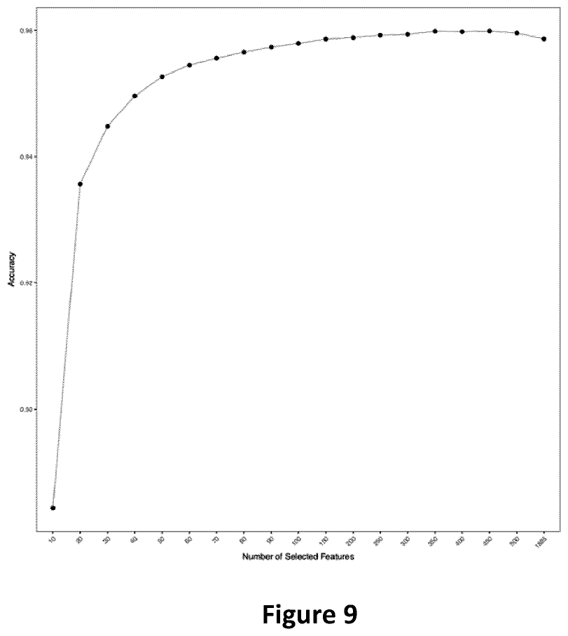

We examined if the pan-cancer carcinogenesis gene expression signature is efficient for the modeling of cancer-specific transcriptional signatures. We compare the performance of a model trained with 30 putative biomarkers identified during the carcinogenic signature, and one trained with 450 genes predicting best transcriptional signature associated with the tumor of origin, for 40 different classes, using the same approach to control for overfitting and parameters as described earlier (FIG. 9). Validation sets consisted of one split into primary tumors and normal tissues, and one containing only metastatic biopsies.

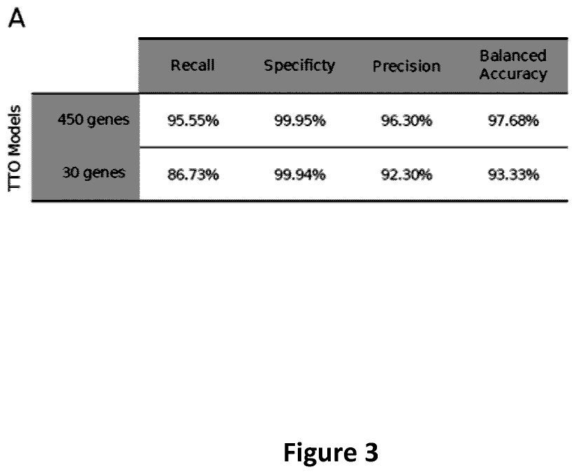

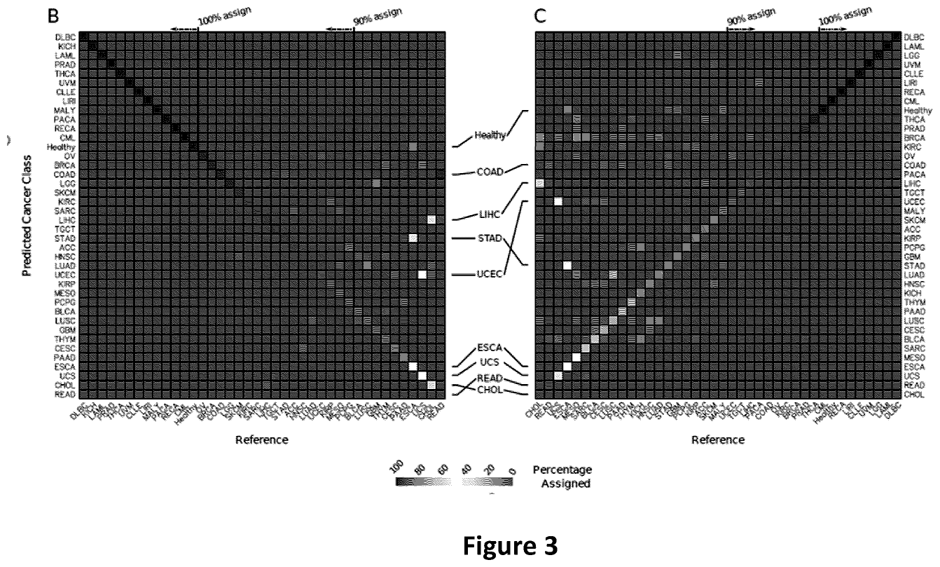

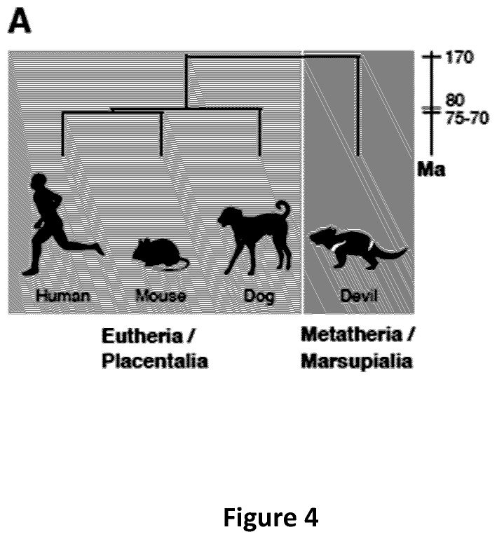

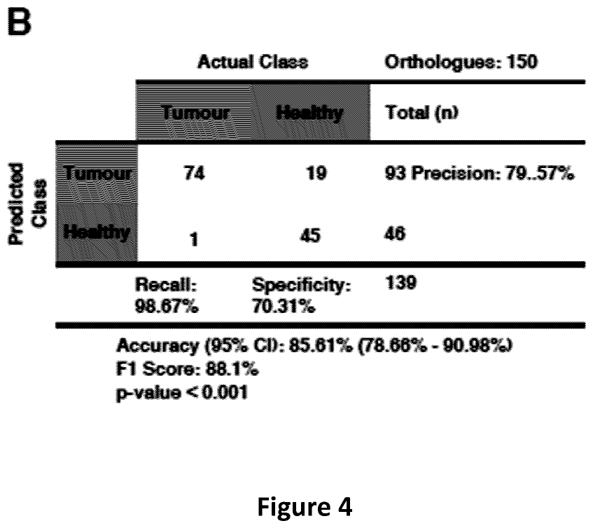

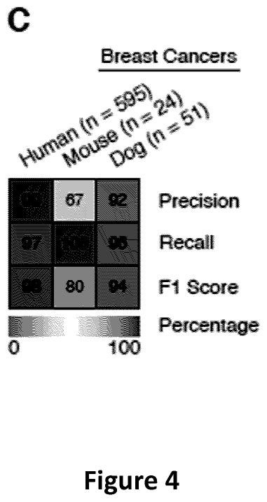

The model using 450 genes (RF450) had a balanced accuracy, controlling for sample size, of 97.68% and very high degree of specificity of 99.95% (FIG. 3A, Table H). The model using 30 genes (RF30) had the same specificity, with a balanced accuracy of 93.77%. The RF450 model classified 31 classes with 90% of validation samples correctly assigned, with 11 classes having 100% assignation success (FIG. 3B). The RF30 model achieved similar result, where 100% of samples were accurately predicted in nine classes, including two controls: a myeloid cell line (CML) and a normal tissue class (NOS) (FIG. 3C). Models had good performances but we suspect that the modeling of the molecular profiles of some cancers may be indiscernible with the number of predictors. Thirty-six percent of the esophageal squamous carcinoma (ESCA), 47% of uterine carcinosarcoma (UCS, complex mixed and stromal neoplasms), 56% of Cholangiocarcinoma (CHOL), and 98% of rectum adenocarcinomas (READ), were respectively classified with the RF450 model as stomach adenocarcinoma (STAD), uterine corpus endometrial carcinoma (UCEC), Liver Hepatocellular Carcinoma (LIHC), or colon adenocarcinomas (COAD). We speculate that the molecular profiles of the two major subtypes of READ: adenocarcinomas (n=150) and mucinous (n=15) are very similar to the COAD. We suspect very close ontological signature between the two uterine carcinomas and classification as well as in the case of STAD and ESCA and CHOL and LIHC. In any case the tissue proximity is respected during classification.

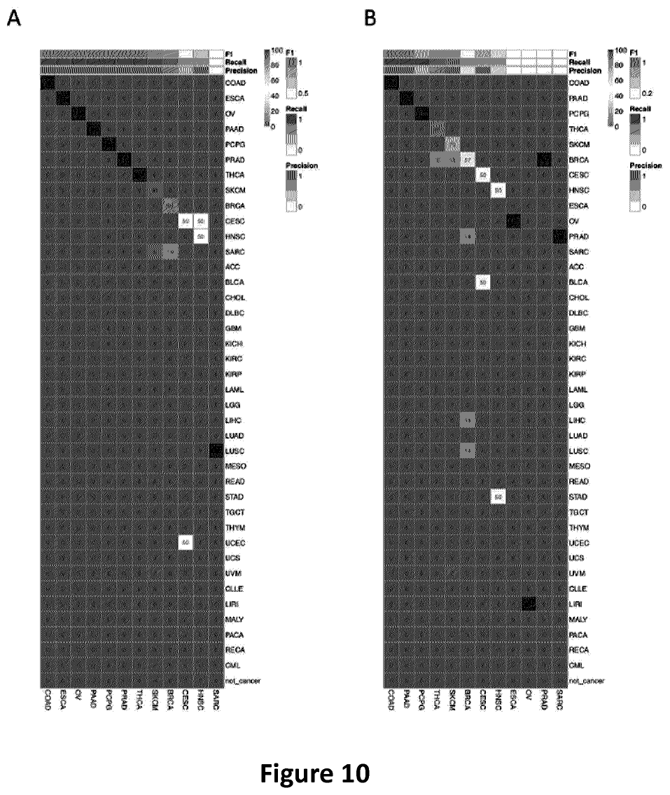

As tumor progress and become more aggressive or metastatic, they acquire novel functions associated with a shift in their gene expression profile to accommodate for example the epithelial to mesenchymal transition, which could affect the classification performances, along with sampling bias (24, 25). We could classify seven and three metastasis tumors to their primary location with 100% recall with the RF450 and RF30 models, respectively (FIG. 10A,B). Interestingly, we had better success than another classifier using that used somatic mutations on the same data set for the metastatic thyroid adenocarcinoma, where they could not correctly identify any of the 13 metastatic samples (26).

We could not rule out the location sampling bias of the biopsy from the transcriptional signature convergence. However, it has been demonstrated that some of the developmental paradigms that govern embryonic development could govern tumor cell phenotypic induction; whereupon the absence of a lineage-specific transcription factor NKX2-1 in non-small cell lung cancer lead to various gut like tissue phenotype (17). Also, the combination of molecular biomarkers signature lead to an estimated reclassification of one out of ten cancer patients (3, 27). Thus, transcriptional signature could be interpreted in terms of a molecular taxonomy, affecting therapeutics strategy. Importantly, the performance of the models for each cancer type is independent of the cancer type used for the DGE analysis, demonstrating that a pan-cancer expression profile is a valid tool for tumor diagnostic of any origin.

We designed a conceptual and analytical framework for the discovery of early and follow-up biomarkers with the potential to detect cancerous cells of any origin, grade or stage. Using transcriptome quantification with a paired design to control for environmental and genetic factors, we uncover a pan-cancer gene set associated with carcinogenesis. Despite the tumoral transcriptomic heterogeneity, we accurately modeled the gene expression signature associated with carcinogenesis in mammals, confirming an evolutionary convergence of cancerous cells towards a common physio-pathological phenotype (30). Furthermore, we accurately model the origin of cancer from the same pan-cancer gene-set. From our knowledge, our classifier is among the most accurate (31-33). Our study lay novel proof of concepts and robust classifier sets providing new venues for personalized medicine.

Further investigations are required to determine whether the transcriptional status of the genes investigated is acquired before diagnostic i.e. stage I or 0. We have confirmed that the expression signature is stable between stage timing, between primary and metastatic tumor location and between patients. Interestingly, the stability of this carcinogenesis transcriptional signature between early and late stage carcinomas suggests that the chromatin landscape is established early in ontogeny of the diseases, and would be a suitable biomarker for both diagnostic and pre-diagnostic early cancer ontogenetic stages (34). We acknowledge that the training with GTEx samples is suboptimal due to enzymatic degradation (35). However, when accounting for the ischemic time and enzymatic degradation as selection criteria for the training set samples, the classification performances on an external testing set were not affected. To establish the utility in clinical settings of our classifiers, large prospective studies of all incident cancer types will be required.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. S. V. Puram, I. Tirosh, A. S. Parikh, A. P. Patel, K. Yizhak, S. Gillespie, C. Rodman, C. L. Luo, E. A. Mroz, K. S. Emerick, D. G. Deschler, M. A. Varvares, R. Mylvaganam, O. Rozenblatt-Rosen, J. W. Rocco, W. C. Faquin, D. T. Lin, A. Regev, B. E. Bernstein, Single-Cell Transcriptomic Analysis of Primary and Metastatic Tumor Ecosystems in Head and Neck Cancer, Cell 171, 1611-1624.e24 (2017).
2. J. Liu, et al., An Integrated TCGA Pan-Cancer Clinical Data Resource to Drive High-Quality Survival Outcome Analytics, Cell 173, 400-416.e11 (2018).
3. K. A. Hoadley, et al., Cell-of-Origin Patterns Dominate the Molecular Classification of 10,000 Tumors from 33 Types of Cancer, Cell 173, 291-304.e6 (2018).
4. N. McGranahan, C. Swanton, Clonal Heterogeneity and Tumor Evolution: Past, Present, and the Future, Cell 168, 613-628 (2017).
5. D. L. Roden, L. A. Baker, B. Elsworth, C.-L. Chan, K. Harvey, N. Deng, S. Wu, A. Cazet, R. Nair, A. Swarbrick, Single cell transcriptomics reveals molecular subtype and functional heterogeneity in models of breast cancer, Biorxiv, 282079 (2018).
6. R. Majeti, M. W. Becker, Q. Tian, T.-L. Lee, X. Yan, R. Liu, J.-H. Chiang, L. Hood, M. F. Clarke, I. L. Weissman, Dysregulated gene expression networks in human acute myelogenous leukemia stem cells, Proceedings of the National Academy of Sciences 106, 3396-3401 (2009).
7. J. L. Carstens, P. de Sampaio, D. Yang, S. Barua, H. Wang, A. Rao, J. P. Allison, V. S. LeBleu, R. Kalluri, Spatial computation of intratumoral T cells correlates with survival of patients with pancreatic cancer, Nat Commun 8, ncomms15095 (2017).
8. S. F. Roerink, N. Sasaki, H. Lee-Six, M. D. Young, L. B. Alexandrov, S. Behjati, T. J. Mitchell, S. Grossmann, H. Lightfoot, D. A. Egan, A. Pronk, N. Smakman, J. van Gorp, E. Anderson, S. J. Gamble, C. Alder, M. van de Wetering, P. J. Campbell, M. R. Stratton, H. Clevers, Intra-tumour diversification in colorectal cancer at the single-cell level, Nature 556, 457-462 (2018).
9. P. Eirew, A. Steif, J. Khattra, G. Ha, D. Yap, H. Farahani, K. Gelmon, S. Chia, C. Mar, A. Wan, E. Laks, J. Biele, K. Shumansky, J. Rosner, A. McPherson, C. Nielsen, A. J. Roth, C. Lefebvre, A. Bashashati, C. de Souza, C. Siu, R. Aniba, J. Brimhall, A. Oloumi, T. Osako, A. Bruna, J. L. Sandoval, T. Algara, W. Greenwood, K. Leung, H. Cheng, H. Xue, Y. Wang, D. Lin, A. J. Mungall, R. Moore, Y. Zhao, J. Lorette, L. Nguyen, D. Huntsman, C. J. Eaves, C. Hansen, M. A. Marra, C. Caldas, S. P. Shah, S. Aparicio, Dynamics of genomic clones in breast cancer patient xenografts at single-cell resolution, Nature 518, 422 (2015).
10. R. M. Hoffman, Screening for Prostate Cancer, New Engl J Medicine 365, 2013-2019 (2011).
11. S. Roy-Chowdhuri, H. Chen, R. R. Singh, S. Krishnamurthy, K. P. Patel, M. J. Routbort, J. Manekia, B. A. Barkoh, H. Yao, S. Sabir, R. R. Broaddus, J. L. Medeiros, G. Staerkel, J. Stewart, R. Luthra, Concurrent fine needle aspirations and core needle biopsies: a comparative study of substrates for next-generation sequencing in solid organ malignancies, Modern Pathol 30, 499 (2017).
12. T. Kilpeläinen, T. Tammela, L. Masttanen, P. Kujala, U.-H. Stenman, M. Ala-Opas, T. Murtola, A. Auvinen, False-positive screening results in the Finnish prostate cancer screening trial, Brit J Cancer 102, 469 (2010).
13. T. Golub, D. Slonim, P. Tamayo, C. Huard, M. Gaasenbeek, J. Mesirov, H. Coller, M. Loh, J. Downing, M. Caligiuri, C. Bloomfield, E. Lander, Molecular Classification of Cancer: Class Discovery and Class Prediction by Gene Expression Monitoring, Science 286, 531-537 (1999).
14. A. Calon, E. Lonardo, A. Berenguer-Llergo, E. Espinet, X. Hernando-Momblona, M. Iglesias, M. Sevillano, S. Palomo-Ponce, D. V. Tauriello, D. Byrom, C. Cortina, C. Morral, C. Barceló, S. Tosi, A. Riera, C. Attolini, D. Rossell, E. Sancho, E. Batlle, Stromal gene expression defines poor-prognosis subtypes in colorectal cancer, Nat Genet 47, ng.3225 (2015).
15. N. Sanati, O. D. lancu, G. Wu, J. E. Jacobs, S. K. McWeeney, Network-Based Predictors of Progression in Head and Neck Squamous Cell Carcinoma, Frontiers Genetics 9, 183 (2018).
16. A. Houle, H. Gibling, F. C. Lamaze, H. A. Edgington, D. Soave, M.-J. Fave, M. Agbessi, V. Bruat, L. D. Stein, P. Awadalla, Aberrant PRDM9 expression impacts the pan-cancer genomic landscape, Genome Res 28, 1611-1620 (2018).
17. P. Tata, R. D. Chow, V. nivas Saladi, A. Tata, A. Konkimalla, A. Bara, D. Montoro, L. P.

Hariri, A. R. Shih, M. Mino-Kenudson, H. Mou, S. Kimura, L. W. Ellisen, J. Rajagopal, Developmental History Provides a Roadmap for the Emergence of Tumor Plasticity, Developmental Cell 44, 679-693.e5 (2018).
18. M.-J. Favé, F. C. Lamaze, D. Soave, A. Hodgkinson, H. Gauvin, V. Bruat, J.-C. Grenier, E. Gbeha, K. Skead, A. Smargiassi, M. Johnson, Y. Idaghdour, P. Awadalla, Gene-by-environment interactions in urban populations modulate risk phenotypes, Nature Communications 9, 827 (2018).
19. M. Melé, P. G. Ferreira, F. Reverter, D. S. DeLuca, J. Monlong, M. Sammeth, T. R. Young, J. M. Goldmann, D. D. Pervouchine, T. J. Sullivan, R. Johnson, A. V. Segre, S. Djebali, A. Niarchou, T. Consortium, F. A. Wright, T. Lappalainen, M. Calvo, G. Getz, E. T. Dermitzakis, K. G. Ardlie, R. Guigó, The human transcriptome across tissues and individuals, Science 348, 660-665 (2015).
20. W. Chung, H. Eum, H.-O. Lee, K.-M. Lee, H.-B. Lee, K.-T. Kim, H. Ryu, S. Kim, J. Lee, Y. Park, Z. Kan, W. Han, W.-Y. Park, Single-cell RNA-seq enables comprehensive tumour and immune cell profiling in primary breast cancer, Nature Communications 8, ncomms15081 (2017).
21. A. M. Gross, J. F. Kreisberg, T. Ideker, Analysis of Matched Tumor and Normal Profiles Reveals Common Transcriptional and Epigenetic Signals Shared across Cancer Types, PLOS ONE 10, e0142618 (2015).
22. P. Kotsantis, L. Silva, S. Irmscher, R. M. Jones, L. Folkes, N. Gromak, E. Petermann, Increased global transcription activity as a mechanism of replication stress in cancer, Nature Communications 7, 13087 (2016).
23. M. Groh, N. Gromak, Out of Balance: R-loops in Human Disease, PLoS Genetics 10, e1004630 (2014).
24. M. B. Kursa, W. R. Rudnicki, Feature Selection with theBorutaPackage, J Stat Softw 36 (2010), doi:10.18637/jss.v036.i11.
25. Y. Cai, R. Nogales-Cadenas, Q. Zhang, J.-R. Lin, W. Zhang, K. O'Brien, C. Montagna, Z. D. Zhang, Transcriptomic dynamics of breast cancer progression in the MMTV-PyMT mouse model, BMC Genomics 18, 185 (2017).
26. A. I. Riker, S. A. Enkemann, O. Fodstad, S. Liu, S. Ren, C. Morris, Y. Xi, P. Howell, B. Metge, R. S. Samant, L. A. Shevde, W. Li, S. Eschrich, A. Daud, J. Ju, J. Matta, The gene expression profiles of primary and metastatic melanoma yields a transition point of tumor progression and metastasis, Bmc Med Genomics 1, 13 (2008).
27. J. Massagué, A. C. Obenauf, Metastatic colonization by circulating tumour cells, Nature 529, 298-306 (2016).
28. W. Jiao, G. Atwal, P. Polak, R. Karlic, E. Cuppen, A. Danyi, J. de Ridder, C. van Herpen, M. P. Lolkema, N. Steeghs, G. Getz, Q. D. Morris, L. D. Stein, P. Grp, I. of Net, A deep learning system can accurately classify primary and metastatic cancers based on patterns of passenger mutations, Biorxiv, 214494 (2019).
29. K. A. Hoadley, C. Yau, D. M. Wolf, A. D. Cherniack, D. Tamborero, S. Ng, M. Leiserson, B. Niu, M. D. McLellan, V. Uzunangelov, J. Zhang, C. Kandoth, R. Akbani, H. Shen, L. Omberg, A. Chu, A. A. Margolin, L. J. Veer, N. Lopez-Bigas, P. W. Laird, B. J. Raphael, L. Ding, G. A. Robertson, L. A. Byers, G. B. Mills, J. N. Weinstein, C. Waes, Z. Chen, E. A. Collisson, T. Network, C. C. Benz, C. M. Perou, J. M. Stuart, Multiplatform Analysis of 12 Cancer Types Reveals Molecular Classification within and across Tissues of Origin, Cell 158, 929-944 (2014).
30. H. Chen, X. He, The Convergent Cancer Evolution toward a Single Cellular Destination, Mol Biol Evol 33, 4-12 (2016).
31. C. F. Basil, Y. Zhao, K. Zavaglia, P. Jin, M. C. Panelli, S. Voiculescu, S. Mandruzzato, H. M. Lee, B. Seliger, R. S. Freedman, P. R. Taylor, N. Hu, P. Zanovello, F. M. Marincola, E. Wang, Common Cancer Biomarkers, Cancer Research 66, 2953-2961 (2006).
32. M. G. Best, N. Sol, I. Kooi, J. Tannous, B. A. Westerman, F. Rustenburg, P. Schellen, H. Verschueren, E. Post, J. Koster, B. Ylstra, N. Ameziane, J. Dorsman, E. F. Smit, H. M. Verheul, D. P. Noske, J. C. Reijneveld, J. A. R. Nilsson, B. A. Tannous, P. Wesseling, T. Wurdinger, RNA-Seq of Tumor-Educated Platelets Enables Blood-Based Pan-Cancer, Multiclass, and Molecular Pathway Cancer Diagnostics, Cancer Cell 28, 666-676 (2015).
33. J. D. Cohen, L. Li, Y. Wang, C. Thoburn, B. Afsari, L. Danilova, C. Douville, A. A. Javed, F. Wong, A. Mattox, R. H. Hruban, C. L. Wolfgang, M. G. Goggins, M. Molin, T.-L. Wang, R. Roden, A. P. Klein, J. Ptak, L. Dobbyn, J. Schaefer, N. Silliman, M. Popoli, J. T. Vogelstein, J. D. Browne, R. E. Schoen, R. E. Brand, J. Tie, P. Gibbs, H.-L. Wong, A. S. Mansfield, J. Jen, S. M. Hanash, M. Falconi, P. J. Allen, S. Zhou, C. Bettegowda, L. Diaz, C. Tomasetti, K. W. Kinzler, B. Vogelstein, A. Lennon, N. Papadopoulos, Detection and localization of surgically resectable cancers with a multi-analyte blood test, Science 359, eaar3247 (2018).
34. S. Shen, R. Singhania, G. Fehringer, A. Chakravarthy, M. H. Roehrl, D. Chadwick, P. C. Zuzarte, A. Borgida, T. Wang, T. Li, O. Kis, Z. Zhao, A. Spreafico, T. da Medina, Y. Wang, D. Roulois, I. Ettayebi, Z. Chen, S. Chow, T. Murphy, A. Arruda, G. M. O'Kane, J. Liu, M. Mansour, J. D. McPherson, C. O'Brien, N. Leighl, P. L. Bedard, N. Fleshner, G. Liu, M. D. Minden, S. Gallinger, A. Goldenberg, T. J. Pugh, M. M. Hoffman, S. V. Bratman, R. J. Hung, D. D. Carvalho, Sensitive tumour detection and classification using plasma cell-free DNA methylomes, Nature 563, 579-583 (2018).
35. P. G. Ferreira, M. Muñoz-Aguirre, F. Reverter, C. P. Godinho, A. Sousa, A. Amadoz, R. Sodaei, M. R. Hidalgo, D. Pervouchine, J. Carbonell-Caballero, R. Nurtdinov, A. Breschi, R. Amador, P. Oliveira, C. Qubuk, J. Curado, F. Aguet, C. Oliveira, J. Dopazo, M. Sammeth, K. G. Ardlie, R. Guigó, The effects of death and post-mortem cold ischemia on human tissue transcriptomes, Nature Communications 9, 490 (2018).
36. P. Group, C. Calabrese, N. R. Davidson, N. A. Fonseca, Y. He, A. Kahles, K.-V. Lehmann, F. Liu, Y. Shiraishi, C. ulette, L. Urban, D. Demircioǧlu, L. Greger, S. Li, D. Liu, M. D. Perry, L. Xiang, F. Zhang, J. Zhang, P. Bailey, S. Erkek, K. A. Hoadley, Y. Hou, H. Kilpinen, J. O. Korbel, M. G. Marin, J. Markowski, T. Nandi, Q. Pan-Hammarström, C. Pedamallu, R. Siebert, S. G. Stark, H. Su, P. Tan, S. M. Waszak, C. Yung, S. Zhu, P. Group, P. Awadalla, C. J. Creighton, M. Meyerson, B. F. Ouellette, K. Wu, H. Yang, I. of Network, A. Brazma, A. N. Brooks, J. Göke, G. Retsch, R. F. Schwarz, O. Stegle, Z. Zhang, Genomic basis for RNA alterations revealed by whole-genome analyses of 27 cancer types, Biorxiv, 183889 (2018).
37. S. Anders, P. Pyl, W. Huber, HTSeq—a Python framework to work with high-throughput sequencing data, Bioinformatics 31, 166-169 (2015).
38. J. T. Leek, svaseq: removing batch effects and other unwanted noise from sequencing data, Nucleic Acids Res 42, e161-e161 (2014).
39. J. Reimand, T. Arak, P. Adler, L. Kolberg, S. Reisberg, H. Peterson, J. Vilo, g:Profiler—a web server for functional interpretation of gene lists (2016 update), Nucleic Acids Res 44, W83-W89 (2016).

TABLE A
Samples descriptions by consortium and cancer types.

| # | Cancer Types | Primary Site | Acronyms |
|---|---|---|---|
| 1 | Adrenocortical carcinoma, adenocarcinoma | Adrenal gland | ACC |
| 2 | Bladder urothelial carcinoma | Bladder | BLCA |
| 3 | Breast invasive carcinomas | Breast | BRCA |
| 4 | Cervical squamous cell carcinoma and endocervical | Cervix uteri | CESC |
| | adenocarcinoma | | |
| 5 | Cholangiocarcinoma | Gallbladder; Liver [symbol] | CHOL |
| 6 | Chronic lymphocytic leukemia | Blood | CLLE |
| 7 | Colon adenocarcinoma | Colon; Rectosigm [symbol] | COAD |
| 8 | Lymphoid neoplasm diffuse large B-cell lymphoma, Mature | Bones, joints anc [symbol] | DLBC |
| | B-Cell Lymphoma | | |
| 9 | Esophageal carcinoma | EsophagusStoma [symbol] | ESCA |
| 10 | Glioblastoma multiforme | Brain | GBM |
| 11 | Head and neck squamous cell carcinoma | Base of tongue; B [symbol] | HNSC |
| 12 | Kidney chromophobe renal cell carcinoma, adenocarcinoma | Kidney | KICH |
| 13 | Kidney renal clear cell carcinoma, adenocarcinoma | Kidney | KIRC |
| 14 | Kidney renal papillary cell carcinoma, adenocarcinoma | Kidney | KIRP |
| 15 | Acute myeloid leukemia | Hematopoietic a [symbol] | LAML |
| 16 | Brain lower grade glioma | Brain | LGG |
| 17 | Liver hepatocellular carcinoma, adenocarcinoma | Liver and intrahe [symbol] | LIHC |
| 18 | Liver hepatocellular carcinoma (virus associated, HBV and HCV) | Liver | LIRI |
| 19 | Lung adenocarcinoma | Bronchus and lur [symbol] | LUAD |
| 20 | Lung squamous cell carcinoma | Bronchus and lur [symbol] | LUSC |
| 21 | Malignant Lymphoma, germinal-center derived B-cell malignant | Blood | MALY |
| | (non-Hodgkin) lymphoma | | |
| 22 | Mesothelioma | Bronchus and lur [symbol] | MESO |
| 23 | Ovarian Serous Cystadenocarcinoma | Ovary | OV |
| 24 | Pancreatic cancer, Adenocarcinoma | Pancreas | PAAD |
| 25 | Pancreatic cancer, Ductal adenocarcinoma | Pancreas | PACA |
| 26 | Pheochromocytoma and Paraganglioma | Adrenal gland; C [symbol] | PCPG |
| 27 | Prostate Adenocarcinoma | Prostate gland | PRAD |
| 28 | Rectum Adenocarcinoma | Colon; Connectiv [symbol] | READ |
| 29 | Renal cell carcinoma (Focus on but not limited to clear cell | Kidney | RECA |
| | subtype) | | |
| 30 | Sarcoma | Bones, joints an [symbol] | SARC |
| 31 | Skin Cutaneous Melanoma | Skin | SKCM |
| 32 | Stomach Adenocarcinoma | Stomach | STAD |
| 33 | Testicular Germ Cell Tumors | Testis | TGCT |
| 34 | Thyroid Carcinoma | Thyroid gland | THCA |
| 35 | Thymic Epithelial Neoplasms, Thymoma | Heart, mediastin [symbol] | THYM |
| 36 | Uterine Corpus Endometrial Carcinoma | Corpus uteri; Ute [symbol] | UCEC |
| 37 | Uterine Carcinosarcoma, Complex Mixed and Stromal Neoplasms | Uterus, NOS | UCS |
| 38 | Uveal Melanoma | Eye and adnexa [symbol] | UVM |
| 39 | GTEx Control Sample, Cells-leukemia cell line, | | CML |
| | K-562-SM-2D454 | | |
| 40 | GTEx non-cancerous tissues | | GTEx |
| Total | | | |

[symbol]

indicates data missing or illegible when filed

TABLE

| | | | | | | | | Primary | |
|---|---|---|---|---|---|---|---|---|---|
| # | TOTAL | TCGA | PCAWG | GTEx | Metastatic | Normal | Primary | Additional | Recurrent |
| 1 | 79 | 79 | 0 | 0 | 0 | 0 | 79 | 0 | 0 |
| 2 | 435 | 433 | 2 | 0 | 0 | 19 | 416 | 0 | 0 |
| 3 | 1251 | 1246 | 5 | 0 | 7 | 113 | 1131 | 0 | 0 |
| 4 | 309 | 309 | 0 | 0 | 2 | 3 | 304 | 0 | 0 |
| 5 | 45 | 45 | 0 | 0 | 0 | 9 | 36 | 0 | 0 |
| 6 | 67 | 0 | 67 | 0 | 0 | 0 | 67 | 0 | 0 |
| 7 | 546 | 546 | 0 | 0 | 1 | 41 | 503 | 0 | 1 |
| 8 | 48 | 48 | 0 | 0 | 0 | 0 | 48 | 0 | 0 |
| 9 | 198 | 198 | 0 | 0 | 1 | 13 | 184 | 0 | 0 |
| 10 | 175 | 175 | 0 | 0 | 0 | 5 | 157 | 0 | 13 |
| 11 | 548 | 548 | 0 | 0 | 2 | 44 | 502 | 0 | 0 |
| 12 | 91 | 91 | 0 | 0 | 0 | 25 | 66 | 0 | 0 |
| 13 | 616 | 616 | 0 | 0 | 0 | 72 | 543 | 1 | 0 |
| 14 | 323 | 323 | 0 | 0 | 0 | 32 | 290 | 1 | 0 |
| 15 | 126 | 126 | 0 | 0 | 0 | 0 | 126 | 0 | 0 |
| 16 | 532 | 532 | 0 | 0 | 0 | 0 | 514 | 0 | 18 |
| 17 | 424 | 424 | 0 | 0 | 0 | 50 | 371 | 0 | 3 |
| 18 | 118 | 0 | 118 | 0 | 0 | 56 | 62 | 0 | 0 |
| 19 | 601 | 601 | 0 | 0 | 0 | 59 | 540 | 0 | 2 |
| 20 | 555 | 555 | 0 | 0 | 0 | 51 | 504 | 0 | 0 |
| 21 | 90 | 0 | 90 | 0 | 0 | 0 | 90 | 0 | 0 |
| 22 | 87 | 87 | 0 | 0 | 0 | 0 | 87 | 0 | 0 |
| 23 | 493 | 430 | 63 | 0 | 1 | 0 | 472 | 0 | 20 |
| 24 | 183 | 183 | 0 | 0 | 1 | 4 | 178 | 0 | 0 |
| 25 | 57 | 0 | 57 | 0 | 0 | 0 | 57 | 0 | 0 |
| 26 | 187 | 187 | 0 | 0 | 2 | 3 | 179 | 3 | 0 |
| 27 | 558 | 558 | 0 | 0 | 1 | 52 | 505 | 0 | 0 |
| 28 | 177 | 177 | 0 | 0 | 0 | 10 | 166 | 0 | 1 |
| 29 | 68 | 0 | 68 | 0 | 0 | 25 | 43 | 0 | 0 |
| 30 | 265 | 265 | 0 | 0 | 1 | 2 | 259 | 0 | 3 |
| 31 | 473 | 473 | 0 | 0 | 369 | 1 | 103 | 0 | 0 |
| 32 | 453 | 453 | 0 | 0 | 0 | 37 | 416 | 0 | 0 |
| 33 | 156 | 156 | 0 | 0 | 0 | 0 | 150 | 6 | 0 |
| 34 | 592 | 572 | 20 | 0 | 8 | 63 | 521 | 0 | 0 |
| 35 | 122 | 122 | 0 | 0 | 0 | 2 | 120 | 0 | 0 |
| 36 | 589 | 589 | 0 | 0 | 0 | 35 | 553 | 0 | 1 |
| 37 | 57 | 57 | 0 | 0 | 0 | 0 | 57 | 0 | 0 |
| 38 | 80 | 80 | 0 | 0 | 0 | 0 | 80 | 0 | 0 |
| 39 | 102 | 0 | 0 | 102 | 0 | 0 | 102 | 0 | 0 |
| 40 | 9115 | 0 | 0 | 9115 | 0 | 9115 | 0 | 0 | 0 |
| Total | 20991 | 11284 | 490 | 9217 | 396 | 9941 | 10581 | 11 | 62 |

TABLE B
Differentially regulated genes betweenpaired healthy and tumor tissue biopsies.
The coefficients (log2 fold change) and the Bonferroni are calculated within each of the 1000 bootsraps.
Only the median value over 1000 bootsrap is reported for the coefficients and Bonferroni values.

| Ensembl gene id | Symbol | Chr | Start | End | Gene Biotype | Coefficient | Bonferroni | housekeeping | bio10 | bio30 | bio100 | bioortho | bio450 |
|---|---|---|---|---|---|---|---|---|---|---|---|---|---|
| ENSG00000060718 | COL11A1 | 1 | 103342023 | 103574052 | protein_coding | 3.700378611 | 7.52E−43 | FALSE | FALSE | FALSE | FALSE | FALSE | FALSE |
| ENSG00000101057 | MYBL2 | 20 | 42295754 | 42345136 | protein_coding | 3.24602094 | 2.76E−92 | FALSE | FALSE | FALSE | FALSE | FALSE | FALSE |
| ENSG00000175063 | UBE2C | 20 | 44441215 | 44445596 | protein_coding | 3.224292631 | 1.13E−104 | FALSE | FALSE | FALSE | FALSE | FALSE | TRUE |
| ENSG00000165304 | MELK | 9 | 36572859 | 36677678 | protein_coding | 3.138808631 | 1.21E−92 | FALSE | FALSE | FALSE | FALSE | FALSE | TRUE |
| ENSG00000099953 | MMP11 | 22 | 24110413 | 24126503 | protein_coding | 3.124396042 | 3.24E−72 | FALSE | TRUE | TRUE | TRUE | TRUE | TRUE |
| ENSG00000126787 | DLGAP5 | 14 | 55614830 | 55658396 | protein_coding | 3.068702908 | 3.06E−84 | FALSE | FALSE | FALSE | FALSE | FALSE | TRUE |
| ENSG00000186185 | KIF18B | 17 | 43002077 | 43025082 | protein_coding | 3.038595549 | 1.33E−89 | FALSE | FALSE | FALSE | FALSE | FALSE | FALSE |
| ENSG00000147889 | CDKN2A | 9 | 21967751 | 21995300 | protein_coding | 3.005436502 | 5.93E−65 | FALSE | FALSE | FALSE | TRUE | FALSE | FALSE |
| ENSG00000109674 | NEIL3 | 4 | 178230990 | 178284097 | protein_coding | 2.982939453 | 2.52E−68 | FALSE | FALSE | FALSE | FALSE | FALSE | FALSE |
| ENSG00000158402 | CDC25C | 5 | 137620954 | 137674044 | protein_coding | 2.977998205 | 4.47E−82 | FALSE | FALSE | FALSE | FALSE | FALSE | FALSE |
| ENSG00000123500 | COL10A1 | 6 | 116440086 | 116479910 | protein_coding | 2.96425707 | 1.98E−31 | FALSE | FALSE | TRUE | TRUE | FALSE | TRUE |
| ENSG00000123485 | HJURP | 2 | 234742062 | 234763212 | protein_coding | 2.956710293 | 8.22E−95 | FALSE | FALSE | FALSE | FALSE | FALSE | FALSE |
| ENSG00000187730 | GABRD | 1 | 1950780 | 1962192 | protein_coding | 2.953121717 | 1.65E−65 | FALSE | FALSE | TRUE | TRUE | TRUE | FALSE |
| ENSG00000090889 | KIF4A | X | 69509879 | 69640682 | protein_coding | 2.94364154 | 3.50E−97 | FALSE | FALSE | FALSE | FALSE | FALSE | TRUE |
| ENSG00000182379 | NXPH4 | 12 | 57610578 | 57620232 | protein_coding | 2.939489087 | 1.39E−52 | FALSE | FALSE | FALSE | FALSE | FALSE | FALSE |
| ENSG00000131747 | TOP2A | 17 | 38544768 | 38574202 | protein_coding | 2.936641292 | 7.54E−91 | FALSE | FALSE | FALSE | FALSE | TRUE | TRUE |
| ENSG00000117650 | NEK2 | 1 | 211836114 | 211848960 | protein_coding | 2.905128233 | 2.11E−77 | FALSE | FALSE | FALSE | FALSE | FALSE | TRUE |
| ENSG00000135451 | TROAP | 12 | 49717019 | 49725514 | protein_coding | 2.898078211 | 8.36E−92 | FALSE | FALSE | FALSE | FALSE | FALSE | FALSE |
| ENSG00000115163 | CENPA | 2 | 26987157 | 27023935 | protein_coding | 2.862918001 | 6.00E−76 | FALSE | FALSE | FALSE | FALSE | FALSE | TRUE |
| ENSG00000066279 | ASPM | 1 | 197053258 | 197115824 | protein_coding | 2.848892395 | 1.34E−81 | FALSE | FALSE | FALSE | FALSE | FALSE | FALSE |
| ENSG00000183856 | IQGAP3 | 1 | 156495197 | 156542396 | protein_coding | 2.839366333 | 2.03E−91 | FALSE | FALSE | FALSE | FALSE | FALSE | FALSE |
| ENSG00000034063 | UHRF1 | 19 | 4903092 | 4962165 | processed_transc [symbol] | 2.829297118 | 5.54E−91 | FALSE | FALSE | FALSE | FALSE | FALSE | FALSE |
| ENSG00000118193 | KIF14 | 1 | 200520628 | 200589862 | protein_coding | 2.807250187 | 1.10E−82 | FALSE | FALSE | FALSE | FALSE | FALSE | FALSE |
| ENSG00000093009 | CDC45 | 22 | 19466982 | 19508135 | protein_coding | 2.783578132 | 1.51E−78 | FALSE | FALSE | FALSE | FALSE | FALSE | FALSE |
| ENSG00000136231 | IGF2BP3 | 7 | 23349828 | 23510086 | protein_coding | 2.781410644 | 6.98E−35 | FALSE | FALSE | FALSE | FALSE | FALSE | TRUE |
| ENSG00000127564 | PKMYT1 | 16 | 3018025 | 3030540 | protein_coding | 2.774487656 | 4.19E−82 | FALSE | FALSE | FALSE | FALSE | FALSE | FALSE |
| ENSG00000169679 | BUB1 | 2 | 111395275 | 111435691 | protein_coding | 2.772355048 | 4.08E−95 | FALSE | FALSE | FALSE | FALSE | FALSE | TRUE |
| ENSG00000112984 | KIF20A | 5 | 137514408 | 137523404 | protein_coding | 2.763276953 | 5.30E−85 | FALSE | FALSE | FALSE | FALSE | FALSE | TRUE |
| ENSG00000107159 | CA9 | 9 | 35673853 | 35681156 | protein_coding | 2.757223 | 2.28E−20 | FALSE | FALSE | FALSE | FALSE | FALSE | FALSE |
| ENSG00000148773 | MKI67 | 10 | 129894923 | 129924649 | protein_coding | 2.7562257 | 9.42E−88 | FALSE | FALSE | FALSE | FALSE | FALSE | FALSE |
| ENSG00000164283 | ESM1 | 5 | 54273692 | 54318499 | protein_coding | 2.74466117 | 5.42E−49 | FALSE | TRUE | TRUE | TRUE | FALSE | TRUE |
| ENSG00000196611 | MMP1 | 11 | 102660651 | 102668891 | protein_coding | 2.740088268 | 5.63E−23 | FALSE | FALSE | FALSE | FALSE | FALSE | FALSE |
| ENSG00000256663 | | 12 | 20704524 | 20705946 | pseudogene | 2.715786331 | 5.37E−70 | FALSE | FALSE | FALSE | FALSE | FALSE | TRUE |
| ENSG00000089685 | BIRC5 | 17 | 76210267 | 76221717 | protein_coding | 2.711046498 | 6.05E−79 | FALSE | FALSE | FALSE | FALSE | FALSE | FALSE |
| ENSG00000065328 | MCM10 | 10 | 13203554 | 13253104 | protein_coding | 2.675944977 | 2.73E−73 | FALSE | FALSE | FALSE | FALSE | FALSE | FALSE |
| ENSG00000184661 | CDCA2 | 8 | 25316513 | 25365436 | protein_coding | 2.672422155 | 1.91E−59 | FALSE | FALSE | FALSE | FALSE | FALSE | TRUE |
| ENSG00000168078 | PBK | 8 | 27667137 | 27695612 | protein_coding | 2.666600739 | 1.55E−64 | FALSE | FALSE | FALSE | FALSE | FALSE | TRUE |
| ENSG00000117724 | CENPF | 1 | 214776538 | 214837931 | protein_coding | 2.646433637 | 7.46E−90 | FALSE | FALSE | FALSE | FALSE | FALSE | TRUE |
| ENSG00000109805 | NCAPG | 4 | 17812525 | 17846485 | protein_coding | 2.64509562 | 5.17E−76 | FALSE | FALSE | FALSE | FALSE | FALSE | FALSE |
| ENSG00000011426 | ANLN | 7 | 36429415 | 36493400 | protein_coding | 2.638050449 | 3.38E−80 | FALSE | FALSE | FALSE | FALSE | FALSE | FALSE |
| ENSG00000178999 | AURKB | 17 | 8108056 | 8113918 | protein_coding | 2.617563789 | 5.22E−67 | FALSE | FALSE | FALSE | FALSE | FALSE | FALSE |
| ENSG00000024526 | DEPDC1 | 1 | 68939835 | 68962904 | protein_coding | 2.616364468 | 2.38E−59 | FALSE | FALSE | FALSE | FALSE | FALSE | TRUE |
| ENSG00000088325 | TPX2 | 20 | 30327074 | 30389608 | protein_coding | 2.614236767 | 2.93E−82 | FALSE | FALSE | FALSE | FALSE | FALSE | TRUE |
| ENSG00000138180 | CEP55 | 10 | 95256389 | 95288849 | protein_coding | 2.596024972 | 4.19E−77 | FALSE | FALSE | FALSE | FALSE | FALSE | TRUE |
| ENSG00000258947 | | 16 | 89987800 | 90005169 | protein_coding | 2.588061792 | 7.31E−42 | FALSE | FALSE | FALSE | FALSE | FALSE | FALSE |
| ENSG00000164932 | CTHRC1 | 8 | 104383743 | 104395225 | protein_coding | 2.5852282 | 1.82E−61 | FALSE | FALSE | FALSE | FALSE | TRUE | FALSE |
| ENSG00000129173 | E2F8 | 11 | 19245610 | 19263167 | protein_coding | 2.566179954 | 5.85E−51 | FALSE | FALSE | FALSE | FALSE | FALSE | FALSE |
| ENSG00000143228 | NUF2 | 1 | 163236366 | 163325554 | protein_coding | 2.554005109 | 1.74E−66 | FALSE | FALSE | FALSE | FALSE | FALSE | TRUE |
| ENSG00000169607 | CKAP2L | 2 | 113493930 | 113522254 | protein_coding | 2.545910843 | 1.29E−76 | FALSE | FALSE | FALSE | FALSE | FALSE | FALSE |
| ENSG00000075218 | GTSE1 | 22 | 46692638 | 46726707 | protein_coding | 2.528178244 | 3.54E−83 | FALSE | FALSE | FALSE | FALSE | FALSE | FALSE |
| ENSG00000112742 | TTK | 6 | 80713604 | 80752244 | protein_coding | 2.517300522 | 7.17E−66 | FALSE | FALSE | FALSE | FALSE | FALSE | TRUE |
| ENSG00000189057 | FAM111B | 11 | 58874658 | 58894883 | protein_coding | 2.497710829 | 1.07E−72 | FALSE | FALSE | FALSE | TRUE | FALSE | TRUE |
| ENSG00000154839 | SKA1 | 18 | 47901365 | 47920543 | protein_coding | 2.484173797 | 4.04E−67 | FALSE | FALSE | FALSE | FALSE | FALSE | FALSE |
| ENSG00000111206 | FOXM1 | 12 | 2966847 | 2986206 | protein_coding | 2.478857364 | 3.72E−80 | FALSE | FALSE | FALSE | FALSE | FALSE | FALSE |
| ENSG00000156970 | BUB1B | 15 | 40453224 | 40513337 | protein_coding | 2.47172128 | 5.77E−69 | FALSE | FALSE | FALSE | FALSE | FALSE | TRUE |
| ENSG00000165891 | E2F7 | 12 | 77415027 | 77459360 | protein_coding | 2.465168855 | 3.59E−60 | FALSE | FALSE | FALSE | FALSE | FALSE | FALSE |
| ENSG00000206195 | | 22 | 16147979 | 16193004 | processed_transc [symbol] | 2.454273944 | 2.00E−52 | FALSE | FALSE | FALSE | TRUE | FALSE | FALSE |
| ENSG00000171848 | RRM2 | 2 | 10262455 | 10271545 | protein_coding | 2.443624743 | 3.28E−63 | FALSE | FALSE | FALSE | FALSE | FALSE | TRUE |
| ENSG00000174371 | EXO1 | 1 | 242011269 | 242058450 | protein_coding | 2.432405336 | 1.14E−62 | FALSE | FALSE | FALSE | FALSE | FALSE | TRUE |
| ENSG00000187456 | RDM1 | 17 | 34245070 | 34257777 | protein_coding | 2.42837613 | 9.97E−43 | FALSE | FALSE | FALSE | FALSE | FALSE | FALSE |
| ENSG00000157456 | CCNB2 | 15 | 59397277 | 59417244 | protein_coding | 2.383460879 | 8.29E−71 | FALSE | FALSE | FALSE | FALSE | FALSE | FALSE |
| ENSG00000165480 | SKA3 | 13 | 21727734 | 21750741 | protein_coding | 2.378649395 | 4.53E−69 | FALSE | FALSE | FALSE | FALSE | TRUE | TRUE |
| ENSG00000225210 | | 14 | 19650018 | 19718563 | lincRNA | 2.373635543 | 4.98E−40 | FALSE | FALSE | FALSE | TRUE | FALSE | FALSE |
| ENSG00000129195 | FAM64A | 17 | 6347735 | 6354789 | protein_coding | 2.370513923 | 2.24E−64 | FALSE | FALSE | FALSE | FALSE | FALSE | FALSE |
| ENSG00000121152 | NCAPH | 2 | 97001525 | 97039583 | protein_coding | 2.367609324 | 2.23E−68 | FALSE | FALSE | FALSE | FALSE | FALSE | FALSE |
| ENSG00000094804 | CDC6 | 17 | 38443885 | 38459171 | protein_coding | 2.346292455 | 1.57E−72 | FALSE | FALSE | FALSE | FALSE | FALSE | FALSE |
| ENSG00000144452 | ABCA12 | 2 | 215796266 | 216003151 | protein_coding | 2.344122398 | 6.98E−23 | FALSE | FALSE | FALSE | FALSE | FALSE | TRUE |
| ENSG00000143476 | DTL | 1 | 212208919 | 212280742 | protein_coding | 2.332500761 | 1.27E−72 | FALSE | FALSE | FALSE | FALSE | TRUE | TRUE |
| ENSG00000117399 | CDC20 | 1 | 43824626 | 43828874 | protein_coding | 2.320876426 | 3.25E−53 | FALSE | FALSE | FALSE | FALSE | FALSE | FALSE |
| ENSG00000092853 | CLSPN | 1 | 36185819 | 36235568 | protein_coding | 2.317162237 | 2.77E−64 | FALSE | FALSE | FALSE | FALSE | FALSE | FALSE |
| ENSG00000131650 | KREMEN2 | 16 | 3013945 | 3018384 | protein_coding | 2.315641672 | 1.28E−34 | FALSE | FALSE | FALSE | FALSE | FALSE | FALSE |
| ENSG00000167513 | CDT1 | 16 | 88869621 | 88875666 | protein_coding | 2.301352374 | 5.38E−76 | FALSE | FALSE | FALSE | FALSE | FALSE | FALSE |
| ENSG00000051341 | POLQ | 3 | 121150278 | 121264853 | protein_coding | 2.285468527 | 1.97E−59 | FALSE | FALSE | FALSE | FALSE | FALSE | FALSE |
| ENSG00000244306 | | 14 | 19854098 | 19925348 | lincRNA | 2.261379907 | 1.49E−45 | FALSE | FALSE | FALSE | TRUE | FALSE | FALSE |
| ENSG00000175874 | CREG2 | 2 | 101962013 | 102004057 | protein_coding | 2.251505356 | 2.32E−28 | FALSE | FALSE | FALSE | FALSE | FALSE | FALSE |
| ENSG00000166851 | PLK1 | 16 | 23688977 | 23701688 | protein_coding | 2.247782539 | 8.43E−65 | FALSE | FALSE | FALSE | FALSE | FALSE | FALSE |
| ENSG00000100985 | MMP9 | 20 | 44637547 | 44645200 | protein_coding | 2.239634426 | 1.27E−33 | FALSE | FALSE | FALSE | FALSE | FALSE | FALSE |
| ENSG00000105011 | ASF1B | 19 | 14230321 | 14247768 | protein_coding | 2.225494364 | 3.80E−75 | FALSE | FALSE | FALSE | FALSE | FALSE | TRUE |
| ENSG00000134028 | ADAMDEC1 | 8 | 24241798 | 24263526 | protein_coding | 2.21732651 | 5.14E−19 | FALSE | FALSE | FALSE | FALSE | FALSE | FALSE |
| ENSG00000169258 | GPRIN1 | 5 | 176022803 | 176037134 | protein_coding | 2.213232875 | 1.21E−69 | FALSE | FALSE | FALSE | FALSE | TRUE | FALSE |
| ENSG00000100526 | CDKN3 | 14 | 54863567 | 54886936 | protein_coding | 2.206754905 | 3.30E−63 | FALSE | FALSE | FALSE | FALSE | FALSE | FALSE |
| ENSG00000142945 | KIF2C | 1 | 45205490 | 45233439 | protein_coding | 2.206633534 | 4.84E−57 | FALSE | FALSE | FALSE | FALSE | FALSE | FALSE |
| ENSG00000146670 | CDCA5 | 11 | 64833772 | 64851636 | protein_coding | 2.18793376 | 1.44E−72 | FALSE | FALSE | FALSE | FALSE | FALSE | FALSE |
| ENSG00000215784 | FAM72D | 1 | 143896452 | 143913143 | protein_coding | 2.151941264 | 3.94E−45 | FALSE | FALSE | FALSE | FALSE | FALSE | FALSE |
| ENSG00000161888 | SPC24 | 19 | 11242196 | 11266484 | protein_coding | 2.149395786 | 2.97E−77 | FALSE | FALSE | FALSE | FALSE | FALSE | FALSE |
| ENSG00000121211 | MND1 | 4 | 154265801 | 154336270 | protein_coding | 2.149060772 | 9.50E−53 | FALSE | FALSE | FALSE | FALSE | FALSE | TRUE |
| ENSG00000186871 | ERCC6L | X | 71424510 | 71458897 | protein_coding | 2.138925085 | 1.58E−64 | FALSE | FALSE | FALSE | FALSE | FALSE | TRUE |
| ENSG00000151012 | SLC7A11 | 4 | 139085251 | 139163503 | protein_coding | 2.137100609 | 6.16E−34 | FALSE | FALSE | FALSE | FALSE | FALSE | FALSE |
| ENSG00000237649 | KIFC1 | 6 | 33359313 | 33377701 | protein_coding | 2.131722501 | 4.42E−62 | FALSE | FALSE | FALSE | TRUE | FALSE | TRUE |
| ENSG00000157766 | ACAN | 15 | 89346674 | 89418585 | protein_coding | 2.126407565 | 4.78E−31 | FALSE | FALSE | FALSE | FALSE | TRUE | FALSE |
| ENSG00000100162 | CENPM | 22 | 42334725 | 42343168 | protein_coding | 2.123618312 | 2.86E−55 | FALSE | FALSE | FALSE | FALSE | FALSE | TRUE |
| ENSG00000152253 | SPC25 | 2 | 169690642 | 169769881 | protein_coding | 2.118254845 | 9.43E−56 | FALSE | FALSE | FALSE | FALSE | FALSE | FALSE |
| ENSG00000072571 | HMMR | 5 | 162887209 | 162918947 | protein_coding | 2.115745374 | 1.75E−51 | FALSE | FALSE | FALSE | FALSE | FALSE | FALSE |
| ENSG00000101412 | E2F1 | 20 | 32263489 | 32274210 | protein_coding | 2.114575879 | 4.59E−78 | FALSE | FALSE | FALSE | FALSE | FALSE | FALSE |
| ENSG00000145386 | CCNA2 | 4 | 122737599 | 122745087 | protein_coding | 2.110058808 | 2.48E−61 | FALSE | FALSE | FALSE | FALSE | FALSE | FALSE |
| ENSG00000134690 | CDCA8 | 1 | 38158090 | 38175391 | protein_coding | 2.090937802 | 1.94E−60 | FALSE | FALSE | FALSE | FALSE | FALSE | FALSE |
| ENSG00000080986 | NDC80 | 18 | 2571510 | 2616634 | protein_coding | 2.076081589 | 6.04E−50 | FALSE | FALSE | FALSE | FALSE | FALSE | FALSE |
| ENSG00000118785 | SPP1 | 4 | 88896819 | 88904562 | protein_coding | 2.059146777 | 4.78E−25 | FALSE | FALSE | FALSE | FALSE | TRUE | FALSE |
| ENSG00000102384 | CENPI | X | 100353178 | 100418670 | protein_coding | 2.053535692 | 1.13E−62 | FALSE | FALSE | FALSE | FALSE | FALSE | TRUE |
| ENSG00000166803 | KIAA0101 | 15 | 64657193 | 64679886 | protein_coding | 2.040657652 | 1.85E−53 | FALSE | FALSE | FALSE | FALSE | TRUE | TRUE |
| ENSG00000071539 | TRIP13 | 5 | 892758 | 919472 | protein_coding | 2.024192506 | 5.83E−61 | FALSE | FALSE | FALSE | FALSE | FALSE | TRUE |
| ENSG00000095970 | TREM2 | 6 | 41126244 | 41130924 | protein_coding | 2.014475818 | 3.81E−29 | FALSE | FALSE | FALSE | TRUE | TRUE | TRUE |
| ENSG00000187583 | PLEKHN1 | 1 | 901877 | 911245 | protein_coding | 1.98232552 | 4.04E−29 | FALSE | FALSE | FALSE | TRUE | FALSE | FALSE |
| ENSG00000162062 | C16orf59 | 16 | 2510081 | 2514964 | protein_coding | 1.980856223 | 3.03E−54 | FALSE | FALSE | FALSE | FALSE | FALSE | FALSE |
| ENSG00000138778 | CENPE | 4 | 104026963 | 104119566 | protein_coding | 1.980495736 | 1.56E−60 | FALSE | FALSE | FALSE | FALSE | FALSE | FALSE |
| ENSG00000129810 | SGOL1 | 3 | 20202085 | 20227784 | protein_coding | 1.978439649 | 1.84E−43 | FALSE | FALSE | FALSE | FALSE | FALSE | FALSE |
| ENSG00000154920 | EME1 | 17 | 48450581 | 48458844 | protein_coding | 1.970230389 | 2.67E−64 | FALSE | FALSE | FALSE | FALSE | FALSE | FALSE |
| ENSG00000167619 | TMEM145 | 19 | 42817477 | 42829214 | protein_coding | 1.961401435 | 7.29E−26 | FALSE | FALSE | FALSE | FALSE | FALSE | FALSE |
| ENSG00000126778 | SIX1 | 14 | 61110133 | 61124977 | protein_coding | 1.958334947 | 3.16E−24 | FALSE | FALSE | FALSE | FALSE | TRUE | TRUE |
| ENSG00000164611 | PTTG1 | 5 | 159848829 | 159855748 | protein_coding | 1.952283175 | 4.86E−49 | FALSE | FALSE | FALSE | FALSE | FALSE | FALSE |
| ENSG00000007968 | E2F2 | 1 | 23832922 | 23857712 | protein_coding | 1.951755786 | 7.37E−36 | FALSE | FALSE | FALSE | FALSE | FALSE | FALSE |
| ENSG00000148848 | ADAM12 | 10 | 127700950 | 128077024 | protein_coding | 1.938198285 | 1.08E−29 | FALSE | FALSE | FALSE | FALSE | TRUE | FALSE |
| ENSG00000137804 | NUSAP1 | 15 | 41624892 | 41673248 | protein_coding | 1.932547666 | 1.19E−53 | FALSE | FALSE | FALSE | FALSE | FALSE | FALSE |
| ENSG00000171320 | ESCO2 | 8 | 27629466 | 27670157 | protein_coding | 1.925265494 | 2.86E−41 | FALSE | FALSE | FALSE | FALSE | FALSE | TRUE |
| ENSG00000091651 | ORC6 | 16 | 46723555 | 46732306 | protein_coding | 1.921604735 | 8.60E−58 | FALSE | FALSE | FALSE | FALSE | FALSE | FALSE |
| ENSG00000196584 | XRCC2 | 7 | 152341864 | 152373250 | protein_coding | 1.916458735 | 7.69E−65 | FALSE | FALSE | FALSE | FALSE | FALSE | FALSE |
| ENSG00000163888 | CAMK2N2 | 3 | 183977001 | 183979251 | protein_coding | 1.90286131 | 8.38E−29 | FALSE | FALSE | FALSE | FALSE | FALSE | FALSE |
| ENSG00000169495 | HTRA4 | 8 | 38831683 | 38846181 | protein_coding | 1.89501971 | 2.80E−17 | FALSE | FALSE | FALSE | FALSE | FALSE | FALSE |
| ENSG00000108821 | COL1A1 | 17 | 48260650 | 48278993 | protein_coding | 1.886677091 | 9.03E−30 | FALSE | FALSE | FALSE | FALSE | TRUE | FALSE |
| ENSG00000151725 | CENPU | 4 | 185615772 | 185655287 | protein_coding | 1.885299104 | 3.47E−63 | FALSE | FALSE | FALSE | FALSE | FALSE | FALSE |
| ENSG00000272405 | | 1 | 156611458 | 156614679 | antisense | 1.877046092 | 2.23E−20 | FALSE | FALSE | FALSE | TRUE | FALSE | FALSE |
| ENSG00000078098 | FAP | 2 | 163027194 | 163101661 | protein_coding | 1.867036535 | 1.34E−24 | FALSE | FALSE | FALSE | FALSE | FALSE | FALSE |
| ENSG00000229953 | | 1 | 156616299 | 156631216 | antisense | 1.865563415 | 1.23E−18 | FALSE | FALSE | FALSE | TRUE | FALSE | TRUE |
| ENSG00000138316 | ADAMTS14 | 10 | 72432559 | 72522197 | protein_coding | 1.85912925 | 4.94E−31 | FALSE | FALSE | FALSE | FALSE | FALSE | FALSE |
| ENSG00000249859 | PVT1 | 8 | 128806779 | 129113499 | processed_transc [symbol] | 1.852858657 | 1.71E−37 | FALSE | FALSE | FALSE | FALSE | FALSE | FALSE |
| ENSG00000140534 | TICRR | 15 | 90118713 | 90174287 | protein_coding | 1.852121782 | 2.18E−42 | FALSE | FALSE | FALSE | FALSE | FALSE | FALSE |
| ENSG00000174939 | ASPHD1 | 16 | 29911696 | 29931185 | protein_coding | 1.849632444 | 1.95E−27 | FALSE | FALSE | FALSE | FALSE | FALSE | FALSE |
| ENSG00000146410 | MTFR2 | 6 | 136552162 | 136571473 | protein_coding | 1.84489342 | 1.48E−52 | FALSE | FALSE | FALSE | FALSE | FALSE | FALSE |
| ENSG00000163808 | KIF15 | 3 | 44803209 | 44914868 | protein_coding | 1.843100237 | 9.54E−43 | FALSE | FALSE | FALSE | FALSE | FALSE | FALSE |
| ENSG00000174938 | SEZ6L2 | 16 | 29882480 | 29910868 | protein_coding | 1.837840566 | 1.44E−26 | FALSE | FALSE | FALSE | TRUE | TRUE | FALSE |
| ENSG00000137825 | ITPKA | 15 | 41785591 | 41795747 | protein_coding | 1.83610884 | 2.76E−21 | FALSE | FALSE | FALSE | FALSE | FALSE | FALSE |
| ENSG00000077152 | UBE2T | 1 | 202300785 | 202311108 | protein_coding | 1.835476065 | 3.90E−50 | FALSE | FALSE | FALSE | FALSE | FALSE | TRUE |
| ENSG00000085999 | RAD54L | 1 | 46713360 | 46744145 | protein_coding | 1.834928495 | 4.02E−52 | FALSE | FALSE | FALSE | FALSE | FALSE | FALSE |
| ENSG00000198901 | PRC1 | 15 | 91509270 | 91538859 | protein_coding | 1.82858805 | 1.37E−58 | FALSE | FALSE | FALSE | FALSE | FALSE | FALSE |
| ENSG00000008300 | CELSR3 | 3 | 48673902 | 48700348 | protein_coding | 1.814293436 | 9.89E−45 | FALSE | FALSE | FALSE | FALSE | FALSE | FALSE |
| ENSG00000087586 | AURKA | 20 | 54944445 | 54967393 | protein_coding | 1.811708536 | 1.13E−55 | FALSE | FALSE | FALSE | FALSE | FALSE | TRUE |
| ENSG00000068489 | PRR11 | 17 | 57232860 | 57282066 | protein_coding | 1.795145304 | 7.36E−52 | FALSE | FALSE | FALSE | FALSE | TRUE | TRUE |
| ENSG00000214145 | LINC00887 | 3 | 194014254 | 194030592 | lincRNA | 1.793569078 | 5.49E−10 | FALSE | FALSE | FALSE | FALSE | FALSE | TRUE |
| ENSG00000111665 | CDCA3 | 12 | 6953957 | 6961230 | protein_coding | 1.777021878 | 4.35E−44 | FALSE | FALSE | FALSE | FALSE | FALSE | FALSE |
| ENSG00000272068 | | 1 | 156607575 | 156610796 | lincRNA | 1.771367287 | 8.08E−13 | FALSE | FALSE | FALSE | FALSE | FALSE | FALSE |
| ENSG00000131153 | GINS2 | 16 | 85709804 | 85723679 | protein_coding | 1.76836063 | 4.57E−52 | FALSE | FALSE | FALSE | FALSE | TRUE | TRUE |
| ENSG00000230061 | | 21 | 45834473 | 45845155 | antisense | 1.767875985 | 9.91E−14 | FALSE | FALSE | FALSE | FALSE | FALSE | FALSE |
| ENSG00000177602 | GSG2 | 17 | 3627211 | 3630067 | protein_coding | 1.76269331 | 4.32E−41 | FALSE | FALSE | FALSE | FALSE | FALSE | FALSE |
| ENSG00000162073 | PAQR4 | 16 | 3019246 | 3023490 | protein_coding | 1.758739701 | 9.65E−63 | FALSE | FALSE | FALSE | FALSE | FALSE | FALSE |
| ENSG00000186193 | SAPCD2 | 9 | 139956581 | 139965040 | protein_coding | 1.758219591 | 2.60E−29 | FALSE | FALSE | FALSE | FALSE | FALSE | FALSE |
| ENSG00000265415 | | 17 | 57280038 | 57281190 | antisense | 1.756192129 | 4.41E−37 | FALSE | FALSE | FALSE | FALSE | FALSE | TRUE |
| ENSG00000123219 | CENPK | 5 | 64813593 | 64858998 | protein_coding | 1.749938452 | 4.99E−43 | FALSE | FALSE | FALSE | FALSE | FALSE | FALSE |
| ENSG00000198826 | ARHGAP11A | 15 | 32907345 | 32932150 | protein_coding | 1.746547587 | 3.25E−60 | FALSE | FALSE | FALSE | FALSE | FALSE | FALSE |
| ENSG00000133063 | CHIT1 | 1 | 203181955 | 203242769 | protein_coding | 1.742396624 | 5.13E−11 | FALSE | FALSE | FALSE | FALSE | FALSE | FALSE |
| ENSG00000170312 | CDK1 | 10 | 62538089 | 62554610 | protein_coding | 1.740964035 | 4.07E−36 | FALSE | FALSE | FALSE | FALSE | FALSE | TRUE |
| ENSG00000183010 | PYCR1 | 17 | 79890260 | 79900288 | protein_coding | 1.737299964 | 5.56E−35 | FALSE | FALSE | FALSE | FALSE | FALSE | TRUE |
| ENSG00000240498 | CDKN2B-AS1 | 9 | 21994777 | 22121096 | antisense | 1.733510348 | 1.28E−07 | FALSE | FALSE | FALSE | FALSE | FALSE | FALSE |
| ENSG00000051180 | RAD51 | 15 | 40986972 | 41024354 | protein_coding | 1.720584502 | 1.86E−49 | FALSE | FALSE | FALSE | FALSE | FALSE | FALSE |
| ENSG00000006118 | TMEM132A | 11 | 60691935 | 60704631 | protein_coding | 1.718736825 | 7.82E−49 | FALSE | FALSE | FALSE | FALSE | FALSE | FALSE |
| ENSG00000136492 | BRIP1 | 17 | 59758627 | 59940882 | protein_coding | 1.712825943 | 1.47E−44 | FALSE | FALSE | FALSE | FALSE | FALSE | FALSE |
| ENSG00000106462 | EZH2 | 7 | 148504475 | 148581413 | protein_coding | 1.707740695 | 1.28E−61 | FALSE | FALSE | FALSE | FALSE | FALSE | FALSE |
| ENSG00000149948 | HMGA2 | 12 | 66217911 | 66360075 | protein_coding | 1.702906944 | 1.28E−07 | FALSE | FALSE | FALSE | FALSE | FALSE | TRUE |
| ENSG00000105464 | GRIN2D | 19 | 48898132 | 48948188 | protein_coding | 1.699864206 | 2.34E−21 | FALSE | FALSE | FALSE | FALSE | FALSE | FALSE |
| ENSG00000130208 | APOC1 | 19 | 45417504 | 45422606 | protein_coding | 1.694336325 | 4.74E−14 | FALSE | FALSE | FALSE | FALSE | FALSE | TRUE |
| ENSG00000075702 | WDR62 | 19 | 36545783 | 36596008 | protein_coding | 1.68404802 | 1.47E−43 | FALSE | FALSE | FALSE | FALSE | FALSE | FALSE |
| ENSG00000104147 | OIP5 | 15 | 41601466 | 41624819 | protein_coding | 1.672305008 | 1.86E−41 | FALSE | FALSE | FALSE | FALSE | FALSE | FALSE |
| ENSG00000121621 | KIF18A | 11 | 28042167 | 28129855 | protein_coding | 1.665664338 | 6.70E−39 | FALSE | FALSE | FALSE | FALSE | FALSE | TRUE |
| ENSG00000035499 | DEPDC1B | 5 | 59892739 | 59996017 | protein_coding | 1.665049762 | 1.37E−19 | FALSE | FALSE | FALSE | FALSE | FALSE | FALSE |
| ENSG00000189410 | SH2D5 | 1 | 21046225 | 21059330 | protein_coding | 1.65863476 | 5.95E−14 | FALSE | FALSE | FALSE | FALSE | FALSE | FALSE |
| ENSG00000140525 | FANCI | 15 | 89787180 | 89860492 | protein_coding | 1.647831374 | 2.97E−68 | FALSE | FALSE | FALSE | FALSE | FALSE | FALSE |
| ENSG00000187741 | FANCA | 16 | 89803957 | 89883065 | protein_coding | 1.637442672 | 6.48E−53 | FALSE | FALSE | FALSE | FALSE | FALSE | TRUE |
| ENSG00000137807 | KIF23 | 15 | 69706585 | 69740764 | protein_coding | 1.637390615 | 8.68E−30 | FALSE | FALSE | FALSE | FALSE | FALSE | TRUE |
| ENSG00000183150 | GPR19 | 12 | 12813825 | 12849141 | protein_coding | 1.636535031 | 3.27E−29 | FALSE | FALSE | FALSE | FALSE | FALSE | FALSE |
| ENSG00000106327 | TFR2 | 7 | 100218039 | 100240402 | protein_coding | 1.636137018 | 2.07E−15 | FALSE | FALSE | FALSE | FALSE | FALSE | TRUE |
| ENSG00000111247 | RAD51AP1 | 12 | 4647950 | 4669214 | protein_coding | 1.632589844 | 8.02E−50 | FALSE | FALSE | FALSE | FALSE | FALSE | TRUE |
| ENSG00000197299 | BLM | 15 | 91260558 | 91358859 | protein_coding | 1.619618891 | 5.37E−47 | FALSE | FALSE | FALSE | FALSE | FALSE | FALSE |
| ENSG00000088340 | FER1L4 | 20 | 34146507 | 34195484 | pseudogene | 1.619381886 | 1.13E−14 | FALSE | FALSE | FALSE | FALSE | FALSE | FALSE |
| ENSG00000105173 | CCNE1 | 19 | 30302805 | 30315215 | protein_coding | 1.615384756 | 1.01E−38 | FALSE | FALSE | FALSE | FALSE | FALSE | FALSE |
| ENSG00000105664 | COMP | 19 | 18893583 | 18902123 | protein_coding | 1.613575912 | 3.68E−07 | FALSE | FALSE | FALSE | FALSE | FALSE | FALSE |
| ENSG00000103253 | HAGHL | 16 | 776936 | 785525 | protein_coding | 1.612033652 | 1.32E−24 | FALSE | FALSE | FALSE | FALSE | FALSE | FALSE |
| ENSG00000134057 | CCNB1 | 5 | 68462837 | 68474072 | protein_coding | 1.611874969 | 7.57E−40 | FALSE | FALSE | FALSE | FALSE | FALSE | TRUE |
| ENSG00000169248 | CXCL11 | 4 | 76954835 | 76962568 | protein_coding | 1.610428479 | 2.12E−15 | FALSE | FALSE | FALSE | FALSE | FALSE | TRUE |
| ENSG00000086717 | PPEF1 | X | 18694029 | 18846039 | protein_coding | 1.606875161 | 3.67E−19 | FALSE | FALSE | FALSE | FALSE | FALSE | FALSE |
| ENSG00000113739 | STC2 | 5 | 172741716 | 172756506 | protein_coding | 1.606143089 | 9.33E−18 | FALSE | FALSE | FALSE | FALSE | FALSE | TRUE |
| ENSG00000173894 | CBX2 | 17 | 77751931 | 77761782 | protein_coding | 1.606059038 | 5.25E−29 | FALSE | FALSE | FALSE | FALSE | FALSE | FALSE |
| ENSG00000164935 | DCSTAMP | 8 | 105351315 | 105368917 | protein_coding | 1.605892444 | 4.14E−07 | FALSE | FALSE | FALSE | FALSE | FALSE | FALSE |
| ENSG00000119547 | ONECUT2 | 18 | 55102917 | 55158529 | protein_coding | 1.600613355 | 7.01E−13 | FALSE | FALSE | FALSE | FALSE | TRUE | TRUE |
| ENSG00000085840 | ORC1 | 1 | 52838501 | 52870131 | protein_coding | 1.574943943 | 2.25E−28 | FALSE | FALSE | FALSE | FALSE | FALSE | FALSE |
| ENSG00000143590 | EFNA3 | 1 | 155036224 | 155060014 | protein_coding | 1.56824043 | 5.06E−31 | FALSE | FALSE | FALSE | FALSE | FALSE | FALSE |
| ENSG00000227992 | | 2 | 111961541 | 111963792 | pseudogene | 1.563100081 | 2.38E−31 | FALSE | FALSE | FALSE | FALSE | FALSE | FALSE |
| ENSG00000101003 | GINS1 | 20 | 25388363 | 25433264 | protein_coding | 1.562332807 | 1.16E−33 | FALSE | FALSE | FALSE | FALSE | FALSE | TRUE |
| ENSG00000255624 | | 10 | 124639246 | 124658230 | pseudogene | 1.560158089 | 2.69E−19 | FALSE | FALSE | FALSE | FALSE | FALSE | FALSE |
| ENSG00000180785 | OR51E1 | 11 | 4664650 | 4676718 | protein_coding | 1.552275075 | 4.95E−18 | FALSE | FALSE | FALSE | TRUE | TRUE | TRUE |
| ENSG00000123473 | STIL | 1 | 47715811 | 47779819 | protein_coding | 1.549841243 | 7.04E−47 | FALSE | FALSE | FALSE | FALSE | FALSE | FALSE |
| ENSG00000213420 | GPC2 | 7 | 99767229 | 99774995 | protein_coding | 1.549008184 | 1.71E−27 | FALSE | FALSE | FALSE | FALSE | FALSE | FALSE |
| ENSG00000167900 | TK1 | 17 | 76170160 | 76183314 | protein_coding | 1.545449786 | 4.10E−29 | FALSE | FALSE | FALSE | FALSE | FALSE | TRUE |
| ENSG00000101115 | SALL4 | 20 | 50400581 | 50419059 | protein_coding | 1.545094417 | 2.33E−11 | FALSE | FALSE | FALSE | FALSE | FALSE | FALSE |
| ENSG00000228168 | HNRNPA1P21 | 3 | 39376470 | 39377430 | pseudogene | 1.529737348 | 1.60E−21 | FALSE | FALSE | FALSE | TRUE | FALSE | TRUE |
| ENSG00000267374 | LINC00669 | 18 | 36786888 | 37380282 | lincRNA | 1.52739589 | 3.84E−11 | FALSE | FALSE | FALSE | FALSE | FALSE | FALSE |
| ENSG00000138160 | KIF11 | 10 | 94353043 | 94415150 | protein_coding | 1.527249722 | 1.97E−35 | FALSE | FALSE | FALSE | FALSE | FALSE | TRUE |
| ENSG00000172927 | MYEOV | 11 | 69061605 | 69182494 | protein_coding | 1.526327478 | 6.47E−07 | FALSE | FALSE | FALSE | FALSE | FALSE | FALSE |
| ENSG00000181218 | HIST3H2A | 1 | 228645065 | 228645560 | protein_coding | 1.524236621 | 1.73E−21 | FALSE | FALSE | FALSE | FALSE | FALSE | FALSE |
| ENSG00000101255 | TRIB3 | 20 | 361261 | 378203 | protein_coding | 1.523865317 | 1.11E−26 | FALSE | FALSE | FALSE | FALSE | FALSE | FALSE |
| ENSG00000272666 | | 22 | 50980734 | 50981335 | lincRNA | 1.518711429 | 3.43E−18 | FALSE | FALSE | FALSE | FALSE | FALSE | FALSE |
| ENSG00000254726 | MEX3A | 1 | 156041804 | 156051789 | protein_coding | 1.507047018 | 5.86E−25 | FALSE | TRUE | TRUE | TRUE | TRUE | TRUE |
| ENSG00000144354 | CDCA7 | 2 | 174219548 | 174233725 | protein_coding | 1.505410664 | 4.00E−20 | FALSE | FALSE | FALSE | FALSE | FALSE | TRUE |
| ENSG00000160957 | RECQL4 | 8 | 145736667 | 145743229 | protein_coding | 1.504359772 | 1.62E−36 | FALSE | FALSE | FALSE | FALSE | FALSE | FALSE |
| ENSG00000178773 | CPNE7 | 16 | 89642176 | 89663654 | protein_coding | 1.496996345 | 7.20E−10 | FALSE | FALSE | FALSE | FALSE | FALSE | FALSE |
| ENSG00000131015 | ULBP2 | 6 | 150263136 | 150270371 | protein_coding | 1.492008205 | 4.26E−19 | FALSE | FALSE | FALSE | FALSE | FALSE | FALSE |
| ENSG00000142185 | TRPM2 | 21 | 45770046 | 45862964 | protein_coding | 1.490058203 | 1.02E−32 | FALSE | FALSE | FALSE | FALSE | FALSE | FALSE |
| ENSG00000171208 | NETO2 | 16 | 47111614 | 47177908 | protein_coding | 1.486697441 | 2.50E−22 | FALSE | FALSE | FALSE | FALSE | FALSE | FALSE |
| ENSG00000134013 | LOXL2 | 8 | 23154702 | 23282841 | protein_coding | 1.48417541 | 9.56E−28 | FALSE | FALSE | FALSE | FALSE | TRUE | FALSE |
| ENSG00000104899 | AMH | 19 | 2249308 | 2252072 | protein_coding | 1.479675885 | 8.86E−15 | FALSE | FALSE | FALSE | FALSE | FALSE | FALSE |
| ENSG00000104951 | IL411 | 19 | 50392911 | 50432796 | protein_coding | 1.474292411 | 2.36E−21 | FALSE | FALSE | FALSE | FALSE | FALSE | FALSE |
| ENSG00000006074 | CCL18 | 17 | 34391640 | 34399392 | protein_coding | 1.471945335 | 4.12E−07 | FALSE | FALSE | FALSE | FALSE | FALSE | FALSE |
| ENSG00000147509 | RGS20 | 8 | 54764368 | 54871863 | protein_coding | 1.471876993 | 1.78E−11 | FALSE | FALSE | FALSE | FALSE | FALSE | FALSE |
| ENSG00000137135 | ARHGEF39 | 9 | 35658872 | 35675863 | protein_coding | 1.465635206 | 8.96E−47 | FALSE | FALSE | FALSE | FALSE | FALSE | FALSE |
| ENSG00000137868 | STRA6 | 15 | 74471807 | 74504608 | protein_coding | 1.464160098 | 0.000131711 | FALSE | FALSE | FALSE | FALSE | FALSE | FALSE |
| ENSG00000186891 | TNFRSF18 | 1 | 1138888 | 1142071 | protein_coding | 1.463722149 | 1.36E−17 | FALSE | FALSE | FALSE | FALSE | FALSE | FALSE |
| ENSG00000125319 | C17orf53 | 17 | 42219274 | 42239844 | protein_coding | 1.461244322 | 4.70E−39 | FALSE | FALSE | FALSE | FALSE | FALSE | FALSE |
| ENSG00000114346 | ECT2 | 3 | 172468472 | 172539264 | protein_coding | 1.461222954 | 6.87E−42 | FALSE | FALSE | FALSE | FALSE | FALSE | TRUE |
| ENSG00000196550 | FAM72A | 1 | 206136916 | 206155151 | protein_coding | 1.456368124 | 2.53E−31 | FALSE | FALSE | FALSE | FALSE | FALSE | FALSE |
| ENSG00000237686 | | 6 | 43963460 | 44042389 | antisense | 1.456304079 | 5.20E−11 | FALSE | FALSE | FALSE | FALSE | FALSE | FALSE |
| ENSG00000061656 | SPAG4 | 20 | 34203814 | 34208971 | protein_coding | 1.453847143 | 1.71E−22 | FALSE | FALSE | FALSE | FALSE | FALSE | FALSE |
| ENSG00000270547 | | 9 | 13406379 | 13433052 | lincRNA | 1.445254528 | 4.95E−10 | FALSE | FALSE | FALSE | FALSE | FALSE | FALSE |
| ENSG00000164109 | MAD2L1 | 4 | 120976763 | 120988229 | protein_coding | 1.436554497 | 3.53E−41 | FALSE | FALSE | FALSE | FALSE | FALSE | TRUE |
| ENSG00000146070 | PLA2G7 | 6 | 46671938 | 46703430 | protein_coding | 1.435950197 | 7.53E−14 | FALSE | FALSE | FALSE | FALSE | TRUE | FALSE |
| ENSG00000169245 | CXCL10 | 4 | 76942273 | 76944650 | protein_coding | 1.435132699 | 1.59E−14 | FALSE | FALSE | FALSE | FALSE | TRUE | TRUE |
| ENSG00000123610 | TNFAIP6 | 2 | 152214106 | 152236560 | protein_coding | 1.428687915 | 2.18E−08 | FALSE | FALSE | FALSE | FALSE | FALSE | FALSE |
| ENSG00000160161 | CILP2 | 19 | 19649057 | 19657468 | protein_coding | 1.425254337 | 6.32E−13 | FALSE | FALSE | FALSE | FALSE | TRUE | FALSE |
| ENSG00000227036 | LINC00511 | 17 | 70319264 | 70636611 | lincRNA | 1.425059124 | 3.63E−13 | FALSE | FALSE | FALSE | FALSE | FALSE | TRUE |
| ENSG00000073111 | MCM2 | 3 | 127317066 | 127341276 | protein_coding | 1.423661625 | 4.00E−44 | FALSE | FALSE | FALSE | FALSE | FALSE | TRUE |
| ENSG00000233818 | | 21 | 37818029 | 37904706 | antisense | 1.412308639 | 1.33E−16 | FALSE | FALSE | FALSE | FALSE | FALSE | FALSE |
| ENSG00000086991 | NOX4 | 11 | 89057524 | 89322779 | protein_coding | 1.411395204 | 1.22E−11 | FALSE | FALSE | FALSE | TRUE | TRUE | FALSE |
| ENSG00000110492 | MDK | 11 | 46402306 | 46405375 | protein_coding | 1.409734313 | 4.01E−22 | FALSE | FALSE | FALSE | TRUE | FALSE | TRUE |
| ENSG00000196460 | RFX8 | 2 | 102013823 | 102091165 | protein_coding | 1.406207531 | 3.09E−15 | FALSE | FALSE | FALSE | FALSE | FALSE | FALSE |
| ENSG00000100479 | POLE2 | 14 | 50110273 | 50155140 | protein_coding | 1.404742291 | 3.11E−41 | FALSE | FALSE | FALSE | FALSE | FALSE | FALSE |
| ENSG00000272620 | AFAP1-AS1 | 4 | 7755817 | 7780655 | antisense | 1.404126004 | 1.08E−05 | FALSE | FALSE | FALSE | FALSE | FALSE | FALSE |
| ENSG00000131188 | PRR7 | 5 | 176873446 | 176883283 | protein_coding | 1.398652822 | 2.87E−33 | FALSE | FALSE | FALSE | FALSE | FALSE | FALSE |
| ENSG00000256940 | | 11 | 64013436 | 64015689 | antisense | 1.397002272 | 2.65E−19 | FALSE | FALSE | FALSE | FALSE | FALSE | FALSE |
| ENSG00000133466 | C1QTNF6 | 22 | 37576207 | 37595425 | protein_coding | 1.395493704 | 2.09E−35 | FALSE | FALSE | FALSE | FALSE | FALSE | FALSE |
| ENSG00000251003 | | 8 | 106792474 | 107072752 | processed_transc [symbol] | 1.392441843 | 2.17E−07 | FALSE | FALSE | FALSE | FALSE | FALSE | FALSE |
| ENSG00000259807 | | 16 | 29228491 | 29231352 | lincRNA | 1.385129781 | 4.65E−14 | FALSE | FALSE | FALSE | FALSE | FALSE | FALSE |
| ENSG00000237517 | DGCR5 | 22 | 18958027 | 19018755 | antisense | 1.384462124 | 2.38E−10 | FALSE | FALSE | FALSE | FALSE | FALSE | FALSE |
| ENSG00000197757 | HOXC6 | 12 | 54384408 | 54424607 | protein_coding | 1.382101988 | 6.23E−10 | FALSE | FALSE | FALSE | FALSE | FALSE | TRUE |
| ENSG00000140451 | PIF1 | 15 | 65107831 | 65117867 | protein_coding | 1.378008548 | 2.23E−30 | FALSE | FALSE | FALSE | FALSE | FALSE | FALSE |
| ENSG00000234380 | | 21 | 36118054 | 36157183 | antisense | 1.376970769 | 6.61E−11 | FALSE | FALSE | FALSE | TRUE | FALSE | FALSE |
| ENSG00000203813 | HIST1H3H | 6 | 27777842 | 27778314 | protein_coding | 1.371068023 | 6.03E−16 | FALSE | FALSE | FALSE | FALSE | FALSE | FALSE |
| ENSG00000156509 | FBXO43 | 8 | 101145588 | 101158028 | protein_coding | 1.369768906 | 3.26E−18 | FALSE | FALSE | FALSE | FALSE | FALSE | FALSE |
| ENSG00000103888 | KIAA1199 | 15 | 81071684 | 81244117 | protein_coding | 1.357842921 | 4.76E−13 | FALSE | FALSE | FALSE | FALSE | FALSE | FALSE |
| ENSG00000122952 | ZWINT | 10 | 58116989 | 58121036 | protein_coding | 1.356798551 | 8.96E−28 | FALSE | FALSE | FALSE | FALSE | FALSE | TRUE |
| ENSG00000049249 | TNFRSF9 | 1 | 7979907 | 8000926 | protein_coding | 1.354408239 | 1.05E−10 | FALSE | FALSE | FALSE | FALSE | TRUE | FALSE |
| ENSG00000171241 | SHCBP1 | 16 | 46614466 | 46655538 | protein_coding | 1.349973521 | 2.28E−16 | FALSE | FALSE | FALSE | FALSE | FALSE | FALSE |
| ENSG00000134668 | SPOCD1 | 1 | 32256023 | 32281652 | protein_coding | 1.345581906 | 3.78E−10 | FALSE | FALSE | FALSE | FALSE | FALSE | FALSE |
| ENSG00000258227 | CLEC5A | 7 | 141627157 | 141646807 | protein_coding | 1.341396915 | 7.69E−14 | FALSE | FALSE | FALSE | FALSE | FALSE | FALSE |
| ENSG00000175305 | CCNE2 | 8 | 95891998 | 95908906 | protein_coding | 1.337574359 | 2.39E−34 | FALSE | FALSE | FALSE | FALSE | FALSE | FALSE |
| ENSG00000108813 | DLX4 | 17 | 48046334 | 48052321 | protein_coding | 1.335904 | 2.89E−13 | FALSE | FALSE | FALSE | FALSE | FALSE | FALSE |
| ENSG00000169684 | CHRNA5 | 15 | 78857862 | 78887611 | protein_coding | 1.332151802 | 1.04E−20 | FALSE | FALSE | FALSE | TRUE | TRUE | FALSE |
| ENSG00000175643 | RMI2 | 16 | 11343476 | 11445619 | protein_coding | 1.331691056 | 2.53E−31 | FALSE | FALSE | FALSE | FALSE | FALSE | TRUE |
| ENSG00000113763 | UNC5A | 5 | 176237478 | 176307897 | protein_coding | 1.331097872 | 3.23E−07 | FALSE | FALSE | FALSE | FALSE | FALSE | FALSE |
| ENSG00000205426 | KRT81 | 12 | 52679697 | 52685318 | protein_coding | 1.328531679 | 5.28E−08 | FALSE | FALSE | FALSE | FALSE | FALSE | FALSE |
| ENSG00000171388 | APLN | X | 128779240 | 128788933 | protein_coding | 1.324632567 | 6.67E−12 | FALSE | FALSE | FALSE | FALSE | FALSE | FALSE |
| ENSG00000175832 | ETV4 | 17 | 41605212 | 41656988 | protein_coding | 1.324094084 | 4.86E−08 | FALSE | FALSE | FALSE | FALSE | TRUE | FALSE |
| ENSG00000142731 | PLK4 | 4 | 128802016 | 128820350 | protein_coding | 1.323486491 | 4.34E−39 | FALSE | FALSE | FALSE | FALSE | FALSE | FALSE |
| ENSG00000179750 | APOBEC3B | 22 | 39378352 | 39388809 | protein_coding | 1.321640493 | 1.32E−16 | FALSE | FALSE | FALSE | FALSE | FALSE | FALSE |
| ENSG00000139734 | DIAPH3 | 13 | 60239717 | 60738121 | protein_coding | 1.319911775 | 2.34E−19 | FALSE | FALSE | FALSE | FALSE | FALSE | TRUE |
| ENSG00000013810 | TACC3 | 4 | 1723227 | 1746898 | protein_coding | 1.319368805 | 1.14E−40 | FALSE | FALSE | FALSE | FALSE | FALSE | FALSE |
| ENSG00000162745 | OLFML2B | 1 | 161952982 | 161993644 | protein_coding | 1.316592671 | 6.57E−21 | FALSE | FALSE | FALSE | FALSE | TRUE | FALSE |
| ENSG00000117394 | SLC2A1 | 1 | 43391052 | 43424530 | protein_coding | 1.312604153 | 3.35E−15 | FALSE | FALSE | FALSE | FALSE | FALSE | TRUE |
| ENSG00000188610 | FAM72B | 1 | 120837756 | 120855681 | protein_coding | 1.311684667 | 2.13E−24 | FALSE | FALSE | FALSE | FALSE | FALSE | FALSE |
| ENSG00000139618 | BRCA2 | 13 | 32889611 | 32973805 | protein_coding | 1.311191928 | 5.76E−29 | FALSE | FALSE | FALSE | FALSE | FALSE | FALSE |
| ENSG00000162063 | CCNF | 16 | 2479395 | 2508855 | protein_coding | 1.308644581 | 2.19E−40 | FALSE | FALSE | FALSE | FALSE | FALSE | FALSE |
| ENSG00000099194 | SCD | 10 | 102106881 | 102124591 | protein_coding | 1.301922672 | 1.27E−13 | FALSE | FALSE | FALSE | FALSE | FALSE | FALSE |
| ENSG00000155265 | GOLGA7B | 10 | 99609996 | 99631337 | protein_coding | 1.298277448 | 2.20E−11 | FALSE | FALSE | FALSE | FALSE | FALSE | FALSE |
| ENSG00000187951 | ARHGAP11B | 15 | 30916697 | 31065196 | protein_coding | 1.295086795 | 1.16E−38 | FALSE | FALSE | FALSE | FALSE | FALSE | FALSE |
| ENSG00000256694 | | 12 | 273830 | 275487 | antisense | 1.290977068 | 3.59E−12 | FALSE | FALSE | FALSE | FALSE | FALSE | FALSE |
| ENSG00000162004 | CCDC78 | 16 | 772582 | 776954 | protein_coding | 1.287430304 | 6.19E−10 | FALSE | FALSE | FALSE | FALSE | FALSE | FALSE |
| ENSG00000087494 | PTHLH | 12 | 28111017 | 28125638 | protein_coding | 1.285098029 | 0.006354184 | FALSE | FALSE | FALSE | FALSE | FALSE | TRUE |
| ENSG00000038427 | VCAN | 5 | 82767284 | 82878122 | protein_coding | 1.283149106 | 1.85E−13 | FALSE | FALSE | FALSE | FALSE | FALSE | FALSE |
| ENSG00000204262 | COL5A2 | 2 | 189896622 | 190044605 | protein_coding | 1.281417182 | 1.88E−20 | FALSE | FALSE | FALSE | FALSE | FALSE | FALSE |
| ENSG00000213886 | UBD | 6 | 29523292 | 29527702 | protein_coding | 1.279572287 | 5.69E−07 | FALSE | FALSE | FALSE | TRUE | FALSE | TRUE |
| ENSG00000185567 | AHNAK2 | 14 | 105403581 | 105444694 | protein_coding | 1.278453234 | 1.49E−07 | FALSE | FALSE | FALSE | FALSE | FALSE | FALSE |
| ENSG00000128683 | GAD1 | 2 | 171669723 | 171717661 | protein_coding | 1.274551206 | 7.08E−05 | FALSE | FALSE | FALSE | FALSE | FALSE | FALSE |
| ENSG00000156802 | ATAD2 | 8 | 124332090 | 124428590 | protein_coding | 1.274265142 | 1.62E−47 | FALSE | FALSE | FALSE | FALSE | FALSE | FALSE |
| ENSG00000139572 | GPR84 | 12 | 54756229 | 54758271 | protein_coding | 1.271985352 | 9.03E−16 | FALSE | FALSE | FALSE | FALSE | FALSE | FALSE |
| ENSG00000176890 | TYMS | 18 | 657604 | 673578 | protein_coding | 1.268098746 | 2.87E−31 | FALSE | FALSE | FALSE | FALSE | FALSE | FALSE |
| ENSG00000187642 | C1orf170 | 1 | 910579 | 917497 | protein_coding | 1.267375485 | 2.16E−07 | FALSE | FALSE | FALSE | FALSE | TRUE | FALSE |
| ENSG00000184445 | KNTC1 | 12 | 123011793 | 123110943 | protein_coding | 1.264458262 | 1.39E−50 | FALSE | FALSE | FALSE | FALSE | FALSE | FALSE |
| ENSG00000196787 | HIST1H2AG | 6 | 27100832 | 27103070 | protein_coding | 1.261738524 | 5.32E−16 | FALSE | FALSE | FALSE | FALSE | FALSE | FALSE |
| ENSG00000122641 | INHBA | 7 | 41724712 | 41742706 | protein_coding | 1.259740346 | 1.51E−09 | FALSE | FALSE | FALSE | FALSE | FALSE | FALSE |
| ENSG00000113368 | LMNB1 | 5 | 126112315 | 126172712 | protein_coding | 1.255519127 | 5.82E−28 | FALSE | FALSE | FALSE | FALSE | FALSE | FALSE |
| ENSG00000198518 | HIST1H4E | 6 | 26204858 | 26206266 | protein_coding | 1.252433382 | 2.52E−10 | FALSE | FALSE | FALSE | FALSE | FALSE | FALSE |
| ENSG00000121904 | CSMD2 | 1 | 33979609 | 34631443 | protein_coding | 1.249971092 | 4.58E−13 | FALSE | FALSE | FALSE | FALSE | FALSE | FALSE |
| ENSG00000185480 | PARPBP | 12 | 102513956 | 102591298 | protein_coding | 1.249241701 | 9.99E−28 | FALSE | FALSE | FALSE | FALSE | FALSE | FALSE |
| ENSG00000245614 | DDX11-AS1 | 12 | 31173697 | 31226781 | antisense | 1.248182612 | 3.91E−28 | FALSE | FALSE | FALSE | FALSE | FALSE | FALSE |
| ENSG00000245522 | | 11 | 9776317 | 9781080 | lincRNA | 1.245123155 | 1.72E−22 | FALSE | FALSE | FALSE | FALSE | FALSE | FALSE |
| ENSG00000185633 | NDUFA4L2 | 12 | 57628686 | 57634498 | protein_coding | 1.244143398 | 1.37E−08 | FALSE | FALSE | FALSE | FALSE | TRUE | TRUE |
| ENSG00000253293 | HOXA10 | 7 | 27210210 | 27219880 | protein_coding | 1.243236868 | 4.40E−07 | FALSE | FALSE | FALSE | FALSE | FALSE | TRUE |
| ENSG00000169385 | RNASE2 | 14 | 21423611 | 21424595 | protein_coding | 1.242952589 | 5.48E−11 | FALSE | FALSE | FALSE | FALSE | FALSE | FALSE |
| ENSG00000147536 | GINS4 | 8 | 41386725 | 41402565 | protein_coding | 1.239718596 | 2.21E−26 | FALSE | FALSE | FALSE | FALSE | FALSE | FALSE |
| ENSG00000270933 | | 7 | 25988277 | 25989023 | lincRNA | 1.238568409 | 3.76E−12 | FALSE | FALSE | FALSE | FALSE | FALSE | FALSE |
| ENSG00000136982 | DSCC1 | 8 | 120846216 | 120868250 | protein_coding | 1.236553778 | 8.16E−36 | FALSE | FALSE | FALSE | FALSE | FALSE | FALSE |
| ENSG00000161800 | RACGAP1 | 12 | 50370706 | 50426919 | protein_coding | 1.231238201 | 1.52E−41 | FALSE | FALSE | FALSE | FALSE | FALSE | TRUE |
| ENSG00000179528 | LBX2 | 2 | 74724644 | 74730443 | protein_coding | 1.231027615 | 3.80E−18 | FALSE | FALSE | FALSE | FALSE | FALSE | FALSE |
| ENSG00000214826 | DDX12P | 12 | 9570309 | 9600825 | pseudogene | 1.227247158 | 1.14E−31 | FALSE | FALSE | FALSE | FALSE | FALSE | FALSE |
| ENSG00000224126 | UBE2SP2 | 17 | 18580574 | 18581070 | pseudogene | 1.226057661 | 9.52E−32 | FALSE | FALSE | FALSE | FALSE | FALSE | FALSE |
| ENSG00000186827 | TNFRSF4 | 1 | 1146706 | 1149518 | protein_coding | 1.225407903 | 1.65E−17 | FALSE | FALSE | FALSE | FALSE | FALSE | FALSE |
| ENSG00000077935 | SMC1B | 22 | 45739944 | 45809500 | protein_coding | 1.221367034 | 2.73E−10 | FALSE | FALSE | FALSE | FALSE | FALSE | FALSE |
| ENSG00000181544 | FANCB | X | 14861529 | 14891191 | protein_coding | 1.215479681 | 2.08E−23 | FALSE | FALSE | FALSE | FALSE | FALSE | FALSE |
| ENSG00000120254 | MTHFD1L | 6 | 151186685 | 151423023 | protein_coding | 1.212579338 | 1.50E−40 | FALSE | FALSE | FALSE | FALSE | FALSE | FALSE |
| ENSG00000050344 | NFE2L3 | 7 | 26191860 | 26226745 | protein_coding | 1.211292401 | 1.37E−22 | FALSE | FALSE | FALSE | FALSE | FALSE | FALSE |
| ENSG00000138755 | CXCL9 | 4 | 76922428 | 76928641 | protein_coding | 1.208015449 | 1.21E−08 | FALSE | FALSE | FALSE | FALSE | FALSE | FALSE |
| ENSG00000165490 | C11orf82 | 11 | 82611017 | 82669319 | protein_coding | 1.207898496 | 1.94E−27 | FALSE | FALSE | FALSE | FALSE | FALSE | TRUE |
| ENSG00000163507 | KIAA1524 | 3 | 108268716 | 108308491 | protein_coding | 1.207558176 | 5.87E−26 | FALSE | FALSE | FALSE | FALSE | FALSE | FALSE |
| ENSG00000151388 | ADAMTS12 | 5 | 33523640 | 33892297 | protein_coding | 1.205996127 | 6.50E−12 | FALSE | FALSE | FALSE | FALSE | FALSE | TRUE |
| ENSG00000135245 | HILPDA | 7 | 128095903 | 128098472 | protein_coding | 1.203101081 | 9.13E−12 | FALSE | FALSE | FALSE | FALSE | FALSE | FALSE |
| ENSG00000138028 | CGREF1 | 2 | 27321757 | 27341995 | protein_coding | 1.20275533 | 2.22E−07 | FALSE | FALSE | FALSE | FALSE | FALSE | FALSE |
| ENSG00000172965 | MIR4435-1HC | 2 | 111953927 | 112252677 | lincRNA | 1.200738631 | 2.68E−32 | FALSE | FALSE | FALSE | FALSE | FALSE | FALSE |
| ENSG00000153044 | CENPH | 5 | 68485375 | 68506184 | protein_coding | 1.196462961 | 9.15E−43 | FALSE | FALSE | FALSE | FALSE | FALSE | FALSE |
| ENSG00000128165 | ADM2 | 22 | 50919985 | 50924869 | protein_coding | 1.196050302 | 7.23E−10 | FALSE | FALSE | FALSE | FALSE | FALSE | FALSE |
| ENSG00000154040 | CABYR | 18 | 21718942 | 21741567 | protein_coding | 1.193775544 | 7.77E−12 | FALSE | FALSE | FALSE | FALSE | FALSE | FALSE |
| ENSG00000206630 | SNORD60 | 16 | 2205024 | 2205106 | snoRNA | 1.192946553 | 5.75E−18 | FALSE | FALSE | FALSE | FALSE | FALSE | FALSE |
| ENSG00000183763 | TRAIP | 3 | 49866034 | 49894007 | protein_coding | 1.192256758 | 8.49E−31 | FALSE | FALSE | FALSE | FALSE | FALSE | FALSE |
| ENSG00000182272 | B4GALNT4 | 11 | 369796 | 382116 | protein_coding | 1.190598205 | 0.00018244 | FALSE | FALSE | FALSE | FALSE | FALSE | FALSE |
| ENSG00000243449 | C4orf48 | 4 | 2043689 | 2045697 | protein_coding | 1.190463011 | 5.88E−12 | FALSE | FALSE | FALSE | FALSE | FALSE | FALSE |
| ENSG00000273032 | DGCR9 | 22 | 19005347 | 19007761 | lincRNA | 1.190127823 | 9.86E−09 | FALSE | FALSE | FALSE | FALSE | FALSE | FALSE |
| ENSG00000150337 | FCGR1A | 1 | 149754227 | 149764074 | protein_coding | 1.185229671 | 3.45E−10 | FALSE | FALSE | FALSE | FALSE | FALSE | FALSE |
| ENSG00000178752 | FAM132B | 2 | 239067623 | 239077541 | protein_coding | 1.184728034 | 3.24E−06 | FALSE | FALSE | FALSE | FALSE | FALSE | FALSE |
| ENSG00000135476 | ESPL1 | 12 | 53662083 | 53687427 | protein_coding | 1.183432617 | 2.85E−10 | FALSE | FALSE | FALSE | FALSE | FALSE | FALSE |
| ENSG00000213186 | TRIM59 | 3 | 160150233 | 160203561 | protein_coding | 1.181236164 | 1.83E−38 | FALSE | FALSE | FALSE | FALSE | FALSE | FALSE |
| ENSG00000119969 | HELLS | 10 | 96305547 | 96373662 | protein_coding | 1.180162819 | 2.85E−21 | FALSE | FALSE | FALSE | FALSE | FALSE | FALSE |
| ENSG00000213070 | HMGB3P6 | 1 | 164326004 | 164326601 | pseudogene | 1.179383142 | 3.47E−13 | FALSE | FALSE | TRUE | TRUE | FALSE | TRUE |
| ENSG00000108106 | UBE2S | 19 | 55912652 | 55919145 | protein_coding | 1.178122563 | 8.65E−36 | FALSE | FALSE | FALSE | FALSE | FALSE | FALSE |
| ENSG00000272183 | | 2 | 74728844 | 74729492 | antisense | 1.177952732 | 1.73E−17 | FALSE | FALSE | FALSE | FALSE | FALSE | FALSE |
| ENSG00000124140 | SLC12A5 | 20 | 44650356 | 44688784 | protein_coding | 1.177136936 | 4.67E−13 | FALSE | FALSE | FALSE | FALSE | FALSE | FALSE |
| ENSG00000160949 | TONSL | 8 | 145654165 | 145669827 | protein_coding | 1.173687206 | 1.26E−31 | FALSE | FALSE | FALSE | FALSE | FALSE | FALSE |
| ENSG00000230479 | | 21 | 37802658 | 37853368 | antisense | 1.173428153 | 6.97E−11 | FALSE | FALSE | FALSE | FALSE | FALSE | FALSE |
| ENSG00000186818 | LILRB4 | 19 | 55155340 | 55181810 | protein_coding | 1.173169637 | 1.99E−11 | FALSE | FALSE | FALSE | FALSE | FALSE | FALSE |
| ENSG00000147570 | DNAJC5B | 8 | 66933795 | 67012751 | protein_coding | 1.172626184 | 0.000182583 | FALSE | FALSE | FALSE | FALSE | FALSE | FALSE |
| ENSG00000080573 | COL5A3 | 19 | 10070237 | 10121147 | protein_coding | 1.167287705 | 2.50E−13 | FALSE | FALSE | FALSE | FALSE | FALSE | FALSE |
| ENSG00000175294 | CATSPER1 | 11 | 65784223 | 65793988 | protein_coding | 1.166901997 | 7.36E−07 | FALSE | FALSE | FALSE | FALSE | FALSE | FALSE |
| ENSG00000133110 | POSTN | 13 | 38136720 | 38172981 | protein_coding | 1.166636722 | 1.01E−07 | FALSE | FALSE | FALSE | FALSE | FALSE | TRUE |
| ENSG00000105613 | MAST1 | 19 | 12944765 | 12985765 | protein_coding | 1.160835131 | 1.83E−10 | FALSE | FALSE | FALSE | FALSE | FALSE | FALSE |
| ENSG00000198535 | C2CD4A | 15 | 62359176 | 62363116 | protein_coding | 1.159840134 | 0.000782568 | FALSE | FALSE | FALSE | FALSE | FALSE | FALSE |
| ENSG00000172167 | MTBP | 8 | 121457640 | 121554373 | protein_coding | 1.158100901 | 3.67E−31 | FALSE | FALSE | FALSE | FALSE | FALSE | FALSE |
| ENSG00000151790 | TDO2 | 4 | 156775890 | 156841558 | protein_coding | 1.157363786 | 0.000143198 | FALSE | FALSE | FALSE | FALSE | FALSE | FALSE |
| ENSG00000136295 | TTYH3 | 7 | 2671585 | 2704436 | protein_coding | 1.155790532 | 2.20E−29 | FALSE | FALSE | FALSE | FALSE | FALSE | FALSE |
| ENSG00000233966 | UBE2SP1 | 17 | 15607546 | 15608214 | pseudogene | 1.155636441 | 5.65E−32 | FALSE | FALSE | FALSE | FALSE | FALSE | FALSE |
| ENSG00000100167 | 3-Sep | 22 | 42372276 | 42394225 | protein_coding | 1.155497139 | 2.21E−10 | FALSE | FALSE | FALSE | FALSE | FALSE | FALSE |
| ENSG00000011332 | DPF1 | 19 | 38701646 | 38720354 | protein_coding | 1.155488155 | 3.26E−13 | FALSE | FALSE | FALSE | FALSE | FALSE | FALSE |
| ENSG00000123975 | CKS2 | 9 | 91926113 | 91931618 | protein_coding | 1.154311869 | 2.42E−25 | FALSE | FALSE | FALSE | FALSE | FALSE | TRUE |
| ENSG00000167550 | RHEBL1 | 12 | 49458468 | 49463808 | protein_coding | 1.153380525 | 1.30E−25 | FALSE | FALSE | FALSE | FALSE | FALSE | FALSE |
| ENSG00000237988 | OR211P | 6 | 29520996 | 29521943 | pseudogene | 1.1528557 | 4.05E−06 | FALSE | FALSE | FALSE | FALSE | FALSE | FALSE |
| ENSG00000237424 | FOXD2-AS1 | 1 | 47897805 | 47900313 | antisense | 1.150686824 | 1.04E−21 | FALSE | FALSE | FALSE | FALSE | FALSE | FALSE |
| ENSG00000198554 | WDHD1 | 14 | 55405668 | 55493823 | protein_coding | 1.147602855 | 1.17E−36 | FALSE | FALSE | FALSE | FALSE | FALSE | TRUE |
| ENSG00000159259 | CHAF1B | 21 | 37757676 | 37791313 | protein_coding | 1.14729946 | 4.32E−29 | FALSE | FALSE | FALSE | FALSE | FALSE | FALSE |
| ENSG00000130635 | COL5A1 | 9 | 137533620 | 137736686 | protein_coding | 1.146318635 | 5.31E−12 | FALSE | FALSE | FALSE | FALSE | FALSE | FALSE |
| ENSG00000163673 | DCLK3 | 3 | 36753913 | 36781352 | protein_coding | 1.14591052 | 7.30E−11 | FALSE | FALSE | FALSE | FALSE | FALSE | FALSE |
| ENSG00000125657 | TNFSF9 | 19 | 6531010 | 6535931 | protein_coding | 1.143990764 | 6.71E−07 | FALSE | FALSE | FALSE | FALSE | FALSE | FALSE |
| ENSG00000101842 | VSIG1 | X | 107288200 | 107322414 | protein_coding | 1.141300257 | 0.000528911 | FALSE | FALSE | FALSE | FALSE | FALSE | FALSE |
| ENSG00000188372 | ZP3 | 7 | 76026835 | 76071388 | protein_coding | 1.13804313 | 2.64E−25 | FALSE | FALSE | FALSE | FALSE | FALSE | FALSE |
| ENSG00000135119 | RNFT2 | 12 | 117176096 | 117291436 | protein_coding | 1.137621419 | 9.68E−21 | FALSE | FALSE | FALSE | FALSE | TRUE | FALSE |
| ENSG00000256540 | | 12 | 276022 | 291565 | lincRNA | 1.136500241 | 1.46E−08 | FALSE | FALSE | FALSE | FALSE | FALSE | FALSE |
| ENSG00000127586 | CHTF18 | 16 | 838046 | 850737 | protein_coding | 1.136227661 | 1.86E−42 | FALSE | FALSE | FALSE | FALSE | FALSE | FALSE |
| ENSG00000106236 | NPTX2 | 7 | 98246609 | 98259180 | protein_coding | 1.135630061 | 0.044197968 | FALSE | FALSE | FALSE | FALSE | FALSE | FALSE |
| ENSG00000092607 | TBX15 | 1 | 119425669 | 119532179 | protein_coding | 1.132192516 | 5.01E−05 | FALSE | FALSE | FALSE | FALSE | FALSE | FALSE |
| ENSG00000186340 | THBS2 | 6 | 169615875 | 169654139 | protein_coding | 1.130508271 | 3.64E−09 | FALSE | FALSE | FALSE | FALSE | FALSE | FALSE |
| ENSG00000149554 | CHEK1 | 11 | 125495036 | 125546150 | protein_coding | 1.12994115 | 3.40E−30 | FALSE | FALSE | FALSE | FALSE | TRUE | TRUE |
| ENSG00000135454 | B4GALNT1 | 12 | 58017193 | 58027138 | protein_coding | 1.128208702 | 2.09E−09 | FALSE | FALSE | FALSE | FALSE | FALSE | FALSE |
| ENSG00000203760 | CENPW | 6 | 126661320 | 126670021 | protein_coding | 1.126460041 | 1.29E−23 | FALSE | FALSE | FALSE | FALSE | FALSE | FALSE |
| ENSG00000105327 | BBC3 | 19 | 47724081 | 47736023 | protein_coding | 1.123837101 | 4.21E−28 | FALSE | FALSE | FALSE | FALSE | FALSE | FALSE |
| ENSG00000144554 | FANCD2 | 3 | 10068098 | 10143614 | protein_coding | 1.120817101 | 7.34E−27 | FALSE | FALSE | FALSE | FALSE | FALSE | FALSE |
| ENSG00000198339 | HIST1H4I | 6 | 27107076 | 27108418 | protein_coding | 1.120514423 | 4.56E−14 | FALSE | FALSE | FALSE | FALSE | FALSE | FALSE |
| ENSG00000197409 | HIST1H3D | 6 | 26197068 | 26199521 | protein_coding | 1.117806641 | 5.14E−10 | FALSE | FALSE | FALSE | FALSE | FALSE | FALSE |
| ENSG00000266835 | | 18 | 3466248 | 3478976 | lincRNA | 1.115536435 | 1.39E−07 | FALSE | FALSE | FALSE | FALSE | FALSE | FALSE |
| ENSG00000104889 | RNASEH2A | 19 | 12917394 | 12924452 | protein_coding | 1.109899209 | 3.09E−50 | FALSE | FALSE | FALSE | TRUE | TRUE | TRUE |
| ENSG00000163013 | FBXO41 | 2 | 73481810 | 73511559 | protein_coding | 1.108810744 | 1.03E−17 | FALSE | FALSE | FALSE | FALSE | FALSE | FALSE |
| ENSG00000140678 | ITGAX | 16 | 31366455 | 31394318 | protein_coding | 1.108338185 | 1.21E−09 | FALSE | FALSE | FALSE | TRUE | TRUE | FALSE |
| ENSG00000267473 | | 19 | 38888821 | 38890521 | lincRNA | 1.107520153 | 2.19E−15 | FALSE | FALSE | FALSE | FALSE | FALSE | FALSE |
| ENSG00000179593 | ALOX15B | 17 | 7942335 | 7952452 | protein_coding | 1.104887963 | 0.012538331 | FALSE | FALSE | FALSE | FALSE | FALSE | FALSE |
| ENSG00000137812 | CASC5 | 15 | 40886218 | 40956540 | protein_coding | 1.104225865 | 2.07E−09 | FALSE | FALSE | FALSE | FALSE | FALSE | TRUE |
| ENSG00000138346 | DNA2 | 10 | 70173821 | 70231879 | protein_coding | 1.097729393 | 1.24E−25 | FALSE | FALSE | FALSE | FALSE | FALSE | FALSE |
| ENSG00000225614 | ZNF469 | 16 | 88493879 | 88507165 | protein_coding | 1.097657952 | 4.82E−14 | FALSE | FALSE | FALSE | FALSE | FALSE | FALSE |
| ENSG00000169783 | LINGO1 | 15 | 77905369 | 78113242 | protein_coding | 1.097642058 | 4.80E−13 | FALSE | FALSE | FALSE | FALSE | FALSE | FALSE |
| ENSG00000251442 | LINC01094 | 4 | 79567057 | 79603853 | lincRNA | 1.096727788 | 4.08E−12 | FALSE | FALSE | FALSE | FALSE | FALSE | FALSE |
| ENSG00000163689 | C3orf67 | 3 | 58703092 | 59035810 | protein_coding | 1.094384856 | 6.43E−10 | FALSE | FALSE | FALSE | FALSE | FALSE | FALSE |
| ENSG00000079462 | PAFAH1B3 | 19 | 42801185 | 42807698 | protein_coding | 1.092073955 | 6.96E−26 | FALSE | FALSE | FALSE | TRUE | TRUE | TRUE |
| ENSG00000107105 | ELAVL2 | 9 | 23690102 | 23826335 | protein_coding | 1.089630542 | 0.001482448 | FALSE | FALSE | FALSE | FALSE | FALSE | FALSE |
| ENSG00000172031 | EPHX4 | 1 | 92495539 | 92529093 | protein_coding | 1.089097148 | 1.10E−06 | FALSE | FALSE | FALSE | FALSE | FALSE | FALSE |
| ENSG00000163568 | AIM2 | 1 | 159032274 | 159116886 | protein_coding | 1.088104822 | 1.37E−05 | FALSE | FALSE | FALSE | FALSE | FALSE | FALSE |
| ENSG00000173227 | SYT12 | 11 | 66774249 | 66818334 | protein_coding | 1.08701921 | 0.000452117 | FALSE | FALSE | FALSE | FALSE | FALSE | FALSE |
| ENSG00000141526 | SLC16A3 | 17 | 80186273 | 80219005 | protein_coding | 1.085735477 | 8.77E−13 | FALSE | FALSE | FALSE | FALSE | FALSE | FALSE |
| ENSG00000167747 | C19orf48 | 19 | 51300961 | 51308186 | protein_coding | 1.085302484 | 1.55E−39 | FALSE | FALSE | FALSE | FALSE | FALSE | FALSE |
| ENSG00000149380 | P4HA3 | 11 | 73946846 | 74022702 | protein_coding | 1.083908123 | 7.12E−10 | FALSE | FALSE | FALSE | FALSE | FALSE | FALSE |
| ENSG00000240204 | SMKR1 | 7 | 129142320 | 129152773 | protein_coding | 1.08331565 | 1.50E−07 | FALSE | FALSE | FALSE | FALSE | FALSE | FALSE |
| ENSG00000129667 | RHBDF2 | 17 | 74466973 | 74497872 | protein_coding | 1.080020909 | 1.58E−30 | FALSE | FALSE | FALSE | FALSE | FALSE | FALSE |
| ENSG00000136378 | ADAMTS7 | 15 | 79051545 | 79103773 | protein_coding | 1.078027629 | 1.82E−18 | FALSE | FALSE | FALSE | FALSE | FALSE | FALSE |
| ENSG00000078900 | TP73 | 1 | 3569084 | 3652765 | protein_coding | 1.073465044 | 1.49E−11 | FALSE | FALSE | FALSE | FALSE | FALSE | FALSE |
| ENSG00000267750 | | 17 | 42376942 | 42392683 | antisense | 1.071758849 | 0.000154369 | FALSE | FALSE | FALSE | FALSE | FALSE | FALSE |
| ENSG00000131187 | F12 | 5 | 176829141 | 176836577 | protein_coding | 1.071163388 | 1.51E−05 | FALSE | FALSE | FALSE | FALSE | FALSE | FALSE |
| ENSG00000266088 | | 17 | 38673278 | 38683254 | lincRNA | 1.070821222 | 3.99E−05 | FALSE | FALSE | FALSE | FALSE | FALSE | FALSE |
| ENSG00000235884 | LINC00941 | 12 | 30947977 | 30955645 | lincRNA | 1.069843273 | 0.000117553 | FALSE | FALSE | FALSE | FALSE | FALSE | FALSE |
| ENSG00000261373 | VPS9D1-AS1 | 16 | 89778264 | 89784573 | antisense | 1.068352942 | 2.20E−11 | FALSE | FALSE | FALSE | FALSE | FALSE | FALSE |
| ENSG00000164045 | CDC25A | 3 | 48198636 | 48229892 | protein_coding | 1.067584134 | 1.20E−13 | FALSE | FALSE | FALSE | FALSE | FALSE | FALSE |
| ENSG00000168274 | HIST1H2AE | 6 | 26217165 | 26217711 | protein_coding | 1.067184976 | 1.42E−09 | FALSE | FALSE | FALSE | FALSE | FALSE | FALSE |
| ENSG00000203819 | HIST2H2BC | 1 | 149821760 | 149822339 | pseudogene | 1.066995648 | 2.96E−13 | FALSE | FALSE | FALSE | FALSE | FALSE | FALSE |
| ENSG00000162849 | KIF26B | 1 | 245318287 | 245872733 | protein_coding | 1.066718448 | 5.31E−07 | FALSE | FALSE | FALSE | TRUE | TRUE | FALSE |
| ENSG00000188282 | RUFY4 | 2 | 218899683 | 218955304 | protein_coding | 1.065982913 | 2.03E−05 | FALSE | FALSE | FALSE | FALSE | FALSE | FALSE |
| ENSG00000176749 | CDK5R1 | 17 | 30813637 | 30818274 | protein_coding | 1.065373131 | 1.12E−17 | FALSE | FALSE | FALSE | FALSE | FALSE | FALSE |
| ENSG00000215270 | | 22 | 16122720 | 16123768 | pseudogene | 1.064078426 | 3.11E−18 | FALSE | FALSE | FALSE | FALSE | FALSE | TRUE |
| ENSG00000128578 | STRIP2 | 7 | 129074274 | 129128240 | protein_coding | 1.061654833 | 4.06E−07 | FALSE | FALSE | FALSE | FALSE | FALSE | FALSE |
| ENSG00000179294 | C17orf96 | 17 | 36827961 | 36831187 | protein_coding | 1.060100008 | 6.32E−14 | FALSE | FALSE | FALSE | FALSE | FALSE | FALSE |
| ENSG00000170727 | BOP1 | 8 | 145486055 | 145515082 | protein_coding | 1.059364504 | 4.34E−21 | FALSE | FALSE | FALSE | FALSE | FALSE | FALSE |
| ENSG00000002079 | MYH16 | 7 | 98836417 | 98908753 | pseudogene | 1.05286495 | 4.64E−06 | FALSE | FALSE | FALSE | FALSE | FALSE | FALSE |
| ENSG00000157227 | MMP14 | 14 | 23305766 | 23318236 | protein_coding | 1.051568144 | 1.94E−24 | FALSE | FALSE | FALSE | FALSE | FALSE | FALSE |
| ENSG00000239672 | NME1 | 17 | 49230897 | 49239789 | protein_coding | 1.049822599 | 1.45E−32 | FALSE | FALSE | FALSE | FALSE | FALSE | FALSE |
| ENSG00000182481 | KPNA2 | 17 | 66031635 | 66042958 | protein_coding | 1.049367928 | 2.38E−25 | FALSE | FALSE | FALSE | FALSE | FALSE | TRUE |
| ENSG00000101194 | SLC17A9 | 20 | 61584052 | 61599949 | protein_coding | 1.04908634 | 3.40E−09 | FALSE | FALSE | FALSE | FALSE | FALSE | FALSE |
| ENSG00000169436 | COL22A1 | 8 | 139600478 | 139926249 | protein_coding | 1.046963373 | 0.001932418 | FALSE | FALSE | FALSE | FALSE | FALSE | FALSE |
| ENSG00000144485 | HES6 | 2 | 239146908 | 239149303 | protein_coding | 1.046846683 | 1.76E−17 | FALSE | FALSE | FALSE | FALSE | TRUE | TRUE |
| ENSG00000006634 | DBF4 | 7 | 87505531 | 87538856 | protein_coding | 1.04467258 | 6.30E−41 | FALSE | FALSE | FALSE | FALSE | FALSE | FALSE |
| ENSG00000122254 | HS3ST2 | 16 | 22825498 | 22927659 | protein_coding | 1.044356164 | 0.002263593 | FALSE | FALSE | FALSE | FALSE | FALSE | FALSE |
| ENSG00000203747 | FCGR3A | 1 | 161511549 | 161600917 | protein_coding | 1.043983777 | 4.00E−07 | FALSE | FALSE | FALSE | FALSE | TRUE | FALSE |
| ENSG00000188368 | PRR19 | 19 | 42806250 | 42814973 | protein_coding | 1.043697973 | 1.44E−16 | FALSE | FALSE | FALSE | FALSE | FALSE | TRUE |
| ENSG00000272468 | | 6 | 27090436 | 27091000 | lincRNA | 1.040509315 | 2.24E−08 | FALSE | FALSE | FALSE | FALSE | FALSE | FALSE |
| ENSG00000124635 | HIST1H2BJ | 6 | 27093676 | 27100541 | protein_coding | 1.035195909 | 2.17E−15 | FALSE | FALSE | FALSE | FALSE | FALSE | FALSE |
| ENSG00000159399 | HK2 | 2 | 75061108 | 75120486 | protein_coding | 1.033343755 | 6.91E−08 | FALSE | FALSE | FALSE | FALSE | FALSE | FALSE |
| ENSG00000125895 | TMEM74B | 20 | 1161205 | 1166059 | protein_coding | 1.03283419 | 8.51E−07 | FALSE | FALSE | FALSE | FALSE | FALSE | FALSE |
| ENSG00000168496 | FEN1 | 11 | 61560109 | 61564716 | protein_coding | 1.032828978 | 2.59E−42 | FALSE | FALSE | FALSE | FALSE | TRUE | FALSE |
| ENSG00000144810 | COL8A1 | 3 | 99357319 | 99518070 | protein_coding | 1.030696232 | 7.77E−09 | FALSE | FALSE | FALSE | FALSE | FALSE | FALSE |
| ENSG00000270195 | | 4 | 1714548 | 1715349 | lincRNA | 1.028895204 | 1.69E−13 | FALSE | FALSE | FALSE | FALSE | FALSE | FALSE |
| ENSG00000150551 | LYPD1 | 2 | 133402426 | 133429152 | protein_coding | 1.028773184 | 0.000116187 | FALSE | FALSE | FALSE | FALSE | FALSE | TRUE |
| ENSG00000088305 | DNMT3B | 20 | 31350191 | 31397162 | protein_coding | 1.025857861 | 2.04E−15 | FALSE | FALSE | FALSE | FALSE | FALSE | FALSE |
| ENSG00000136108 | CKAP2 | 13 | 53029564 | 53050763 | protein_coding | 1.025696883 | 4.42E−35 | FALSE | FALSE | FALSE | FALSE | FALSE | FALSE |
| ENSG00000138795 | LEF1 | 4 | 108968701 | 109090112 | protein_coding | 1.025366422 | 1.14E−09 | FALSE | FALSE | FALSE | FALSE | FALSE | FALSE |
| ENSG00000029993 | HMGB3 | X | 150148982 | 150159248 | protein_coding | 1.025273447 | 7.31E−16 | FALSE | FALSE | FALSE | FALSE | FALSE | FALSE |
| ENSG00000164694 | FNDC1 | 6 | 159590429 | 159693141 | protein_coding | 1.024794347 | 0.000201801 | FALSE | FALSE | FALSE | FALSE | TRUE | TRUE |
| ENSG00000173457 | PPP1R14B | 11 | 64011956 | 64014413 | protein_coding | 1.024711603 | 8.57E−40 | FALSE | FALSE | FALSE | FALSE | FALSE | FALSE |
| ENSG00000104738 | MCM4 | 8 | 48872745 | 48890720 | protein_coding | 1.023721493 | 1.65E−24 | FALSE | FALSE | FALSE | FALSE | FALSE | TRUE |
| ENSG00000091879 | ANGPT2 | 8 | 6357172 | 6420930 | protein_coding | 1.022327282 | 5.44E−10 | FALSE | FALSE | FALSE | FALSE | FALSE | FALSE |
| ENSG00000271550 | | 7 | 64139332 | 64147771 | pseudogene | 1.021319175 | 4.34E−14 | FALSE | FALSE | FALSE | FALSE | FALSE | FALSE |
| ENSG00000127423 | AUNIP | 1 | 26158414 | 26185903 | protein_coding | 1.021188011 | 2.75E−13 | FALSE | FALSE | FALSE | FALSE | FALSE | FALSE |
| ENSG00000172061 | LRRC15 | 3 | 194075976 | 194090472 | protein_coding | 1.020560533 | 0.029866718 | FALSE | FALSE | FALSE | FALSE | FALSE | TRUE |
| ENSG00000203499 | FAM83H-AS1 | 8 | 144816310 | 144828507 | lincRNA | 1.019699556 | 6.36E−08 | FALSE | FALSE | FALSE | FALSE | FALSE | TRUE |
| ENSG00000260920 | | 1 | 40929991 | 40932436 | sense_overlapping | 1.017151037 | 5.74E−33 | FALSE | FALSE | FALSE | TRUE | FALSE | FALSE |
| ENSG00000204624 | PTCHD2 | 1 | 11539223 | 11597641 | protein_coding | 1.01673975 | 5.49E−05 | FALSE | FALSE | FALSE | FALSE | FALSE | FALSE |
| ENSG00000222041 | LINC00152 | 2 | 87754887 | 87906324 | lincRNA | 1.016671535 | 1.14E−15 | FALSE | FALSE | FALSE | FALSE | FALSE | FALSE |
| ENSG00000197670 | | 20 | 52169309 | 52191847 | antisense | 1.016465619 | 2.79E−17 | FALSE | FALSE | FALSE | FALSE | FALSE | FALSE |
| ENSG00000146918 | NCAPG2 | 7 | 158424003 | 158497520 | protein_coding | 1.016079738 | 2.27E−34 | FALSE | FALSE | FALSE | FALSE | FALSE | FALSE |
| ENSG00000025708 | TYMP | 22 | 50964181 | 50968485 | protein_coding | 1.015302451 | 1.96E−13 | FALSE | FALSE | FALSE | FALSE | FALSE | FALSE |
| ENSG00000141756 | FKBP10 | 17 | 39968932 | 39979465 | protein_coding | 1.013639682 | 1.85E−10 | FALSE | FALSE | FALSE | FALSE | FALSE | FALSE |
| ENSG00000198720 | ANKRD13B | 17 | 27916787 | 27941779 | protein_coding | 1.013275697 | 1.24E−15 | FALSE | FALSE | FALSE | FALSE | FALSE | FALSE |
| ENSG00000187990 | HIST1H2BG | 6 | 26216428 | 26216872 | protein_coding | 1.011009219 | 4.39E−06 | FALSE | FALSE | FALSE | FALSE | FALSE | FALSE |
| ENSG00000097046 | CDC7 | 1 | 91966408 | 91991321 | protein_coding | 1.010264143 | 5.32E−24 | FALSE | FALSE | FALSE | FALSE | FALSE | FALSE |
| ENSG00000132000 | PODNL1 | 19 | 14042000 | 14064204 | protein_coding | 1.00984091 | 2.10E−09 | FALSE | FALSE | FALSE | FALSE | FALSE | FALSE |
| ENSG00000013573 | DDX11 | 12 | 31226779 | 31257725 | protein_coding | 1.009056115 | 1.49E−32 | FALSE | FALSE | FALSE | FALSE | FALSE | FALSE |
| ENSG00000272711 | | 2 | 75059782 | 75061114 | lincRNA | 1.008914542 | 3.21E−09 | FALSE | FALSE | FALSE | FALSE | FALSE | FALSE |
| ENSG00000029153 | ARNTL2 | 12 | 27485787 | 27576241 | protein_coding | 1.008912915 | 8.97E−10 | FALSE | FALSE | FALSE | FALSE | FALSE | TRUE |
| ENSG00000137573 | SULF1 | 8 | 70378859 | 70573150 | protein_coding | 1.005397069 | 4.87E−08 | FALSE | FALSE | FALSE | FALSE | TRUE | FALSE |
| ENSG00000106683 | LIMK1 | 7 | 73497263 | 73536855 | protein_coding | 1.005304742 | 1.21E−41 | FALSE | FALSE | FALSE | FALSE | FALSE | FALSE |
| ENSG00000124766 | SOX4 | 6 | 21593972 | 21598847 | protein_coding | 1.005210507 | 9.82E−18 | FALSE | FALSE | TRUE | TRUE | TRUE | TRUE |
| ENSG00000163535 | SGOL2 | 2 | 201374731 | 201448505 | protein_coding | 1.005032832 | 7.02E−20 | FALSE | FALSE | FALSE | FALSE | FALSE | TRUE |
| ENSG00000141527 | CARD14 | 17 | 78143791 | 78183130 | protein_coding | 1.004265274 | 3.76E−08 | FALSE | FALSE | FALSE | FALSE | TRUE | FALSE |
| ENSG00000120334 | CENPL | 1 | 173768688 | 173793858 | protein_coding | 1.002652633 | 3.96E−37 | FALSE | FALSE | FALSE | FALSE | FALSE | TRUE |
| ENSG00000120708 | TGFBI | 5 | 135364584 | 135399507 | protein_coding | 1.00208808 | 7.90E−08 | FALSE | FALSE | FALSE | FALSE | FALSE | FALSE |
| ENSG00000163794 | UCN | 2 | 27530268 | 27531313 | protein_coding | 1.001907376 | 1.53E−12 | FALSE | FALSE | FALSE | FALSE | FALSE | FALSE |
| ENSG00000213171 | LINGO4 | 1 | 151772740 | 151778630 | protein_coding | −1.000101709 | 1.15E−06 | FALSE | FALSE | FALSE | FALSE | FALSE | FALSE |
| ENSG00000197291 | RAMP2-AS1 | 17 | 40905932 | 40913275 | lincRNA | −1.000272963 | 1.58E−10 | FALSE | FALSE | FALSE | FALSE | FALSE | FALSE |
| ENSG00000227558 | PGM5P2 | 9 | 69080240 | 69147854 | pseudogene | −1.00038357 | 2.48E−15 | FALSE | FALSE | FALSE | FALSE | FALSE | FALSE |
| ENSG00000205106 | | 11 | 45792983 | 45793909 | protein_coding | −1.000452497 | 5.01E−07 | FALSE | FALSE | FALSE | FALSE | FALSE | FALSE |
| ENSG00000137070 | IL11RA | 9 | 34650699 | 34661889 | protein_coding | −1.000601583 | 1.77E−27 | FALSE | FALSE | FALSE | FALSE | FALSE | TRUE |
| ENSG00000162520 | SYNC | 1 | 33145507 | 33169197 | protein_coding | −1.000748506 | 9.40E−10 | FALSE | FALSE | FALSE | FALSE | FALSE | FALSE |
| ENSG00000102053 | ZC3H12B | X | 64708615 | 64727767 | protein_coding | −1.001116782 | 1.55E−15 | FALSE | FALSE | FALSE | FALSE | FALSE | FALSE |
| ENSG00000169302 | STK32A | 5 | 146614526 | 146767415 | protein_coding | −1.001126192 | 0.000678282 | FALSE | FALSE | FALSE | FALSE | FALSE | TRUE |
| ENSG00000158220 | ESYT3 | 3 | 138153428 | 138200528 | protein_coding | −1.001312832 | 2.18E−06 | FALSE | FALSE | FALSE | FALSE | FALSE | TRUE |
| ENSG00000257335 | MGAM | 7 | 141607613 | 141806547 | protein_coding | −1.001558948 | 0.001997183 | FALSE | FALSE | FALSE | FALSE | FALSE | FALSE |
| ENSG00000147573 | TRIM55 | 8 | 67039131 | 67087720 | protein_coding | −1.001722309 | 0.034494695 | FALSE | FALSE | FALSE | FALSE | FALSE | FALSE |
| ENSG00000162407 | PPAP2B | 1 | 56960419 | 57110974 | protein_coding | −1.003175053 | 2.84E−22 | FALSE | FALSE | FALSE | FALSE | FALSE | TRUE |
| ENSG00000169432 | SCN9A | 2 | 167051695 | 167232503 | protein_coding | −1.00354478 | 4.35E−06 | FALSE | FALSE | FALSE | FALSE | FALSE | FALSE |
| ENSG00000261616 | | 15 | 99679522 | 99685575 | antisense | −1.00357726 | 3.75E−07 | FALSE | FALSE | FALSE | FALSE | FALSE | TRUE |
| ENSG00000138759 | FRAS1 | 4 | 78978724 | 79465423 | protein_coding | −1.003874221 | 6.31E−07 | FALSE | FALSE | FALSE | FALSE | FALSE | FALSE |
| ENSG00000139112 | GABARAPL1 | 12 | 10365057 | 10375727 | protein_coding | −1.004774359 | 2.98E−25 | FALSE | FALSE | FALSE | FALSE | FALSE | FALSE |
| ENSG00000170382 | LRRN2 | 1 | 204586298 | 204654861 | protein_coding | −1.00535499 | 2.76E−05 | FALSE | FALSE | FALSE | FALSE | FALSE | TRUE |
| ENSG00000003137 | CYP26B1 | 2 | 72356367 | 72375167 | protein_coding | −1.006234249 | 1.34E−08 | FALSE | FALSE | FALSE | FALSE | FALSE | FALSE |
| ENSG00000075239 | ACAT1 | 11 | 107992243 | 108018503 | protein_coding | −1.006993372 | 1.31E−21 | FALSE | FALSE | FALSE | FALSE | FALSE | FALSE |
| ENSG00000162643 | WDR63 | 1 | 85464830 | 85598821 | protein_coding | −1.008501229 | 5.30E−07 | FALSE | FALSE | FALSE | FALSE | FALSE | FALSE |
| ENSG00000197444 | OGDHL | 10 | 50942689 | 50970425 | protein_coding | −1.009125522 | 0.01006367 | FALSE | FALSE | FALSE | FALSE | FALSE | TRUE |
| ENSG00000272279 | | 6 | 1528599 | 1529146 | lincRNA | −1.009523828 | 7.23E−06 | FALSE | FALSE | FALSE | FALSE | FALSE | FALSE |
| ENSG00000267336 | | 18 | 12912843 | 12913905 | pseudogene | −1.010816477 | 1.45E−07 | FALSE | TRUE | TRUE | TRUE | FALSE | TRUE |
| ENSG00000178187 | ZNF454 | 5 | 178368192 | 178393434 | protein_coding | −1.010867111 | 1.24E−10 | FALSE | FALSE | FALSE | FALSE | FALSE | FALSE |
| ENSG00000169291 | SHE | 1 | 154442248 | 154474589 | protein_coding | −1.011471771 | 3.12E−13 | FALSE | FALSE | FALSE | FALSE | FALSE | FALSE |
| ENSG00000162373 | BEND5 | 1 | 49193195 | 49242641 | protein_coding | −1.011838034 | 1.38E−12 | FALSE | FALSE | FALSE | FALSE | FALSE | FALSE |
| ENSG00000112761 | WISP3 | 6 | 112375275 | 112392171 | protein_coding | −1.011843801 | 0.00021386 | FALSE | FALSE | FALSE | FALSE | FALSE | FALSE |
| ENSG00000140287 | HDC | 15 | 50534144 | 50558223 | protein_coding | −1.012197788 | 3.64E−05 | FALSE | FALSE | FALSE | FALSE | FALSE | FALSE |
| ENSG00000103241 | FOXF1 | 16 | 86544133 | 86548076 | protein_coding | −1.01239164 | 1.07E−08 | FALSE | FALSE | FALSE | FALSE | FALSE | TRUE |
| ENSG00000160180 | TFF3 | 21 | 43731777 | 43735761 | protein_coding | −1.013040166 | 0.012039244 | FALSE | FALSE | FALSE | FALSE | FALSE | TRUE |
| ENSG00000237489 | LINC00959 | 10 | 131864638 | 131909081 | lincRNA | −1.014045177 | 4.78E−31 | FALSE | FALSE | FALSE | FALSE | FALSE | FALSE |
| ENSG00000147036 | LANCL3 | X | 37430822 | 37543716 | protein_coding | −1.014085484 | 5.52E−10 | FALSE | FALSE | FALSE | FALSE | FALSE | FALSE |
| ENSG00000177432 | NAP1L5 | 4 | 89617066 | 89619386 | protein_coding | −1.014143108 | 9.98E−28 | FALSE | FALSE | FALSE | FALSE | FALSE | FALSE |
| ENSG00000112183 | RBM24 | 6 | 17281577 | 17294106 | protein_coding | −1.015071797 | 0.000217386 | FALSE | FALSE | FALSE | FALSE | FALSE | FALSE |
| ENSG00000163644 | PPM1K | 4 | 89178772 | 89205921 | protein_coding | −1.015443813 | 1.39E−28 | FALSE | FALSE | FALSE | FALSE | FALSE | FALSE |
| ENSG00000223573 | TINCR | 19 | 5558178 | 5568045 | lincRNA | −1.015616316 | 0.00175161 | FALSE | FALSE | FALSE | FALSE | FALSE | TRUE |
| ENSG00000130844 | ZNF331 | 19 | 54024235 | 54083523 | protein_coding | −1.016930562 | 1.48E−21 | FALSE | FALSE | FALSE | FALSE | FALSE | FALSE |
| ENSG00000198682 | PAPSS2 | 10 | 89419370 | 89507462 | protein_coding | −1.01700235 | 1.07E−13 | FALSE | FALSE | FALSE | FALSE | FALSE | FALSE |
| ENSG00000264868 | | 7 | 87900207 | 87903065 | lincRNA | −1.017316628 | 9.99E−06 | FALSE | FALSE | FALSE | FALSE | FALSE | FALSE |
| ENSG00000127329 | PTPRB | 12 | 70910630 | 71031220 | protein_coding | −1.017996781 | 1.63E−13 | FALSE | FALSE | FALSE | FALSE | FALSE | FALSE |
| ENSG00000149557 | FEZ1 | 11 | 125315646 | 125366213 | protein_coding | −1.018454234 | 2.65E−15 | FALSE | FALSE | FALSE | FALSE | FALSE | TRUE |
| ENSG00000224848 | | 9 | 99449338 | 99483403 | lincRNA | −1.018908494 | 7.44E−08 | FALSE | FALSE | FALSE | FALSE | FALSE | FALSE |
| ENSG00000188959 | C9orf152 | 9 | 112952328 | 112970469 | protein_coding | −1.019016329 | 0.001860297 | FALSE | FALSE | FALSE | FALSE | FALSE | TRUE |
| ENSG00000162881 | OXER1 | 2 | 42989642 | 42991401 | protein_coding | −1.019034119 | 2.43E−11 | FALSE | FALSE | FALSE | FALSE | FALSE | FALSE |
| ENSG00000113070 | HBEGF | 5 | 139712428 | 139726216 | protein_coding | −1.019108136 | 4.40E−12 | FALSE | FALSE | FALSE | FALSE | FALSE | FALSE |
| ENSG00000215277 | C14orf164 | 14 | 23654525 | 23742686 | protein_coding | −1.019302951 | 0.00909146 | FALSE | FALSE | FALSE | FALSE | FALSE | FALSE |
| ENSG00000175899 | A2M | 12 | 9220260 | 9268825 | protein_coding | −1.019979484 | 7.19E−13 | FALSE | FALSE | FALSE | FALSE | FALSE | FALSE |
| ENSG00000204706 | MAMDC2-AS: | 9 | 72700732 | 72790804 | antisense | −1.021129853 | 6.45E−19 | FALSE | FALSE | FALSE | FALSE | FALSE | FALSE |
| ENSG00000092096 | SLC22A17 | 14 | 23815515 | 23822121 | protein_coding | −1.021232847 | 6.14E−13 | FALSE | FALSE | FALSE | FALSE | FALSE | FALSE |
| ENSG00000118307 | CASC1 | 12 | 25261354 | 25348096 | protein_coding | −1.021314073 | 9.23E−09 | FALSE | FALSE | FALSE | FALSE | FALSE | FALSE |
| ENSG00000232628 | | 1 | 224396449 | 224400981 | sense_intronic | −1.02159871 | 3.99E−06 | FALSE | FALSE | FALSE | FALSE | FALSE | FALSE |
| ENSG00000183023 | SLC8A1 | 2 | 40324410 | 40838193 | protein_coding | −1.022291949 | 4.06E−13 | FALSE | FALSE | FALSE | FALSE | FALSE | FALSE |
| ENSG00000100234 | TIMP3 | 22 | 33197687 | 33259030 | protein_coding | −1.023423331 | 1.69E−13 | FALSE | FALSE | FALSE | FALSE | FALSE | FALSE |
| ENSG00000146021 | KLHL3 | 5 | 136953189 | 137071779 | protein_coding | −1.023596465 | 1.01E−10 | FALSE | FALSE | FALSE | FALSE | FALSE | FALSE |
| ENSG00000144229 | THSD7B | 2 | 137523115 | 138435287 | protein_coding | −1.02364604 | 0.001477103 | FALSE | FALSE | FALSE | FALSE | FALSE | FALSE |
| ENSG00000171368 | TPPP | 5 | 660883 | 693510 | protein_coding | −1.023789291 | 3.12E−08 | FALSE | FALSE | FALSE | FALSE | FALSE | FALSE |
| ENSG00000100079 | LGALS2 | 22 | 37966255 | 37978623 | protein_coding | −1.024509502 | 5.33E−06 | FALSE | FALSE | FALSE | FALSE | FALSE | FALSE |
| ENSG00000238018 | | 2 | 54888148 | 54889929 | antisense | −1.024599762 | 8.00E−08 | FALSE | FALSE | FALSE | FALSE | FALSE | FALSE |
| ENSG00000214491 | SEC14L6 | 22 | 30918786 | 30942669 | protein_coding | −1.025049951 | 0.000421943 | FALSE | FALSE | FALSE | FALSE | FALSE | TRUE |
| ENSG00000183114 | FAM43B | 1 | 20878932 | 20881512 | protein_coding | −1.025221842 | 5.51E−07 | FALSE | FALSE | FALSE | FALSE | FALSE | FALSE |
| ENSG00000173258 | ZNF483 | 9 | 114287439 | 114340124 | protein_coding | −1.025941188 | 2.54E−18 | FALSE | FALSE | FALSE | FALSE | FALSE | FALSE |
| ENSG00000165996 | PTPLA | 10 | 17631958 | 17659376 | protein_coding | −1.026095404 | 3.45E−09 | FALSE | FALSE | FALSE | FALSE | FALSE | FALSE |
| ENSG00000165899 | OTOGL | 12 | 80603233 | 80772870 | protein_coding | −1.02635375 | 0.001130859 | FALSE | FALSE | FALSE | FALSE | FALSE | FALSE |
| ENSG00000125384 | PTGER2 | 14 | 52781023 | 52795324 | protein_coding | −1.026594753 | 1.06E−11 | FALSE | FALSE | FALSE | FALSE | FALSE | FALSE |
| ENSG00000176971 | FIBIN | 11 | 27015628 | 27018630 | protein_coding | −1.027006364 | 2.71E−08 | FALSE | FALSE | FALSE | FALSE | FALSE | FALSE |
| ENSG00000112293 | GPLD1 | 6 | 24424793 | 24495433 | protein_coding | −1.027045514 | 6.63E−12 | FALSE | FALSE | FALSE | FALSE | FALSE | FALSE |
| ENSG00000267280 | | 17 | 59470817 | 59488916 | antisense | −1.027391906 | 2.64E−09 | FALSE | FALSE | FALSE | FALSE | FALSE | FALSE |
| ENSG00000152672 | CLEC4F | 2 | 71035775 | 71047732 | protein_coding | −1.027681907 | 1.03E−05 | FALSE | FALSE | FALSE | FALSE | FALSE | FALSE |
| ENSG00000151715 | TMEM45B | 11 | 129685714 | 129729898 | protein_coding | −1.027871788 | 0.001100624 | FALSE | FALSE | FALSE | FALSE | FALSE | TRUE |
| ENSG00000046889 | PREX2 | 8 | 68864353 | 69149265 | protein_coding | −1.028267449 | 1.87E−10 | FALSE | FALSE | FALSE | FALSE | FALSE | FALSE |
| ENSG00000090530 | LEPREL1 | 3 | 189674517 | 189840226 | protein_coding | −1.028649372 | 4.81E−09 | FALSE | FALSE | FALSE | FALSE | FALSE | FALSE |
| ENSG00000170927 | PKHD1 | 6 | 51480098 | 51952423 | protein_coding | −1.029035282 | 0.002141993 | FALSE | FALSE | FALSE | FALSE | FALSE | TRUE |
| ENSG00000174899 | C3orf55 | 3 | 157261035 | 157395538 | protein_coding | −1.029695158 | 0.000798402 | FALSE | FALSE | FALSE | FALSE | FALSE | FALSE |
| ENSG00000264968 | | 17 | 38083995 | 38095854 | lincRNA | −1.02997055 | 2.22E−10 | FALSE | FALSE | FALSE | FALSE | FALSE | FALSE |
| ENSG00000170608 | FOXA3 | 19 | 46367247 | 46377055 | protein_coding | −1.029982826 | 0.026392328 | FALSE | FALSE | FALSE | FALSE | FALSE | TRUE |
| ENSG00000197776 | KLHDC1 | 14 | 50159823 | 50219870 | protein_coding | −1.030772562 | 2.52E−32 | FALSE | FALSE | FALSE | FALSE | FALSE | TRUE |
| ENSG00000203709 | C1orf132 | 1 | 207974863 | 208042495 | processed_transc [symbol] | −1.030902625 | 3.89E−15 | FALSE | FALSE | FALSE | FALSE | FALSE | TRUE |
| ENSG00000170989 | S1PR1 | 1 | 101702444 | 101707074 | protein_coding | −1.030968414 | 7.58E−14 | FALSE | FALSE | FALSE | FALSE | FALSE | FALSE |
| ENSG00000254667 | | 11 | 123301058 | 123306427 | protein_coding | −1.031120875 | 2.42E−07 | FALSE | FALSE | FALSE | FALSE | FALSE | FALSE |
| ENSG00000114166 | KAT2B | 3 | 20081515 | 20195896 | protein_coding | −1.031133031 | 1.16E−34 | FALSE | FALSE | FALSE | FALSE | FALSE | FALSE |
| ENSG00000115138 | POMC | 2 | 25383722 | 25391772 | protein_coding | −1.031210421 | 1.01E−09 | FALSE | FALSE | FALSE | FALSE | FALSE | FALSE |
| ENSG00000172318 | B3GALT1 | 2 | 168675182 | 168730551 | protein_coding | −1.03166324 | 0.003423341 | FALSE | FALSE | FALSE | FALSE | FALSE | FALSE |
| ENSG00000150281 | CTF1 | 16 | 30907928 | 30914881 | protein_coding | −1.031799385 | 1.48E−15 | FALSE | FALSE | FALSE | FALSE | FALSE | FALSE |
| ENSG00000144668 | ITGA9 | 3 | 37493606 | 37865005 | protein_coding | −1.031852907 | 8.43E−15 | FALSE | FALSE | FALSE | FALSE | FALSE | TRUE |
| ENSG00000159713 | TPPP3 | 16 | 67423712 | 67427438 | protein_coding | −1.031899164 | 1.27E−09 | FALSE | FALSE | FALSE | FALSE | FALSE | FALSE |
| ENSG00000160321 | ZNF208 | 19 | 22115760 | 22193751 | protein_coding | −1.032027487 | 2.83E−07 | FALSE | FALSE | FALSE | FALSE | FALSE | FALSE |
| ENSG00000186105 | LRRC70 | 5 | 61874562 | 61877275 | protein_coding | −1.032426475 | 3.25E−15 | FALSE | FALSE | FALSE | FALSE | FALSE | FALSE |
| ENSG00000003096 | KLHL13 | X | 117031776 | 117251303 | protein_coding | −1.032785845 | 1.32E−09 | FALSE | FALSE | FALSE | FALSE | FALSE | FALSE |
| ENSG00000232415 | | 7 | 73473906 | 73476614 | antisense | −1.032960045 | 1.99E−07 | FALSE | FALSE | FALSE | FALSE | FALSE | FALSE |
| ENSG00000169224 | GCSAML | 1 | 247670360 | 247740992 | protein_coding | −1.033450116 | 0.000161596 | FALSE | FALSE | FALSE | FALSE | FALSE | FALSE |
| ENSG00000139914 | FITM1 | 14 | 24600484 | 24602058 | protein_coding | −1.033879773 | 6.34E−12 | FALSE | FALSE | FALSE | FALSE | FALSE | FALSE |
| ENSG00000164741 | DLC1 | 8 | 12940870 | 13373167 | protein_coding | −1.034033285 | 5.07E−14 | FALSE | FALSE | FALSE | FALSE | FALSE | FALSE |
| ENSG00000234807 | LINC01135 | 1 | 59250823 | 59365384 | lincRNA | −1.034742111 | 1.50E−11 | FALSE | FALSE | FALSE | FALSE | FALSE | FALSE |
| ENSG00000152292 | SH2D6 | 2 | 85645844 | 85664152 | protein_coding | −1.035163866 | 0.000260069 | FALSE | FALSE | FALSE | FALSE | FALSE | FALSE |
| ENSG00000167779 | IGFBP6 | 12 | 53491220 | 53496129 | protein_coding | −1.035190679 | 5.70E−07 | FALSE | FALSE | FALSE | FALSE | FALSE | FALSE |
| ENSG00000117601 | SERPINC1 | 1 | 173872947 | 173886516 | protein_coding | −1.035349339 | 0.000964141 | FALSE | FALSE | FALSE | FALSE | FALSE | TRUE |
| ENSG00000136732 | GYPC | 2 | 127413509 | 127454246 | protein_coding | −1.035549653 | 6.05E−15 | FALSE | FALSE | FALSE | FALSE | FALSE | FALSE |
| ENSG00000246985 | SOCS2-AS1 | 12 | 93936239 | 93965544 | processed_transc [symbol] | −1.037481573 | 1.60E−14 | FALSE | FALSE | FALSE | FALSE | FALSE | FALSE |
| ENSG00000079337 | RAPGEF3 | 12 | 48128455 | 48164823 | protein_coding | −1.038108243 | 1.08E−15 | FALSE | FALSE | FALSE | FALSE | FALSE | TRUE |
| ENSG00000170961 | HAS2 | 8 | 122624356 | 122653630 | protein_coding | −1.038119642 | 9.40E−06 | FALSE | FALSE | FALSE | FALSE | FALSE | FALSE |
| ENSG00000242759 | LINC00882 | 3 | 106555658 | 106959488 | lincRNA | −1.038238556 | 2.35E−10 | FALSE | FALSE | FALSE | FALSE | FALSE | FALSE |
| ENSG00000107485 | GATA3 | 10 | 8095567 | 8117161 | protein_coding | −1.03829602 | 8.47E−05 | FALSE | FALSE | FALSE | FALSE | FALSE | TRUE |
| ENSG00000254122 | PCDHGB7 | 5 | 140797427 | 140892546 | protein_coding | −1.03832244 | 6.09E−12 | FALSE | FALSE | FALSE | FALSE | FALSE | TRUE |
| ENSG00000173567 | GPR113 | 2 | 26531041 | 26569685 | protein_coding | −1.039092127 | 1.48E−12 | FALSE | FALSE | FALSE | FALSE | FALSE | FALSE |
| ENSG00000182795 | C1orf116 | 1 | 207191866 | 207206101 | protein_coding | −1.039432025 | 0.000353026 | FALSE | FALSE | FALSE | FALSE | FALSE | TRUE |
| ENSG00000179023 | KLHDC7A | 1 | 18807424 | 18812478 | protein_coding | −1.039499902 | 2.04E−05 | FALSE | FALSE | FALSE | FALSE | FALSE | TRUE |
| ENSG00000136573 | BLK | 8 | 11351510 | 11422113 | protein_coding | −1.039501846 | 0.006562541 | FALSE | FALSE | FALSE | FALSE | FALSE | FALSE |
| ENSG00000188580 | NKAIN2 | 6 | 124125286 | 125146803 | protein_coding | −1.039505423 | 0.012042986 | FALSE | FALSE | FALSE | FALSE | FALSE | FALSE |
| ENSG00000143257 | NR113 | 1 | 161199456 | 161208092 | protein_coding | −1.039626153 | 1.43E−08 | FALSE | FALSE | FALSE | FALSE | FALSE | TRUE |
| ENSG00000135929 | CYP27A1 | 2 | 219646472 | 219680016 | protein_coding | −1.040835618 | 3.89E−13 | FALSE | FALSE | FALSE | FALSE | FALSE | FALSE |
| ENSG00000182397 | DNM1P46 | 15 | 100330361 | 100347132 | pseudogene | −1.041342086 | 2.09E−07 | FALSE | FALSE | FALSE | FALSE | FALSE | FALSE |
| ENSG00000151224 | MAT1A | 10 | 82031576 | 82049440 | protein_coding | −1.041643038 | 0.001934222 | FALSE | FALSE | FALSE | FALSE | FALSE | TRUE |
| ENSG00000268164 | | 19 | 54753289 | 54753907 | lincRNA | −1.042853822 | 6.03E−08 | FALSE | FALSE | FALSE | FALSE | FALSE | FALSE |
| ENSG00000123689 | GOS2 | 1 | 209848765 | 209849733 | protein_coding | −1.043166889 | 2.05E−06 | FALSE | FALSE | FALSE | FALSE | FALSE | FALSE |
| ENSG00000186231 | KLHL32 | 6 | 97372605 | 97588630 | protein_coding | −1.043413687 | 8.63E−09 | FALSE | FALSE | FALSE | FALSE | FALSE | FALSE |
| ENSG00000127903 | ZNF835 | 19 | 57174020 | 57183151 | protein_coding | −1.043644049 | 3.71E−11 | FALSE | FALSE | FALSE | FALSE | FALSE | FALSE |
| ENSG00000184828 | ZBTB7C | 18 | 45553044 | 45937123 | protein_coding | −1.044198836 | 1.35E−09 | FALSE | FALSE | FALSE | FALSE | FALSE | FALSE |
| ENSG00000164849 | GPR146 | 7 | 1084212 | 1098897 | protein_coding | −1.045253622 | 6.92E−16 | FALSE | FALSE | FALSE | FALSE | FALSE | FALSE |
| ENSG00000158571 | PFKFB1 | X | 54959394 | 55024967 | protein_coding | −1.045829885 | 3.90E−07 | FALSE | FALSE | FALSE | FALSE | FALSE | FALSE |
| ENSG00000183801 | OLFML1 | 11 | 7506619 | 7532608 | protein_coding | −1.04733649 | 6.82E−17 | FALSE | FALSE | FALSE | FALSE | FALSE | FALSE |
| ENSG00000265787 | CYP4F35P | 18 | 14337422 | 14342524 | lincRNA | −1.047660713 | 0.001567648 | FALSE | FALSE | FALSE | FALSE | FALSE | FALSE |
| ENSG00000175928 | LRRN1 | 3 | 3841121 | 3889387 | protein_coding | −1.047822126 | 0.000310001 | FALSE | FALSE | FALSE | FALSE | FALSE | FALSE |
| ENSG00000227192 | | 6 | 143360562 | 143363461 | antisense | −1.048110649 | 5.69E−08 | FALSE | FALSE | FALSE | FALSE | FALSE | FALSE |
| ENSG00000183908 | LRRC55 | 11 | 56949221 | 56959191 | protein_coding | −1.048285148 | 2.17E−05 | FALSE | FALSE | FALSE | FALSE | FALSE | FALSE |
| ENSG00000144031 | ANKRD53 | 2 | 71205510 | 71212626 | protein_coding | −1.048689731 | 6.34E−12 | FALSE | FALSE | FALSE | FALSE | FALSE | FALSE |
| ENSG00000112137 | PHACTR1 | 6 | 12717893 | 13288645 | protein_coding | −1.049137074 | 7.33E−13 | FALSE | FALSE | FALSE | FALSE | FALSE | FALSE |
| ENSG00000172247 | C1QTNF4 | 11 | 47611216 | 47616211 | protein_coding | −1.050130119 | 1.57E−08 | FALSE | FALSE | FALSE | FALSE | FALSE | FALSE |
| ENSG00000197380 | DACT3 | 19 | 47150869 | 47164395 | protein_coding | −1.050160272 | 4.19E−12 | FALSE | FALSE | FALSE | FALSE | FALSE | FALSE |
| ENSG00000231028 | LINC00271 | 6 | 135818489 | 136037193 | lincRNA | −1.050626668 | 9.20E−19 | FALSE | FALSE | FALSE | FALSE | FALSE | FALSE |
| ENSG00000271948 | | 8 | 17082473 | 17082978 | lincRNA | −1.050806537 | 1.85E−27 | FALSE | FALSE | FALSE | FALSE | FALSE | FALSE |
| ENSG00000081853 | PCDHGA2 | 5 | 140718539 | 140892546 | protein_coding | −1.050857946 | 1.63E−09 | FALSE | FALSE | FALSE | FALSE | FALSE | TRUE |
| ENSG00000184515 | BEX5 | X | 101408680 | 101411029 | protein_coding | −1.051452068 | 4.43E−13 | FALSE | FALSE | FALSE | FALSE | FALSE | FALSE |
| ENSG00000198948 | MFAP3L | 4 | 170907748 | 170954182 | protein_coding | −1.051510412 | 3.58E−12 | FALSE | FALSE | FALSE | FALSE | FALSE | FALSE |
| ENSG00000108691 | CCL2 | 17 | 32582304 | 32584222 | protein_coding | −1.052773706 | 1.68E−09 | FALSE | FALSE | FALSE | FALSE | FALSE | FALSE |
| ENSG00000262823 | | 17 | 4385261 | 4389648 | antisense | −1.052866388 | 3.22E−08 | FALSE | FALSE | FALSE | FALSE | FALSE | FALSE |
| ENSG00000134376 | CRB1 | 1 | 197170592 | 197447585 | protein_coding | −1.054468088 | 6.96E−08 | FALSE | FALSE | FALSE | FALSE | FALSE | TRUE |
| ENSG00000106804 | C5 | 9 | 123714616 | 123812554 | protein_coding | −1.054840849 | 5.88E−16 | FALSE | FALSE | FALSE | FALSE | FALSE | TRUE |
| ENSG00000105227 | PRX | 19 | 40899675 | 40919273 | protein_coding | −1.05516197 | 5.88E−14 | FALSE | FALSE | FALSE | FALSE | FALSE | FALSE |
| ENSG00000267868 | | 13 | 113619149 | 113620445 | lincRNA | −1.055543891 | 0.002062253 | FALSE | FALSE | FALSE | FALSE | FALSE | FALSE |
| ENSG00000267107 | | 19 | 41960074 | 42006559 | lincRNA | −1.056391721 | 1.55E−14 | FALSE | FALSE | FALSE | FALSE | FALSE | FALSE |
| ENSG00000104112 | SCG3 | 15 | 51973550 | 52013223 | protein_coding | −1.057904825 | 0.011561624 | FALSE | FALSE | FALSE | FALSE | FALSE | FALSE |
| ENSG00000128594 | LRRC4 | 7 | 127667124 | 127672160 | protein_coding | −1.058212576 | 7.26E−09 | FALSE | FALSE | FALSE | FALSE | FALSE | FALSE |
| ENSG00000091262 | ABCC6 | 16 | 16242785 | 16317379 | protein_coding | −1.058259888 | 1.45E−10 | FALSE | FALSE | FALSE | FALSE | FALSE | TRUE |
| ENSG00000258789 | | 14 | 89017941 | 89018837 | antisense | −1.058886116 | 5.10E−17 | FALSE | FALSE | FALSE | FALSE | FALSE | FALSE |
| ENSG00000261469 | | 16 | 68270748 | 68271570 | sense_intronic | −1.060180165 | 2.21E−06 | FALSE | FALSE | FALSE | FALSE | FALSE | FALSE |
| ENSG00000205832 | C16orf96 | 16 | 4606491 | 4650715 | protein_coding | −1.060464673 | 2.15E−09 | FALSE | FALSE | FALSE | FALSE | FALSE | FALSE |
| ENSG00000143502 | SUSD4 | 1 | 223394161 | 223537544 | protein_coding | −1.060528336 | 0.000442058 | FALSE | FALSE | FALSE | FALSE | FALSE | TRUE |
| ENSG00000258572 | | 14 | 95982473 | 95984248 | protein_coding | −1.060659703 | 8.94E−07 | FALSE | FALSE | FALSE | FALSE | FALSE | FALSE |
| ENSG00000129048 | ACKR4 | 3 | 132316081 | 132337811 | protein_coding | −1.060686837 | 1.56E−08 | FALSE | FALSE | FALSE | FALSE | FALSE | FALSE |
| ENSG00000168405 | CMAHP | 6 | 25081548 | 25166793 | pseudogene | −1.060774877 | 5.82E−18 | FALSE | FALSE | FALSE | FALSE | FALSE | FALSE |
| ENSG00000188153 | COL4A5 | X | 107683074 | 107940775 | protein_coding | −1.060868583 | 3.85E−07 | FALSE | FALSE | FALSE | FALSE | FALSE | FALSE |
| ENSG00000139737 | SLAIN1 | 13 | 78272023 | 78338377 | protein_coding | −1.060897698 | 8.72E−11 | FALSE | FALSE | FALSE | FALSE | FALSE | TRUE |
| ENSG00000122679 | RAMP3 | 7 | 45197390 | 45225901 | protein_coding | −1.061020703 | 4.33E−13 | FALSE | FALSE | FALSE | FALSE | FALSE | FALSE |
| ENSG00000183154 | | 8 | 37592279 | 37594944 | protein_coding | −1.061093849 | 6.24E−17 | FALSE | FALSE | FALSE | FALSE | FALSE | FALSE |
| ENSG00000248079 | DPH6-AS1 | 15 | 35838396 | 36151202 | lincRNA | −1.061596751 | 7.38E−12 | FALSE | FALSE | FALSE | FALSE | FALSE | FALSE |
| ENSG00000102409 | BEX4 | X | 102470020 | 102472174 | protein_coding | −1.061783206 | 6.71E−24 | FALSE | FALSE | FALSE | FALSE | FALSE | FALSE |
| ENSG00000224479 | | 3 | 63727969 | 63813121 | pseudogene | −1.061923478 | 2.23E−12 | FALSE | FALSE | FALSE | FALSE | FALSE | FALSE |
| ENSG00000154655 | L3MBTL4 | 18 | 5954705 | 6415236 | protein_coding | −1.06206662 | 1.07E−13 | FALSE | FALSE | FALSE | FALSE | FALSE | FALSE |
| ENSG00000227082 | | 1 | 121138614 | 121204983 | lincRNA | −1.062478967 | 2.03E−07 | FALSE | FALSE | FALSE | FALSE | FALSE | FALSE |
| ENSG00000152822 | GRM1 | 6 | 146348782 | 146758734 | protein_coding | −1.063493633 | 0.003512454 | FALSE | FALSE | FALSE | FALSE | FALSE | FALSE |
| ENSG00000184313 | MROH7 | 1 | 55107463 | 55175939 | protein_coding | −1.06358519 | 9.90E−09 | FALSE | FALSE | FALSE | FALSE | FALSE | FALSE |
| ENSG00000204186 | ZDBF2 | 2 | 207139387 | 207179148 | protein_coding | −1.064183541 | 8.99E−15 | FALSE | FALSE | FALSE | FALSE | FALSE | FALSE |
| ENSG00000206559 | ZCWPW2 | 3 | 28390637 | 28579613 | protein_coding | −1.064235213 | 1.18E−32 | FALSE | FALSE | FALSE | FALSE | FALSE | FALSE |
| ENSG00000166450 | PRTG | 15 | 55903744 | 56035288 | protein_coding | −1.06433984 | 1.01E−09 | FALSE | FALSE | FALSE | FALSE | FALSE | FALSE |
| ENSG00000166106 | ADAMTS15 | 11 | 130318869 | 130346532 | protein_coding | −1.065383411 | 3.21E−09 | FALSE | FALSE | FALSE | FALSE | FALSE | FALSE |
| ENSG00000213366 | GSTM2 | 1 | 110210644 | 110252171 | protein_coding | −1.066931314 | 5.92E−15 | FALSE | FALSE | FALSE | FALSE | FALSE | TRUE |
| ENSG00000125409 | TEKT3 | 17 | 15207128 | 15244958 | protein_coding | −1.067148608 | 1.43E−07 | FALSE | FALSE | FALSE | FALSE | FALSE | FALSE |
| ENSG00000185630 | PBX1 | 1 | 164524821 | 164868533 | protein_coding | −1.067452242 | 4.69E−20 | FALSE | FALSE | FALSE | FALSE | FALSE | TRUE |
| ENSG00000110786 | PTPN5 | 11 | 18749475 | 18814268 | protein_coding | −1.068032063 | 3.53E−05 | FALSE | FALSE | FALSE | FALSE | FALSE | FALSE |
| ENSG00000150995 | ITPR1 | 3 | 4535032 | 4889524 | protein_coding | −1.069203133 | 6.13E−18 | FALSE | FALSE | FALSE | FALSE | FALSE | FALSE |
| ENSG00000196542 | SPTSSB | 3 | 161062580 | 161090668 | protein_coding | −1.069479968 | 0.00691328 | FALSE | FALSE | FALSE | FALSE | FALSE | FALSE |
| ENSG00000130528 | HRC | 19 | 49654455 | 49658681 | protein_coding | −1.069684066 | 1.84E−10 | FALSE | FALSE | FALSE | FALSE | FALSE | FALSE |
| ENSG00000198478 | SH3BGRL2 | 6 | 80341000 | 80413372 | protein_coding | −1.069693335 | 6.79E−19 | FALSE | FALSE | FALSE | FALSE | FALSE | TRUE |
| ENSG00000120913 | PDLIM2 | 8 | 22435792 | 22455538 | protein_coding | −1.06993378 | 2.31E−21 | FALSE | FALSE | TRUE | TRUE | TRUE | FALSE |
| ENSG00000255248 | | 11 | 121899063 | 121987031 | sense_overlapping | −1.070949587 | 3.78E−13 | FALSE | FALSE | FALSE | FALSE | FALSE | FALSE |
| ENSG00000186994 | KANK3 | 19 | 8387468 | 8408146 | protein_coding | −1.071568314 | 1.12E−15 | FALSE | FALSE | FALSE | FALSE | FALSE | FALSE |
| ENSG00000112619 | PRPH2 | 6 | 42664340 | 42690312 | protein_coding | −1.072274899 | 1.23E−13 | FALSE | FALSE | FALSE | FALSE | FALSE | FALSE |
| ENSG00000169126 | ARMC4 | 10 | 28064115 | 28287977 | protein_coding | −1.073322595 | 0.000956511 | FALSE | FALSE | FALSE | FALSE | FALSE | FALSE |
| ENSG00000271347 | | 15 | 25236633 | 25236900 | sense_intronic | −1.073376957 | 2.54E−10 | FALSE | FALSE | FALSE | FALSE | FALSE | FALSE |
| ENSG00000272327 | | 8 | 32623643 | 32625477 | lincRNA | −1.074202617 | 1.97E−05 | FALSE | FALSE | FALSE | FALSE | FALSE | FALSE |
| ENSG00000116774 | OLFML3 | 1 | 114522063 | 114524876 | protein_coding | −1.074450185 | 3.14E−15 | FALSE | FALSE | FALSE | FALSE | FALSE | FALSE |
| ENSG00000146006 | LRRTM2 | 5 | 138204612 | 138211057 | protein_coding | −1.074748354 | 9.89E−11 | FALSE | FALSE | FALSE | FALSE | FALSE | TRUE |
| ENSG00000135373 | EHF | 11 | 34642640 | 34682604 | protein_coding | −1.07498331 | 0.00506126 | FALSE | FALSE | FALSE | FALSE | TRUE | TRUE |
| ENSG00000131831 | RAI2 | X | 17818169 | 17879457 | protein_coding | −1.075527069 | 1.41E−15 | FALSE | FALSE | FALSE | FALSE | FALSE | TRUE |
| ENSG00000167701 | GPT | 8 | 145728356 | 145732557 | protein_coding | −1.075711715 | 4.72E−06 | FALSE | FALSE | FALSE | FALSE | FALSE | FALSE |
| ENSG00000260686 | | 5 | 122168051 | 122170228 | sense_overlapping | −1.076001809 | 1.83E−29 | FALSE | FALSE | FALSE | FALSE | FALSE | FALSE |
| ENSG00000102003 | SYP | X | 49044269 | 49056718 | protein_coding | −1.076254706 | 1.71E−14 | FALSE | FALSE | FALSE | FALSE | FALSE | FALSE |
| ENSG00000171056 | SOX7 | 8 | 10581278 | 10697357 | protein_coding | −1.077969747 | 8.82E−17 | FALSE | FALSE | FALSE | FALSE | FALSE | TRUE |
| ENSG00000243836 | WDR86-AS1 | 7 | 151106247 | 151110440 | processed_transc [symbol] | −1.079010379 | 7.78E−07 | FALSE | FALSE | FALSE | FALSE | FALSE | FALSE |
| ENSG00000142494 | SLC47A1 | 17 | 19398698 | 19482347 | protein_coding | −1.079163936 | 2.98E−07 | FALSE | FALSE | FALSE | FALSE | FALSE | TRUE |
| ENSG00000099994 | SUSD2 | 22 | 24577227 | 24585078 | protein_coding | −1.07973735 | 1.86E−06 | FALSE | FALSE | FALSE | FALSE | FALSE | FALSE |
| ENSG00000164039 | BDH2 | 4 | 104000592 | 104021040 | protein_coding | −1.080394135 | 1.10E−34 | FALSE | FALSE | FALSE | FALSE | FALSE | FALSE |
| ENSG00000008226 | DLEC1 | 3 | 38080696 | 38165516 | protein_coding | −1.080432918 | 4.56E−10 | FALSE | FALSE | FALSE | FALSE | FALSE | FALSE |
| ENSG00000178031 | ADAMTSL1 | 9 | 18473892 | 18910948 | protein_coding | −1.080654625 | 1.46E−06 | FALSE | FALSE | FALSE | FALSE | FALSE | FALSE |
| ENSG00000107864 | CPEB3 | 10 | 93806449 | 94050844 | protein_coding | −1.080734021 | 1.04E−28 | FALSE | FALSE | FALSE | FALSE | FALSE | FALSE |
| ENSG00000145335 | SNCA | 4 | 90645250 | 90759466 | protein_coding | −1.081700351 | 1.69E−13 | FALSE | FALSE | FALSE | FALSE | FALSE | FALSE |
| ENSG00000250073 | | 11 | 124629025 | 124635832 | antisense | −1.083287302 | 1.95E−11 | FALSE | FALSE | FALSE | FALSE | FALSE | FALSE |
| ENSG00000122025 | FLT3 | 13 | 28577411 | 28674729 | protein_coding | −1.083386815 | 9.90E−07 | FALSE | FALSE | FALSE | FALSE | FALSE | FALSE |
| ENSG00000159176 | CSRP1 | 1 | 201452658 | 201478584 | protein_coding | −1.083390104 | 1.22E−22 | FALSE | FALSE | FALSE | FALSE | FALSE | FALSE |
| ENSG00000272360 | | 3 | 58476557 | 58477018 | lincRNA | −1.083906869 | 4.92E−11 | FALSE | FALSE | FALSE | FALSE | FALSE | FALSE |
| ENSG00000182950 | ODF3L1 | 15 | 76016318 | 76020029 | protein_coding | −1.084383256 | 2.68E−07 | FALSE | FALSE | FALSE | FALSE | FALSE | FALSE |
| ENSG00000073910 | FRY | 13 | 32605437 | 32870794 | protein_coding | −1.084584497 | 2.12E−21 | FALSE | FALSE | FALSE | FALSE | FALSE | FALSE |
| ENSG00000166840 | GLYATL1 | 11 | 58672871 | 58811000 | protein_coding | −1.08469557 | 0.021222792 | FALSE | FALSE | FALSE | FALSE | FALSE | TRUE |
| ENSG00000161594 | KLHL10 | 17 | 39991937 | 40004636 | protein_coding | −1.08654481 | 2.06E−07 | FALSE | FALSE | FALSE | FALSE | FALSE | TRUE |
| ENSG00000147041 | SYTL5 | X | 37865835 | 37988072 | protein_coding | −1.087134598 | 0.007624993 | FALSE | FALSE | FALSE | FALSE | FALSE | FALSE |
| ENSG00000248632 | | 4 | 165889739 | 165891154 | pseudogene | −1.087141563 | 1.15E−11 | FALSE | FALSE | FALSE | FALSE | FALSE | FALSE |
| ENSG00000074706 | IPCEF1 | 6 | 154475631 | 154677926 | protein_coding | −1.087606321 | 3.34E−14 | FALSE | FALSE | FALSE | FALSE | FALSE | FALSE |
| ENSG00000150594 | ADRA2A | 10 | 112836790 | 112840658 | protein_coding | −1.087855537 | 6.11E−08 | FALSE | FALSE | FALSE | FALSE | FALSE | FALSE |
| ENSG00000255471 | | 11 | 86603256 | 86636079 | antisense | −1.08797037 | 7.12E−16 | FALSE | FALSE | FALSE | FALSE | FALSE | FALSE |
| ENSG00000008311 | AASS | 7 | 121715701 | 121784334 | protein_coding | −1.088057087 | 1.31E−17 | FALSE | FALSE | FALSE | FALSE | FALSE | FALSE |
| ENSG00000180155 | LYNX1 | 8 | 143845752 | 143859640 | protein_coding | −1.088068733 | 7.15E−11 | FALSE | FALSE | FALSE | FALSE | FALSE | FALSE |
| ENSG00000239445 | ST3GAL6-AS1 | 3 | 98433174 | 98451495 | antisense | −1.088588634 | 5.53E−11 | FALSE | FALSE | FALSE | FALSE | FALSE | FALSE |
| ENSG00000065675 | PRKCQ | 10 | 6469105 | 6622263 | protein_coding | −1.088649457 | 4.43E−12 | FALSE | FALSE | FALSE | FALSE | FALSE | FALSE |
| ENSG00000108830 | RND2 | 17 | 41177258 | 41184057 | protein_coding | −1.089653916 | 5.99E−09 | FALSE | FALSE | FALSE | FALSE | FALSE | FALSE |
| ENSG00000175556 | LONRF3 | X | 118108581 | 118152318 | protein_coding | −1.090108177 | 2.55E−15 | FALSE | FALSE | FALSE | FALSE | FALSE | TRUE |
| ENSG00000109743 | BST1 | 4 | 15704573 | 15739936 | protein_coding | −1.090409395 | 1.52E−19 | FALSE | FALSE | FALSE | FALSE | FALSE | FALSE |
| ENSG00000236411 | NDUFAF4P3 | 3 | 37831412 | 37831936 | pseudogene | −1.09092817 | 1.73E−15 | FALSE | FALSE | FALSE | FALSE | FALSE | FALSE |
| ENSG00000166317 | SYNPO2L | 10 | 75404639 | 75423561 | protein_coding | −1.091286347 | 4.04E−05 | FALSE | FALSE | FALSE | FALSE | FALSE | FALSE |
| ENSG00000220517 | ASS1P1 | 6 | 25023475 | 25024711 | pseudogene | −1.091491862 | 1.23E−07 | FALSE | FALSE | FALSE | FALSE | FALSE | TRUE |
| ENSG00000229017 | | 6 | 143287558 | 143359214 | antisense | −1.092292887 | 1.12E−12 | FALSE | FALSE | FALSE | FALSE | FALSE | FALSE |
| ENSG00000170482 | SLC23A1 | 5 | 138702885 | 138720242 | protein_coding | −1.093173656 | 1.01E−05 | FALSE | FALSE | FALSE | FALSE | FALSE | FALSE |
| ENSG00000260633 | | 3 | 134066130 | 134068075 | lincRNA | −1.093240966 | 1.01E−11 | FALSE | FALSE | FALSE | FALSE | FALSE | FALSE |
| ENSG00000154479 | CCDC173 | 2 | 170501935 | 170550943 | protein_coding | −1.093274858 | 9.59E−10 | FALSE | FALSE | FALSE | FALSE | FALSE | FALSE |
| ENSG00000065320 | NTN1 | 17 | 8924859 | 9147317 | protein_coding | −1.093569018 | 1.97E−08 | FALSE | FALSE | FALSE | FALSE | FALSE | FALSE |
| ENSG00000141622 | RNF165 | 18 | 43906772 | 44043103 | protein_coding | −1.094673545 | 4.93E−08 | FALSE | FALSE | FALSE | FALSE | FALSE | FALSE |
| ENSG00000205018 | | 16 | 89006197 | 89017932 | protein_coding | −1.095104566 | 1.69E−08 | FALSE | FALSE | FALSE | FALSE | FALSE | TRUE |
| ENSG00000176595 | KBTBD11 | 8 | 1922044 | 1955102 | protein_coding | −1.096245061 | 2.68E−10 | FALSE | FALSE | FALSE | FALSE | FALSE | TRUE |
| ENSG00000100307 | CBX7 | 22 | 39516172 | 39548679 | protein_coding | −1.096522359 | 6.65E−27 | FALSE | FALSE | FALSE | FALSE | FALSE | TRUE |
| ENSG00000197614 | MFAP5 | 12 | 8789942 | 8815484 | protein_coding | −1.096704036 | 0.000624866 | FALSE | FALSE | FALSE | FALSE | FALSE | FALSE |
| ENSG00000234465 | PINLYP | 19 | 44080952 | 44088116 | protein_coding | −1.096767658 | 1.45E−17 | FALSE | FALSE | FALSE | FALSE | FALSE | FALSE |
| ENSG00000198400 | NTRK1 | 1 | 156785432 | 156851642 | protein_coding | −1.09739854 | 2.90E−07 | FALSE | FALSE | FALSE | FALSE | FALSE | FALSE |
| ENSG00000196167 | COLCA1 | 11 | 111164114 | 111175770 | protein_coding | −1.097717808 | 3.96E−07 | FALSE | FALSE | FALSE | FALSE | FALSE | FALSE |
| ENSG00000135406 | PRPH | 12 | 49687035 | 49692465 | protein_coding | −1.098635268 | 0.001212516 | FALSE | FALSE | FALSE | FALSE | FALSE | FALSE |
| ENSG00000111834 | RSPH4A | 6 | 116937642 | 116954148 | protein_coding | −1.09894033 | 2.83E−10 | FALSE | FALSE | FALSE | FALSE | FALSE | FALSE |
| ENSG00000134138 | MEIS2 | 15 | 37181406 | 37393504 | protein_coding | −1.09975366 | 8.34E−20 | FALSE | FALSE | FALSE | FALSE | FALSE | FALSE |
| ENSG00000162551 | ALPL | 1 | 21835858 | 21904905 | protein_coding | −1.099976469 | 2.92E−09 | FALSE | FALSE | FALSE | FALSE | FALSE | FALSE |
| ENSG00000140254 | DUOXA1 | 15 | 45409569 | 45422136 | protein_coding | −1.100044586 | 1.72E−05 | FALSE | FALSE | FALSE | FALSE | TRUE | TRUE |
| ENSG00000198121 | LPAR1 | 9 | 113635543 | 113800981 | protein_coding | −1.100216494 | 2.85E−16 | FALSE | FALSE | FALSE | FALSE | FALSE | FALSE |
| ENSG00000171016 | PYGO1 | 15 | 55831088 | 55881145 | protein_coding | −1.100371019 | 4.52E−14 | FALSE | FALSE | FALSE | FALSE | FALSE | FALSE |
| ENSG00000163032 | VSNL1 | 2 | 17720393 | 17838285 | protein_coding | −1.100603128 | 0.001835403 | FALSE | FALSE | FALSE | FALSE | FALSE | TRUE |
| ENSG00000106772 | PRUNE2 | 9 | 79226292 | 79521003 | protein_coding | −1.100672169 | 4.28E−07 | FALSE | FALSE | FALSE | FALSE | FALSE | FALSE |
| ENSG00000144057 | ST6GAL2 | 2 | 107418056 | 107503564 | protein_coding | −1.102620985 | 1.17E−05 | FALSE | FALSE | FALSE | FALSE | FALSE | TRUE |
| ENSG00000165449 | SLC16A9 | 10 | 61410523 | 61495760 | protein_coding | −1.102932014 | 0.000206002 | FALSE | FALSE | FALSE | FALSE | FALSE | FALSE |
| ENSG00000229619 | MBNL1-AS1 | 3 | 151980405 | 151987344 | antisense | −1.103031286 | 8.13E−16 | FALSE | FALSE | FALSE | FALSE | FALSE | FALSE |
| ENSG00000182636 | NDN | 15 | 23930565 | 23932450 | protein_coding | −1.103089464 | 2.41E−17 | FALSE | FALSE | FALSE | FALSE | FALSE | FALSE |
| ENSG00000147100 | SLC16A2 | X | 73641085 | 73753752 | protein_coding | −1.103812772 | 1.08E−21 | FALSE | FALSE | FALSE | FALSE | FALSE | FALSE |
| ENSG00000150556 | LYPD6B | 2 | 149894621 | 150071776 | protein_coding | −1.103891662 | 0.04613284 | FALSE | FALSE | FALSE | FALSE | FALSE | FALSE |
| ENSG00000151778 | SERP2 | 13 | 44947801 | 44971850 | protein_coding | −1.104936827 | 9.09E−15 | FALSE | FALSE | FALSE | FALSE | FALSE | FALSE |
| ENSG00000172348 | RCAN2 | 6 | 46188475 | 46459709 | protein_coding | −1.105563371 | 2.08E−13 | FALSE | FALSE | FALSE | FALSE | FALSE | FALSE |
| ENSG00000171357 | LURAP1 | 1 | 46669006 | 46686933 | protein_coding | −1.106172021 | 1.56E−19 | FALSE | FALSE | FALSE | FALSE | FALSE | FALSE |
| ENSG00000139910 | NOVA1 | 14 | 26912299 | 27066960 | protein_coding | −1.106259031 | 1.81E−07 | FALSE | FALSE | FALSE | FALSE | FALSE | FALSE |
| ENSG00000148935 | GAS2 | 11 | 22647188 | 22834601 | protein_coding | −1.106310185 | 1.69E−09 | FALSE | FALSE | FALSE | FALSE | FALSE | FALSE |
| ENSG00000234292 | | 5 | 90575914 | 90576959 | lincRNA | −1.106458108 | 2.15E−07 | FALSE | FALSE | FALSE | FALSE | FALSE | FALSE |
| ENSG00000171227 | TMEM37 | 2 | 120187477 | 120196096 | protein_coding | −1.106844529 | 1.92E−13 | FALSE | FALSE | FALSE | FALSE | FALSE | TRUE |
| ENSG00000120693 | SMAD9 | 13 | 37418968 | 37494902 | protein_coding | −1.106903044 | 7.55E−15 | FALSE | FALSE | FALSE | FALSE | FALSE | FALSE |
| ENSG00000255353 | | 11 | 107047012 | 107048256 | pseudogene | −1.107082865 | 2.56E−07 | FALSE | FALSE | TRUE | TRUE | FALSE | TRUE |
| ENSG00000198650 | TAT | 16 | 71599563 | 71611033 | protein_coding | −1.107084347 | 0.00173142 | FALSE | FALSE | FALSE | FALSE | FALSE | TRUE |
| ENSG00000232699 | BDH2P1 | 6 | 99622620 | 99623357 | pseudogene | −1.107854278 | 1.50E−26 | FALSE | FALSE | FALSE | FALSE | FALSE | TRUE |
| ENSG00000184985 | SORCS2 | 4 | 7194265 | 7744554 | protein_coding | −1.108195487 | 1.34E−07 | FALSE | FALSE | FALSE | FALSE | FALSE | FALSE |
| ENSG00000173208 | ABCD2 | 12 | 39943835 | 40013553 | protein_coding | −1.108392282 | 2.10E−09 | FALSE | FALSE | FALSE | FALSE | FALSE | FALSE |
| ENSG00000198756 | COLGALT2 | 1 | 183898796 | 184006863 | protein_coding | −1.108721651 | 4.48E−09 | FALSE | FALSE | FALSE | FALSE | FALSE | FALSE |
| ENSG00000213760 | ATP6V1G2 | 6 | 31512239 | 31516204 | protein_coding | −1.108987805 | 1.15E−18 | FALSE | FALSE | FALSE | FALSE | FALSE | TRUE |
| ENSG00000070601 | FRMPD1 | 9 | 37650997 | 37746901 | protein_coding | −1.109783566 | 3.48E−06 | FALSE | FALSE | FALSE | FALSE | FALSE | FALSE |
| ENSG00000138379 | MSTN | 2 | 190920423 | 190927455 | protein_coding | −1.110051634 | 4.12E−08 | FALSE | FALSE | FALSE | FALSE | FALSE | FALSE |
| ENSG00000170074 | FAM153A | 5 | 177134982 | 177210399 | protein_coding | −1.110452798 | 2.17E−05 | FALSE | FALSE | FALSE | FALSE | FALSE | FALSE |
| ENSG00000255495 | FAM85A | 8 | 12051976 | 12053789 | antisense | −1.112852326 | 9.47E−19 | FALSE | FALSE | FALSE | FALSE | FALSE | FALSE |
| ENSG00000151090 | THRB | 3 | 24158651 | 24536773 | protein_coding | −1.113194517 | 2.67E−17 | FALSE | FALSE | FALSE | FALSE | FALSE | FALSE |
| ENSG00000185437 | SH3BGR | 21 | 40817781 | 40887433 | protein_coding | −1.11347353 | 1.35E−19 | FALSE | FALSE | FALSE | FALSE | FALSE | FALSE |
| ENSG00000196557 | CACNA1H | 16 | 1203241 | 1271771 | protein_coding | −1.113614991 | 3.03E−09 | FALSE | FALSE | FALSE | FALSE | FALSE | FALSE |
| ENSG00000116690 | PRG4 | 1 | 186265405 | 186283694 | protein_coding | −1.113700365 | 7.36E−06 | FALSE | FALSE | FALSE | FALSE | FALSE | FALSE |
| ENSG00000060762 | MPC1 | 6 | 166778407 | 166796486 | protein_coding | −1.114029887 | 1.26E−37 | FALSE | FALSE | FALSE | FALSE | FALSE | FALSE |
| ENSG00000143867 | OSR1 | 2 | 19551246 | 19558414 | protein_coding | −1.114991021 | 1.27E−07 | FALSE | FALSE | FALSE | FALSE | FALSE | FALSE |
| ENSG00000173597 | SULT1B1 | 4 | 70586880 | 70653679 | protein_coding | −1.115696522 | 8.62E−07 | FALSE | FALSE | FALSE | FALSE | FALSE | TRUE |
| ENSG00000258818 | RNASE4 | 14 | 21152259 | 21168761 | protein_coding | −1.11579267 | 1.67E−14 | FALSE | FALSE | FALSE | FALSE | FALSE | TRUE |
| ENSG00000130032 | PRRG3 | X | 150863596 | 150874396 | protein_coding | −1.115856841 | 5.31E−07 | FALSE | FALSE | FALSE | FALSE | FALSE | FALSE |
| ENSG00000174944 | P2RY14 | 3 | 150929905 | 150996255 | protein_coding | −1.116090107 | 7.50E−14 | FALSE | FALSE | FALSE | FALSE | FALSE | FALSE |
| ENSG00000272463 | | 6 | 708592 | 711405 | lincRNA | −1.11610924 | 5.20E−17 | FALSE | FALSE | FALSE | FALSE | FALSE | FALSE |
| ENSG00000183018 | SPNS2 | 17 | 4402133 | 4442330 | protein_coding | −1.11669128 | 3.22E−12 | FALSE | FALSE | FALSE | FALSE | FALSE | FALSE |
| ENSG00000099860 | GADD45B | 19 | 2476120 | 2478257 | protein_coding | −1.116783766 | 4.83E−18 | FALSE | FALSE | FALSE | FALSE | FALSE | FALSE |
| ENSG00000171747 | LGALS4 | 19 | 39292311 | 39304004 | protein_coding | −1.117677115 | 0.000112173 | FALSE | FALSE | FALSE | FALSE | TRUE | TRUE |
| ENSG00000112276 | BVES | 6 | 105544697 | 105585049 | protein_coding | −1.118652968 | 8.64E−12 | FALSE | FALSE | FALSE | FALSE | FALSE | FALSE |
| ENSG00000189221 | MAOA | X | 43515467 | 43606068 | protein_coding | −1.119325454 | 8.42E−16 | FALSE | FALSE | FALSE | FALSE | FALSE | FALSE |
| ENSG00000227117 | | 22 | 30404731 | 30476469 | antisense | −1.120384932 | 1.97E−06 | FALSE | FALSE | FALSE | FALSE | FALSE | FALSE |
| ENSG00000066735 | KIF26A | 14 | 104605060 | 104647231 | protein_coding | −1.12086172 | 6.88E−13 | FALSE | FALSE | FALSE | FALSE | FALSE | FALSE |
| ENSG00000242732 | RGAG4 | X | 71346958 | 71351758 | protein_coding | −1.121615046 | 6.39E−20 | FALSE | FALSE | FALSE | FALSE | FALSE | FALSE |
| ENSG00000125848 | FLRT3 | 20 | 14303634 | 14318262 | protein_coding | −1.12176189 | 7.14E−08 | FALSE | FALSE | FALSE | FALSE | FALSE | FALSE |
| ENSG00000156413 | FUT6 | 19 | 5830621 | 5839742 | protein_coding | −1.122352462 | 0.032468916 | FALSE | FALSE | FALSE | FALSE | FALSE | TRUE |
| ENSG00000175267 | VWA3A | 16 | 22103859 | 22168287 | protein_coding | −1.122766532 | 2.51E−05 | FALSE | FALSE | FALSE | FALSE | FALSE | FALSE |
| ENSG00000185518 | SV2B | 15 | 91643180 | 91844539 | protein_coding | −1.122801671 | 1.34E−06 | FALSE | FALSE | FALSE | FALSE | FALSE | FALSE |
| ENSG00000231367 | | 2 | 38633861 | 38742882 | lincRNA | −1.123294684 | 2.93E−07 | FALSE | FALSE | FALSE | FALSE | FALSE | FALSE |
| ENSG00000147027 | TMEM47 | X | 34645181 | 34675405 | protein_coding | −1.124168478 | 1.05E−21 | FALSE | FALSE | FALSE | FALSE | FALSE | FALSE |
| ENSG00000250889 | | 5 | 74343544 | 74348668 | lincRNA | −1.126694541 | 6.64E−10 | FALSE | FALSE | FALSE | FALSE | FALSE | FALSE |
| ENSG00000185008 | ROBO2 | 3 | 75955846 | 77699115 | protein_coding | −1.126776743 | 6.96E−07 | FALSE | FALSE | FALSE | FALSE | FALSE | FALSE |
| ENSG00000117791 | 2-Mar | 1 | 220921567 | 220958150 | protein_coding | −1.127463959 | 2.81E−27 | FALSE | FALSE | FALSE | FALSE | FALSE | FALSE |
| ENSG00000185052 | SLC24A3 | 20 | 19193290 | 19703581 | protein_coding | −1.127477885 | 1.40E−13 | FALSE | FALSE | FALSE | FALSE | FALSE | FALSE |
| ENSG00000245281 | | 8 | 17942377 | 17953903 | antisense | −1.127561993 | 1.92E−26 | FALSE | FALSE | FALSE | FALSE | FALSE | FALSE |
| ENSG00000229645 | LINC00341 | 14 | 95873606 | 95876427 | lincRNA | −1.127683827 | 5.28E−17 | FALSE | FALSE | FALSE | FALSE | FALSE | FALSE |
| ENSG00000244968 | LIFR-AS1 | 5 | 38556888 | 38671318 | antisense | −1.127916128 | 3.27E−18 | FALSE | FALSE | FALSE | FALSE | FALSE | FALSE |
| ENSG00000121691 | CAT | 11 | 34460472 | 34493609 | protein_coding | −1.128037266 | 4.40E−32 | FALSE | FALSE | FALSE | FALSE | FALSE | FALSE |
| ENSG00000053438 | NNAT | 20 | 36149617 | 36152092 | protein_coding | −1.128911369 | 9.55E−10 | FALSE | FALSE | FALSE | FALSE | FALSE | TRUE |
| ENSG00000232855 | | 21 | 29811667 | 30047170 | lincRNA | −1.13018531 | 6.90E−05 | FALSE | FALSE | FALSE | FALSE | FALSE | TRUE |
| ENSG00000166402 | TUB | 11 | 8040791 | 8127659 | protein_coding | −1.130336506 | 8.37E−14 | FALSE | FALSE | FALSE | FALSE | FALSE | FALSE |
| ENSG00000165410 | CFL2 | 14 | 35179593 | 35184029 | protein_coding | −1.130468819 | 9.29E−20 | FALSE | FALSE | FALSE | FALSE | FALSE | FALSE |
| ENSG00000168679 | SLC16A4 | 1 | 110905470 | 110933704 | protein_coding | −1.130642942 | 1.99E−11 | FALSE | FALSE | FALSE | FALSE | FALSE | TRUE |
| ENSG00000099954 | CECR2 | 22 | 17840837 | 18037850 | protein_coding | −1.131177336 | 2.71E−05 | FALSE | FALSE | FALSE | FALSE | FALSE | FALSE |
| ENSG00000169282 | KCNAB1 | 3 | 155755490 | 156256545 | protein_coding | −1.131319972 | 1.91E−15 | FALSE | FALSE | FALSE | FALSE | FALSE | FALSE |
| ENSG00000179094 | PER1 | 17 | 8043790 | 8059824 | protein_coding | −1.131378614 | 6.65E−28 | FALSE | FALSE | FALSE | FALSE | TRUE | TRUE |
| ENSG00000198865 | CCDC152 | 5 | 42756903 | 42802462 | protein_coding | −1.132578576 | 6.75E−21 | FALSE | FALSE | FALSE | FALSE | FALSE | FALSE |
| ENSG00000115423 | DNAH6 | 2 | 84743579 | 85046713 | protein_coding | −1.132864193 | 1.77E−09 | FALSE | FALSE | FALSE | FALSE | FALSE | FALSE |
| ENSG00000167874 | TMEM88 | 17 | 7758383 | 7759417 | protein_coding | −1.133348876 | 5.28E−18 | FALSE | FALSE | FALSE | FALSE | FALSE | FALSE |
| ENSG00000164099 | PRSS12 | 4 | 119201193 | 119274158 | protein_coding | −1.133528283 | 5.71E−07 | FALSE | FALSE | FALSE | FALSE | FALSE | FALSE |
| ENSG00000233355 | CHRM3-AS2 | 1 | 239866684 | 239893765 | processed_transc [symbol] | −1.133868301 | 1.57E−06 | FALSE | FALSE | FALSE | FALSE | FALSE | FALSE |
| ENSG00000180592 | SKIDA1 | 10 | 21802407 | 21814611 | protein_coding | −1.134641814 | 1.44E−13 | FALSE | FALSE | FALSE | FALSE | FALSE | FALSE |
| ENSG00000186377 | CYP4X1 | 1 | 47427036 | 47516423 | protein_coding | −1.135146726 | 4.16E−10 | FALSE | FALSE | FALSE | FALSE | FALSE | FALSE |
| ENSG00000185046 | ANKS1B | 12 | 99120235 | 100378432 | protein_coding | −1.135384936 | 2.56E−07 | FALSE | FALSE | FALSE | FALSE | FALSE | FALSE |
| ENSG00000114654 | EFCC1 | 3 | 128720472 | 128759585 | protein_coding | −1.136523687 | 9.74E−15 | FALSE | FALSE | FALSE | FALSE | FALSE | FALSE |
| ENSG00000161835 | GRASP | 12 | 52400724 | 52409673 | protein_coding | −1.136717064 | 7.76E−21 | FALSE | FALSE | FALSE | FALSE | FALSE | FALSE |
| ENSG00000016402 | IL20RA | 6 | 137321108 | 137366298 | protein_coding | −1.136828904 | 4.33E−05 | FALSE | FALSE | FALSE | FALSE | FALSE | FALSE |
| ENSG00000064787 | BCAS1 | 20 | 52553316 | 52687304 | protein_coding | −1.138044359 | 0.000313031 | FALSE | FALSE | FALSE | FALSE | FALSE | FALSE |
| ENSG00000254245 | PCDHGA3 | 5 | 140723601 | 140892546 | protein_coding | −1.138625822 | 1.10E−11 | FALSE | FALSE | FALSE | FALSE | FALSE | TRUE |
| ENSG00000138772 | ANXA3 | 4 | 79472673 | 79531597 | protein_coding | −1.138664138 | 5.44E−10 | FALSE | FALSE | FALSE | FALSE | FALSE | FALSE |
| ENSG00000187867 | PALM3 | 19 | 14164177 | 14169971 | protein_coding | −1.139032566 | 4.61E−06 | FALSE | FALSE | FALSE | FALSE | FALSE | FALSE |
| ENSG00000168621 | GDNF | 5 | 37812779 | 37839788 | protein_coding | −1.140798548 | 0.005369661 | FALSE | FALSE | FALSE | FALSE | FALSE | FALSE |
| ENSG00000184454 | NCMAP | 1 | 24882602 | 24935819 | protein_coding | −1.142232788 | 3.14E−07 | FALSE | FALSE | FALSE | FALSE | FALSE | TRUE |
| ENSG00000136457 | CHAD | 17 | 48541857 | 48546327 | protein_coding | −1.142566391 | 3.63E−08 | FALSE | FALSE | FALSE | FALSE | TRUE | TRUE |
| ENSG00000169744 | LDB2 | 4 | 16503164 | 16900432 | protein_coding | −1.14293017 | 2.50E−19 | FALSE | FALSE | FALSE | FALSE | FALSE | FALSE |
| ENSG00000258819 | | 14 | 77535523 | 77542687 | lincRNA | −1.143189528 | 1.41E−12 | FALSE | FALSE | FALSE | FALSE | FALSE | FALSE |
| ENSG00000162840 | MT2P1 | 4 | 69242041 | 69242226 | pseudogene | −1.143500957 | 3.64E−09 | FALSE | FALSE | FALSE | FALSE | FALSE | TRUE |
| ENSG00000220563 | PKMP3 | 6 | 86369610 | 86370324 | pseudogene | −1.145777769 | 1.04E−20 | FALSE | FALSE | FALSE | FALSE | FALSE | TRUE |
| ENSG00000163749 | CCDC158 | 4 | 77234154 | 77343021 | protein_coding | −1.146258913 | 1.17E−14 | FALSE | FALSE | FALSE | FALSE | FALSE | FALSE |
| ENSG00000258484 | SPESP1 | 15 | 69110560 | 69239150 | protein_coding | −1.146555762 | 4.04E−09 | FALSE | FALSE | FALSE | FALSE | FALSE | FALSE |
| ENSG00000187634 | SAMD11 | 1 | 860260 | 879955 | protein_coding | −1.146856754 | 1.68E−07 | FALSE | FALSE | FALSE | FALSE | FALSE | FALSE |
| ENSG00000140807 | NKD1 | 16 | 50582241 | 50670647 | protein_coding | −1.147230211 | 1.04E−05 | FALSE | FALSE | FALSE | FALSE | FALSE | FALSE |
| ENSG00000196338 | NLGN3 | X | 70364681 | 70391051 | protein_coding | −1.147266982 | 2.74E−20 | FALSE | FALSE | FALSE | FALSE | FALSE | TRUE |
| ENSG00000250508 | | 11 | 68638132 | 68642010 | lincRNA | −1.147391442 | 1.28E−09 | FALSE | FALSE | FALSE | FALSE | FALSE | FALSE |
| ENSG00000131386 | GALNT15 | 3 | 16216156 | 16273499 | protein_coding | −1.147407678 | 4.43E−12 | FALSE | FALSE | FALSE | FALSE | FALSE | TRUE |
| ENSG00000175164 | ABO | 9 | 136125788 | 136150617 | processed_transc [symbol] | −1.147482183 | 4.86E−08 | FALSE | FALSE | FALSE | FALSE | FALSE | FALSE |
| ENSG00000160870 | CYP3A7 | 7 | 99302660 | 99332819 | protein_coding | −1.147643571 | 6.31E−07 | FALSE | FALSE | FALSE | FALSE | FALSE | FALSE |
| ENSG00000175806 | MSRA | 8 | 9911778 | 10286401 | protein_coding | −1.148446923 | 8.70E−30 | FALSE | FALSE | FALSE | FALSE | FALSE | FALSE |
| ENSG00000204385 | SLC44A4 | 6 | 31830969 | 31846823 | protein_coding | −1.148828362 | 0.00033594 | FALSE | FALSE | FALSE | FALSE | TRUE | TRUE |
| ENSG00000019169 | MARCO | 2 | 119699742 | 119752236 | protein_coding | −1.150056316 | 0.019866439 | FALSE | FALSE | FALSE | FALSE | FALSE | FALSE |
| ENSG00000250986 | | 4 | 3760475 | 3765117 | lincRNA | −1.150547972 | 3.20E−06 | FALSE | FALSE | FALSE | FALSE | FALSE | FALSE |
| ENSG00000055813 | CCDC85A | 2 | 56411258 | 56613308 | protein_coding | −1.152011136 | 9.71E−08 | FALSE | FALSE | FALSE | FALSE | FALSE | FALSE |
| ENSG00000165092 | ALDH1A1 | 9 | 75515578 | 75695358 | protein_coding | −1.152323505 | 3.24E−11 | FALSE | FALSE | FALSE | FALSE | FALSE | FALSE |
| ENSG00000129270 | MMP28 | 17 | 34083268 | 34122711 | protein_coding | −1.152350447 | 8.23E−10 | FALSE | FALSE | FALSE | FALSE | FALSE | FALSE |
| ENSG00000087495 | PHACTR3 | 20 | 58152564 | 58422766 | protein_coding | −1.152579146 | 9.97E−05 | FALSE | FALSE | FALSE | FALSE | FALSE | FALSE |
| ENSG00000172159 | FRMD3 | 9 | 85857905 | 86153461 | protein_coding | −1.152903671 | 1.81E−12 | FALSE | FALSE | FALSE | FALSE | FALSE | TRUE |
| ENSG00000130234 | ACE2 | X | 15579156 | 15620271 | protein_coding | −1.153057271 | 7.83E−05 | FALSE | FALSE | FALSE | FALSE | FALSE | FALSE |
| ENSG00000183715 | OPCML | 11 | 132284871 | 133402414 | protein_coding | −1.153403121 | 0.000102965 | FALSE | FALSE | FALSE | FALSE | FALSE | FALSE |
| ENSG00000133640 | LRRIQ1 | 12 | 85430092 | 85657002 | protein_coding | −1.154428641 | 1.59E−06 | FALSE | FALSE | FALSE | FALSE | FALSE | FALSE |
| ENSG00000162733 | DDR2 | 1 | 162601163 | 162757190 | protein_coding | −1.154441342 | 9.86E−20 | FALSE | FALSE | FALSE | FALSE | FALSE | TRUE |
| ENSG00000157168 | NRG1 | 8 | 31496902 | 32622548 | protein_coding | −1.154987606 | 5.32E−07 | FALSE | FALSE | FALSE | FALSE | FALSE | TRUE |
| ENSG00000120279 | MYCT1 | 6 | 153019030 | 153045702 | protein_coding | −1.155046816 | 1.29E−20 | FALSE | FALSE | FALSE | FALSE | FALSE | FALSE |
| ENSG00000135960 | EDAR | 2 | 109510927 | 109605828 | protein_coding | −1.156062996 | 0.000536184 | FALSE | FALSE | FALSE | FALSE | FALSE | FALSE |
| ENSG00000149571 | KIRREL3 | 11 | 126293254 | 126873355 | protein_coding | −1.15619867 | 3.71E−10 | FALSE | FALSE | FALSE | FALSE | FALSE | FALSE |
| ENSG00000124713 | GNMT | 6 | 42928496 | 42931618 | protein_coding | −1.156731393 | 1.93E−09 | FALSE | FALSE | FALSE | FALSE | FALSE | TRUE |
| ENSG00000106823 | ECM2 | 9 | 95256365 | 95298937 | protein_coding | −1.158930155 | 5.03E−20 | FALSE | FALSE | FALSE | FALSE | FALSE | FALSE |
| ENSG00000144406 | UNC80 | 2 | 210636717 | 210864024 | protein_coding | −1.159558565 | 2.81E−05 | FALSE | FALSE | FALSE | FALSE | FALSE | FALSE |
| ENSG00000215187 | FAM166B | 9 | 35561828 | 35563896 | protein_coding | −1.159819872 | 3.43E−12 | FALSE | FALSE | FALSE | FALSE | FALSE | FALSE |
| ENSG00000260583 | LINC00515 | 21 | 26955082 | 26955638 | antisense | −1.15992782 | 5.84E−17 | FALSE | FALSE | FALSE | FALSE | FALSE | FALSE |
| ENSG00000079156 | OSBPL6 | 2 | 179059208 | 179264160 | protein_coding | −1.159976408 | 5.48E−14 | FALSE | FALSE | FALSE | FALSE | FALSE | FALSE |
| ENSG00000069431 | ABCC9 | 12 | 21950335 | 22094336 | protein_coding | −1.160186918 | 6.94E−14 | FALSE | FALSE | FALSE | FALSE | FALSE | FALSE |
| ENSG00000102547 | CAB39L | 13 | 49882786 | 50018262 | protein_coding | −1.160626659 | 1.48E−29 | FALSE | FALSE | FALSE | FALSE | FALSE | TRUE |
| ENSG00000125148 | MT2A | 16 | 56642111 | 56643409 | protein_coding | −1.160917546 | 9.12E−11 | FALSE | FALSE | FALSE | FALSE | FALSE | FALSE |
| ENSG00000167912 | | 8 | 60031599 | 60033905 | antisense | −1.161413994 | 1.40E−10 | FALSE | FALSE | FALSE | FALSE | FALSE | FALSE |
| ENSG00000118523 | CTGF | 6 | 132269316 | 132272513 | protein_coding | −1.161597396 | 1.20E−15 | FALSE | FALSE | FALSE | FALSE | FALSE | FALSE |
| ENSG00000183044 | ABAT | 16 | 8768422 | 8878432 | protein_coding | −1.161855441 | 2.20E−11 | FALSE | FALSE | FALSE | FALSE | FALSE | FALSE |
| ENSG00000186354 | C9orf47 | 9 | 91605778 | 91611055 | protein_coding | −1.163329632 | 1.28E−13 | FALSE | FALSE | FALSE | FALSE | FALSE | FALSE |
| ENSG00000108405 | P2RX1 | 17 | 3799886 | 3819794 | protein_coding | −1.164265649 | 3.16E−10 | FALSE | FALSE | FALSE | TRUE | TRUE | TRUE |
| ENSG00000228412 | | 6 | 19802395 | 19804983 | lincRNA | −1.164269551 | 1.62E−08 | FALSE | FALSE | FALSE | FALSE | FALSE | FALSE |
| ENSG00000183454 | GRIN2A | 16 | 9852376 | 10276611 | protein_coding | −1.164300747 | 0.000110781 | FALSE | FALSE | FALSE | FALSE | FALSE | FALSE |
| ENSG00000144278 | GALNT13 | 2 | 154728426 | 155310361 | protein_coding | −1.164430256 | 2.91E−05 | FALSE | FALSE | FALSE | FALSE | FALSE | FALSE |
| ENSG00000176399 | DMRTA1 | 9 | 22446840 | 22455739 | protein_coding | −1.164478138 | 3.06E−10 | FALSE | FALSE | FALSE | FALSE | FALSE | FALSE |
| ENSG00000108187 | PBLD | 10 | 70042417 | 70092806 | protein_coding | −1.164858359 | 2.84E−16 | FALSE | FALSE | FALSE | FALSE | FALSE | TRUE |
| ENSG00000255310 | | 8 | 10965298 | 10967236 | sense_intronic | −1.166121336 | 2.30E−16 | FALSE | FALSE | FALSE | FALSE | FALSE | FALSE |
| ENSG00000082397 | EPB41L3 | 18 | 5392383 | 5630699 | protein_coding | −1.166172319 | 6.72E−23 | FALSE | FALSE | FALSE | FALSE | FALSE | FALSE |
| ENSG00000163239 | TDRD10 | 1 | 154474695 | 154520623 | protein_coding | −1.168118577 | 1.29E−11 | FALSE | FALSE | FALSE | FALSE | FALSE | TRUE |
| ENSG00000174099 | MSRB3 | 12 | 65672423 | 65882024 | protein_coding | −1.16878395 | 1.54E−16 | FALSE | FALSE | FALSE | FALSE | FALSE | FALSE |
| ENSG00000270638 | | 6 | 146056706 | 146058354 | lincRNA | −1.174710488 | 9.50E−17 | FALSE | FALSE | FALSE | FALSE | FALSE | FALSE |
| ENSG00000017427 | IGF1 | 12 | 102789645 | 102874423 | protein_coding | −1.175226897 | 2.00E−09 | FALSE | FALSE | FALSE | FALSE | FALSE | FALSE |
| ENSG00000148057 | IDNK | 9 | 86237964 | 86259045 | protein_coding | −1.176528451 | 3.24E−35 | FALSE | FALSE | FALSE | FALSE | FALSE | FALSE |
| ENSG00000086696 | HSD17B2 | 16 | 82068609 | 82132139 | protein_coding | −1.177401652 | 4.86E−05 | FALSE | FALSE | FALSE | FALSE | FALSE | TRUE |
| ENSG00000245025 | | 8 | 22842109 | 22876848 | antisense | −1.17740191 | 2.63E−34 | FALSE | FALSE | FALSE | FALSE | FALSE | FALSE |
| ENSG00000175906 | ARL4D | 17 | 41476327 | 41478505 | protein_coding | −1.177687347 | 1.82E−15 | FALSE | FALSE | FALSE | FALSE | FALSE | TRUE |
| ENSG00000164591 | MYOZ3 | 5 | 150040436 | 150058927 | protein_coding | −1.177897322 | 5.44E−13 | FALSE | FALSE | FALSE | FALSE | FALSE | FALSE |
| ENSG00000127074 | RGS13 | 1 | 192605275 | 192629390 | protein_coding | −1.177925773 | 4.95E−05 | FALSE | FALSE | FALSE | FALSE | FALSE | FALSE |
| ENSG00000012171 | SEMA3B | 3 | 50304990 | 50314977 | processed_transc [symbol] | −1.178959373 | 1.78E−12 | FALSE | FALSE | FALSE | FALSE | FALSE | FALSE |
| ENSG00000077157 | PPP1R12B | 1 | 202317827 | 202561834 | protein_coding | −1.17922621 | 1.28E−13 | FALSE | FALSE | FALSE | FALSE | TRUE | FALSE |
| ENSG00000162882 | HAAO | 2 | 42994229 | 43019733 | protein_coding | −1.179465533 | 1.23E−15 | FALSE | FALSE | FALSE | FALSE | FALSE | FALSE |
| ENSG00000235903 | CPB2-AS1 | 13 | 46626941 | 46687467 | antisense | −1.179564376 | 2.17E−12 | FALSE | FALSE | FALSE | FALSE | FALSE | FALSE |
| ENSG00000197245 | FAM110D | 1 | 26485511 | 26489119 | protein_coding | −1.180415268 | 1.32E−16 | FALSE | FALSE | FALSE | FALSE | FALSE | FALSE |
| ENSG00000169760 | NLGN1 | 3 | 173114074 | 174004434 | protein_coding | −1.180976725 | 3.96E−05 | FALSE | FALSE | FALSE | TRUE | FALSE | TRUE |
| ENSG00000171817 | ZNF540 | 19 | 38042308 | 38104998 | protein_coding | −1.181111992 | 3.13E−32 | FALSE | FALSE | FALSE | FALSE | FALSE | FALSE |
| ENSG00000139946 | PELI2 | 14 | 56584532 | 56768244 | protein_coding | −1.181731291 | 2.65E−26 | FALSE | FALSE | FALSE | FALSE | FALSE | FALSE |
| ENSG00000175745 | NR2F1 | 5 | 92919043 | 92930321 | protein_coding | −1.182353933 | 7.91E−18 | FALSE | FALSE | FALSE | FALSE | FALSE | FALSE |
| ENSG00000111058 | ACSS3 | 12 | 81331594 | 81650533 | protein_coding | −1.182455838 | 1.30E−12 | FALSE | FALSE | FALSE | FALSE | FALSE | FALSE |
| ENSG00000197992 | CLEC9A | 12 | 10183276 | 10218565 | protein_coding | −1.182612675 | 1.09E−09 | FALSE | FALSE | FALSE | FALSE | FALSE | FALSE |
| ENSG00000120586 | MRC1 | 10 | 18098352 | 18200091 | protein_coding | −1.182864614 | 3.65E−09 | FALSE | FALSE | FALSE | FALSE | FALSE | FALSE |
| ENSG00000153902 | LGI4 | 19 | 35615417 | 35633355 | protein_coding | −1.18296216 | 1.63E−09 | FALSE | FALSE | FALSE | FALSE | TRUE | TRUE |
| ENSG00000180769 | WDFY3-AS2 | 4 | 85887538 | 85932430 | processed_transc [symbol] | −1.18308636 | 9.39E−42 | FALSE | FALSE | FALSE | FALSE | FALSE | TRUE |
| ENSG00000164736 | SOX17 | 8 | 55370495 | 55373448 | protein_coding | −1.184602939 | 6.05E−14 | FALSE | FALSE | FALSE | FALSE | FALSE | TRUE |
| ENSG00000157551 | KCNJ15 | 21 | 39529128 | 39679279 | protein_coding | −1.184792162 | 2.04E−09 | FALSE | FALSE | FALSE | FALSE | FALSE | TRUE |
| ENSG00000196159 | FAT4 | 4 | 126237554 | 126414087 | protein_coding | −1.184819725 | 3.07E−19 | FALSE | FALSE | FALSE | FALSE | FALSE | FALSE |
| ENSG00000163879 | DNALI1 | 1 | 38022520 | 38032458 | protein_coding | −1.186550448 | 3.76E−14 | FALSE | FALSE | FALSE | FALSE | FALSE | FALSE |
| ENSG00000186529 | CYP4F3 | 19 | 15751707 | 15773635 | protein_coding | −1.186652571 | 0.000151458 | FALSE | FALSE | FALSE | FALSE | FALSE | TRUE |
| ENSG00000173662 | TAS1R1 | 1 | 6615241 | 6639817 | protein_coding | −1.18740123 | 6.15E−08 | FALSE | FALSE | FALSE | FALSE | FALSE | FALSE |
| ENSG00000080709 | KCNN2 | 5 | 113696642 | 113832337 | protein_coding | −1.187904985 | 1.32E−08 | FALSE | FALSE | FALSE | FALSE | FALSE | TRUE |
| ENSG00000162614 | NEXN | 1 | 78354198 | 78409580 | protein_coding | −1.18807744 | 7.98E−15 | FALSE | FALSE | FALSE | FALSE | FALSE | FALSE |
| ENSG00000102230 | PCYT1B | X | 24576204 | 24690794 | protein_coding | −1.188482061 | 4.64E−07 | FALSE | FALSE | FALSE | FALSE | FALSE | FALSE |
| ENSG00000135218 | CD36 | 7 | 79998891 | 80308593 | protein_coding | −1.18856519 | 2.24E−06 | FALSE | FALSE | FALSE | FALSE | FALSE | FALSE |
| ENSG00000066185 | ZMYND12 | 1 | 42896000 | 42921938 | protein_coding | −1.189894595 | 3.76E−21 | FALSE | FALSE | FALSE | FALSE | FALSE | FALSE |
| ENSG00000185070 | FLRT2 | 14 | 85996488 | 86095034 | protein_coding | 1.18998662 | 1.42E−12 | FALSE | FALSE | FALSE | FALSE | FALSE | FALSE |
| ENSG00000183775 | KCTD16 | 5 | 143550396 | 143865249 | protein_coding | −1.190213598 | 1.93E−06 | FALSE | FALSE | FALSE | FALSE | FALSE | FALSE |
| ENSG00000135144 | DTX1 | 12 | 113494514 | 113535833 | protein_coding | −1.190430489 | 1.50E−12 | FALSE | FALSE | FALSE | FALSE | FALSE | FALSE |
| ENSG00000112425 | EPM2A | 6 | 145822719 | 146057160 | protein_coding | −1.190666963 | 2.91E−39 | FALSE | FALSE | FALSE | FALSE | FALSE | FALSE |
| ENSG00000198515 | CNGA1 | 4 | 47937994 | 48018689 | protein_coding | −1.190869282 | 7.07E−09 | FALSE | FALSE | FALSE | FALSE | FALSE | FALSE |
| ENSG00000066230 | SLC9A3 | 5 | 473425 | 524447 | protein_coding | −1.19088175 | 0.000565211 | FALSE | FALSE | FALSE | FALSE | FALSE | FALSE |
| ENSG00000233554 | | 9 | 33166973 | 33179981 | antisense | −1.191338295 | 1.84E−36 | FALSE | FALSE | FALSE | FALSE | FALSE | FALSE |
| ENSG00000245293 | | 4 | 108784635 | 108899955 | antisense | −1.191631978 | 1.03E−10 | FALSE | FALSE | FALSE | FALSE | FALSE | FALSE |
| ENSG00000116176 | TPSG1 | 16 | 1271651 | 1275257 | protein_coding | −1.193584992 | 8.05E−05 | FALSE | FALSE | FALSE | FALSE | FALSE | FALSE |
| ENSG00000240602 | | 3 | 151488216 | 151502682 | pseudogene | −1.193966633 | 5.58E−06 | FALSE | FALSE | FALSE | FALSE | FALSE | FALSE |
| ENSG00000141433 | ADCYAP1 | 18 | 904944 | 912173 | protein_coding | −1.195449654 | 0.000114126 | FALSE | FALSE | FALSE | FALSE | FALSE | FALSE |
| ENSG00000180871 | CXCR2 | 2 | 218990012 | 219001976 | protein_coding | −1.195959846 | 3.53E−09 | FALSE | FALSE | TRUE | TRUE | FALSE | TRUE |
| ENSG00000091831 | ESR1 | 6 | 151977826 | 152450754 | protein_coding | −1.196119681 | 5.02E−11 | FALSE | FALSE | FALSE | FALSE | FALSE | TRUE |
| ENSG00000230630 | DNM3OS | 1 | 172109461 | 172113934 | antisense | −1.196181878 | 9.06E−14 | FALSE | FALSE | FALSE | FALSE | FALSE | FALSE |
| ENSG00000157927 | RADIL | 7 | 4836686 | 4923350 | protein_coding | −1.197011529 | 1.17E−12 | FALSE | FALSE | FALSE | FALSE | FALSE | FALSE |
| ENSG00000137731 | FXYD2 | 11 | 117671559 | 117699413 | protein_coding | −1.197075211 | 1.71E−08 | FALSE | FALSE | FALSE | FALSE | FALSE | TRUE |
| ENSG00000228791 | THRB-AS1 | 3 | 24535578 | 24541833 | processed_transc [symbol] | −1.197105999 | 3.06E−18 | FALSE | FALSE | FALSE | FALSE | FALSE | FALSE |
| ENSG00000100170 | SLC5A1 | 22 | 32439019 | 32509016 | protein_coding | −1.19797705 | 0.039368354 | FALSE | FALSE | FALSE | FALSE | FALSE | TRUE |
| ENSG00000114378 | HYAL1 | 3 | 50337320 | 50349812 | protein_coding | −1.198497602 | 6.61E−17 | FALSE | FALSE | FALSE | FALSE | FALSE | TRUE |
| ENSG00000170837 | GPR27 | 3 | 71803201 | 71805647 | protein_coding | −1.199087328 | 1.66E−08 | FALSE | FALSE | FALSE | FALSE | FALSE | FALSE |
| ENSG00000163637 | PRICKLE2 | 3 | 64079543 | 64431152 | protein_coding | −1.199839144 | 2.24E−25 | FALSE | FALSE | FALSE | FALSE | FALSE | FALSE |
| ENSG00000149591 | TAGLN | 11 | 117070037 | 117075498 | protein_coding | −1.20136284 | 3.51E−12 | FALSE | FALSE | FALSE | FALSE | FALSE | FALSE |
| ENSG00000197580 | BCO2 | 11 | 112046190 | 112095422 | protein_coding | −1.202238177 | 5.66E−09 | FALSE | FALSE | FALSE | FALSE | TRUE | TRUE |
| ENSG00000236882 | C5orf27 | 5 | 95187936 | 95195837 | protein_coding | −1.202920391 | 0.001067397 | FALSE | FALSE | FALSE | FALSE | FALSE | TRUE |
| ENSG00000181856 | SLC2A4 | 17 | 7184986 | 7191576 | protein_coding | −1.20343551 | 2.55E−08 | FALSE | FALSE | FALSE | FALSE | FALSE | FALSE |
| ENSG00000171522 | PTGER4 | 5 | 40679600 | 40693837 | protein_coding | −1.203500289 | 1.41E−26 | FALSE | FALSE | FALSE | FALSE | FALSE | FALSE |
| ENSG00000106511 | MEOX2 | 7 | 15650837 | 15726437 | protein_coding | −1.203758365 | 3.78E−08 | FALSE | FALSE | FALSE | FALSE | FALSE | FALSE |
| ENSG00000146521 | C6orf123 | 6 | 168185217 | 168197539 | protein_coding | −1.20379813 | 2.44E−11 | FALSE | FALSE | FALSE | FALSE | FALSE | FALSE |
| ENSG00000198075 | SULT1C4 | 2 | 108994367 | 109004513 | protein_coding | −1.204488111 | 1.15E−09 | FALSE | FALSE | FALSE | FALSE | FALSE | FALSE |
| ENSG00000144218 | AFF3 | 2 | 100162323 | 100759201 | protein_coding | −1.204791551 | 2.60E−07 | FALSE | FALSE | FALSE | FALSE | FALSE | TRUE |
| ENSG00000244607 | CCDC13 | 3 | 42734155 | 42814745 | protein_coding | −1.205470699 | 5.23E−21 | FALSE | FALSE | FALSE | FALSE | FALSE | FALSE |
| ENSG00000198947 | DMD | X | 31115794 | 33357558 | protein_coding | −1.205950983 | 1.05E−14 | FALSE | FALSE | FALSE | FALSE | FALSE | FALSE |
| ENSG00000171509 | RXFP1 | 4 | 159236463 | 159574524 | protein_coding | −1.20715928 | 9.55E−09 | FALSE | FALSE | FALSE | FALSE | FALSE | TRUE |
| ENSG00000084674 | APOB | 2 | 21224301 | 21266945 | protein_coding | −1.207491862 | 0.000102419 | FALSE | FALSE | FALSE | FALSE | FALSE | TRUE |
| ENSG00000205502 | C2CD4B | 15 | 62455734 | 62457482 | protein_coding | −1.207932935 | 2.10E−09 | FALSE | FALSE | FALSE | FALSE | FALSE | FALSE |
| ENSG00000073712 | FERMT2 | 14 | 53323986 | 53419153 | protein_coding | −1.208067231 | 1.31E−28 | FALSE | FALSE | FALSE | FALSE | FALSE | FALSE |
| ENSG00000118596 | SLC16A7 | 12 | 59989848 | 60176395 | protein_coding | −1.208748286 | 1.64E−13 | FALSE | FALSE | FALSE | FALSE | TRUE | FALSE |
| ENSG00000170011 | MYRIP | 3 | 39850405 | 40301812 | protein_coding | −1.208865452 | 2.59E−08 | FALSE | FALSE | FALSE | FALSE | FALSE | FALSE |
| ENSG00000092529 | CAPN3 | 15 | 42640301 | 42704516 | protein_coding | −1.208912994 | 1.59E−11 | FALSE | TRUE | TRUE | TRUE | TRUE | TRUE |
| ENSG00000183508 | FAM46C | 1 | 118148556 | 118170994 | protein_coding | −1.209205242 | 3.07E−20 | FALSE | FALSE | FALSE | FALSE | FALSE | FALSE |
| ENSG00000162367 | TAL1 | 1 | 47681962 | 47697892 | protein_coding | −1.209652746 | 3.27E−20 | FALSE | FALSE | FALSE | FALSE | FALSE | TRUE |
| ENSG00000260833 | | 17 | 36606638 | 36608688 | processed_transc [symbol] | −1.209767918 | 8.13E−10 | FALSE | FALSE | FALSE | FALSE | FALSE | FALSE |
| ENSG00000144648 | ACKR2 | 3 | 42846244 | 42929466 | protein_coding | −1.209862286 | 4.09E−16 | FALSE | FALSE | FALSE | FALSE | FALSE | FALSE |
| ENSG00000144410 | CPO | 2 | 207804278 | 207834198 | protein_coding | −1.210680817 | 4.24E−14 | FALSE | FALSE | FALSE | FALSE | FALSE | FALSE |
| ENSG00000272636 | DOC2B | 17 | 5810 | 31427 | protein_coding | −1.210835684 | 6.25E−10 | FALSE | FALSE | FALSE | FALSE | FALSE | FALSE |
| ENSG00000111254 | AKAP3 | 12 | 4724674 | 4758213 | protein_coding | −1.211863248 | 3.53E−28 | FALSE | FALSE | FALSE | FALSE | FALSE | FALSE |
| ENSG00000101542 | CDH20 | 18 | 59000815 | 59223006 | protein_coding | −1.212140737 | 4.01E−08 | FALSE | FALSE | FALSE | FALSE | FALSE | TRUE |
| ENSG00000178662 | CSRNP3 | 2 | 166326157 | 166545917 | protein_coding | −1.212201568 | 1.26E−17 | FALSE | FALSE | FALSE | FALSE | FALSE | FALSE |
| ENSG00000145284 | SCD5 | 4 | 83550692 | 83720010 | protein_coding | −1.212400425 | 7.82E−14 | FALSE | FALSE | FALSE | FALSE | FALSE | TRUE |
| ENSG00000105784 | RUNDC3B | 7 | 87256864 | 87461611 | protein_coding | −1.213030208 | 5.00E−17 | FALSE | FALSE | FALSE | FALSE | FALSE | FALSE |
| ENSG00000255408 | PCDHA3 | 5 | 140180783 | 140391929 | protein_coding | −1.213064903 | 6.24E−09 | FALSE | FALSE | FALSE | FALSE | FALSE | FALSE |
| ENSG00000214652 | | 7 | 63505821 | 63545179 | pseudogene | −1.214153721 | 9.62E−10 | FALSE | FALSE | FALSE | FALSE | FALSE | FALSE |
| ENSG00000180537 | RNF182 | 6 | 13924677 | 13980533 | protein_coding | −1.216477943 | 2.50E−07 | FALSE | FALSE | FALSE | FALSE | FALSE | FALSE |
| ENSG00000188906 | LRRK2 | 12 | 40590546 | 40763087 | protein_coding | −1.217700843 | 6.49E−09 | FALSE | FALSE | FALSE | FALSE | FALSE | TRUE |
| ENSG00000166257 | SCN3B | 11 | 123499895 | 123525952 | protein_coding | −1.218025534 | 3.42E−14 | FALSE | FALSE | FALSE | FALSE | FALSE | FALSE |
| ENSG00000257894 | | 12 | 79734985 | 79897176 | lincRNA | −1.218223873 | 3.29E−05 | FALSE | FALSE | FALSE | FALSE | FALSE | FALSE |
| ENSG00000132514 | CLEC10A | 17 | 6977856 | 6983626 | protein_coding | −1.218288045 | 2.60E−11 | FALSE | FALSE | FALSE | FALSE | FALSE | FALSE |
| ENSG00000170624 | SGCD | 5 | 155297354 | 156194799 | protein_coding | −1.218417328 | 3.03E−12 | FALSE | FALSE | FALSE | FALSE | FALSE | FALSE |
| ENSG00000180481 | GLIPR1L2 | 12 | 75784850 | 75826468 | protein_coding | −1.218596137 | 3.86E−27 | FALSE | FALSE | FALSE | FALSE | FALSE | FALSE |
| ENSG00000111341 | MGP | 12 | 15034115 | 15038860 | protein_coding | −1.219582678 | 4.60E−13 | FALSE | FALSE | FALSE | FALSE | FALSE | TRUE |
| ENSG00000064655 | EYA2 | 20 | 45523263 | 45817492 | protein_coding | −1.220523349 | 9.61E−09 | FALSE | FALSE | FALSE | FALSE | FALSE | TRUE |
| ENSG00000178826 | TMEM139 | 7 | 142977050 | 142985141 | protein_coding | −1.22118805 | 2.84E−09 | FALSE | FALSE | FALSE | FALSE | FALSE | TRUE |
| ENSG00000224940 | PRRT4 | 7 | 127990379 | 128001739 | protein_coding | −1.221317148 | 2.82E−06 | FALSE | FALSE | FALSE | FALSE | FALSE | FALSE |
| ENSG00000005187 | ACSM3 | 16 | 20621565 | 20808903 | protein_coding | −1.221517381 | 2.16E−14 | FALSE | FALSE | FALSE | FALSE | FALSE | TRUE |
| ENSG00000203727 | SAMD5 | 6 | 147830063 | 148058683 | protein_coding | −1.223688971 | 3.49E−11 | FALSE | FALSE | FALSE | FALSE | FALSE | FALSE |
| ENSG00000145020 | AMT | 3 | 49454211 | 49460186 | protein_coding | −1.224037609 | 5.57E−27 | FALSE | FALSE | FALSE | FALSE | TRUE | TRUE |
| ENSG00000179256 | SMCO3 | 12 | 14957584 | 14967116 | protein_coding | −1.224129812 | 1.18E−10 | FALSE | FALSE | FALSE | FALSE | FALSE | FALSE |
| ENSG00000099864 | PALM | 19 | 708953 | 748329 | protein_coding | −1.224632334 | 8.79E−18 | FALSE | FALSE | FALSE | FALSE | FALSE | FALSE |
| ENSG00000183784 | C9orf66 | 9 | 213108 | 215893 | protein_coding | −1.225494071 | 9.04E−12 | FALSE | FALSE | FALSE | FALSE | FALSE | FALSE |
| ENSG00000205683 | DPF3 | 14 | 73086004 | 73360809 | protein_coding | −1.226481398 | 2.16E−15 | FALSE | FALSE | FALSE | FALSE | FALSE | FALSE |
| ENSG00000167107 | ACSF2 | 17 | 48503519 | 48552206 | protein_coding | −1.226732076 | 1.18E−16 | FALSE | FALSE | FALSE | FALSE | FALSE | FALSE |
| ENSG00000165300 | SLITRK5 | 13 | 88324870 | 88331871 | protein_coding | −1.227049166 | 0.00090156 | FALSE | FALSE | FALSE | FALSE | FALSE | FALSE |
| ENSG00000196177 | ACADSB | 10 | 124768495 | 124817827 | protein_coding | −1.228912984 | 2.46E−28 | FALSE | FALSE | FALSE | FALSE | FALSE | TRUE |
| ENSG00000170190 | SLC16A5 | 17 | 73083822 | 73102257 | protein_coding | −1.229072053 | 5.44E−18 | FALSE | FALSE | FALSE | FALSE | FALSE | FALSE |
| ENSG00000159388 | BTG2 | 1 | 203274619 | 203278730 | protein_coding | −1.229136412 | 2.35E−26 | FALSE | FALSE | FALSE | FALSE | FALSE | FALSE |
| ENSG00000169313 | P2RY12 | 3 | 151055168 | 151102600 | protein_coding | −1.229730405 | 9.43E−09 | FALSE | FALSE | FALSE | FALSE | FALSE | FALSE |
| ENSG00000183807 | FAM162B | 6 | 117073363 | 117086886 | protein_coding | −1.230208768 | 1.17E−13 | FALSE | FALSE | FALSE | FALSE | FALSE | TRUE |
| ENSG00000188039 | NWD1 | 19 | 16830787 | 16928774 | protein_coding | −1.230951096 | 2.53E−07 | FALSE | FALSE | FALSE | FALSE | FALSE | TRUE |
| ENSG00000261069 | SNORD116-21 | 15 | 25332808 | 25334043 | lincRNA | −1.232145872 | 1.30E−14 | FALSE | FALSE | FALSE | FALSE | FALSE | FALSE |
| ENSG00000261069 | SNORD116-20 | 15 | 25332808 | 25334043 | lincRNA | −1.232145872 | 1.30E−14 | FALSE | FALSE | FALSE | FALSE | FALSE | FALSE |
| ENSG00000253051 | | 13 | 45910449 | 45910582 | snoRNA | −1.232423552 | 5.03E−27 | FALSE | FALSE | FALSE | FALSE | FALSE | FALSE |
| ENSG00000136002 | ARHGEF4 | 2 | 131594489 | 131804836 | protein_coding | −1.232551132 | 5.45E−10 | FALSE | FALSE | FALSE | FALSE | TRUE | TRUE |
| ENSG00000196136 | SERPINA3 | 14 | 95078714 | 95090392 | protein_coding | −1.233610642 | 2.20E−05 | FALSE | FALSE | FALSE | FALSE | FALSE | TRUE |
| ENSG00000138639 | ARHGAP24 | 4 | 86396267 | 86923823 | protein_coding | −1.234520348 | 1.76E−34 | FALSE | FALSE | FALSE | FALSE | FALSE | FALSE |
| ENSG00000169994 | MYO7B | 2 | 128293378 | 128395304 | protein_coding | −1.23523318 | 3.79E−08 | FALSE | FALSE | FALSE | FALSE | FALSE | TRUE |
| ENSG00000124701 | APOBEC2 | 6 | 41021043 | 41032250 | protein_coding | −1.236200411 | 1.94E−08 | FALSE | FALSE | FALSE | FALSE | FALSE | TRUE |
| ENSG00000180638 | SLC47A2 | 17 | 19581601 | 19622292 | protein_coding | −1.237284007 | 4.15E−05 | FALSE | FALSE | FALSE | FALSE | FALSE | TRUE |
| ENSG00000196972 | LINC00087 | X | 134229058 | 134232664 | lincRNA | −1.237467759 | 5.14E−22 | FALSE | FALSE | FALSE | FALSE | FALSE | FALSE |
| ENSG00000204789 | ZNF204P | 6 | 27325604 | 27339304 | pseudogene | −1.237950404 | 1.16E−23 | FALSE | FALSE | FALSE | FALSE | FALSE | FALSE |
| ENSG00000130518 | KIAA1683 | 19 | 18367908 | 18385319 | protein_coding | −1.238050438 | 1.29E−17 | FALSE | FALSE | FALSE | FALSE | FALSE | FALSE |
| ENSG00000079102 | RUNX1T1 | 8 | 92967203 | 93115514 | protein_coding | −1.238603508 | 2.42E−19 | FALSE | FALSE | FALSE | FALSE | FALSE | FALSE |
| ENSG00000101144 | BMP7 | 20 | 55743804 | 55841685 | protein_coding | −1.238828223 | 0.002860987 | FALSE | FALSE | FALSE | FALSE | FALSE | TRUE |
| ENSG00000256139 | | 12 | 109549023 | 109563399 | sense_overlapping | −1.239852907 | 1.18E−19 | FALSE | FALSE | FALSE | FALSE | FALSE | FALSE |
| ENSG00000163492 | CCDC141 | 2 | 179694484 | 179914813 | protein_coding | −1.239969981 | 7.45E−09 | FALSE | FALSE | FALSE | FALSE | FALSE | FALSE |
| ENSG00000215142 | | 9 | 40491622 | 40633282 | pseudogene | −1.240577392 | 2.81E−09 | FALSE | FALSE | FALSE | FALSE | FALSE | FALSE |
| ENSG00000150656 | CNDP1 | 18 | 72201675 | 72254448 | protein_coding | −1.240916872 | 0.000836077 | FALSE | FALSE | FALSE | FALSE | FALSE | FALSE |
| ENSG00000113389 | NPR3 | 5 | 32689176 | 32791819 | protein_coding | −1.242271819 | 1.13E−09 | FALSE | FALSE | FALSE | FALSE | FALSE | FALSE |
| ENSG00000133328 | HRASLS2 | 11 | 63320242 | 63330855 | protein_coding | −1.243993675 | 4.15E−06 | FALSE | FALSE | FALSE | FALSE | FALSE | FALSE |
| ENSG00000206052 | DOK6 | 18 | 67068291 | 67516323 | protein_coding | −1.244574465 | 1.50E−14 | FALSE | FALSE | FALSE | FALSE | FALSE | FALSE |
| ENSG00000010379 | SLC6A13 | 12 | 329789 | 372039 | protein_coding | −1.244656728 | 1.30E−05 | FALSE | FALSE | FALSE | FALSE | FALSE | TRUE |
| ENSG00000177103 | DSCAML1 | 11 | 117298489 | 117688240 | protein_coding | −1.245507827 | 4.27E−08 | FALSE | FALSE | FALSE | FALSE | FALSE | FALSE |
| ENSG00000267272 | LINC01140 | 1 | 87595448 | 87634881 | lincRNA | −1.245519293 | 4.24E−15 | FALSE | FALSE | FALSE | FALSE | FALSE | FALSE |
| ENSG00000169252 | ADRB2 | 5 | 148206156 | 148208196 | protein_coding | −1.245937903 | 1.77E−12 | FALSE | FALSE | FALSE | FALSE | FALSE | TRUE |
| ENSG00000158481 | CD1C | 1 | 158259576 | 158263420 | protein_coding | −1.24651666 | 8.74E−12 | FALSE | FALSE | FALSE | FALSE | FALSE | FALSE |
| ENSG00000185272 | RBM11 | 21 | 15588451 | 15600693 | protein_coding | −1.24666201 | 3.22E−07 | FALSE | FALSE | FALSE | FALSE | FALSE | FALSE |
| ENSG00000125968 | ID1 | 20 | 30193086 | 30194318 | protein_coding | −1.246732822 | 1.25E−18 | FALSE | FALSE | FALSE | FALSE | FALSE | TRUE |
| ENSG00000176928 | GCNT4 | 5 | 74323289 | 74326724 | protein_coding | −1.24696903 | 9.78E−17 | FALSE | FALSE | FALSE | FALSE | FALSE | FALSE |
| ENSG00000022556 | NLRP2 | 19 | 55464498 | 55512510 | protein_coding | −1.248044065 | 2.81E−06 | FALSE | FALSE | FALSE | FALSE | FALSE | FALSE |
| ENSG00000148339 | SLC25A25 | 9 | 130830480 | 130871524 | protein_coding | −1.248768061 | 4.66E−31 | FALSE | FALSE | FALSE | FALSE | FALSE | FALSE |
| ENSG00000196381 | ZNF781 | 19 | 38158652 | 38183223 | protein_coding | −1.24889874 | 1.29E−22 | FALSE | FALSE | FALSE | FALSE | FALSE | FALSE |
| ENSG00000250327 | | 4 | 187079050 | 187079997 | pseudogene | −1.248997636 | 1.94E−27 | FALSE | FALSE | FALSE | FALSE | FALSE | FALSE |
| ENSG00000174326 | SLC16A11 | 17 | 6944949 | 6947242 | protein_coding | −1.249376752 | 1.57E−11 | FALSE | FALSE | FALSE | FALSE | FALSE | FALSE |
| ENSG00000241399 | CD302 | 2 | 160625364 | 160654753 | protein_coding | −1.249445325 | 6.43E−24 | FALSE | FALSE | FALSE | FALSE | FALSE | FALSE |
| ENSG00000126562 | WNK4 | 17 | 40932696 | 40948954 | protein_coding | −1.252405397 | 1.96E−06 | FALSE | FALSE | FALSE | FALSE | FALSE | TRUE |
| ENSG00000204403 | CASP12 | 11 | 104756445 | 104769397 | protein_coding | −1.252679711 | 4.78E−09 | FALSE | FALSE | FALSE | FALSE | FALSE | FALSE |
| ENSG00000183833 | MAATS1 | 3 | 119421869 | 119485949 | protein_coding | −1.252839184 | 1.42E−12 | FALSE | FALSE | FALSE | FALSE | FALSE | FALSE |
| ENSG00000245468 | | 4 | 7096298 | 7105112 | lincRNA | −1.2545634 | 1.57E−26 | FALSE | FALSE | FALSE | FALSE | FALSE | FALSE |
| ENSG00000260432 | | 16 | 9847272 | 9851895 | lincRNA | −1.254655441 | 6.01E−05 | FALSE | FALSE | FALSE | FALSE | FALSE | FALSE |
| ENSG00000153234 | NR4A2 | 2 | 157180944 | 157198860 | protein_coding | −1.255991177 | 7.78E−17 | FALSE | FALSE | FALSE | FALSE | FALSE | FALSE |
| ENSG00000140279 | DUOX2 | 15 | 45384848 | 45406542 | protein_coding | −1.256655177 | 2.53E−06 | FALSE | FALSE | FALSE | FALSE | FALSE | TRUE |
| ENSG00000128045 | RASL11B | 4 | 53728457 | 53733000 | protein_coding | −1.258036252 | 1.65E−05 | FALSE | FALSE | FALSE | FALSE | FALSE | FALSE |
| ENSG00000021826 | CPS1 | 2 | 211342406 | 211543831 | protein_coding | −1.258431579 | 1.46E−08 | FALSE | FALSE | FALSE | FALSE | FALSE | FALSE |
| ENSG00000180525 | PRR26 | 10 | 695888 | 711109 | protein_coding | −1.259725039 | 6.29E−13 | FALSE | FALSE | FALSE | FALSE | FALSE | TRUE |
| ENSG00000068831 | RASGRP2 | 11 | 64494383 | 64512928 | protein_coding | −1.261323177 | 6.52E−20 | FALSE | FALSE | FALSE | TRUE | TRUE | TRUE |
| ENSG00000184374 | COLEC10 | 8 | 120007691 | 120118821 | protein_coding | −1.26225799 | 2.51E−05 | FALSE | FALSE | FALSE | FALSE | FALSE | FALSE |
| ENSG00000185736 | ADARB2 | 10 | 1228073 | 1779670 | protein_coding | −1.263862557 | 3.41E−08 | FALSE | FALSE | FALSE | FALSE | FALSE | FALSE |
| ENSG00000119711 | ALDH6A1 | 14 | 74523553 | 74551196 | protein_coding | −1.265200978 | 8.31E−22 | FALSE | FALSE | FALSE | FALSE | FALSE | FALSE |
| ENSG00000152527 | PLEKHH2 | 2 | 43864412 | 43995126 | protein_coding | −1.266063333 | 5.04E−18 | FALSE | FALSE | FALSE | FALSE | FALSE | FALSE |
| ENSG00000160282 | FTCD | 21 | 47556176 | 47575481 | protein_coding | −1.266723249 | 4.65E−05 | FALSE | FALSE | FALSE | FALSE | FALSE | FALSE |
| ENSG00000206538 | VGLL3 | 3 | 86987119 | 87040269 | protein_coding | −1.267218682 | 2.94E−12 | FALSE | FALSE | FALSE | FALSE | FALSE | FALSE |
| ENSG00000154678 | PDE1C | 7 | 31790793 | 32338941 | protein_coding | −1.267347038 | 1.76E−09 | FALSE | FALSE | FALSE | FALSE | FALSE | FALSE |
| ENSG00000146411 | SLC2A12 | 6 | 134309835 | 134373774 | protein_coding | −1.267796648 | 6.77E−10 | FALSE | FALSE | FALSE | FALSE | FALSE | TRUE |
| ENSG00000152268 | SPON1 | 11 | 13983914 | 14289646 | processed_transc [symbol] | −1.268220079 | 9.38E−10 | FALSE | FALSE | FALSE | FALSE | FALSE | FALSE |
| ENSG00000178445 | GLDC | 9 | 6532464 | 6645650 | protein_coding | −1.268252833 | 2.73E−05 | FALSE | FALSE | FALSE | FALSE | FALSE | FALSE |
| ENSG00000154734 | ADAMTS1 | 21 | 28208066 | 28217728 | protein_coding | −1.268716518 | 9.13E−19 | FALSE | FALSE | FALSE | FALSE | FALSE | FALSE |
| ENSG00000265158 | LRRC37A7P | 18 | 29304161 | 29306919 | pseudogene | −1.269685938 | 3.72E−08 | FALSE | FALSE | FALSE | FALSE | FALSE | FALSE |
| ENSG00000118777 | ABCG2 | 4 | 89011416 | 89152474 | protein_coding | −1.270073041 | 3.86E−14 | FALSE | FALSE | FALSE | FALSE | FALSE | FALSE |
| ENSG00000185345 | PARK2 | 6 | 161768452 | 163148803 | protein_coding | −1.270174382 | 5.27E−26 | FALSE | FALSE | FALSE | FALSE | FALSE | FALSE |
| ENSG00000105523 | FAM83E | 19 | 49104067 | 49118111 | protein_coding | −1.270534655 | 1.98E−05 | FALSE | FALSE | FALSE | FALSE | FALSE | TRUE |
| ENSG00000106688 | SLC1A1 | 9 | 4490444 | 4587469 | protein_coding | −1.270646671 | 2.30E−12 | FALSE | FALSE | FALSE | FALSE | FALSE | FALSE |
| ENSG00000113578 | FGF1 | 5 | 141971743 | 142077617 | protein_coding | −1.270780403 | 3.42E−09 | FALSE | FALSE | FALSE | FALSE | FALSE | FALSE |
| ENSG00000169116 | PARM1 | 4 | 75858305 | 75975325 | protein_coding | −1.27120824 | 5.18E−20 | FALSE | FALSE | FALSE | FALSE | FALSE | TRUE |
| ENSG00000178162 | FAR2P2 | 2 | 131174328 | 131188936 | pseudogene | −1.272666653 | 3.45E−10 | FALSE | FALSE | FALSE | FALSE | FALSE | FALSE |
| ENSG00000145861 | C1QTNF2 | 5 | 159774758 | 159797648 | protein_coding | −1.273275676 | 3.71E−19 | FALSE | FALSE | FALSE | FALSE | FALSE | FALSE |
| ENSG00000183748 | | 10 | 17851362 | 17953178 | protein_coding | −1.273680072 | 4.75E−11 | FALSE | FALSE | FALSE | FALSE | FALSE | FALSE |
| ENSG00000185737 | NRG3 | 10 | 83635070 | 84746935 | protein_coding | −1.27389303 | 7.93E−05 | FALSE | FALSE | FALSE | FALSE | FALSE | FALSE |
| ENSG00000073756 | PTGS2 | 1 | 186640923 | 186649559 | protein_coding | −1.274468949 | 8.89E−09 | FALSE | FALSE | FALSE | FALSE | FALSE | FALSE |
| ENSG00000267532 | MIR497HG | 17 | 6919137 | 6922978 | antisense | −1.274974935 | 7.81E−20 | FALSE | FALSE | FALSE | FALSE | FALSE | FALSE |
| ENSG00000161055 | SCGB3A1 | 5 | 180017103 | 180018540 | protein_coding | −1.275400156 | 0.001289742 | FALSE | FALSE | FALSE | FALSE | FALSE | FALSE |
| ENSG00000102468 | HTR2A | 13 | 47405685 | 47471169 | protein_coding | −1.275821402 | 5.51E−10 | FALSE | FALSE | FALSE | FALSE | FALSE | FALSE |
| ENSG00000131781 | FMO5 | 1 | 146646930 | 146714700 | protein_coding | −1.276222201 | 2.94E−13 | FALSE | FALSE | FALSE | FALSE | FALSE | TRUE |
| ENSG00000139209 | SLC38A4 | 12 | 47158546 | 47226191 | protein_coding | −1.276518805 | 5.96E−08 | FALSE | FALSE | FALSE | FALSE | FALSE | FALSE |
| ENSG00000257108 | NHLRC4 | 16 | 616996 | 619495 | protein_coding | −1.277382318 | 1.01E−13 | FALSE | FALSE | FALSE | FALSE | FALSE | FALSE |
| ENSG00000184564 | SLITRK6 | 13 | 86366925 | 86373623 | protein_coding | −1.27769664 | 9.30E−05 | FALSE | FALSE | FALSE | FALSE | FALSE | FALSE |
| ENSG00000176907 | C8orf4 | 8 | 40010989 | 40012821 | protein_coding | −1.278056758 | 1.56E−13 | FALSE | FALSE | FALSE | FALSE | FALSE | FALSE |
| ENSG00000065534 | MYLK | 3 | 123328896 | 123603178 | protein_coding | −1.278155251 | 1.34E−18 | FALSE | FALSE | FALSE | FALSE | FALSE | FALSE |
| ENSG00000227591 | | 1 | 209834709 | 209897470 | antisense | −1.278247338 | 2.20E−10 | FALSE | FALSE | FALSE | FALSE | FALSE | FALSE |
| ENSG00000164850 | GPER1 | 7 | 1121844 | 1133451 | protein_coding | −1.280348647 | 3.93E−15 | FALSE | FALSE | FALSE | FALSE | FALSE | FALSE |
| ENSG00000226816 | | 7 | 23245632 | 23247664 | lincRNA | −1.280478931 | 2.53E−10 | FALSE | FALSE | FALSE | FALSE | FALSE | FALSE |
| ENSG00000168824 | | 4 | 4349867 | 4420785 | protein_coding | −1.280514984 | 6.90E−11 | FALSE | FALSE | FALSE | FALSE | FALSE | TRUE |
| ENSG00000166250 | CLMP | 11 | 122943035 | 123065989 | protein_coding | −1.280636216 | 1.47E−13 | FALSE | FALSE | FALSE | FALSE | FALSE | FALSE |
| ENSG00000183778 | B3GALT5 | 21 | 40928369 | 41045064 | protein_coding | −1.280976197 | 5.76E−05 | FALSE | FALSE | FALSE | FALSE | FALSE | FALSE |
| ENSG00000155970 | MICU3 | 8 | 16884747 | 16980153 | protein_coding | −1.281606068 | 1.02E−23 | FALSE | FALSE | FALSE | FALSE | FALSE | FALSE |
| ENSG00000164512 | ANKRD55 | 5 | 55395507 | 55529186 | protein_coding | −1.283500773 | 4.88E−12 | FALSE | FALSE | FALSE | FALSE | TRUE | FALSE |
| ENSG00000239474 | KLHL41 | 2 | 170366212 | 170382772 | protein_coding | −1.284117227 | 2.62E−11 | FALSE | FALSE | FALSE | FALSE | FALSE | TRUE |
| ENSG00000106714 | CNTNAP3 | 9 | 39072764 | 39288312 | protein_coding | −1.286062925 | 1.74E−11 | FALSE | FALSE | FALSE | FALSE | FALSE | FALSE |
| ENSG00000079101 | CLUL1 | 18 | 596988 | 650334 | protein_coding | −1.286389851 | 1.49E−09 | FALSE | FALSE | FALSE | FALSE | FALSE | TRUE |
| ENSG00000172367 | PDZD3 | 11 | 119056166 | 119060932 | protein_coding | −1.28645327 | 6.54E−06 | FALSE | FALSE | FALSE | FALSE | FALSE | TRUE |
| ENSG00000109846 | CRYAB | 11 | 111779289 | 111794446 | protein_coding | −1.286478866 | 5.98E−11 | FALSE | FALSE | FALSE | FALSE | FALSE | TRUE |
| ENSG00000166770 | ZNF667-AS1 | 19 | 56988619 | 57012035 | lincRNA | −1.286565963 | 2.64E−22 | FALSE | FALSE | FALSE | FALSE | FALSE | FALSE |
| ENSG00000144642 | RBMS3 | 3 | 29322473 | 30051886 | protein_coding | −1.286605414 | 1.42E−28 | FALSE | FALSE | FALSE | FALSE | FALSE | FALSE |
| ENSG00000254602 | | 11 | 57405497 | 57420263 | sense_overlapping | −1.289251552 | 3.17E−16 | FALSE | FALSE | FALSE | FALSE | FALSE | FALSE |
| ENSG00000068615 | REEP1 | 2 | 86441116 | 86565206 | protein_coding | −1.290499372 | 4.26E−13 | FALSE | FALSE | FALSE | FALSE | FALSE | FALSE |
| ENSG00000071967 | CYBRD1 | 2 | 172378757 | 172414643 | protein_coding | −1.291730759 | 4.38E−30 | FALSE | FALSE | FALSE | FALSE | FALSE | FALSE |
| ENSG00000238078 | | 1 | 221002597 | 221005771 | lincRNA | −1.292487384 | 1.59E−13 | FALSE | FALSE | FALSE | FALSE | FALSE | FALSE |
| ENSG00000166148 | AVPR1A | 12 | 63539014 | 63544722 | protein_coding | −1.293894824 | 1.80E−07 | FALSE | FALSE | FALSE | FALSE | FALSE | FALSE |
| ENSG00000174502 | SLC26A9 | 1 | 205882176 | 205912588 | protein_coding | −1.295239581 | 7.12E−05 | FALSE | FALSE | FALSE | FALSE | FALSE | TRUE |
| ENSG00000174348 | PODN | 1 | 53527854 | 53551174 | protein_coding | −1.295373168 | 5.93E−13 | FALSE | FALSE | FALSE | FALSE | FALSE | FALSE |
| ENSG00000154188 | ANGPT1 | 8 | 108261721 | 108510283 | protein_coding | −1.295517591 | 1.88E−14 | FALSE | FALSE | FALSE | FALSE | FALSE | FALSE |
| ENSG00000173811 | CCDC13-AS1 | 3 | 42774067 | 42788260 | antisense | −1.29557301 | 6.51E−19 | FALSE | FALSE | FALSE | FALSE | FALSE | FALSE |
| ENSG00000233947 | | 9 | 80793179 | 80794428 | pseudogene | −1.298389224 | 4.26E−09 | FALSE | FALSE | FALSE | FALSE | FALSE | FALSE |
| ENSG00000095637 | SORBS1 | 10 | 97071528 | 97321171 | protein_coding | −1.301233997 | 2.09E−16 | FALSE | FALSE | FALSE | FALSE | FALSE | TRUE |
| ENSG00000164398 | ACSL6 | 5 | 131142683 | 131347936 | protein_coding | −1.301718976 | 2.85E−05 | FALSE | FALSE | FALSE | FALSE | FALSE | FALSE |
| ENSG00000085741 | WNT11 | 11 | 75897369 | 75921780 | protein_coding | −1.301963698 | 4.83E−09 | FALSE | FALSE | FALSE | FALSE | FALSE | FALSE |
| ENSG00000146122 | DAAM2 | 6 | 39760142 | 39872648 | protein_coding | −1.302363299 | 4.41E−24 | FALSE | FALSE | FALSE | FALSE | TRUE | TRUE |
| ENSG00000179761 | PIPOX | 17 | 27277531 | 27384234 | protein_coding | −1.304849077 | 1.15E−08 | FALSE | FALSE | FALSE | FALSE | FALSE | FALSE |
| ENSG00000236304 | | 11 | 76368100 | 76374910 | antisense | −1.305026305 | 9.21E−15 | FALSE | FALSE | FALSE | FALSE | FALSE | FALSE |
| ENSG00000101977 | MCF2 | X | 138663929 | 138790386 | protein_coding | −1.305748869 | 1.25E−08 | FALSE | FALSE | FALSE | FALSE | FALSE | FALSE |
| ENSG00000117834 | SLC5A9 | 1 | 48688357 | 48714316 | protein_coding | −1.306346815 | 3.08E−11 | FALSE | FALSE | FALSE | FALSE | TRUE | TRUE |
| ENSG00000108370 | RGS9 | 17 | 63133549 | 63223821 | protein_coding | −1.306389152 | 6.73E−15 | FALSE | FALSE | FALSE | FALSE | FALSE | FALSE |
| ENSG00000207949 | MIR214 | 1 | 172107938 | 172108047 | miRNA | −1.306409951 | 1.69E−16 | FALSE | FALSE | FALSE | FALSE | FALSE | FALSE |
| ENSG00000116147 | TNR | 1 | 175284330 | 175712906 | protein_coding | −1.306483359 | 2.92E−05 | FALSE | FALSE | FALSE | FALSE | FALSE | TRUE |
| ENSG00000186310 | NAP1L3 | X | 92925929 | 92928567 | protein_coding | −1.306734316 | 3.10E−20 | FALSE | FALSE | FALSE | FALSE | FALSE | FALSE |
| ENSG00000180929 | GPR62 | 3 | 51989330 | 51991509 | protein_coding | −1.306748871 | 2.22E−13 | FALSE | FALSE | FALSE | FALSE | FALSE | FALSE |
| ENSG00000241978 | AKAP2 | 9 | 112542769 | 112934792 | protein_coding | −1.308824545 | 3.36E−12 | FALSE | FALSE | FALSE | FALSE | FALSE | TRUE |
| ENSG00000063127 | SLC6A16 | 19 | 49792895 | 49828482 | protein_coding | −1.30938609 | 1.80E−25 | FALSE | FALSE | FALSE | FALSE | FALSE | FALSE |
| ENSG00000118514 | ALDH8A1 | 6 | 135238528 | 135271260 | protein_coding | −1.311196958 | 7.89E−12 | FALSE | FALSE | FALSE | FALSE | FALSE | TRUE |
| ENSG00000119138 | KLF9 | 9 | 72999503 | 73029540 | protein_coding | −1.312132548 | 1.10E−46 | FALSE | FALSE | FALSE | FALSE | FALSE | FALSE |
| ENSG00000228798 | | 21 | 18003146 | 18013002 | lincRNA | −1.312419926 | 1.95E−15 | FALSE | FALSE | FALSE | FALSE | FALSE | FALSE |
| ENSG00000159212 | CLIC6 | 21 | 36041688 | 36090525 | protein_coding | −1.313228404 | 1.99E−09 | FALSE | FALSE | FALSE | FALSE | FALSE | FALSE |
| ENSG00000136826 | KLF4 | 9 | 110247133 | 110252763 | protein_coding | −1.314606575 | 1.29E−22 | FALSE | FALSE | TRUE | TRUE | TRUE | FALSE |
| ENSG00000182983 | ZNF662 | 3 | 42947223 | 42960825 | protein_coding | −1.314911492 | 7.57E−24 | FALSE | FALSE | FALSE | FALSE | FALSE | FALSE |
| ENSG00000154654 | NCAM2 | 21 | 22370633 | 22915650 | protein_coding | −1.316547298 | 3.14E−10 | FALSE | FALSE | FALSE | FALSE | FALSE | FALSE |
| ENSG00000183690 | EFHC2 | X | 44007128 | 44202918 | protein_coding | −1.31702692 | 2.63E−09 | FALSE | FALSE | FALSE | FALSE | FALSE | FALSE |
| ENSG00000157445 | CACNA2D3 | 3 | 54156574 | 55108584 | protein_coding | −1.317094597 | 1.97E−13 | FALSE | FALSE | FALSE | FALSE | FALSE | FALSE |
| ENSG00000224063 | | 2 | 187867947 | 188419390 | antisense | −1.317315443 | 1.61E−10 | FALSE | FALSE | FALSE | FALSE | FALSE | FALSE |
| ENSG00000164344 | KLKB1 | 4 | 187130133 | 187179625 | protein_coding | −1.317985067 | 1.31E−15 | FALSE | FALSE | FALSE | FALSE | FALSE | TRUE |
| ENSG00000198624 | CCDC69 | 5 | 150560613 | 150603706 | protein_coding | −1.318642047 | 2.74E−27 | FALSE | FALSE | FALSE | TRUE | TRUE | TRUE |
| ENSG00000127528 | KLF2 | 19 | 16435628 | 16438685 | protein_coding | −1.319299007 | 4.94E−25 | FALSE | FALSE | FALSE | FALSE | FALSE | FALSE |
| ENSG00000130707 | ASS1 | 9 | 133320316 | 133376661 | protein_coding | −1.319523029 | 1.79E−12 | FALSE | FALSE | FALSE | FALSE | FALSE | FALSE |
| ENSG00000143001 | TMEM61 | 1 | 55446465 | 55457966 | protein_coding | −1.320480402 | 4.01E−06 | FALSE | FALSE | FALSE | FALSE | FALSE | FALSE |
| ENSG00000179921 | GPBAR1 | 2 | 219124219 | 219128582 | protein_coding | −1.320579427 | 1.59E−16 | FALSE | FALSE | FALSE | FALSE | FALSE | FALSE |
| ENSG00000117525 | F3 | 1 | 94994781 | 95007356 | protein_coding | −1.320790826 | 2.02E−16 | FALSE | FALSE | FALSE | FALSE | FALSE | FALSE |
| ENSG00000062524 | LTK | 15 | 41795836 | 41806085 | protein_coding | −1.321111701 | 1.36E−10 | FALSE | FALSE | FALSE | FALSE | FALSE | FALSE |
| ENSG00000253953 | PCDHGB4 | 5 | 140767452 | 140892546 | protein_coding | −1.321610893 | 3.03E−15 | FALSE | FALSE | FALSE | FALSE | FALSE | TRUE |
| ENSG00000196263 | ZNF471 | 19 | 57019212 | 57041590 | protein_coding | −1.321635095 | 3.43E−25 | FALSE | FALSE | FALSE | FALSE | FALSE | FALSE |
| ENSG00000144619 | CNTN4 | 3 | 2140497 | 3099645 | protein_coding | −1.322672359 | 6.25E−21 | FALSE | FALSE | FALSE | FALSE | FALSE | FALSE |
| ENSG00000138685 | FGF2 | 4 | 123747863 | 123819391 | protein_coding | −1.322712029 | 5.24E−17 | FALSE | FALSE | FALSE | FALSE | FALSE | FALSE |
| ENSG00000129151 | BBOX1 | 11 | 27062272 | 27149356 | protein_coding | −1.323690984 | 1.12E−05 | FALSE | FALSE | FALSE | FALSE | FALSE | TRUE |
| ENSG00000087258 | GNA01 | 16 | 56225302 | 56391356 | protein_coding | −1.324031037 | 3.72E−13 | FALSE | FALSE | FALSE | FALSE | FALSE | FALSE |
| ENSG00000231419 | LINC00689 | 7 | 158799213 | 158822886 | processed_transc [symbol] | −1.324119658 | 2.17E−07 | FALSE | FALSE | FALSE | FALSE | FALSE | FALSE |
| ENSG00000134463 | ECHDC3 | 10 | 11784365 | 11806069 | protein_coding | −1.324969378 | 1.54E−20 | FALSE | FALSE | FALSE | FALSE | FALSE | FALSE |
| ENSG00000107242 | PIP5K1B | 9 | 71320575 | 71624092 | protein_coding | −1.325283809 | 2.53E−15 | FALSE | FALSE | FALSE | FALSE | FALSE | TRUE |
| ENSG00000104921 | FCER2 | 19 | 7753644 | 7767032 | protein_coding | −1.32571354 | 9.51E−05 | FALSE | FALSE | FALSE | FALSE | FALSE | FALSE |
| ENSG00000172724 | CCL19 | 9 | 34689564 | 34691274 | protein_coding | −1.327374124 | 8.97E−06 | FALSE | FALSE | FALSE | FALSE | FALSE | FALSE |
| ENSG00000114790 | ARHGEF26 | 3 | 153838792 | 153975616 | protein_coding | −1.327764634 | 7.97E−17 | FALSE | FALSE | FALSE | FALSE | FALSE | TRUE |
| ENSG00000082482 | KCNK2 | 1 | 215179118 | 215410436 | protein_coding | −1.328498169 | 7.02E−05 | FALSE | FALSE | FALSE | FALSE | FALSE | FALSE |
| ENSG00000081052 | COL4A4 | 2 | 227867427 | 228028829 | protein_coding | −1.328729847 | 6.35E−18 | FALSE | FALSE | FALSE | FALSE | FALSE | FALSE |
| ENSG00000153162 | BMP6 | 6 | 7727030 | 7881655 | protein_coding | −1.329538997 | 3.85E−17 | FALSE | FALSE | FALSE | FALSE | FALSE | FALSE |
| ENSG00000115468 | EFHD1 | 2 | 233470767 | 233547491 | protein_coding | −1.330141329 | 2.56E−17 | FALSE | FALSE | FALSE | FALSE | FALSE | TRUE |
| ENSG00000138798 | EGF | 4 | 110834040 | 110933422 | protein_coding | −1.330320738 | 0.000140023 | FALSE | FALSE | FALSE | FALSE | FALSE | FALSE |
| ENSG00000138696 | BMPR1B | 4 | 95679119 | 96079599 | protein_coding | −1.331990535 | 1.63E−05 | FALSE | FALSE | FALSE | TRUE | FALSE | TRUE |
| ENSG00000184144 | CNTN2 | 1 | 205012325 | 205047627 | protein_coding | −1.333226442 | 2.08E−07 | FALSE | FALSE | FALSE | FALSE | FALSE | FALSE |
| ENSG00000164627 | KIF6 | 6 | 39297766 | 39693181 | protein_coding | −1.334826848 | 2.06E−17 | FALSE | FALSE | FALSE | FALSE | FALSE | FALSE |
| ENSG00000132970 | WASF3 | 13 | 27131840 | 27263085 | protein_coding | −1.334839388 | 5.29E−26 | FALSE | FALSE | FALSE | FALSE | FALSE | FALSE |
| ENSG00000042062 | FAM65C | 20 | 49202645 | 49308065 | protein_coding | −1.335175324 | 9.86E−17 | FALSE | FALSE | FALSE | TRUE | TRUE | TRUE |
| ENSG00000171433 | GLOD5 | X | 48620154 | 48632064 | protein_coding | −1.33681653 | 4.95E−08 | FALSE | FALSE | FALSE | FALSE | FALSE | TRUE |
| ENSG00000273328 | | 3 | 42850906 | 42949597 | processed_transc [symbol] | −1.337016912 | 1.29E−12 | FALSE | FALSE | FALSE | FALSE | FALSE | FALSE |
| ENSG00000149090 | PAMR1 | 11 | 35453370 | 35551848 | protein_coding | −1.337072838 | 1.21E−17 | FALSE | FALSE | FALSE | FALSE | FALSE | FALSE |
| ENSG00000136160 | EDNRB | 13 | 78469616 | 78493903 | protein_coding | −1.339963789 | 7.66E−17 | FALSE | FALSE | FALSE | FALSE | FALSE | FALSE |
| ENSG00000109794 | FAM149A | 4 | 187025573 | 187093821 | protein_coding | −1.342332451 | 2.77E−24 | FALSE | FALSE | FALSE | FALSE | FALSE | TRUE |
| ENSG00000126803 | HSPA2 | 14 | 65002623 | 65009955 | protein_coding | −1.342699728 | 8.23E−17 | FALSE | FALSE | FALSE | FALSE | FALSE | FALSE |
| ENSG00000157502 | MUM1L1 | X | 105412298 | 105452949 | protein_coding | −1.344382647 | 1.34E−06 | FALSE | FALSE | FALSE | FALSE | FALSE | FALSE |
| ENSG00000186952 | TMEM232 | 5 | 109624934 | 110074657 | protein_coding | −1.344418303 | 5.69E−27 | FALSE | FALSE | FALSE | FALSE | FALSE | FALSE |
| ENSG00000111863 | ADTRP | 6 | 11712287 | 11807279 | protein_coding | −1.344903103 | 4.66E−09 | FALSE | FALSE | FALSE | FALSE | FALSE | FALSE |
| ENSG00000187479 | C11orf96 | 11 | 43946892 | 43965888 | protein_coding | −1.345414562 | 2.99E−18 | FALSE | FALSE | FALSE | FALSE | FALSE | FALSE |
| ENSG00000122035 | RASL11A | 13 | 27844464 | 27847827 | protein_coding | −1.346424976 | 3.42E−31 | FALSE | FALSE | FALSE | FALSE | FALSE | FALSE |
| ENSG00000104327 | CALB1 | 8 | 91070836 | 91107703 | protein_coding | −1.346876739 | 0.004514459 | FALSE | FALSE | FALSE | FALSE | FALSE | FALSE |
| ENSG00000154556 | SORBS2 | 4 | 186506598 | 186877806 | protein_coding | −1.349362068 | 3.27E−19 | FALSE | FALSE | FALSE | FALSE | FALSE | FALSE |
| ENSG00000236581 | STARD13-AS | 13 | 33845516 | 33855471 | processed_transc [symbol] | −1.350632807 | 7.75E−21 | FALSE | FALSE | FALSE | FALSE | FALSE | FALSE |
| ENSG00000154027 | AK5 | 1 | 77747736 | 78025651 | protein_coding | −1.352880078 | 1.01E−06 | FALSE | FALSE | FALSE | FALSE | FALSE | FALSE |
| ENSG00000153246 | PLA2R1 | 2 | 160788519 | 160919121 | protein_coding | −1.353790853 | 2.32E−14 | FALSE | FALSE | FALSE | TRUE | TRUE | FALSE |
| ENSG00000165807 | PPP1R36 | 14 | 65016620 | 65056098 | protein_coding | −1.355279222 | 1.69E−13 | FALSE | FALSE | FALSE | FALSE | FALSE | FALSE |
| ENSG00000185020 | | 9 | 40293025 | 40339528 | pseudogene | −1.35546928 | 5.28E−11 | FALSE | FALSE | FALSE | FALSE | FALSE | FALSE |
| ENSG00000064270 | ATP2C2 | 16 | 84402133 | 84497793 | protein_coding | −1.35618079 | 3.05E−08 | FALSE | FALSE | FALSE | FALSE | FALSE | TRUE |
| ENSG00000101445 | PPP1R16B | 20 | 37434348 | 37551667 | protein_coding | −1.356348468 | 1.31E−25 | FALSE | FALSE | FALSE | FALSE | FALSE | FALSE |
| ENSG00000131016 | AKAP12 | 6 | 151561134 | 151679692 | protein_coding | −1.356403273 | 2.34E−18 | FALSE | FALSE | FALSE | FALSE | FALSE | FALSE |
| ENSG00000224307 | | 9 | 132044737 | 132048007 | lincRNA | −1.356841552 | 2.08E−22 | FALSE | FALSE | FALSE | FALSE | FALSE | FALSE |
| ENSG00000225308 | ASS1P11 | 7 | 21259832 | 21261047 | pseudogene | −1.357148866 | 2.13E−09 | FALSE | FALSE | FALSE | FALSE | FALSE | TRUE |
| ENSG00000124343 | XG | X | 2670091 | 2734539 | protein_coding | −1.357910556 | 1.19E−08 | FALSE | FALSE | FALSE | FALSE | FALSE | FALSE |
| ENSG00000162383 | SLC1A7 | 1 | 53552855 | 53608289 | protein_coding | −1.358340615 | 4.90E−11 | FALSE | FALSE | FALSE | FALSE | FALSE | FALSE |
| ENSG00000122877 | EGR2 | 10 | 64571756 | 64679660 | protein_coding | −1.358485651 | 3.59E−17 | FALSE | FALSE | FALSE | FALSE | FALSE | FALSE |
| ENSG00000250722 | SEPP1 | 5 | 42799982 | 42887494 | protein_coding | −1.358936279 | 7.14E−25 | FALSE | FALSE | FALSE | FALSE | FALSE | FALSE |
| ENSG00000109339 | MAPK10 | 4 | 86936276 | 87515284 | protein_coding | −1.359084765 | 1.07E−25 | FALSE | FALSE | FALSE | FALSE | FALSE | FALSE |
| ENSG00000118004 | COLEC11 | 2 | 3642426 | 3692048 | protein_coding | −1.359094655 | 1.35E−15 | FALSE | FALSE | FALSE | FALSE | FALSE | FALSE |
| ENSG00000197415 | VEPH1 | 3 | 156977531 | 157251408 | protein_coding | −1.359713989 | 3.75E−13 | FALSE | FALSE | FALSE | FALSE | FALSE | FALSE |
| ENSG00000151655 | ITIH2 | 10 | 7745232 | 7791483 | protein_coding | −1.359793247 | 2.19E−10 | FALSE | FALSE | FALSE | FALSE | FALSE | TRUE |
| ENSG00000114698 | PLSCR4 | 3 | 145910126 | 145968966 | protein_coding | −1.360023638 | 1.98E−40 | FALSE | FALSE | FALSE | FALSE | FALSE | FALSE |
| ENSG00000144847 | IGSF11 | 3 | 118619404 | 118864915 | protein_coding | −1.363633342 | 6.99E−07 | FALSE | FALSE | FALSE | FALSE | FALSE | TRUE |
| ENSG00000126759 | CFP | X | 47483612 | 47489704 | protein_coding | −1.363978518 | 4.01E−14 | FALSE | FALSE | FALSE | TRUE | TRUE | FALSE |
| ENSG00000066382 | MPPED2 | 11 | 30406040 | 30608419 | protein_coding | −1.364551545 | 1.02E−11 | FALSE | FALSE | FALSE | FALSE | FALSE | FALSE |
| ENSG00000225704 | | 1 | 171314228 | 171314459 | pseudogene | −1.366391177 | 9.31E−19 | FALSE | FALSE | FALSE | FALSE | FALSE | FALSE |
| ENSG00000120833 | SOCS2 | 12 | 93963590 | 93977263 | protein_coding | −1.36756418 | 7.06E−31 | FALSE | FALSE | FALSE | FALSE | FALSE | FALSE |
| ENSG00000135519 | KCNH3 | 12 | 49932940 | 49952091 | protein_coding | −1.367966418 | 7.56E−12 | FALSE | FALSE | FALSE | FALSE | FALSE | FALSE |
| ENSG00000130558 | OLFM1 | 9 | 137967268 | 138013025 | protein_coding | −1.36801104 | 2.59E−15 | FALSE | FALSE | FALSE | FALSE | FALSE | FALSE |
| ENSG00000229116 | | 10 | 44787706 | 44789157 | antisense | −1.36802641 | 1.65E−07 | FALSE | FALSE | FALSE | FALSE | FALSE | FALSE |
| ENSG00000138135 | CH25H | 10 | 90965694 | 90967071 | protein_coding | −1.369673884 | 1.53E−15 | FALSE | FALSE | FALSE | FALSE | FALSE | FALSE |
| ENSG00000217791 | ASS1P9 | 5 | 53154996 | 53156231 | pseudogene | −1.372251705 | 1.08E−09 | FALSE | FALSE | FALSE | FALSE | FALSE | TRUE |
| ENSG00000120729 | MYOT | 5 | 137203480 | 137223540 | protein_coding | −1.374167865 | 6.11E−10 | FALSE | FALSE | FALSE | FALSE | TRUE | FALSE |
| ENSG00000149970 | CNKSR2 | X | 21392536 | 21672813 | protein_coding | −1.374735388 | 2.43E−10 | FALSE | FALSE | FALSE | FALSE | FALSE | FALSE |
| ENSG00000214548 | MEG3 | 14 | 101245747 | 101327368 | lincRNA | −1.375428385 | 4.14E−17 | FALSE | FALSE | FALSE | TRUE | FALSE | TRUE |
| ENSG00000132465 | IGJ | 4 | 71521258 | 71547534 | protein_coding | −1.376051133 | 3.86E−07 | FALSE | FALSE | FALSE | FALSE | FALSE | FALSE |
| ENSG00000007402 | CACNA2D2 | 3 | 50400233 | 50541675 | protein_coding | −1.376361725 | 9.98E−15 | FALSE | FALSE | FALSE | FALSE | FALSE | FALSE |
| ENSG00000184185 | KCNJ12 | 17 | 21279509 | 21323179 | protein_coding | −1.378302841 | 6.32E−16 | FALSE | FALSE | FALSE | FALSE | FALSE | FALSE |
| ENSG00000112320 | SOBP | 6 | 107811162 | 107981357 | protein_coding | −1.378367857 | 3.76E−25 | FALSE | FALSE | FALSE | FALSE | FALSE | FALSE |
| ENSG00000162722 | TRIM58 | 1 | 248020501 | 248041507 | protein_coding | −1.378847289 | 2.17E−10 | FALSE | FALSE | FALSE | FALSE | FALSE | FALSE |
| ENSG00000100678 | SLC8A3 | 14 | 70510934 | 70655787 | protein_coding | −1.37895815 | 1.98E−12 | FALSE | FALSE | FALSE | FALSE | FALSE | FALSE |
| ENSG00000232352 | SEMA3B-AS1 | 3 | 50304073 | 50304803 | antisense | −1.380959221 | 1.46E−13 | FALSE | FALSE | FALSE | FALSE | FALSE | FALSE |
| ENSG00000171766 | GATM | 15 | 45653322 | 45694525 | protein_coding | −1.38126193 | 2.82E−16 | FALSE | FALSE | FALSE | FALSE | FALSE | TRUE |
| ENSG00000143028 | SYPL2 | 1 | 110009180 | 110024759 | protein_coding | −1.381848004 | 8.12E−18 | FALSE | FALSE | FALSE | FALSE | FALSE | FALSE |
| ENSG00000120915 | EPHX2 | 8 | 27348296 | 27403081 | protein_coding | −1.382196181 | 2.54E−34 | FALSE | FALSE | FALSE | FALSE | FALSE | FALSE |
| ENSG00000120129 | DUSP1 | 5 | 172195093 | 172198198 | protein_coding | −1.38316231 | 2.33E−25 | FALSE | FALSE | FALSE | FALSE | FALSE | FALSE |
| ENSG00000267454 | ZNF582-AS1 | 19 | 56905025 | 56910541 | lincRNA | −1.385673962 | 7.13E−32 | FALSE | FALSE | FALSE | FALSE | FALSE | FALSE |
| ENSG00000078549 | ADCYAP1R1 | 7 | 31092076 | 31151089 | protein_coding | −1.386786506 | 2.79E−08 | FALSE | FALSE | FALSE | FALSE | FALSE | TRUE |
| ENSG00000102010 | BMX | X | 15482369 | 15574652 | protein_coding | −1.387060576 | 1.73E−15 | FALSE | FALSE | FALSE | FALSE | TRUE | FALSE |
| ENSG00000128016 | ZFP36 | 19 | 39897453 | 39900052 | protein_coding | −1.387214877 | 2.83E−27 | FALSE | FALSE | FALSE | FALSE | TRUE | FALSE |
| ENSG00000236155 | | 1 | 26536232 | 26556331 | pseudogene | −1.38873777 | 3.01E−11 | FALSE | FALSE | FALSE | FALSE | FALSE | TRUE |
| ENSG00000168032 | ENTPD3 | 3 | 40428647 | 40470110 | protein_coding | −1.389473446 | 1.06E−10 | FALSE | FALSE | FALSE | FALSE | FALSE | TRUE |
| ENSG00000145147 | SLIT2 | 4 | 20254883 | 20622184 | protein_coding | −1.389621063 | 2.05E−13 | FALSE | FALSE | FALSE | FALSE | FALSE | FALSE |
| ENSG00000102271 | KLHL4 | X | 86772752 | 86925050 | protein_coding | −1.39025839 | 1.01E−15 | FALSE | FALSE | FALSE | FALSE | FALSE | FALSE |
| ENSG00000164197 | RNF180 | 5 | 63461671 | 63668696 | protein_coding | −1.390760146 | 6.19E−35 | FALSE | FALSE | FALSE | FALSE | FALSE | FALSE |
| ENSG00000077522 | ACTN2 | 1 | 236849754 | 236927931 | protein_coding | −1.390936018 | 2.04E−06 | FALSE | FALSE | FALSE | TRUE | TRUE | TRUE |
| ENSG00000019991 | HGF | 7 | 81328322 | 81399754 | protein_coding | −1.391111221 | 1.61E−21 | FALSE | FALSE | FALSE | FALSE | FALSE | FALSE |
| ENSG00000144681 | STAC | 3 | 36421836 | 36589499 | protein_coding | −1.391609528 | 1.48E−10 | FALSE | FALSE | FALSE | FALSE | FALSE | FALSE |
| ENSG00000272823 | | 1 | 221002018 | 221002553 | lincRNA | −1.391748751 | 4.39E−13 | FALSE | FALSE | FALSE | FALSE | FALSE | FALSE |
| ENSG00000150045 | KLRF1 | 12 | 9980077 | 9997606 | protein_coding | −1.392220727 | 4.43E−17 | FALSE | FALSE | FALSE | FALSE | FALSE | FALSE |
| ENSG00000171246 | NPTX1 | 17 | 78440948 | 78451643 | protein_coding | −1.39359924 | 6.77E−05 | FALSE | FALSE | FALSE | FALSE | FALSE | FALSE |
| ENSG00000111249 | CUX2 | 12 | 111471828 | 111788358 | protein_coding | −1.393783859 | 4.84E−09 | FALSE | FALSE | FALSE | FALSE | FALSE | TRUE |
| ENSG00000248441 | | 15 | 95819690 | 95870358 | lincRNA | −1.394620663 | 1.31E−24 | FALSE | FALSE | FALSE | FALSE | FALSE | FALSE |
| ENSG00000151789 | ZNF385D | 3 | 21459915 | 22414812 | protein_coding | −1.395204919 | 8.92E−15 | FALSE | FALSE | FALSE | FALSE | FALSE | FALSE |
| ENSG00000233608 | TWIST2 | 2 | 239756673 | 239795893 | protein_coding | −1.395595482 | 5.43E−13 | FALSE | FALSE | FALSE | FALSE | FALSE | FALSE |
| ENSG00000223764 | | 1 | 852245 | 856396 | lincRNA | −1.396813946 | 5.06E−13 | FALSE | FALSE | FALSE | FALSE | FALSE | FALSE |
| ENSG00000146966 | DENND2A | 7 | 140218220 | 140373793 | protein_coding | −1.397805657 | 6.04E−30 | FALSE | FALSE | FALSE | FALSE | FALSE | FALSE |
| ENSG00000198846 | TOX | 8 | 59717977 | 60031767 | protein_coding | −1.397839688 | 2.38E−18 | FALSE | FALSE | FALSE | FALSE | FALSE | FALSE |
| ENSG00000157150 | TIMP4 | 3 | 12194551 | 12200851 | protein_coding | −1.397935101 | 9.23E−15 | FALSE | FALSE | FALSE | FALSE | FALSE | FALSE |
| ENSG00000250305 | KIAA1456 | 8 | 12803151 | 12889012 | protein_coding | −1.398273051 | 8.97E−20 | FALSE | FALSE | FALSE | FALSE | FALSE | FALSE |
| ENSG00000129214 | SHBG | 17 | 7517382 | 7536700 | protein_coding | −1.398822147 | 8.59E−16 | FALSE | FALSE | FALSE | FALSE | FALSE | FALSE |
| ENSG00000050555 | LAMC3 | 9 | 133884469 | 133969860 | protein_coding | −1.398944651 | 3.33E−15 | FALSE | FALSE | FALSE | FALSE | FALSE | FALSE |
| ENSG00000115112 | TFCP2L1 | 2 | 121974163 | 122042783 | protein_coding | −1.399566841 | 4.24E−09 | FALSE | FALSE | FALSE | FALSE | FALSE | FALSE |
| ENSG00000128573 | FOXP2 | 7 | 113726382 | 114333827 | protein_coding | −1.400871989 | 2.67E−11 | FALSE | FALSE | FALSE | FALSE | FALSE | FALSE |
| ENSG00000256612 | CYP2B7P | 19 | 41430124 | 41456565 | pseudogene | −1.401209584 | 3.81E−07 | FALSE | FALSE | FALSE | FALSE | FALSE | TRUE |
| ENSG00000203710 | CR1 | 1 | 207669492 | 207813992 | protein_coding | −1.401636818 | 3.46E−13 | FALSE | FALSE | FALSE | FALSE | TRUE | FALSE |
| ENSG00000188803 | SHISA6 | 17 | 11144580 | 11467380 | protein_coding | −1.401945253 | 2.21E−07 | FALSE | FALSE | FALSE | FALSE | FALSE | FALSE |
| ENSG00000235997 | | 2 | 30569525 | 30575297 | lincRNA | −1.402632572 | 4.99E−15 | FALSE | FALSE | FALSE | FALSE | FALSE | TRUE |
| ENSG00000104888 | SLC17A7 | 19 | 49932658 | 49945617 | protein_coding | −1.40306225 | 3.19E−19 | FALSE | FALSE | FALSE | FALSE | TRUE | TRUE |
| ENSG00000138669 | PRKG2 | 4 | 82009837 | 82136218 | protein_coding | −1.4033863 | 3.76E−10 | FALSE | FALSE | FALSE | FALSE | FALSE | FALSE |
| ENSG00000185432 | METTL7A | 12 | 51317255 | 51326300 | protein_coding | −1.403596041 | 2.29E−37 | FALSE | FALSE | FALSE | FALSE | FALSE | FALSE |
| ENSG00000197465 | GYPE | 4 | 144792020 | 144826716 | protein_coding | −1.403740807 | 7.38E−18 | FALSE | FALSE | FALSE | FALSE | FALSE | FALSE |
| ENSG00000225792 | | 7 | 26411764 | 26416321 | antisense | −1.406527647 | 1.61E−14 | FALSE | FALSE | FALSE | FALSE | FALSE | FALSE |
| ENSG00000185565 | LSAMP | 3 | 115521235 | 117716095 | protein_coding | −1.406991958 | 5.00E−11 | FALSE | FALSE | FALSE | FALSE | FALSE | FALSE |
| ENSG00000115252 | PDE1A | 2 | 183004763 | 183387919 | protein_coding | −1.407604919 | 1.99E−20 | FALSE | FALSE | FALSE | FALSE | FALSE | TRUE |
| ENSG00000117643 | MAN1C1 | 1 | 25943959 | 26112698 | protein_coding | −1.408848836 | 5.18E−24 | FALSE | FALSE | FALSE | FALSE | FALSE | FALSE |
| ENSG00000116741 | RGS2 | 1 | 192778169 | 192781403 | protein_coding | −1.409308704 | 6.51E−30 | FALSE | FALSE | FALSE | FALSE | FALSE | FALSE |
| ENSG00000263586 | | 17 | 72966799 | 72971823 | antisense | −1.409330077 | 3.87E−16 | FALSE | FALSE | FALSE | FALSE | FALSE | FALSE |
| ENSG00000229481 | | 19 | 38320191 | 38322797 | lincRNA | −1.409373181 | 3.93E−15 | FALSE | FALSE | FALSE | FALSE | FALSE | FALSE |
| ENSG00000231170 | | 7 | 95225994 | 95243031 | antisense | −1.409684371 | 1.22E−24 | FALSE | FALSE | FALSE | FALSE | FALSE | FALSE |
| ENSG00000128482 | RNF112 | 17 | 19314438 | 19320589 | protein_coding | −1.410787625 | 7.62E−27 | FALSE | FALSE | FALSE | TRUE | TRUE | TRUE |
| ENSG00000147724 | FAM135B | 8 | 139142266 | 139509065 | protein_coding | −1.411029717 | 1.59E−06 | FALSE | FALSE | FALSE | FALSE | FALSE | FALSE |
| ENSG00000148204 | CRB2 | 9 | 126118449 | 126142603 | protein_coding | −1.411546251 | 2.02E−09 | FALSE | FALSE | FALSE | FALSE | FALSE | TRUE |
| ENSG00000067840 | PDZD4 | X | 153067621 | 153096020 | protein_coding | −1.41180828 | 1.30E−22 | FALSE | FALSE | FALSE | FALSE | FALSE | TRUE |
| ENSG00000250120 | PCDHA10 | 5 | 140235595 | 140391929 | protein_coding | −1.411918384 | 2.18E−11 | FALSE | FALSE | FALSE | FALSE | FALSE | FALSE |
| ENSG00000050628 | PTGER3 | 1 | 71318036 | 71513491 | protein_coding | −1.412153836 | 1.81E−11 | FALSE | FALSE | FALSE | FALSE | FALSE | TRUE |
| ENSG00000074527 | NTN4 | 12 | 96051583 | 96184930 | protein_coding | −1.412631368 | 2.20E−33 | FALSE | FALSE | FALSE | FALSE | FALSE | FALSE |
| ENSG00000106633 | GCK | 7 | 44183872 | 44237769 | protein_coding | −1.412940166 | 4.19E−15 | FALSE | FALSE | FALSE | FALSE | FALSE | FALSE |
| ENSG00000228549 | | 1 | 17197440 | 17200587 | lincRNA | −1.413942382 | 1.14E−09 | FALSE | FALSE | FALSE | FALSE | FALSE | FALSE |
| ENSG00000257151 | PWAR6 | 15 | 25277020 | 25281637 | lincRNA | −1.415108044 | 1.70E−26 | FALSE | FALSE | FALSE | FALSE | FALSE | FALSE |
| ENSG00000126500 | FLRT1 | 11 | 63870660 | 63886645 | protein_coding | −1.416045477 | 4.22E−10 | FALSE | FALSE | FALSE | FALSE | TRUE | FALSE |
| ENSG00000079308 | TNS1 | 2 | 218664512 | 218867718 | protein_coding | −1.416582204 | 1.12E−24 | FALSE | FALSE | FALSE | FALSE | FALSE | FALSE |
| ENSG00000077616 | NAALAD2 | 11 | 89864683 | 89926062 | protein_coding | −1.417679285 | 8.15E−36 | FALSE | FALSE | FALSE | FALSE | FALSE | FALSE |
| ENSG00000173175 | ADCY5 | 3 | 123001143 | 123168605 | protein_coding | −1.418112492 | 1.16E−15 | FALSE | FALSE | FALSE | FALSE | FALSE | FALSE |
| ENSG00000164825 | DEFB1 | 8 | 6728097 | 6735544 | protein_coding | −1.418813198 | 4.72E−09 | FALSE | FALSE | FALSE | FALSE | FALSE | FALSE |
| ENSG00000137726 | FXYD6 | 11 | 117707693 | 117748201 | protein_coding | −1.421048144 | 1.56E−26 | FALSE | FALSE | FALSE | FALSE | FALSE | FALSE |
| ENSG00000144908 | ALDH1L1 | 3 | 125822412 | 125916837 | protein_coding | −1.421865961 | 6.50E−11 | FALSE | FALSE | FALSE | FALSE | FALSE | FALSE |
| ENSG00000254944 | ATP5F1P5 | 11 | 122831035 | 122831794 | pseudogene | −1.422389815 | 1.07E−26 | FALSE | FALSE | FALSE | FALSE | FALSE | FALSE |
| ENSG00000090006 | LTBP4 | 19 | 41098789 | 41135725 | protein_coding | −1.422549136 | 1.19E−34 | FALSE | FALSE | FALSE | FALSE | FALSE | FALSE |
| ENSG00000109072 | VTN | 17 | 26691290 | 26700110 | protein_coding | −1.425296275 | 2.96E−12 | FALSE | FALSE | FALSE | FALSE | FALSE | TRUE |
| ENSG00000109072 | SEBOX | 17 | 26691290 | 26700110 | protein_coding | −1.425296275 | 2.96E−12 | FALSE | FALSE | FALSE | FALSE | FALSE | TRUE |
| ENSG00000165140 | FBP1 | 9 | 97365415 | 97402531 | protein_coding | −1.425825513 | 2.19E−13 | FALSE | FALSE | FALSE | FALSE | FALSE | FALSE |
| ENSG00000150471 | LPHN3 | 4 | 62066976 | 62944053 | protein_coding | −1.426281396 | 3.39E−17 | FALSE | FALSE | FALSE | FALSE | FALSE | FALSE |
| ENSG00000186594 | MIR22HG | 17 | 1614805 | 1620468 | lincRNA | −1.427074779 | 5.90E−51 | FALSE | FALSE | FALSE | TRUE | FALSE | FALSE |
| ENSG00000159200 | RCAN1 | 21 | 35885440 | 35987441 | protein_coding | −1.427662165 | 6.93E−38 | FALSE | FALSE | FALSE | FALSE | FALSE | FALSE |
| ENSG00000111275 | ALDH2 | 12 | 112204691 | 112247782 | protein_coding | −1.428687522 | 2.47E−39 | FALSE | FALSE | FALSE | FALSE | FALSE | FALSE |
| ENSG00000077942 | FBLN1 | 22 | 45898118 | 45997015 | protein_coding | −1.429725354 | 7.34E−20 | FALSE | FALSE | FALSE | FALSE | FALSE | FALSE |
| ENSG00000135324 | MRAP2 | 6 | 84743475 | 84800600 | protein_coding | −1.431660032 | 1.03E−08 | FALSE | FALSE | FALSE | FALSE | FALSE | FALSE |
| ENSG00000214456 | PLIN5 | 19 | 4522543 | 4535236 | protein_coding | −1.432203427 | 4.63E−15 | FALSE | FALSE | FALSE | TRUE | FALSE | TRUE |
| ENSG00000163406 | SLC15A2 | 3 | 121612936 | 121662949 | protein_coding | −1.432582513 | 1.68E−17 | FALSE | FALSE | FALSE | FALSE | FALSE | FALSE |
| ENSG00000259319 | | 14 | 75890386 | 75894444 | antisense | −1.4328108 | 1.05E−45 | FALSE | FALSE | FALSE | FALSE | FALSE | FALSE |
| ENSG00000221866 | PLXNA4 | 7 | 131808091 | 132333447 | protein_coding | −1.433136283 | 3.24E−12 | FALSE | FALSE | FALSE | FALSE | FALSE | FALSE |
| ENSG00000012223 | LTF | 3 | 46477136 | 46526724 | protein_coding | −1.434214818 | 9.78E−07 | FALSE | FALSE | FALSE | FALSE | FALSE | FALSE |
| ENSG00000228214 | LINC00693 | 3 | 28616282 | 28799831 | antisense | −1.435356393 | 6.42E−14 | FALSE | FALSE | FALSE | FALSE | FALSE | FALSE |
| ENSG00000103175 | WFDC1 | 16 | 84328252 | 84363450 | protein_coding | −1.436002377 | 3.50E−23 | FALSE | FALSE | FALSE | FALSE | FALSE | FALSE |
| ENSG00000135472 | FAIM2 | 12 | 50260679 | 50298000 | protein_coding | −1.436015011 | 8.07E−14 | FALSE | FALSE | FALSE | FALSE | FALSE | FALSE |
| ENSG00000158458 | NRG2 | 5 | 139226364 | 139422884 | protein_coding | −1.437013331 | 1.91E−10 | FALSE | FALSE | FALSE | FALSE | FALSE | FALSE |
| ENSG00000133067 | LGR6 | 1 | 202163029 | 202288909 | protein_coding | −1.438468305 | 4.85E−15 | FALSE | FALSE | FALSE | FALSE | FALSE | FALSE |
| ENSG00000182389 | CACNB4 | 2 | 152689290 | 152955593 | protein_coding | −1.439305481 | 2.07E−16 | FALSE | FALSE | FALSE | FALSE | FALSE | FALSE |
| ENSG00000246223 | C14orf64 | 14 | 98391947 | 98444461 | protein_coding | −1.44048588 | 7.58E−19 | FALSE | FALSE | FALSE | FALSE | FALSE | FALSE |
| ENSG00000168589 | DYNLRB2 | 16 | 80574631 | 80584657 | protein_coding | −1.44378622 | 1.16E−15 | FALSE | FALSE | FALSE | FALSE | FALSE | FALSE |
| ENSG00000171408 | PDE7B | 6 | 136172834 | 136516712 | protein_coding | −1.444206902 | 1.50E−34 | FALSE | FALSE | FALSE | FALSE | FALSE | FALSE |
| ENSG00000137441 | FGFBP2 | 4 | 15961866 | 15970932 | protein_coding | −1.446168908 | 3.08E−12 | FALSE | FALSE | FALSE | FALSE | FALSE | FALSE |
| ENSG00000103316 | CRYM | 16 | 21250195 | 21314404 | protein_coding | −1.446346717 | 7.09E−13 | FALSE | FALSE | FALSE | FALSE | FALSE | FALSE |
| ENSG00000260396 | | 2 | 20877569 | 20879005 | lincRNA | −1.447819948 | 2.25E−21 | FALSE | FALSE | FALSE | FALSE | FALSE | FALSE |
| ENSG00000162461 | SLC25A34 | 1 | 16062900 | 16067891 | protein_coding | −1.449049801 | 6.90E−23 | FALSE | FALSE | FALSE | FALSE | TRUE | TRUE |
| ENSG00000077943 | ITGA8 | 10 | 15555948 | 15762124 | protein_coding | −1.449434443 | 2.13E−20 | FALSE | FALSE | FALSE | FALSE | FALSE | FALSE |
| ENSG00000260105 | AOC4P | 17 | 41017939 | 41026386 | pseudogene | −1.449445929 | 7.10E−17 | FALSE | FALSE | FALSE | FALSE | FALSE | FALSE |
| ENSG00000124107 | SLPI | 20 | 43880880 | 43883205 | protein_coding | −1.450999433 | 1.34E−08 | FALSE | FALSE | FALSE | FALSE | FALSE | FALSE |
| ENSG00000117477 | CCDC181 | 1 | 169364108 | 169429907 | protein_coding | −1.451992592 | 2.42E−16 | FALSE | FALSE | FALSE | FALSE | FALSE | FALSE |
| ENSG00000129757 | CDKN1C | 11 | 2904443 | 2907111 | protein_coding | −1.453377853 | 1.03E−32 | FALSE | FALSE | FALSE | FALSE | FALSE | FALSE |
| ENSG00000005981 | ASB4 | 7 | 95107756 | 95169544 | protein_coding | −1.453393676 | 6.21E−09 | FALSE | FALSE | FALSE | FALSE | FALSE | FALSE |
| ENSG00000171873 | ADRA1D | 20 | 4201329 | 4229721 | protein_coding | −1.455679003 | 1.78E−10 | FALSE | FALSE | FALSE | FALSE | FALSE | FALSE |
| ENSG00000187955 | COL14A1 | 8 | 121072019 | 121384275 | protein_coding | −1.455799862 | 1.02E−20 | FALSE | FALSE | FALSE | FALSE | FALSE | FALSE |
| ENSG00000170500 | LONRF2 | 2 | 100889753 | 100939195 | protein_coding | −1.457399591 | 3.34E−11 | FALSE | FALSE | FALSE | FALSE | FALSE | FALSE |
| ENSG00000132561 | MATN2 | 8 | 98881068 | 99048944 | protein_coding | −1.457504872 | 1.93E−25 | FALSE | FALSE | FALSE | FALSE | FALSE | FALSE |
| ENSG00000175093 | SPSB4 | 3 | 140770244 | 140867453 | protein_coding | −1.457785633 | 1.29E−08 | FALSE | FALSE | FALSE | FALSE | FALSE | FALSE |
| ENSG00000258545 | | X | 119170201 | 119280760 | antisense | −1.457860668 | 1.49E−14 | FALSE | FALSE | FALSE | FALSE | FALSE | FALSE |
| ENSG00000099937 | SERPIND1 | 22 | 21128167 | 21142008 | protein_coding | −1.457999151 | 8.46E−10 | FALSE | FALSE | FALSE | FALSE | FALSE | TRUE |
| ENSG00000188176 | SMTNL2 | 17 | 4487294 | 4511614 | protein_coding | −1.460772205 | 4.03E−11 | FALSE | FALSE | FALSE | FALSE | FALSE | FALSE |
| ENSG00000138769 | CDKL2 | 4 | 76503215 | 76555900 | protein_coding | −1.462241493 | 1.27E−18 | FALSE | FALSE | FALSE | FALSE | FALSE | TRUE |
| ENSG00000249669 | MIR143HG | 5 | 148786252 | 148808241 | lincRNA | −1.462253408 | 1.57E−16 | FALSE | FALSE | FALSE | TRUE | FALSE | TRUE |
| ENSG00000196169 | KIF19 | 17 | 72322349 | 72351959 | protein_coding | −1.462384017 | 4.16E−14 | FALSE | FALSE | FALSE | FALSE | FALSE | FALSE |
| ENSG00000145934 | TENM2 | 5 | 166711804 | 167691162 | protein_coding | −1.466341038 | 4.54E−09 | FALSE | FALSE | FALSE | FALSE | FALSE | TRUE |
| ENSG00000151079 | KCNA6 | 12 | 4918342 | 4960277 | protein_coding | −1.466402672 | 4.69E−14 | FALSE | FALSE | FALSE | FALSE | FALSE | FALSE |
| ENSG00000187699 | C2orf88 | 2 | 190744335 | 191068210 | protein_coding | −1.4665848 | 2.40E−25 | FALSE | FALSE | FALSE | FALSE | FALSE | TRUE |
| ENSG00000226252 | | 1 | 47691469 | 47696422 | antisense | −1.466998024 | 3.20E−16 | FALSE | FALSE | FALSE | FALSE | FALSE | TRUE |
| ENSG00000198774 | RASSF9 | 12 | 86198331 | 86230348 | protein_coding | −1.467292523 | 2.63E−20 | FALSE | FALSE | FALSE | FALSE | FALSE | FALSE |
| ENSG00000165795 | NDRG2 | 14 | 21484922 | 21539031 | protein_coding | −1.4681608 | 3.50E−43 | FALSE | FALSE | FALSE | TRUE | TRUE | TRUE |
| ENSG00000160097 | FNDC5 | 1 | 33327869 | 33338083 | protein_coding | −1.468850272 | 3.40E−13 | FALSE | FALSE | FALSE | FALSE | FALSE | FALSE |
| ENSG00000264016 | | 17 | 10644584 | 10672333 | lincRNA | −1.469671791 | 1.40E−29 | FALSE | FALSE | FALSE | FALSE | FALSE | FALSE |
| ENSG00000121440 | PDZRN3 | 3 | 73431584 | 73674091 | protein_coding | −1.469814846 | 2.94E−25 | FALSE | FALSE | FALSE | FALSE | FALSE | FALSE |
| ENSG00000196581 | AJAP1 | 1 | 4714792 | 4852594 | protein_coding | −1.470369066 | 1.13E−15 | FALSE | FALSE | FALSE | FALSE | FALSE | FALSE |
| ENSG00000166816 | LDHD | 16 | 75145758 | 75150669 | protein_coding | −1.470529874 | 9.27E−20 | FALSE | FALSE | FALSE | FALSE | FALSE | FALSE |
| ENSG00000112981 | NME5 | 5 | 137450866 | 137475132 | protein_coding | −1.470824971 | 9.19E−18 | FALSE | FALSE | FALSE | FALSE | FALSE | TRUE |
| ENSG00000176533 | GNG7 | 19 | 2511217 | 2702707 | protein_coding | −1.472194064 | 1.31E−33 | FALSE | FALSE | FALSE | FALSE | FALSE | FALSE |
| ENSG00000092850 | TEKT2 | 1 | 36549676 | 36553876 | protein_coding | −1.472595709 | 2.46E−12 | FALSE | FALSE | FALSE | FALSE | FALSE | TRUE |
| ENSG00000225342 | | 12 | 40579811 | 40617605 | antisense | −1.473258468 | 4.95E−10 | FALSE | FALSE | FALSE | FALSE | FALSE | TRUE |
| ENSG00000164199 | GPR98 | 5 | 89825161 | 90460038 | protein_coding | −1.474338946 | 5.48E−12 | FALSE | FALSE | FALSE | FALSE | FALSE | FALSE |
| ENSG00000104059 | FAM189A1 | 15 | 29412457 | 29862927 | protein_coding | −1.474490674 | 2.41E−10 | FALSE | FALSE | FALSE | FALSE | FALSE | FALSE |
| ENSG00000115380 | EFEMP1 | 2 | 56093102 | 56151274 | protein_coding | −1.474781145 | 3.11E−21 | FALSE | FALSE | FALSE | FALSE | FALSE | FALSE |
| ENSG00000171811 | TTC40 | 10 | 134621896 | 134756327 | protein_coding | −1.476657055 | 2.63E−14 | FALSE | FALSE | FALSE | FALSE | FALSE | FALSE |
| ENSG00000231437 | | 1 | 112532392 | 112541464 | lincRNA | −1.478354826 | 2.79E−14 | FALSE | FALSE | FALSE | FALSE | FALSE | FALSE |
| ENSG00000136267 | DGKB | 7 | 14184674 | 15014402 | protein_coding | −1.479396737 | 4.60E−08 | FALSE | FALSE | FALSE | FALSE | FALSE | FALSE |
| ENSG00000171596 | NMUR1 | 2 | 232387871 | 232395206 | protein_coding | −1.480221135 | 4.04E−22 | FALSE | FALSE | FALSE | FALSE | FALSE | FALSE |
| ENSG00000198483 | ANKRD35 | 1 | 145549230 | 145568526 | protein_coding | −1.48061464 | 1.41E−22 | FALSE | FALSE | FALSE | TRUE | TRUE | FALSE |
| ENSG00000116678 | LEPR | 1 | 65886248 | 66107242 | protein_coding | −1.480731351 | 4.36E−28 | FALSE | FALSE | FALSE | FALSE | FALSE | FALSE |
| ENSG00000178538 | CA8 | 8 | 61099906 | 61193971 | protein_coding | −1.481154862 | 9.03E−11 | FALSE | FALSE | FALSE | FALSE | FALSE | FALSE |
| ENSG00000078725 | BRINP1 | 9 | 121915736 | 122131745 | protein_coding | −1.481458444 | 8.50E−11 | FALSE | FALSE | FALSE | FALSE | FALSE | FALSE |
| ENSG00000198643 | FAM3D | 3 | 58619673 | 58652575 | protein_coding | −1.481908793 | 2.22E−08 | FALSE | FALSE | FALSE | FALSE | FALSE | TRUE |
| ENSG00000144655 | CSRNP1 | 3 | 39183346 | 39196053 | protein_coding | −1.481981052 | 5.69E−40 | FALSE | FALSE | FALSE | TRUE | FALSE | FALSE |
| ENSG00000171724 | VAT1L | 16 | 77822427 | 78014004 | protein_coding | −1.484061053 | 3.92E−13 | FALSE | FALSE | FALSE | FALSE | FALSE | FALSE |
| ENSG00000100024 | UPB1 | 22 | 24863206 | 24924358 | protein_coding | −1.485792647 | 2.23E−12 | FALSE | FALSE | FALSE | FALSE | FALSE | TRUE |
| ENSG00000161281 | COX7A1 | 19 | 36641824 | 36643771 | protein_coding | −1.486961998 | 1.42E−21 | FALSE | FALSE | FALSE | FALSE | FALSE | FALSE |
| ENSG00000108551 | RASD1 | 17 | 17397751 | 17399709 | protein_coding | −1.488096501 | 4.29E−16 | FALSE | FALSE | FALSE | FALSE | FALSE | FALSE |
| ENSG00000130294 | KIF1A | 2 | 241653181 | 241759725 | protein_coding | −1.488689153 | 1.42E−05 | FALSE | FALSE | FALSE | FALSE | FALSE | FALSE |
| ENSG00000053328 | METTL24 | 6 | 110567131 | 110679475 | protein_coding | −1.491300626 | 2.12E−16 | FALSE | FALSE | FALSE | FALSE | FALSE | FALSE |
| ENSG00000134532 | SOX5 | 12 | 23682440 | 24103966 | protein_coding | −1.492051418 | 2.53E−30 | FALSE | FALSE | FALSE | FALSE | FALSE | FALSE |
| ENSG00000126878 | AIF1L | 9 | 133971863 | 133998539 | protein_coding | −1.492482507 | 9.62E−21 | FALSE | FALSE | FALSE | FALSE | FALSE | FALSE |
| ENSG00000117707 | PROX1 | 1 | 214156524 | 214214595 | protein_coding | −1.492602429 | 7.33E−15 | FALSE | FALSE | FALSE | FALSE | FALSE | TRUE |
| ENSG00000227051 | C14orf132 | 14 | 96505661 | 96560417 | protein_coding | −1.494878317 | 6.13E−26 | FALSE | FALSE | FALSE | FALSE | FALSE | FALSE |
| ENSG00000123496 | IL13RA2 | X | 114238538 | 114254540 | protein_coding | −1.495394351 | 3.39E−10 | FALSE | FALSE | FALSE | FALSE | FALSE | FALSE |
| ENSG00000154263 | ABCA10 | 17 | 67143355 | 67240987 | protein_coding | −1.501128785 | 1.08E−24 | FALSE | TRUE | TRUE | TRUE | FALSE | TRUE |
| ENSG00000204653 | ASPDH | 19 | 51014857 | 51017947 | protein_coding | −1.501767774 | 9.87E−12 | FALSE | FALSE | FALSE | FALSE | FALSE | FALSE |
| ENSG00000260549 | MT1L | 16 | 56651388 | 56652730 | pseudogene | −1.504661389 | 1.17E−19 | FALSE | FALSE | FALSE | FALSE | FALSE | FALSE |
| ENSG00000169562 | GJB1 | X | 70435044 | 70445366 | protein_coding | −1.506200882 | 3.13E−08 | FALSE | FALSE | FALSE | FALSE | FALSE | TRUE |
| ENSG00000178222 | RNF212 | 4 | 1050038 | 1107350 | protein_coding | −1.506572488 | 1.11E−16 | FALSE | FALSE | FALSE | FALSE | FALSE | FALSE |
| ENSG00000188257 | PLA2G2A | 1 | 20301925 | 20306932 | protein_coding | −1.506890817 | 0.000153533 | FALSE | FALSE | TRUE | TRUE | FALSE | TRUE |
| ENSG00000141161 | UNC45B | 17 | 33474836 | 33516364 | protein_coding | −1.507836982 | 2.94E−11 | FALSE | FALSE | FALSE | FALSE | FALSE | FALSE |
| ENSG00000197165 | SULT1A2 | 16 | 28603264 | 28608430 | protein_coding | −1.510058909 | 7.32E−17 | FALSE | FALSE | FALSE | FALSE | FALSE | FALSE |
| ENSG00000183798 | EMILIN3 | 20 | 39988606 | 39995467 | protein_coding | −1.510199932 | 4.33E−15 | FALSE | FALSE | FALSE | FALSE | FALSE | FALSE |
| ENSG00000184113 | CLDN5 | 22 | 19510547 | 19515068 | protein_coding | −1.510654132 | 2.13E−27 | FALSE | FALSE | FALSE | FALSE | FALSE | FALSE |
| ENSG00000142871 | CYR61 | 1 | 86046444 | 86049645 | protein_coding | −1.512074972 | 4.09E−28 | FALSE | FALSE | FALSE | FALSE | FALSE | FALSE |
| ENSG00000172346 | CSDC2 | 22 | 41956767 | 41973745 | protein_coding | −1.513033249 | 1.50E−13 | FALSE | FALSE | FALSE | FALSE | FALSE | FALSE |
| ENSG00000185739 | SRL | 16 | 4239375 | 4292081 | protein_coding | −1.514160197 | 1.37E−21 | FALSE | FALSE | FALSE | FALSE | FALSE | TRUE |
| ENSG00000162772 | ATF3 | 1 | 212738676 | 212794119 | protein_coding | −1.51501629 | 1.46E−28 | FALSE | FALSE | FALSE | FALSE | FALSE | FALSE |
| ENSG00000115896 | PLCL1 | 2 | 198669426 | 199437305 | protein_coding | −1.516517412 | 3.15E−25 | FALSE | FALSE | FALSE | FALSE | FALSE | FALSE |
| ENSG00000240583 | AQP1 | 7 | 30893010 | 30965131 | protein_coding | −1.518287489 | 2.82E−21 | FALSE | FALSE | FALSE | FALSE | FALSE | FALSE |
| ENSG00000134202 | GSTM3 | 1 | 110276554 | 110284384 | protein_coding | −1.518590042 | 2.23E−20 | FALSE | FALSE | FALSE | FALSE | FALSE | FALSE |
| ENSG00000198932 | GPRASP1 | X | 101906294 | 101914011 | protein_coding | −1.51911778 | 6.76E−44 | FALSE | FALSE | FALSE | FALSE | FALSE | TRUE |
| ENSG00000183117 | CSMD1 | 8 | 2792875 | 4852494 | protein_coding | −1.519241943 | 3.16E−07 | FALSE | FALSE | FALSE | FALSE | FALSE | FALSE |
| ENSG00000091513 | TF | 3 | 133464800 | 133497850 | protein_coding | −1.519295318 | 4.76E−11 | FALSE | FALSE | FALSE | FALSE | FALSE | TRUE |
| ENSG00000196549 | MME | 3 | 154741913 | 154901497 | protein_coding | −1.519819196 | 1.56E−09 | FALSE | FALSE | FALSE | FALSE | FALSE | FALSE |
| ENSG00000150510 | FAM124A | 13 | 51796503 | 51858377 | protein_coding | −1.520024326 | 1.27E−32 | FALSE | FALSE | FALSE | FALSE | FALSE | FALSE |
| ENSG00000144649 | FAM198A | 3 | 43020759 | 43101703 | protein_coding | −1.520259765 | 2.86E−24 | FALSE | FALSE | FALSE | FALSE | FALSE | FALSE |
| ENSG00000173930 | SLCO4C1 | 5 | 101569690 | 101632253 | protein_coding | −1.521968124 | 1.43E−09 | FALSE | FALSE | FALSE | FALSE | FALSE | TRUE |
| ENSG00000081041 | CXCL2 | 4 | 74962752 | 74965010 | protein_coding | −1.523274757 | 1.05E−12 | FALSE | FALSE | FALSE | FALSE | FALSE | FALSE |
| ENSG00000177807 | KCNJ10 | 1 | 160007257 | 160040038 | protein_coding | −1.523423447 | 1.70E−07 | FALSE | FALSE | FALSE | FALSE | FALSE | TRUE |
| ENSG00000162687 | KCNT2 | 1 | 196194909 | 196578355 | protein_coding | −1.524590193 | 6.89E−18 | FALSE | FALSE | FALSE | FALSE | FALSE | FALSE |
| ENSG00000263400 | | 17 | 10633094 | 10718481 | antisense | −1.526451405 | 2.90E−38 | FALSE | FALSE | FALSE | FALSE | FALSE | FALSE |
| ENSG00000102383 | ZDHHC15 | X | 74588262 | 74743337 | protein_coding | −1.526913743 | 1.32E−27 | FALSE | FALSE | FALSE | FALSE | FALSE | FALSE |
| ENSG00000126838 | PZP | 12 | 9301436 | 9360966 | protein_coding | −1.526993341 | 1.49E−11 | FALSE | FALSE | FALSE | FALSE | FALSE | TRUE |
| ENSG00000177363 | LRRN4CL | 11 | 62453874 | 62457371 | protein_coding | −1.52704686 | 1.82E−15 | FALSE | FALSE | FALSE | FALSE | FALSE | FALSE |
| ENSG00000197769 | MAP1LC3C | 1 | 242158792 | 242162375 | protein_coding | −1.532231601 | 7.40E−11 | FALSE | FALSE | FALSE | FALSE | FALSE | FALSE |
| ENSG00000134343 | ANO3 | 11 | 26210829 | 26684835 | protein_coding | −1.533194422 | 1.88E−13 | FALSE | FALSE | FALSE | FALSE | FALSE | FALSE |
| ENSG00000091622 | PITPNM3 | 17 | 6354584 | 6459814 | protein_coding | −1.533412071 | 2.43E−27 | FALSE | FALSE | FALSE | FALSE | TRUE | TRUE |
| ENSG00000230587 | | 2 | 43324884 | 43329829 | lincRNA | −1.53390412 | 6.46E−18 | FALSE | FALSE | FALSE | FALSE | FALSE | FALSE |
| ENSG00000259884 | | 12 | 52452243 | 52453287 | lincRNA | −1.535930656 | 1.37E−16 | FALSE | FALSE | FALSE | FALSE | FALSE | FALSE |
| ENSG00000184347 | SLIT3 | 5 | 168088745 | 168728133 | protein_coding | −1.538093587 | 9.43E−25 | FALSE | FALSE | FALSE | FALSE | FALSE | FALSE |
| ENSG00000077264 | PAK3 | X | 110187513 | 110470589 | protein_coding | −1.538123192 | 3.24E−16 | FALSE | FALSE | FALSE | FALSE | FALSE | FALSE |
| ENSG00000134258 | VTCN1 | 1 | 117686209 | 117753556 | protein_coding | −1.538366922 | 9.76E−05 | FALSE | FALSE | FALSE | FALSE | FALSE | TRUE |
| ENSG00000173406 | DAB1 | 1 | 57460451 | 59012406 | protein_coding | −1.539045506 | 3.40E−15 | FALSE | FALSE | FALSE | FALSE | FALSE | FALSE |
| ENSG00000266010 | GATA6-AS1 | 18 | 19746859 | 19748929 | lincRNA | −1.539055512 | 3.23E−14 | FALSE | FALSE | FALSE | TRUE | FALSE | TRUE |
| ENSG00000184005 | ST6GALNAC3 | 1 | 76540404 | 77100286 | protein_coding | −1.539207145 | 3.69E−38 | FALSE | FALSE | FALSE | FALSE | FALSE | FALSE |
| ENSG00000271474 | | 4 | 96470280 | 96473608 | antisense | −1.544519991 | 2.24E−25 | FALSE | FALSE | FALSE | FALSE | FALSE | TRUE |
| ENSG00000078596 | ITM2A | X | 78615881 | 78623164 | protein_coding | −1.545622919 | 2.14E−30 | FALSE | FALSE | FALSE | FALSE | FALSE | FALSE |
| ENSG00000160862 | AZGP1 | 7 | 99564343 | 99573780 | protein_coding | −1.546905898 | 8.41E−09 | FALSE | FALSE | FALSE | FALSE | FALSE | TRUE |
| ENSG00000233929 | MT1XP1 | 1 | 16567708 | 16567893 | pseudogene | −1.548637708 | 1.54E−18 | FALSE | FALSE | FALSE | FALSE | FALSE | FALSE |
| ENSG00000243069 | ARHGEF26-AS | 3 | 153742190 | 153839121 | processed_transc [symbol] | −1.549014139 | 2.28E−15 | FALSE | FALSE | FALSE | FALSE | FALSE | TRUE |
| ENSG00000131018 | SYNE1 | 6 | 152442819 | 152958936 | protein_coding | −1.54985309 | 8.11E−45 | FALSE | FALSE | FALSE | FALSE | FALSE | FALSE |
| ENSG00000112214 | FHL5 | 6 | 97010424 | 97064512 | protein_coding | −1.550025926 | 1.79E−18 | FALSE | FALSE | FALSE | FALSE | FALSE | FALSE |
| ENSG00000167244 | IGF2 | 11 | 2150342 | 2170833 | protein_coding | −1.551379989 | 4.09E−10 | FALSE | FALSE | FALSE | FALSE | TRUE | FALSE |
| ENSG00000180354 | MTURN | 7 | 30174426 | 30202378 | protein_coding | −1.55218216 | 1.14E−43 | FALSE | FALSE | FALSE | FALSE | FALSE | FALSE |
| ENSG00000144712 | CAND2 | 3 | 12837971 | 12913415 | protein_coding | −1.552668278 | 1.69E−31 | FALSE | FALSE | FALSE | FALSE | FALSE | FALSE |
| ENSG00000204176 | SYT15 | 10 | 46955444 | 46971400 | protein_coding | −1.554153687 | 1.42E−26 | FALSE | FALSE | FALSE | FALSE | FALSE | FALSE |
| ENSG00000225706 | | 9 | 8858130 | 8862255 | lincRNA | −1.554195467 | 6.68E−16 | FALSE | FALSE | FALSE | FALSE | FALSE | FALSE |
| ENSG00000259969 | | 14 | 58460263 | 58461243 | sense_overlapping | −1.55737776 | 1.75E−26 | FALSE | FALSE | FALSE | FALSE | FALSE | FALSE |
| ENSG00000120645 | IQSEC3 | 12 | 175931 | 287626 | protein_coding | −1.559756348 | 2.66E−21 | FALSE | FALSE | FALSE | FALSE | FALSE | FALSE |
| ENSG00000131730 | CKMT2 | 5 | 80529104 | 80562216 | protein_coding | −1.560894513 | 3.04E−12 | FALSE | FALSE | FALSE | FALSE | FALSE | FALSE |
| ENSG00000182732 | RGS6 | 14 | 72399156 | 73030654 | protein_coding | −1.56262544 | 9.66E−15 | FALSE | FALSE | FALSE | FALSE | FALSE | FALSE |
| ENSG00000154721 | JAM2 | 21 | 27011584 | 27089874 | protein_coding | −1.562980411 | 3.50E−36 | FALSE | FALSE | FALSE | FALSE | FALSE | FALSE |
| ENSG00000148948 | LRRC4C | 11 | 40135753 | 41481323 | protein_coding | −1.566290272 | 7.65E−19 | FALSE | FALSE | FALSE | FALSE | FALSE | FALSE |
| ENSG00000187824 | TMEM220 | 17 | 10602332 | 10633633 | protein_coding | −1.566982493 | 1.04E−45 | FALSE | FALSE | FALSE | FALSE | FALSE | FALSE |
| ENSG00000072163 | LIMS2 | 2 | 128395956 | 128439360 | protein_coding | −1.569265463 | 6.42E−32 | FALSE | FALSE | FALSE | FALSE | FALSE | FALSE |
| ENSG00000215018 | COL28A1 | 7 | 7395834 | 7575484 | protein_coding | −1.570909378 | 4.78E−19 | FALSE | FALSE | FALSE | FALSE | FALSE | FALSE |
| ENSG00000170271 | FAXDC2 | 5 | 154198051 | 154238812 | protein_coding | −1.572065172 | 4.66E−38 | FALSE | FALSE | FALSE | FALSE | FALSE | FALSE |
| ENSG00000137727 | ARHGAP20 | 11 | 110447766 | 110583912 | protein_coding | −1.572196331 | 4.46E−30 | FALSE | FALSE | FALSE | FALSE | FALSE | FALSE |
| ENSG00000146477 | SLC22A3 | 6 | 160769300 | 160876014 | protein_coding | −1.572740124 | 1.13E−17 | FALSE | FALSE | FALSE | FALSE | FALSE | FALSE |
| ENSG00000137033 | IL33 | 9 | 6215805 | 6257983 | protein_coding | −1.57530241 | 7.59E−22 | FALSE | FALSE | FALSE | FALSE | FALSE | FALSE |
| ENSG00000080007 | DDX43 | 6 | 74104471 | 74127292 | protein_coding | −1.576608682 | 1.66E−16 | FALSE | FALSE | FALSE | FALSE | FALSE | FALSE |
| ENSG00000156049 | GNA14 | 9 | 80037995 | 80263223 | protein_coding | −1.577241221 | 3.34E−34 | FALSE | FALSE | FALSE | FALSE | FALSE | FALSE |
| ENSG00000198523 | PLN | 6 | 118869461 | 118881893 | protein_coding | −1.578832593 | 3.33E−16 | FALSE | FALSE | FALSE | FALSE | FALSE | FALSE |
| ENSG00000134198 | TSPAN2 | 1 | 115590632 | 115632121 | protein_coding | −1.58056749 | 1.98E−28 | FALSE | FALSE | FALSE | FALSE | FALSE | FALSE |
| ENSG00000154529 | CNTNAP3B | 9 | 43684902 | 43924049 | protein_coding | −1.581910428 | 3.54E−20 | FALSE | FALSE | FALSE | FALSE | FALSE | FALSE |
| ENSG00000196569 | LAMA2 | 6 | 129204342 | 129837714 | protein_coding | −1.584942093 | 1.01E−26 | FALSE | FALSE | FALSE | FALSE | TRUE | FALSE |
| ENSG00000129654 | FOXJ1 | 17 | 74132414 | 74137380 | protein_coding | −1.585912397 | 9.69E−07 | FALSE | FALSE | FALSE | FALSE | FALSE | TRUE |
| ENSG00000204175 | GPRIN2 | 10 | 46994087 | 47005643 | protein_coding | −1.586440667 | 2.18E−12 | FALSE | FALSE | FALSE | FALSE | FALSE | FALSE |
| ENSG00000205363 | C15orf59 | 15 | 74032141 | 74045088 | protein_coding | −1.587130686 | 6.04E−15 | FALSE | FALSE | FALSE | FALSE | FALSE | FALSE |
| ENSG00000123454 | DBH | 9 | 136501482 | 136524466 | protein_coding | −1.589121885 | 6.06E−19 | FALSE | FALSE | FALSE | FALSE | TRUE | FALSE |
| ENSG00000035720 | STAP1 | 4 | 68424446 | 68473055 | protein_coding | −1.590117021 | 2.36E−11 | FALSE | FALSE | FALSE | FALSE | FALSE | FALSE |
| ENSG00000134962 | KLB | 4 | 39408473 | 39453156 | protein_coding | −1.590435729 | 4.60E−17 | FALSE | FALSE | FALSE | FALSE | FALSE | TRUE |
| ENSG00000188338 | SLC38A3 | 3 | 50242679 | 50258411 | processed_transc [symbol] | −1.590744005 | 4.19E−10 | FALSE | FALSE | FALSE | FALSE | FALSE | TRUE |
| ENSG00000010319 | SEMA3G | 3 | 52467069 | 52479101 | protein_coding | −1.591239651 | 1.85E−25 | FALSE | FALSE | FALSE | FALSE | FALSE | FALSE |
| ENSG00000007174 | DNAH9 | 17 | 11501748 | 11873065 | protein_coding | −1.592086381 | 4.41E−15 | FALSE | FALSE | FALSE | FALSE | FALSE | FALSE |
| ENSG00000125675 | GRIA3 | X | 122318006 | 122624766 | protein_coding | −1.593345291 | 6.75E−15 | FALSE | FALSE | FALSE | FALSE | FALSE | TRUE |
| ENSG00000076555 | ACACB | 12 | 109554400 | 109706031 | protein_coding | −1.595759121 | 3.38E−39 | FALSE | FALSE | FALSE | FALSE | FALSE | FALSE |
| ENSG00000111405 | ENDOU | 12 | 48103517 | 48119350 | protein_coding | −1.596505949 | 8.28E−20 | FALSE | FALSE | TRUE | TRUE | TRUE | FALSE |
| ENSG00000099769 | IGFALS | 16 | 1840414 | 1844972 | protein_coding | −1.598429929 | 2.51E−12 | FALSE | FALSE | FALSE | FALSE | FALSE | FALSE |
| ENSG00000085563 | ABCB1 | 7 | 87133175 | 87342611 | protein_coding | −1.600034244 | 4.09E−23 | FALSE | FALSE | FALSE | FALSE | FALSE | FALSE |
| ENSG00000107518 | ATRNL1 | 10 | 116853124 | 117708503 | protein_coding | −1.600645412 | 5.93E−13 | FALSE | FALSE | FALSE | FALSE | FALSE | FALSE |
| ENSG00000146250 | PRSS35 | 6 | 84222194 | 84235423 | protein_coding | −1.600649751 | 2.19E−16 | FALSE | FALSE | FALSE | FALSE | FALSE | FALSE |
| ENSG00000165272 | AQP3 | 9 | 33441152 | 33447609 | protein_coding | −1.601251977 | 2.14E−17 | FALSE | FALSE | FALSE | FALSE | TRUE | TRUE |
| ENSG00000136531 | SCN2A | 2 | 166095912 | 166248818 | protein_coding | −1.601691223 | 6.27E−14 | FALSE | FALSE | FALSE | FALSE | FALSE | FALSE |
| ENSG00000206384 | COL6A6 | 3 | 130279178 | 130396999 | protein_coding | −1.604514163 | 1.31E−15 | FALSE | FALSE | FALSE | FALSE | FALSE | FALSE |
| ENSG00000078053 | AMPH | 7 | 38423305 | 38671167 | protein_coding | −1.604707166 | 5.81E−17 | FALSE | FALSE | FALSE | FALSE | FALSE | FALSE |
| ENSG00000156218 | ADAMTSL3 | 15 | 84322838 | 84708594 | protein_coding | −1.604852668 | 4.64E−21 | FALSE | FALSE | FALSE | FALSE | FALSE | FALSE |
| ENSG00000180447 | GAS1 | 9 | 89559279 | 89562104 | protein_coding | −1.606285463 | 4.32E−25 | FALSE | FALSE | FALSE | FALSE | FALSE | FALSE |
| ENSG00000171533 | MAP6 | 11 | 75297963 | 75380165 | protein_coding | −1.606843883 | 3.76E−27 | FALSE | FALSE | FALSE | FALSE | FALSE | FALSE |
| ENSG00000146233 | CYP39A1 | 6 | 46517541 | 46620523 | protein_coding | −1.607663033 | 3.68E−22 | FALSE | FALSE | FALSE | FALSE | FALSE | FALSE |
| ENSG00000109610 | SOD3 | 4 | 24791534 | 24802464 | protein_coding | −1.610104896 | 3.72E−27 | FALSE | FALSE | FALSE | FALSE | FALSE | FALSE |
| ENSG00000132464 | ENAM | 4 | 71494461 | 71552533 | protein_coding | −1.611199235 | 1.21E−16 | FALSE | FALSE | FALSE | FALSE | FALSE | FALSE |
| ENSG00000106927 | AMBP | 9 | 116822407 | 116840752 | protein_coding | −1.612158169 | 1.68E−12 | FALSE | FALSE | FALSE | FALSE | FALSE | TRUE |
| ENSG00000165379 | LRFN5 | 14 | 42076773 | 42373752 | protein_coding | −1.612218587 | 6.94E−12 | FALSE | FALSE | FALSE | FALSE | FALSE | FALSE |
| ENSG00000136244 | IL6 | 7 | 22765503 | 22771621 | protein_coding | −1.612235881 | 7.15E−11 | FALSE | FALSE | FALSE | FALSE | FALSE | FALSE |
| ENSG00000116254 | CHD5 | 1 | 6161853 | 6240183 | protein_coding | −1.613439246 | 2.99E−11 | FALSE | FALSE | FALSE | FALSE | FALSE | FALSE |
| ENSG00000127951 | FGL2 | 7 | 76822688 | 76829143 | protein_coding | −1.613941556 | 1.74E−39 | FALSE | FALSE | FALSE | FALSE | FALSE | FALSE |
| ENSG00000228962 | HCG23 | 6 | 32358287 | 32361463 | antisense | −1.614136518 | 5.31E−22 | FALSE | FALSE | FALSE | FALSE | FALSE | FALSE |
| ENSG00000215386 | LINC00478 | 21 | 17442842 | 17999716 | lincRNA | −1.614476098 | 2.76E−30 | FALSE | FALSE | FALSE | FALSE | FALSE | FALSE |
| ENSG00000163661 | PTX3 | 3 | 157154578 | 157161417 | protein_coding | −1.614531306 | 3.97E−22 | FALSE | FALSE | FALSE | FALSE | TRUE | FALSE |
| ENSG00000164418 | GRIK2 | 6 | 101846664 | 102517958 | protein_coding | −1.615367216 | 1.42E−12 | FALSE | FALSE | FALSE | FALSE | FALSE | FALSE |
| ENSG00000164972 | C9orf24 | 9 | 34379017 | 34397830 | protein_coding | −1.615515485 | 8.37E−15 | FALSE | FALSE | FALSE | FALSE | FALSE | FALSE |
| ENSG00000114812 | VIPR1 | 3 | 42530791 | 42579059 | protein_coding | −1.615672984 | 2.97E−19 | FALSE | FALSE | FALSE | FALSE | TRUE | TRUE |
| ENSG00000025423 | HSD17B6 | 12 | 57145945 | 57181574 | protein_coding | −1.617351811 | 1.59E−16 | FALSE | FALSE | FALSE | FALSE | FALSE | TRUE |
| ENSG00000172955 | ADH6 | 4 | 100123795 | 100140694 | protein_coding | −1.618071177 | 5.01E−15 | FALSE | FALSE | FALSE | FALSE | FALSE | TRUE |
| ENSG00000007908 | SELE | 1 | 169691781 | 169733846 | protein_coding | −1.618395402 | 1.82E−14 | FALSE | FALSE | FALSE | FALSE | FALSE | FALSE |
| ENSG00000105737 | GRIK5 | 19 | 42502473 | 42573650 | protein_coding | −1.619514727 | 8.45E−15 | FALSE | FALSE | FALSE | FALSE | FALSE | FALSE |
| ENSG00000138678 | AGPAT9 | 4 | 84457067 | 84527028 | protein_coding | −1.621061647 | 2.92E−28 | FALSE | FALSE | FALSE | FALSE | TRUE | FALSE |
| ENSG00000163531 | NFASC | 1 | 204797779 | 204991950 | protein_coding | −1.622132671 | 3.17E−28 | FALSE | FALSE | FALSE | FALSE | FALSE | FALSE |
| ENSG00000167641 | PPP1R14A | 19 | 38741877 | 38747231 | protein_coding | −1.622960953 | 3.63E−30 | FALSE | FALSE | FALSE | FALSE | FALSE | FALSE |
| ENSG00000099957 | P2RX6 | 22 | 21364097 | 21383119 | protein_coding | −1.626220663 | 6.75E−25 | FALSE | FALSE | FALSE | FALSE | FALSE | FALSE |
| ENSG00000196090 | PTPRT | 20 | 40701392 | 41818610 | protein_coding | −1.628447507 | 2.85E−10 | FALSE | FALSE | FALSE | FALSE | FALSE | TRUE |
| ENSG00000132437 | DDC | 7 | 50526134 | 50633154 | protein_coding | −1.629878166 | 7.83E−09 | FALSE | FALSE | FALSE | FALSE | FALSE | TRUE |
| ENSG00000234456 | MAGI2-AS3 | 7 | 79082198 | 79100524 | processed_transc [symbol] | −1.630480797 | 4.44E−38 | FALSE | FALSE | FALSE | FALSE | FALSE | FALSE |
| ENSG00000130413 | STK33 | 11 | 8413418 | 8615836 | protein_coding | −1.632386127 | 1.17E−17 | FALSE | FALSE | FALSE | FALSE | FALSE | TRUE |
| ENSG00000147576 | ADHFE1 | 8 | 67342420 | 67383836 | protein_coding | −1.633832757 | 7.31E−36 | FALSE | FALSE | FALSE | FALSE | FALSE | TRUE |
| ENSG00000186462 | NAP1L2 | X | 72432135 | 72434684 | protein_coding | −1.634046357 | 1.98E−23 | FALSE | FALSE | FALSE | FALSE | FALSE | FALSE |
| ENSG00000178965 | C1orf173 | 1 | 75033795 | 75139422 | protein_coding | −1.634422737 | 4.56E−07 | FALSE | FALSE | FALSE | FALSE | FALSE | FALSE |
| ENSG00000132837 | DMGDH | 5 | 78293438 | 78531861 | protein_coding | −1.63558486 | 1.96E−26 | FALSE | FALSE | FALSE | FALSE | FALSE | TRUE |
| ENSG00000009765 | IYD | 6 | 150690028 | 150727105 | protein_coding | −1.636489332 | 2.04E−09 | FALSE | FALSE | FALSE | FALSE | FALSE | TRUE |
| ENSG00000164879 | CA3 | 8 | 86285665 | 86361269 | protein_coding | −1.6385805 | 1.90E−17 | FALSE | FALSE | FALSE | FALSE | TRUE | FALSE |
| ENSG00000233237 | LINC00472 | 6 | 72054047 | 72130472 | lincRNA | −1.638581701 | 1.75E−31 | FALSE | FALSE | FALSE | FALSE | FALSE | FALSE |
| ENSG00000148123 | | 9 | 103790991 | 104087417 | protein_coding | −1.639608365 | 1.61E−07 | FALSE | FALSE | FALSE | FALSE | FALSE | FALSE |
| ENSG00000153292 | GPR110 | 6 | 46965440 | 47010099 | protein_coding | −1.639845364 | 3.55E−07 | FALSE | FALSE | FALSE | FALSE | FALSE | TRUE |
| ENSG00000197406 | DIO3 | 14 | 102027688 | 102029789 | protein_coding | −1.641839138 | 4.11E−14 | FALSE | FALSE | FALSE | FALSE | FALSE | FALSE |
| ENSG00000171385 | KCND3 | 1 | 112313284 | 112531777 | protein_coding | −1.64196746 | 1.18E−22 | FALSE | FALSE | FALSE | FALSE | FALSE | FALSE |
| ENSG00000168490 | PHYHIP | 8 | 22077222 | 22089854 | protein_coding | −1.642297317 | 4.34E−22 | FALSE | FALSE | FALSE | FALSE | FALSE | TRUE |
| ENSG00000080166 | DCT | 13 | 95089558 | 95131936 | protein_coding | −1.642782108 | 1.62E−15 | FALSE | FALSE | FALSE | FALSE | FALSE | FALSE |
| ENSG00000135063 | FAM189A2 | 9 | 71939488 | 72007371 | protein_coding | −1.643128363 | 1.67E−20 | FALSE | FALSE | FALSE | FALSE | FALSE | FALSE |
| ENSG00000172201 | ID4 | 6 | 19837617 | 19840915 | protein_coding | −1.644191147 | 1.35E−35 | FALSE | FALSE | FALSE | FALSE | FALSE | TRUE |
| ENSG00000168306 | ACOX2 | 3 | 58490863 | 58523046 | protein_coding | −1.645715767 | 2.12E−28 | FALSE | FALSE | FALSE | FALSE | FALSE | FALSE |
| ENSG00000227544 | | 7 | 35754644 | 35774497 | lincRNA | −1.646992384 | 1.26E−27 | FALSE | FALSE | FALSE | FALSE | FALSE | FALSE |
| ENSG00000179388 | EGR3 | 8 | 22545172 | 22550815 | protein_coding | −1.647274729 | 9.83E−25 | FALSE | FALSE | FALSE | FALSE | FALSE | FALSE |
| ENSG00000240654 | C1QTNF9 | 13 | 24881304 | 24896673 | protein_coding | −1.648636396 | 4.64E−16 | FALSE | FALSE | FALSE | FALSE | FALSE | FALSE |
| ENSG00000261888 | | 17 | 81061205 | 81063860 | lincRNA | −1.64885954 | 1.03E−32 | FALSE | FALSE | FALSE | FALSE | FALSE | FALSE |
| ENSG00000130037 | KCNA5 | 12 | 5153085 | 5155949 | protein_coding | −1.649630577 | 8.79E−20 | FALSE | FALSE | FALSE | FALSE | FALSE | FALSE |
| ENSG00000131771 | PPP1R1B | 17 | 37782993 | 37792879 | protein_coding | −1.649723785 | 1.81E−07 | FALSE | FALSE | FALSE | FALSE | FALSE | TRUE |
| ENSG00000186198 | SLC51B | 15 | 65337708 | 65345734 | protein_coding | −1.64996058 | 2.53E−17 | FALSE | FALSE | FALSE | FALSE | FALSE | FALSE |
| ENSG00000141744 | PNMT | 17 | 37824234 | 37826728 | protein_coding | −1.651312399 | 2.98E−10 | FALSE | FALSE | FALSE | FALSE | TRUE | FALSE |
| ENSG00000168658 | VWA3B | 2 | 98703579 | 98929762 | protein_coding | −1.657848413 | 1.75E−12 | FALSE | FALSE | FALSE | FALSE | FALSE | FALSE |
| ENSG00000122367 | LDB3 | 10 | 88428206 | 88495825 | protein_coding | −1.660383191 | 5.72E−22 | FALSE | FALSE | FALSE | FALSE | TRUE | TRUE |
| ENSG00000213088 | DARC | 1 | 159173097 | 159176290 | protein_coding | −1.661119891 | 4.67E−15 | FALSE | FALSE | FALSE | FALSE | TRUE | FALSE |
| ENSG00000117322 | CR2 | 1 | 207627575 | 207663240 | protein_coding | −1.661172274 | 1.72E−06 | FALSE | FALSE | FALSE | FALSE | FALSE | FALSE |
| ENSG00000241935 | HOGA1 | 10 | 99344080 | 99372559 | protein_coding | −1.666925534 | 1.23E−20 | FALSE | FALSE | FALSE | FALSE | FALSE | FALSE |
| ENSG00000204740 | MALRD1 | 10 | 19492779 | 20079330 | protein_coding | −1.667166841 | 3.65E−13 | FALSE | FALSE | FALSE | FALSE | FALSE | FALSE |
| ENSG00000104044 | OCA2 | 15 | 28000021 | 28344504 | protein_coding | −1.667613011 | 1.47E−08 | FALSE | FALSE | FALSE | FALSE | FALSE | FALSE |
| ENSG00000174514 | MFSD4 | 1 | 205538013 | 205572046 | protein_coding | −1.667797589 | 8.13E−19 | FALSE | FALSE | FALSE | FALSE | TRUE | FALSE |
| ENSG00000156475 | PPP2R2B | 5 | 145967936 | 146464347 | protein_coding | −1.667971627 | 5.61E−22 | FALSE | FALSE | FALSE | FALSE | FALSE | FALSE |
| ENSG00000126218 | F10 | 13 | 113777128 | 113803843 | protein_coding | −1.668184369 | 3.37E−24 | FALSE | FALSE | FALSE | FALSE | FALSE | FALSE |
| ENSG00000169418 | NPR1 | 1 | 153651113 | 153666468 | protein_coding | −1.668315677 | 3.55E−36 | FALSE | FALSE | FALSE | FALSE | FALSE | TRUE |
| ENSG00000150672 | DLG2 | 11 | 83166055 | 85338966 | protein_coding | −1.671263924 | 5.54E−30 | FALSE | FALSE | FALSE | FALSE | FALSE | FALSE |
| ENSG00000186094 | AGBL4 | 1 | 48998527 | 50489585 | protein_coding | −1.672495121 | 1.68E−17 | FALSE | FALSE | FALSE | FALSE | FALSE | FALSE |
| ENSG00000248309 | MEF2C-AS1 | 5 | 88179145 | 88762215 | antisense | −1.673839724 | 1.59E−27 | FALSE | FALSE | FALSE | FALSE | FALSE | FALSE |
| ENSG00000157404 | KIT | 4 | 55524085 | 55606881 | protein_coding | −1.675518503 | 8.89E−23 | FALSE | FALSE | FALSE | FALSE | FALSE | FALSE |
| ENSG00000162989 | KCNJ3 | 2 | 155554811 | 155714863 | protein_coding | −1.676143236 | 3.62E−09 | FALSE | FALSE | FALSE | FALSE | FALSE | TRUE |
| ENSG00000154645 | CHODL | 21 | 19273580 | 19639690 | protein_coding | −1.677326446 | 1.11E−13 | FALSE | FALSE | FALSE | FALSE | FALSE | FALSE |
| ENSG00000089199 | CHGB | 20 | 5892076 | 5906007 | protein_coding | −1.678324981 | 1.26E−08 | FALSE | FALSE | FALSE | TRUE | TRUE | FALSE |
| ENSG00000142611 | PRDM16 | 1 | 2985732 | 3355185 | protein_coding | −1.680062033 | 8.06E−18 | FALSE | FALSE | FALSE | FALSE | FALSE | FALSE |
| ENSG00000111879 | FAM184A | 6 | 119280928 | 119470552 | protein_coding | −1.680951836 | 6.09E−32 | FALSE | FALSE | FALSE | FALSE | FALSE | FALSE |
| ENSG00000150627 | WDR17 | 4 | 176986985 | 177103978 | protein_coding | −1.682088069 | 5.95E−26 | FALSE | FALSE | FALSE | FALSE | FALSE | FALSE |
| ENSG00000270093 | | 21 | 18015848 | 18017384 | lincRNA | −1.682146158 | 5.01E−21 | FALSE | FALSE | FALSE | FALSE | FALSE | FALSE |
| ENSG00000163075 | | 2 | 120302008 | 120419827 | protein_coding | −1.682343709 | 4.73E−17 | FALSE | FALSE | FALSE | FALSE | FALSE | TRUE |
| ENSG00000128849 | CGNL1 | 15 | 57668165 | 57842925 | protein_coding | −1.684677046 | 6.64E−31 | FALSE | FALSE | FALSE | FALSE | FALSE | FALSE |
| ENSG00000154319 | FAM167A | 8 | 11278972 | 11332224 | protein_coding | −1.687280174 | 7.04E−18 | FALSE | FALSE | FALSE | FALSE | FALSE | FALSE |
| ENSG00000141431 | ASXL3 | 18 | 31158579 | 31331156 | protein_coding | −1.687999145 | 1.82E−21 | FALSE | FALSE | FALSE | FALSE | FALSE | FALSE |
| ENSG00000204323 | SMIM5 | 17 | 73629514 | 73637484 | protein_coding | −1.690697939 | 2.02E−18 | FALSE | FALSE | FALSE | FALSE | FALSE | FALSE |
| ENSG00000144331 | ZNF385B | 2 | 180306709 | 180726232 | protein_coding | −1.691849775 | 1.12E−16 | FALSE | FALSE | FALSE | FALSE | FALSE | FALSE |
| ENSG00000110195 | FOLR1 | 11 | 71900602 | 71907345 | protein_coding | −1.693769715 | 5.91E−12 | FALSE | FALSE | FALSE | FALSE | FALSE | TRUE |
| ENSG00000186204 | CYP4F12 | 19 | 15783567 | 15807984 | protein_coding | −1.696051433 | 7.59E−24 | FALSE | FALSE | FALSE | FALSE | FALSE | TRUE |
| ENSG00000123572 | NRK | X | 105066536 | 105202602 | protein_coding | −1.699056028 | 3.31E−10 | FALSE | FALSE | FALSE | FALSE | FALSE | FALSE |
| ENSG00000164106 | SCRG1 | 4 | 174305852 | 174327531 | protein_coding | −1.70117361 | 6.53E−13 | FALSE | FALSE | FALSE | FALSE | FALSE | TRUE |
| ENSG00000184434 | LRRC19 | 9 | 26993134 | 27005691 | protein_coding | −1.701756387 | 4.32E−14 | FALSE | FALSE | FALSE | FALSE | FALSE | TRUE |
| ENSG00000172425 | TTC36 | 11 | 118398187 | 118401912 | protein_coding | −1.702757671 | 6.58E−15 | FALSE | FALSE | FALSE | FALSE | FALSE | TRUE |
| ENSG00000164035 | EMCN | 4 | 101316498 | 101801283 | protein_coding | −1.704150592 | 9.11E−34 | FALSE | FALSE | FALSE | FALSE | FALSE | FALSE |
| ENSG00000163884 | KLF15 | 3 | 126061478 | 126076285 | protein_coding | −1.705790873 | 8.63E−27 | FALSE | FALSE | FALSE | FALSE | FALSE | FALSE |
| ENSG00000182916 | TCEAL7 | X | 102585124 | 102587254 | protein_coding | −1.706624485 | 9.42E−43 | FALSE | FALSE | FALSE | FALSE | FALSE | FALSE |
| ENSG00000123243 | ITIHS | 10 | 7601232 | 7708961 | protein_coding | −1.710531995 | 6.88E−24 | FALSE | FALSE | FALSE | FALSE | FALSE | FALSE |
| ENSG00000259134 | LINC00924 | 15 | 95976324 | 96051076 | lincRNA | −1.713110718 | 1.08E−33 | FALSE | FALSE | FALSE | FALSE | FALSE | FALSE |
| ENSG00000145692 | BHMT | 5 | 78407602 | 78428108 | protein_coding | −1.71466988 | 3.46E−10 | FALSE | FALSE | FALSE | FALSE | FALSE | TRUE |
| ENSG00000131471 | AOC3 | 17 | 41003201 | 41010147 | protein_coding | −1.714830858 | 1.26E−32 | FALSE | FALSE | FALSE | FALSE | FALSE | FALSE |
| ENSG00000156414 | TDRD9 | 14 | 104394799 | 104519004 | protein_coding | −1.71602375 | 1.23E−18 | FALSE | FALSE | FALSE | FALSE | FALSE | FALSE |
| ENSG00000165659 | DACH1 | 13 | 72012098 | 72441330 | protein_coding | −1.718146698 | 1.75E−20 | FALSE | FALSE | FALSE | FALSE | FALSE | FALSE |
| ENSG00000140285 | FGF7 | 15 | 49715293 | 49780972 | protein_coding | −1.718862268 | 1.90E−20 | FALSE | FALSE | FALSE | FALSE | FALSE | FALSE |
| ENSG00000143416 | SELENBP1 | 1 | 151336778 | 151345209 | protein_coding | −1.720636918 | 7.29E−38 | FALSE | FALSE | FALSE | FALSE | FALSE | TRUE |
| ENSG00000250056 | LINC01018 | 5 | 6582249 | 6588612 | lincRNA | −1.720714531 | 1.41E−12 | FALSE | FALSE | FALSE | FALSE | FALSE | FALSE |
| ENSG00000214575 | CPEB1 | 15 | 83211951 | 83317612 | protein_coding | −1.721711916 | 2.52E−15 | FALSE | FALSE | FALSE | FALSE | FALSE | FALSE |
| ENSG00000082175 | PGR | 11 | 100900355 | 101001255 | protein_coding | −1.722123713 | 1.34E−20 | FALSE | FALSE | FALSE | FALSE | FALSE | TRUE |
| ENSG00000133116 | KL | 13 | 33590207 | 33640282 | protein_coding | −1.728299018 | 8.05E−25 | FALSE | FALSE | FALSE | FALSE | FALSE | TRUE |
| ENSG00000261496 | | 4 | 86933449 | 86935027 | sense_overlapping | −1.728350177 | 1.66E−34 | FALSE | FALSE | FALSE | FALSE | FALSE | FALSE |
| ENSG00000099960 | SLC7A4 | 22 | 21383007 | 21387129 | protein_coding | −1.728358242 | 4.67E−14 | FALSE | FALSE | FALSE | FALSE | FALSE | FALSE |
| ENSG00000177791 | MYOZ1 | 10 | 75391412 | 75401515 | protein_coding | −1.730156602 | 2.17E−23 | FALSE | FALSE | FALSE | FALSE | FALSE | FALSE |
| ENSG00000198417 | MT1F | 16 | 56691606 | 56694610 | protein_coding | −1.730565295 | 3.72E−16 | FALSE | FALSE | FALSE | FALSE | FALSE | FALSE |
| ENSG00000183549 | ACSM5 | 16 | 20420856 | 20452658 | protein_coding | −1.730879658 | 3.26E−22 | FALSE | FALSE | FALSE | FALSE | FALSE | TRUE |
| ENSG00000086159 | AQP6 | 12 | 50360977 | 50370922 | protein_coding | −1.731770438 | 1.48E−06 | FALSE | FALSE | FALSE | FALSE | FALSE | FALSE |
| ENSG00000203685 | C1orf95 | 1 | 226736501 | 226796915 | protein_coding | −1.731887208 | 1.19E−18 | FALSE | FALSE | FALSE | FALSE | FALSE | FALSE |
| ENSG00000164764 | SBSPON | 8 | 73976775 | 74036323 | protein_coding | −1.732305893 | 7.22E−23 | FALSE | FALSE | FALSE | FALSE | FALSE | FALSE |
| ENSG00000172478 | C2orf54 | 2 | 241825465 | 241836306 | protein_coding | −1.73257761 | 3.01E−08 | FALSE | FALSE | FALSE | TRUE | FALSE | FALSE |
| ENSG00000173114 | LRRN3 | 7 | 110731062 | 110765510 | protein_coding | −1.733577343 | 1.49E−28 | FALSE | FALSE | FALSE | FALSE | FALSE | FALSE |
| ENSG00000005249 | PRKAR2B | 7 | 106685094 | 106802256 | protein_coding | −1.736075243 | 2.05E−44 | FALSE | FALSE | FALSE | FALSE | FALSE | FALSE |
| ENSG00000138615 | CILP | 15 | 65488337 | 65503826 | protein_coding | −1.738838515 | 6.12E−16 | FALSE | FALSE | FALSE | FALSE | FALSE | TRUE |
| ENSG00000064205 | WISP2 | 20 | 43343485 | 43357150 | protein_coding | −1.740840665 | 1.51E−12 | FALSE | FALSE | FALSE | FALSE | TRUE | TRUE |
| ENSG00000179639 | FCER1A | 1 | 159259504 | 159278014 | protein_coding | −1.742978537 | 1.85E−22 | FALSE | FALSE | FALSE | FALSE | FALSE | FALSE |
| ENSG00000272789 | | 2 | 128383572 | 128384423 | antisense | −1.745655006 | 1.19E−22 | FALSE | FALSE | TRUE | TRUE | FALSE | TRUE |
| ENSG00000129244 | ATP1B2 | 17 | 7549945 | 7561086 | protein_coding | −1.746615009 | 3.13E−40 | FALSE | FALSE | FALSE | FALSE | FALSE | TRUE |
| ENSG00000240747 | KRBOX1 | 3 | 42850975 | 42984284 | protein_coding | −1.747180831 | 3.64E−21 | FALSE | FALSE | FALSE | FALSE | FALSE | FALSE |
| ENSG00000120156 | TEK | 9 | 27109139 | 27230173 | protein_coding | −1.74782856 | 1.90E−38 | FALSE | FALSE | FALSE | FALSE | FALSE | FALSE |
| ENSG00000185156 | MFSD6L | 17 | 8700436 | 8702667 | protein_coding | −1.748302354 | 3.44E−13 | FALSE | FALSE | FALSE | FALSE | FALSE | FALSE |
| ENSG00000168546 | GFRA2 | 8 | 21547915 | 21669869 | protein_coding | −1.751415349 | 6.17E−33 | FALSE | FALSE | FALSE | FALSE | FALSE | FALSE |
| ENSG00000156298 | TSPAN7 | X | 38420623 | 38548169 | protein_coding | −1.75181211 | 8.13E−32 | FALSE | FALSE | FALSE | FALSE | FALSE | FALSE |
| ENSG00000132874 | SLC14A2 | 18 | 42792960 | 43263072 | protein_coding | −1.751990144 | 8.28E−11 | FALSE | FALSE | FALSE | FALSE | FALSE | FALSE |
| ENSG00000135917 | SLC19A3 | 2 | 228549926 | 228582728 | protein_coding | −1.751996094 | 4.37E−21 | FALSE | FALSE | FALSE | FALSE | FALSE | FALSE |
| ENSG00000105894 | PTN | 7 | 136912088 | 137028611 | protein_coding | −1.752083363 | 9.27E−25 | FALSE | FALSE | FALSE | FALSE | FALSE | FALSE |
| ENSG00000188517 | COL25A1 | 4 | 109731877 | 110223813 | protein_coding | −1.75335032 | 3.21E−16 | FALSE | FALSE | FALSE | FALSE | FALSE | FALSE |
| ENSG00000164619 | BMPER | 7 | 33944523 | 34195484 | protein_coding | −1.754667406 | 2.20E−18 | FALSE | FALSE | FALSE | FALSE | FALSE | FALSE |
| ENSG00000007001 | UPP2 | 2 | 158733214 | 158992666 | protein_coding | −1.756011479 | 2.79E−12 | FALSE | FALSE | FALSE | FALSE | FALSE | FALSE |
| ENSG00000100626 | GALNT16 | 14 | 69725994 | 69821183 | protein_coding | −1.757442319 | 1.42E−27 | FALSE | FALSE | FALSE | FALSE | FALSE | FALSE |
| ENSG00000188487 | INSC | 11 | 15133970 | 15268754 | protein_coding | −1.757794471 | 3.48E−17 | FALSE | FALSE | FALSE | FALSE | FALSE | FALSE |
| ENSG00000228401 | | 9 | 139543062 | 139554873 | antisense | −1.758835593 | 1.26E−27 | FALSE | FALSE | FALSE | FALSE | FALSE | FALSE |
| ENSG00000162391 | FAM151A | 1 | 55074855 | 55089229 | protein_coding | −1.759067546 | 4.57E−13 | FALSE | FALSE | FALSE | FALSE | FALSE | TRUE |
| ENSG00000180815 | MAP3K15 | X | 19378174 | 19533379 | protein_coding | −1.76035044 | 8.86E−20 | FALSE | FALSE | FALSE | FALSE | FALSE | FALSE |
| ENSG00000174175 | SELP | 1 | 169558087 | 169599431 | protein_coding | −1.762460959 | 1.45E−29 | FALSE | FALSE | FALSE | FALSE | FALSE | FALSE |
| ENSG00000140795 | MYLK3 | 16 | 46740891 | 46824319 | protein_coding | −1.762863712 | 7.93E−16 | FALSE | FALSE | FALSE | FALSE | FALSE | FALSE |
| ENSG00000267640 | | 19 | 38307999 | 38317278 | lincRNA | −1.764710719 | 1.66E−22 | FALSE | FALSE | FALSE | FALSE | FALSE | FALSE |
| ENSG00000158258 | CLSTN2 | 3 | 139654027 | 140296239 | protein_coding | −1.765582199 | 3.35E−22 | FALSE | FALSE | FALSE | FALSE | FALSE | TRUE |
| ENSG00000143869 | GDF7 | 2 | 20866424 | 20873418 | protein_coding | −1.766229988 | 2.41E−25 | FALSE | FALSE | FALSE | FALSE | FALSE | FALSE |
| ENSG00000136011 | STAB2 | 12 | 103981051 | 104160505 | protein_coding | −1.766523437 | 3.19E−15 | FALSE | FALSE | FALSE | TRUE | TRUE | FALSE |
| ENSG00000148357 | HMCN2 | 9 | 133046882 | 133309510 | protein_coding | −1.768695678 | 1.18E−25 | FALSE | FALSE | FALSE | FALSE | FALSE | FALSE |
| ENSG00000069535 | MAOB | X | 43625858 | 43741693 | protein_coding | −1.768978052 | 1.96E−27 | FALSE | FALSE | FALSE | FALSE | FALSE | FALSE |
| ENSG00000242600 | MBL1P | 10 | 81664654 | 81710092 | pseudogene | −1.773521818 | 2.30E−24 | FALSE | FALSE | FALSE | FALSE | FALSE | TRUE |
| ENSG00000126950 | TMEM35 | X | 100333709 | 100351353 | protein_coding | −1.775523367 | 3.06E−18 | FALSE | FALSE | FALSE | FALSE | FALSE | FALSE |
| ENSG00000227467 | | 11 | 72281704 | 72284273 | lincRNA | −1.776151005 | 2.33E−34 | FALSE | FALSE | FALSE | FALSE | FALSE | FALSE |
| ENSG00000162595 | DIRAS3 | 1 | 68511645 | 68517314 | protein_coding | −1.781399776 | 2.09E−29 | FALSE | FALSE | FALSE | FALSE | FALSE | FALSE |
| ENSG00000127249 | ATP13A4 | 3 | 193119866 | 193310900 | protein_coding | −1.781684364 | 2.19E−16 | FALSE | FALSE | FALSE | FALSE | TRUE | TRUE |
| ENSG00000168481 | LG13 | 8 | 22004338 | 22014597 | protein_coding | −1.786431715 | 1.50E−11 | FALSE | FALSE | FALSE | FALSE | FALSE | TRUE |
| ENSG00000115353 | TACR1 | 2 | 75273590 | 75426826 | protein_coding | −1.786535908 | 3.76E−26 | FALSE | FALSE | FALSE | FALSE | FALSE | FALSE |
| ENSG00000169031 | COL4A3 | 2 | 228029281 | 228179508 | protein_coding | −1.787174414 | 5.62E−28 | FALSE | FALSE | FALSE | FALSE | FALSE | TRUE |
| ENSG00000123358 | NR4A1 | 12 | 52416616 | 52453291 | protein_coding | −1.78727072 | 6.92E−31 | FALSE | FALSE | FALSE | FALSE | FALSE | FALSE |
| ENSG00000153823 | PID1 | 2 | 229715242 | 230136001 | protein_coding | −1.787870891 | 3.23E−44 | FALSE | FALSE | FALSE | FALSE | FALSE | FALSE |
| ENSG00000228314 | CYP4F29P | 21 | 15215454 | 15220685 | pseudogene | −1.790538723 | 2.04E−16 | FALSE | FALSE | FALSE | TRUE | FALSE | FALSE |
| ENSG00000166819 | PLIN1 | 15 | 90207596 | 90222658 | protein_coding | −1.790763081 | 6.98E−18 | FALSE | FALSE | FALSE | FALSE | TRUE | TRUE |
| ENSG00000106018 | VIPR2 | 7 | 158820866 | 158937649 | protein_coding | −1.791421916 | 1.56E−22 | FALSE | FALSE | FALSE | FALSE | FALSE | FALSE |
| ENSG00000189129 | PLAC9 | 10 | 81891438 | 81905115 | protein_coding | −1.791799261 | 2.87E−30 | FALSE | FALSE | FALSE | FALSE | FALSE | FALSE |
| ENSG00000134042 | MRO | 18 | 48324574 | 48351772 | protein_coding | −1.793902608 | 7.43E−19 | FALSE | FALSE | FALSE | FALSE | FALSE | FALSE |
| ENSG00000140459 | CYP11A1 | 15 | 74630100 | 74660081 | protein_coding | −1.79485453 | 1.06E−14 | FALSE | FALSE | FALSE | FALSE | FALSE | FALSE |
| ENSG00000197083 | ZNF300P1 | 5 | 150310207 | 150325851 | pseudogene | −1.795189264 | 1.04E−32 | FALSE | FALSE | FALSE | FALSE | FALSE | FALSE |
| ENSG00000138650 | PCDH10 | 4 | 134070470 | 134129356 | protein_coding | −1.795316943 | 1.07E−14 | FALSE | FALSE | FALSE | FALSE | FALSE | FALSE |
| ENSG00000186479 | RGS7BP | 5 | 63802084 | 63908139 | protein_coding | −1.802802253 | 3.55E−26 | FALSE | FALSE | FALSE | FALSE | FALSE | FALSE |
| ENSG00000162706 | CADM3 | 1 | 159141399 | 159173103 | protein_coding | −1.803598503 | 1.25E−14 | FALSE | FALSE | FALSE | FALSE | FALSE | FALSE |
| ENSG00000160111 | CPAMD8 | 19 | 17003758 | 17137625 | protein_coding | −1.804195343 | 5.18E−31 | FALSE | FALSE | FALSE | FALSE | FALSE | FALSE |
| ENSG00000092051 | JPH4 | 14 | 24037244 | 24048009 | protein_coding | −1.805447289 | 9.26E−43 | FALSE | FALSE | FALSE | FALSE | FALSE | FALSE |
| ENSG00000248810 | | 4 | 142240604 | 142253772 | lincRNA | −1.80671275 | 6.66E−16 | FALSE | FALSE | FALSE | FALSE | FALSE | FALSE |
| ENSG00000120738 | EGR1 | 5 | 137801179 | 137805004 | protein_coding | −1.811001893 | 2.98E−38 | FALSE | FALSE | FALSE | FALSE | FALSE | FALSE |
| ENSG00000272511 | | 3 | 21453714 | 21455436 | lincRNA | −1.811530238 | 1.27E−19 | FALSE | FALSE | FALSE | FALSE | FALSE | FALSE |
| ENSG00000197991 | PCDH20 | 13 | 61983818 | 62002220 | protein_coding | −1.812236511 | 3.65E−16 | FALSE | FALSE | FALSE | FALSE | FALSE | FALSE |
| ENSG00000171303 | KCNK3 | 2 | 26915619 | 26956288 | protein_coding | −1.814629185 | 9.34E−18 | FALSE | FALSE | FALSE | FALSE | FALSE | FALSE |
| ENSG00000254510 | | 11 | 66176629 | 66184608 | processed_transc [symbol] | −1.815452463 | 2.79E−20 | FALSE | FALSE | FALSE | FALSE | FALSE | FALSE |
| ENSG00000167676 | PLIN4 | 19 | 4502204 | 4517716 | protein_coding | −1.815596175 | 2.01E−18 | FALSE | FALSE | TRUE | TRUE | TRUE | TRUE |
| ENSG00000184845 | DRD1 | 5 | 174867042 | 174871211 | protein_coding | −1.816450092 | 3.04E−20 | FALSE | FALSE | FALSE | FALSE | FALSE | FALSE |
| ENSG00000137960 | GIPC2 | 1 | 78445226 | 78604133 | protein_coding | −1.816725548 | 3.25E−29 | FALSE | FALSE | FALSE | FALSE | FALSE | TRUE |
| ENSG00000115361 | ACADL | 2 | 211052663 | 211090215 | protein_coding | −1.821152606 | 5.34E−17 | FALSE | FALSE | FALSE | FALSE | FALSE | TRUE |
| ENSG00000231768 | | 1 | 234663637 | 234667525 | lincRNA | −1.821160632 | 1.72E−25 | FALSE | FALSE | FALSE | FALSE | FALSE | FALSE |
| ENSG00000132840 | BHMT2 | 5 | 78365540 | 78385289 | protein_coding | −1.823721259 | 1.97E−25 | FALSE | FALSE | FALSE | FALSE | FALSE | TRUE |
| ENSG00000189134 | NKAPL | 6 | 28227098 | 28228736 | protein_coding | −1.826800794 | 3.91E−42 | FALSE | FALSE | FALSE | FALSE | FALSE | FALSE |
| ENSG00000186642 | PDE2A | 11 | 72287185 | 72385635 | protein_coding | −1.828109436 | 7.89E−46 | FALSE | FALSE | FALSE | FALSE | FALSE | TRUE |
| ENSG00000248890 | HHIP-AS1 | 4 | 145564074 | 145582509 | antisense | −1.8284498 | 2.93E−29 | FALSE | FALSE | FALSE | FALSE | FALSE | FALSE |
| ENSG00000166206 | GABRB3 | 15 | 26788693 | 27184686 | protein_coding | −1.83129499 | 9.05E−16 | FALSE | FALSE | FALSE | FALSE | FALSE | FALSE |
| ENSG00000165124 | SVEP1 | 9 | 113127531 | 113342160 | protein_coding | −1.831434637 | 5.41E−30 | FALSE | FALSE | FALSE | FALSE | FALSE | FALSE |
| ENSG00000036448 | MYOM2 | 8 | 1993155 | 2113475 | protein_coding | −1.831720273 | 1.29E−41 | FALSE | FALSE | FALSE | FALSE | FALSE | FALSE |
| ENSG00000255090 | | 11 | 122026130 | 122293579 | processed_transc [symbol] | −1.83767701 | 1.78E−23 | FALSE | FALSE | FALSE | FALSE | FALSE | FALSE |
| ENSG00000253250 | C8orf88 | 8 | 91970865 | 91997485 | protein_coding | −1.839043874 | 2.76E−26 | FALSE | FALSE | FALSE | FALSE | FALSE | FALSE |
| ENSG00000185652 | NTF3 | 12 | 5541278 | 5630702 | protein_coding | −1.840244826 | 2.60E−30 | FALSE | FALSE | FALSE | FALSE | FALSE | FALSE |
| ENSG00000148795 | CYP17A1 | 10 | 104590288 | 104597290 | protein_coding | −1.841514047 | 1.95E−15 | FALSE | FALSE | FALSE | FALSE | TRUE | TRUE |
| ENSG00000112964 | GHR | 5 | 42423879 | 42721979 | protein_coding | −1.841941729 | 2.98E−35 | FALSE | FALSE | FALSE | FALSE | FALSE | FALSE |
| ENSG00000132854 | KANK4 | 1 | 62702651 | 62785085 | protein_coding | −1.842555414 | 2.93E−20 | FALSE | FALSE | FALSE | FALSE | FALSE | FALSE |
| ENSG00000189058 | APOD | 3 | 195295573 | 195311076 | protein_coding | −1.842775498 | 1.02E−22 | FALSE | FALSE | FALSE | FALSE | FALSE | TRUE |
| ENSG00000261685 | | 16 | 50679720 | 50683160 | lincRNA | −1.843958047 | 2.58E−23 | FALSE | FALSE | FALSE | FALSE | FALSE | FALSE |
| ENSG00000137872 | SEMA6D | 15 | 47476298 | 48066420 | protein_coding | −1.848622236 | 1.74E−39 | FALSE | FALSE | FALSE | FALSE | FALSE | FALSE |
| ENSG00000272143 | FGF14-AS2 | 13 | 103046980 | 103048053 | lincRNA | −1.849957568 | 6.73E−47 | FALSE | FALSE | FALSE | FALSE | FALSE | FALSE |
| ENSG00000046653 | GPM6B | X | 13789150 | 13956757 | protein_coding | −1.850838729 | 3.13E−49 | FALSE | FALSE | FALSE | FALSE | FALSE | TRUE |
| ENSG00000204099 | NEU4 | 2 | 242749920 | 242758739 | protein_coding | −1.851399865 | 1.27E−15 | FALSE | FALSE | FALSE | FALSE | FALSE | FALSE |
| ENSG00000113594 | LIFR | 5 | 38475065 | 38608456 | protein_coding | −1.854398446 | 3.95E−41 | FALSE | FALSE | FALSE | FALSE | FALSE | FALSE |
| ENSG00000157765 | SLC34A2 | 4 | 25656923 | 25680370 | protein_coding | −1.856065726 | 1.63E−08 | FALSE | FALSE | FALSE | FALSE | FALSE | TRUE |
| ENSG00000248485 | PCP4L1 | 1 | 161228517 | 161255240p | rotein_coding | −1.857304144 | 1.49E−21 | FALSE | FALSE | FALSE | FALSE | FALSE | FALSE |
| ENSG00000116983 | HPCAL4 | 1 | 40144320 | 40157361 | protein_coding | −1.85832122 | 2.24E−12 | FALSE | FALSE | FALSE | FALSE | FALSE | FALSE |
| ENSG00000262061 | | 17 | 180996 | 183279 | processed_transc [symbol] | −1.859478707 | 2.18E−44 | FALSE | FALSE | FALSE | FALSE | FALSE | FALSE |
| ENSG00000151623 | NR3C2 | 4 | 148999913 | 149365850 | protein_coding | −1.859559611 | 2.18E−42 | FALSE | FALSE | FALSE | FALSE | FALSE | FALSE |
| ENSG00000043591 | ADRB1 | 10 | 115803806 | 115806667 | protein_coding | −1.861101173 | 3.97E−19 | FALSE | FALSE | FALSE | FALSE | FALSE | TRUE |
| ENSG00000106278 | PTPRZ1 | 7 | 121513143 | 121702090 | protein_coding | −1.86263652 | 6.82E−13 | FALSE | FALSE | FALSE | TRUE | TRUE | TRUE |
| ENSG00000101605 | MYOM1 | 18 | 3066805 | 3220106 | protein_coding | −1.863636355 | 3.61E−30 | FALSE | FALSE | FALSE | FALSE | FALSE | FALSE |
| ENSG00000069702 | TGFBR3 | 1 | 92145902 | 92371892 | protein_coding | −1.864233976 | 1.68E−60 | FALSE | FALSE | FALSE | FALSE | TRUE | FALSE |
| ENSG00000184012 | TMPRSS2 | 21 | 42836478 | 42903043 | protein_coding | −1.870809526 | 1.91E−14 | FALSE | FALSE | FALSE | FALSE | TRUE | TRUE |
| ENSG00000162669 | HFM1 | 1 | 91726323 | 91870426 | protein_coding | −1.871051932 | 2.23E−23 | FALSE | FALSE | FALSE | FALSE | FALSE | FALSE |
| ENSG00000034239 | EFCAB1 | 8 | 49623348 | 49647870 | protein_coding | −1.871431642 | 1.68E−23 | FALSE | FALSE | FALSE | FALSE | FALSE | FALSE |
| ENSG00000158445 | KCNB1 | 20 | 47980414 | 48099184 | protein_coding | −1.872528768 | 5.69E−20 | FALSE | FALSE | FALSE | FALSE | FALSE | FALSE |
| ENSG00000146469 | VIP | 6 | 153071933 | 153080900 | protein_coding | −1.872529899 | 9.42E−19 | FALSE | FALSE | FALSE | FALSE | FALSE | TRUE |
| ENSG00000110675 | ELMOD1 | 11 | 107461817 | 107537505 | protein_coding | −1.874786641 | 2.19E−17 | FALSE | FALSE | FALSE | FALSE | FALSE | FALSE |
| ENSG00000105852 | PON3 | 7 | 94989256 | 95025680 | protein_coding | −1.875315698 | 1.44E−19 | FALSE | FALSE | FALSE | FALSE | FALSE | TRUE |
| ENSG00000109819 | PPARGC1A | 4 | 23756664 | 23905712 | protein_coding | −1.875804545 | 1.75E−31 | FALSE | FALSE | FALSE | FALSE | FALSE | FALSE |
| ENSG00000147606 | SLC26A7 | 8 | 92221722 | 92410378 | protein_coding | −1.877331179 | 1.26E−14 | FALSE | FALSE | FALSE | FALSE | FALSE | FALSE |
| ENSG00000150893 | FREM2 | 13 | 39261266 | 39460074 | protein_coding | −1.877744811 | 5.38E−15 | FALSE | FALSE | FALSE | FALSE | FALSE | FALSE |
| ENSG00000197584 | KCNMB2 | 3 | 177990720 | 178562217 | protein_coding | −1.877901636 | 4.09E−23 | FALSE | FALSE | FALSE | FALSE | FALSE | FALSE |
| ENSG00000140092 | FBLN5 | 14 | 92335756 | 92414331 | protein_coding | −1.879471964 | 7.69E−49 | FALSE | FALSE | FALSE | FALSE | FALSE | FALSE |
| ENSG00000134121 | CHL1 | 3 | 238279 | 451090 | protein_coding | −1.880551424 | 2.04E−18 | FALSE | FALSE | FALSE | FALSE | FALSE | FALSE |
| ENSG00000124212 | PTGIS | 20 | 48120411 | 48184683 | protein_coding | −1.884187192 | 5.16E−28 | FALSE | FALSE | FALSE | FALSE | FALSE | FALSE |
| ENSG00000101955 | SRPX | X | 38008589 | 38080696 | protein_coding | −1.884320695 | 2.27E−38 | FALSE | FALSE | FALSE | FALSE | FALSE | FALSE |
| ENSG00000153993 | SEMA3D | 7 | 84624869 | 84816171 | protein_coding | −1.885203563 | 2.15E−24 | FALSE | FALSE | FALSE | FALSE | FALSE | FALSE |
| ENSG00000213494 | CCL14 | 17 | 34310327 | 34329100 | protein_coding | −1.887355202 | 7.41E−28 | FALSE | FALSE | FALSE | FALSE | FALSE | FALSE |
| ENSG00000127241 | MASP1 | 3 | 186935942 | 187009810 | protein_coding | −1.889063337 | 5.31E−21 | FALSE | FALSE | FALSE | FALSE | FALSE | FALSE |
| ENSG00000153253 | SCN3A | 2 | 165944032 | 166060577 | protein_coding | −1.891977919 | 5.19E−29 | FALSE | FALSE | FALSE | FALSE | FALSE | FALSE |
| ENSG00000189233 | NUGGC | 8 | 27879481 | 27941388 | protein_coding | −1.892201144 | 1.73E−21 | FALSE | FALSE | FALSE | FALSE | FALSE | FALSE |
| ENSG00000183346 | C10orf107 | 10 | 63422719 | 63526524 | protein_coding | −1.894184799 | 3.12E−22 | FALSE | FALSE | FALSE | FALSE | FALSE | FALSE |
| ENSG00000254528 | | 11 | 117704434 | 117709657 | antisense | −1.8942923 | 3.14E−19 | FALSE | FALSE | FALSE | FALSE | FALSE | TRUE |
| ENSG00000068976 | PYGM | 11 | 64513861 | 64527769 | protein_coding | −1.898341341 | 3.96E−26 | FALSE | FALSE | FALSE | TRUE | TRUE | TRUE |
| ENSG00000118194 | TNNT2 | 1 | 201328136 | 201346890 | protein_coding | −1.898715752 | 4.04E−10 | FALSE | FALSE | FALSE | FALSE | TRUE | FALSE |
| ENSG00000187193 | MT1X | 16 | 56716336 | 56718108 | protein_coding | −1.899421745 | 7.41E−30 | FALSE | FALSE | FALSE | FALSE | FALSE | FALSE |
| ENSG00000111452 | GPR133 | 12 | 131438452 | 131626014 | protein_coding | −1.900630039 | 2.62E−29 | FALSE | FALSE | FALSE | FALSE | FALSE | TRUE |
| ENSG00000162877 | PM20D1 | 1 | 205797150 | 205819260 | protein_coding | −1.906838863 | 1.94E−21 | FALSE | FALSE | FALSE | FALSE | FALSE | FALSE |
| ENSG00000161640 | SIGLEC11 | 19 | 50452242 | 50464429 | protein_coding | −1.907113167 | 3.58E−32 | FALSE | FALSE | FALSE | FALSE | TRUE | FALSE |
| ENSG00000106034 | CPED1 | 7 | 120628731 | 120937498 | protein_coding | −1.907643137 | 5.27E−43 | FALSE | FALSE | FALSE | FALSE | FALSE | FALSE |
| ENSG00000013297 | CLDN11 | 3 | 170136653 | 170578169 | protein_coding | −1.908020425 | 5.89E−31 | FALSE | FALSE | FALSE | FALSE | FALSE | FALSE |
| ENSG00000211452 | DIO1 | 1 | 54356912 | 54376759 | protein_coding | −1.911776297 | 1.75E−12 | FALSE | FALSE | FALSE | FALSE | FALSE | FALSE |
| ENSG00000072133 | RPS6KA6 | X | 83318984 | 83442933 | protein_coding | −1.914022857 | 2.22E−19 | FALSE | FALSE | FALSE | FALSE | FALSE | FALSE |
| ENSG00000186715 | MST1L | 1 | 17081405 | 17096732 | pseudogene | −1.915455573 | 6.30E−22 | FALSE | FALSE | FALSE | FALSE | FALSE | FALSE |
| ENSG00000036672 | USP2 | 11 | 119225925 | 119252436 | protein_coding | −1.920205091 | 4.18E−44 | FALSE | FALSE | FALSE | FALSE | FALSE | FALSE |
| ENSG00000094755 | GABRP | 5 | 170190354 | 170241051 | protein_coding | −1.92117704 | 5.31E−13 | FALSE | FALSE | FALSE | FALSE | FALSE | FALSE |
| ENSG00000197766 | CFD | 19 | 859453 | 863453 | protein_coding | −1.923231906 | 8.50E−27 | FALSE | FALSE | FALSE | TRUE | TRUE | TRUE |
| ENSG00000119121 | TRPM6 | 9 | 77337411 | 77503010 | protein_coding | −1.923344686 | 5.76E−21 | FALSE | FALSE | FALSE | FALSE | TRUE | FALSE |
| ENSG00000134873 | CLDN10 | 13 | 96085858 | 96232013 | protein_coding | −1.93220889 | 1.35E−10 | FALSE | FALSE | FALSE | FALSE | FALSE | FALSE |
| ENSG00000136842 | TMOD1 | 9 | 100263462 | 100364030 | protein_coding | −1.934242851 | 8.60E−33 | FALSE | FALSE | FALSE | FALSE | FALSE | FALSE |
| ENSG00000185274 | WBSCR17 | 7 | 70597155 | 71178585 | protein_coding | −1.934532432 | 1.42E−17 | FALSE | FALSE | FALSE | FALSE | FALSE | FALSE |
| ENSG00000001626 | CFTR | 7 | 117105838 | 117356025 | protein_coding | −1.93465624 | 9.73E−13 | FALSE | FALSE | FALSE | FALSE | FALSE | TRUE |
| ENSG00000205795 | CYS1 | 2 | 10196907 | 10221071 | protein_coding | −1.935941501 | 3.00E−27 | FALSE | FALSE | FALSE | FALSE | FALSE | TRUE |
| ENSG00000225873 | LINC00694 | 3 | 44462619 | 44465499 | lincRNA | −1.937671678 | 7.02E−28 | FALSE | FALSE | FALSE | FALSE | FALSE | FALSE |
| ENSG00000060709 | RIMBP2 | 12 | 130880682 | 131200826 | protein_coding | −1.940464993 | 4.95E−18 | FALSE | FALSE | FALSE | FALSE | FALSE | FALSE |
| ENSG00000152779 | SLC16A12 | 10 | 91190051 | 91316398 | protein_coding | −1.942462075 | 1.87E−27 | FALSE | FALSE | FALSE | FALSE | FALSE | TRUE |
| ENSG00000130988 | RGN | X | 46937775 | 46952712 | protein_coding | −1.94378361 | 1.80E−42 | FALSE | FALSE | FALSE | FALSE | FALSE | FALSE |
| ENSG00000101425 | BPI | 20 | 36888551 | 36965907 | protein_coding | −1.945534566 | 2.97E−22 | FALSE | FALSE | FALSE | FALSE | TRUE | FALSE |
| ENSG00000196482 | ESRRG | 1 | 216676588 | 217311097 | protein_coding | −1.945819402 | 5.55E−26 | FALSE | FALSE | FALSE | FALSE | FALSE | FALSE |
| ENSG00000163431 | LMOD1 | 1 | 201865580 | 201915715 | protein_coding | −1.94649451 | 6.98E−31 | FALSE | FALSE | FALSE | FALSE | TRUE | FALSE |
| ENSG00000134853 | PDGFRA | 4 | 55095264 | 55164414 | protein_coding | −1.947253218 | 3.51E−30 | FALSE | FALSE | FALSE | FALSE | TRUE | FALSE |
| ENSG00000158246 | FAM46B | 1 | 27331511 | 27339327 | protein_coding | −1.947620808 | 1.08E−31 | FALSE | FALSE | FALSE | FALSE | FALSE | TRUE |
| ENSG00000135333 | EPHA7 | 6 | 93949738 | 94129265 | protein_coding | −1.947835117 | 1.59E−14 | FALSE | FALSE | FALSE | FALSE | FALSE | FALSE |
| ENSG00000244953 | | 11 | 43965337 | 43968756 | lincRNA | −1.950845053 | 2.51E−26 | FALSE | FALSE | FALSE | FALSE | FALSE | FALSE |
| ENSG00000144230 | GPR17 | 2 | 128403439 | 128410213 | protein_coding | −1.952537307 | 2.91E−34 | FALSE | FALSE | TRUE | TRUE | TRUE | TRUE |
| ENSG00000213424 | KRT222 | 17 | 38810917 | 38821433 | protein_coding | −1.962778187 | 4.13E−22 | FALSE | FALSE | FALSE | FALSE | FALSE | FALSE |
| ENSG00000196109 | ZNF676 | 19 | 22361893 | 22379753 | protein_coding | −1.963516354 | 6.30E−19 | FALSE | FALSE | FALSE | FALSE | FALSE | FALSE |
| ENSG00000168356 | SCN11A | 3 | 38887260 | 38992052 | protein_coding | −1.96619153 | 2.55E−39 | FALSE | FALSE | FALSE | FALSE | FALSE | FALSE |
| ENSG00000169862 | CTNND2 | 5 | 10971952 | 11904155 | protein_coding | −1.968970886 | 3.46E−17 | FALSE | FALSE | FALSE | FALSE | FALSE | TRUE |
| ENSG00000113805 | CNTN3 | 3 | 74311719 | 74570291 | protein_coding | −1.973349759 | 2.93E−19 | FALSE | FALSE | FALSE | FALSE | FALSE | FALSE |
| ENSG00000168004 | HRASLS5 | 11 | 63228876 | 63258666 | protein_coding | −1.976505999 | 1.69E−19 | FALSE | FALSE | FALSE | FALSE | FALSE | FALSE |
| ENSG00000170345 | FOS | 14 | 75745477 | 75748933 | protein_coding | −1.976610064 | 5.59E−43 | FALSE | FALSE | FALSE | FALSE | FALSE | FALSE |
| ENSG00000148488 | ST8SIA6 | 10 | 17360382 | 17496329 | protein_coding | −1.978072095 | 1.36E−22 | FALSE | FALSE | FALSE | FALSE | FALSE | TRUE |
| ENSG00000255085 | | 8 | 145925738 | 145933902 | pseudogene | −1.980154112 | 1.24E−30 | FALSE | FALSE | FALSE | FALSE | FALSE | FALSE |
| ENSG00000100181 | TPTEP1 | 22 | 17082777 | 17179632 | lincRNA | −1.98219549 | 7.27E−35 | FALSE | FALSE | FALSE | FALSE | FALSE | FALSE |
| ENSG00000161896 | IP6K3 | 6 | 33689444 | 33714762 | protein_coding | −1.982492528 | 3.56E−21 | FALSE | FALSE | FALSE | FALSE | FALSE | FALSE |
| ENSG00000152092 | ASTN1 | 1 | 176826438 | 177134109 | protein_coding | −1.984035839 | 1.27E−19 | FALSE | FALSE | FALSE | FALSE | TRUE | FALSE |
| ENSG00000137878 | GCOM1 | 15 | 57884106 | 58006943 | protein_coding | −1.985132683 | 7.96E−25 | FALSE | FALSE | FALSE | TRUE | TRUE | TRUE |
| ENSG00000123119 | NECAB1 | 8 | 91803778 | 91971636 | protein_coding | −1.986827859 | 9.52E−39 | FALSE | FALSE | FALSE | FALSE | FALSE | FALSE |
| ENSG00000103647 | CORO2B | 15 | 68871308 | 69020145 | protein_coding | −1.986830162 | 3.17E−45 | FALSE | FALSE | FALSE | FALSE | FALSE | FALSE |
| ENSG00000134548 | C12orf39 | 12 | 21679241 | 21690311 | protein_coding | −1.988016117 | 1.85E−21 | FALSE | FALSE | FALSE | FALSE | FALSE | FALSE |
| ENSG00000139874 | SSTR1 | 14 | 38677204 | 38682272 | protein_coding | −1.988034402 | 3.00E−27 | FALSE | FALSE | FALSE | FALSE | FALSE | FALSE |
| ENSG00000080293 | SCTR | 2 | 120197419 | 120282070 | protein_coding | −1.991559195 | 1.23E−21 | FALSE | FALSE | FALSE | FALSE | FALSE | TRUE |
| ENSG00000112530 | PACRG | 6 | 163148164 | 163736524 | protein_coding | −1.994227036 | 4.73E−26 | FALSE | FALSE | FALSE | FALSE | FALSE | FALSE |
| ENSG00000108823 | SGCA | 17 | 48241575 | 48253292 | protein_coding | −1.994265077 | 5.70E−29 | FALSE | FALSE | FALSE | FALSE | FALSE | FALSE |
| ENSG00000118729 | CASQ2 | 1 | 116242628 | 116311402 | protein_coding | −1.996723149 | 1.80E−18 | FALSE | FALSE | FALSE | FALSE | FALSE | FALSE |
| ENSG00000145777 | TSLP | 5 | 110405760 | 110413722 | protein_coding | −1.999901663 | 1.11E−35 | FALSE | FALSE | FALSE | FALSE | FALSE | FALSE |
| ENSG00000241644 | INMT | 7 | 30737601 | 30797218 | protein_coding | −2.003179798 | 1.56E−33 | FALSE | FALSE | FALSE | FALSE | FALSE | FALSE |
| ENSG00000189292 | FAM150B | 2 | 279558 | 288851 | protein_coding | −2.004080694 | 7.48E−24 | FALSE | FALSE | FALSE | FALSE | FALSE | FALSE |
| ENSG00000154175 | ABI3BP | 3 | 100468000 | 100712359 | protein_coding | −2.005736568 | 1.02E−30 | FALSE | FALSE | FALSE | FALSE | FALSE | FALSE |
| ENSG00000162630 | B3GALT2 | 1 | 193148175 | 193155784 | protein_coding | −2.007072069 | 9.39E−42 | FALSE | FALSE | FALSE | FALSE | FALSE | FALSE |
| ENSG00000169218 | RSPO1 | 1 | 38076951 | 38100595 | protein_coding | −2.008208045 | 8.07E−30 | FALSE | FALSE | FALSE | FALSE | FALSE | TRUE |
| ENSG00000128918 | ALDH1A2 | 15 | 58245622 | 58790065 | protein_coding | −2.00892194 | 1.09E−32 | FALSE | FALSE | FALSE | FALSE | FALSE | FALSE |
| ENSG00000168497 | SDPR | 2 | 192699028 | 192711981 | protein_coding | −2.014693562 | 1.45E−43 | FALSE | FALSE | FALSE | FALSE | FALSE | FALSE |
| ENSG00000186510 | CLCNKA | 1 | 16345370 | 16360545 | protein_coding | −2.016484333 | 4.77E−14 | FALSE | FALSE | FALSE | FALSE | FALSE | FALSE |
| ENSG00000159307 | SCUBE1 | 22 | 43593289 | 43739394 | protein_coding | −2.019667124 | 3.83E−26 | FALSE | FALSE | FALSE | FALSE | FALSE | FALSE |
| ENSG00000164100 | NDST3 | 4 | 118954773 | 119179803 | protein_coding | −2.021464663 | 1.14E−18 | FALSE | FALSE | FALSE | FALSE | FALSE | FALSE |
| ENSG00000180432 | CYP8B1 | 3 | 42897497 | 42917633 | protein_coding | −2.022603791 | 6.87E−20 | FALSE | FALSE | FALSE | FALSE | FALSE | FALSE |
| ENSG00000225194 | LINC00092 | 9 | 98782014 | 98790247 | lincRNA | −2.023811592 | 4.81E−46 | FALSE | FALSE | FALSE | FALSE | FALSE | FALSE |
| ENSG00000072657 | TRHDE | 12 | 72481046 | 73059422 | protein_coding | −2.027413869 | 3.82E−19 | FALSE | FALSE | FALSE | FALSE | FALSE | FALSE |
| ENSG00000263812 | LINC00908 | 18 | 74240612 | 74322925 | protein_coding | −2.034806442 | 2.51E−32 | FALSE | FALSE | FALSE | FALSE | FALSE | FALSE |
| ENSG00000257877 | | 12 | 112250597 | 112251224 | lincRNA | −2.038114625 | 7.06E−48 | FALSE | FALSE | FALSE | FALSE | FALSE | FALSE |
| ENSG00000154262 | ABCA6 | 17 | 67074843 | 67138029 | protein_coding | −2.044386718 | 4.12E−46 | FALSE | FALSE | FALSE | FALSE | FALSE | FALSE |
| ENSG00000205929 | C21orf62 | 21 | 34162985 | 34186053 | protein_coding | −2.045063543 | 2.10E−27 | FALSE | FALSE | FALSE | FALSE | FALSE | FALSE |
| ENSG00000170509 | HSD17B13 | 4 | 88224941 | 88244058 | protein_coding | −2.046748129 | 4.17E−21 | FALSE | FALSE | FALSE | FALSE | FALSE | FALSE |
| ENSG00000118137 | APOA1 | 11 | 116706467 | 116708666 | protein_coding | −2.047333792 | 1.37E−20 | FALSE | FALSE | FALSE | FALSE | FALSE | TRUE |
| ENSG00000119715 | ESRRB | 14 | 76776957 | 76968178 | protein_coding | −2.05183467 | 7.29E−17 | FALSE | FALSE | FALSE | FALSE | FALSE | FALSE |
| ENSG00000198910 | L1CAM | X | 153126969 | 153174677 | protein_coding | −2.052141115 | 5.73E−19 | FALSE | FALSE | FALSE | FALSE | TRUE | FALSE |
| ENSG00000188536 | HBA2 | 16 | 222846 | 223709 | protein_coding | −2.054898852 | 4.03E−33 | FALSE | FALSE | FALSE | TRUE | FALSE | FALSE |
| ENSG00000170153 | RNF150 | 4 | 141780961 | 142134031 | protein_coding | −2.056929037 | 8.74E−39 | FALSE | FALSE | FALSE | FALSE | FALSE | FALSE |
| ENSG00000268388 | FENDRR | 16 | 86508135 | 86542705 | lincRNA | −2.060318694 | 1.46E−23 | FALSE | FALSE | FALSE | FALSE | FALSE | TRUE |
| ENSG00000146374 | RSPO3 | 6 | 127439749 | 127518910 | protein_coding | −2.060819144 | 6.95E−30 | FALSE | FALSE | FALSE | FALSE | FALSE | FALSE |
| ENSG00000122420 | PTGFR | 1 | 78769568 | 79005434 | protein_coding | −2.062667323 | 4.26E−28 | FALSE | FALSE | FALSE | FALSE | FALSE | FALSE |
| ENSG00000168874 | ATOH8 | 2 | 85978467 | 86015189 | protein_coding | −2.063196998 | 2.32E−39 | FALSE | FALSE | FALSE | FALSE | FALSE | FALSE |
| ENSG00000022267 | FHL1 | X | 135229559 | 135293518 | protein_coding | −2.064744812 | 1.85E−29 | FALSE | FALSE | FALSE | FALSE | FALSE | FALSE |
| ENSG00000152154 | TMEM178A | 2 | 39892122 | 39945103 | protein_coding | −2.070807128 | 1.23E−35 | FALSE | FALSE | FALSE | FALSE | FALSE | FALSE |
| ENSG00000136014 | USP44 | 12 | 95910336 | 95945266 | protein_coding | −2.073356261 | 9.13E−46 | FALSE | FALSE | FALSE | FALSE | FALSE | FALSE |
| ENSG00000243955 | GSTA1 | 6 | 52656462 | 52668708 | protein_coding | −2.075810692 | 7.04E−13 | FALSE | FALSE | FALSE | FALSE | FALSE | FALSE |
| ENSG00000114854 | TNNC1 | 3 | 52485118 | 52488086 | protein_coding | −2.077638292 | 2.24E−18 | FALSE | FALSE | TRUE | TRUE | TRUE | TRUE |
| ENSG00000157152 | SYN2 | 3 | 12045876 | 12232900 | processed_transc [symbol] | −2.078341674 | 1.38E−31 | FALSE | FALSE | FALSE | FALSE | TRUE | FALSE |
| ENSG00000211445 | GPX3 | 5 | 150400124 | 150408554 | protein_coding | −2.079737012 | 7.19E−45 | FALSE | FALSE | FALSE | FALSE | FALSE | TRUE |
| ENSG00000116183 | PAPPA2 | 1 | 176432307 | 176814735 | protein_coding | −2.084865604 | 7.24E−17 | FALSE | FALSE | FALSE | FALSE | FALSE | FALSE |
| ENSG00000107562 | CXCL12 | 10 | 44793038 | 44881941 | protein_coding | −2.085464133 | 1.45E−59 | FALSE | FALSE | FALSE | FALSE | FALSE | FALSE |
| ENSG00000160868 | CYP3A4 | 7 | 99354604 | 99381888 | protein_coding | −2.087729009 | 9.99E−16 | FALSE | FALSE | FALSE | FALSE | FALSE | TRUE |
| ENSG00000183230 | CTNNA3 | 10 | 67672276 | 69455927 | protein_coding | −2.088267152 | 2.24E−19 | FALSE | FALSE | FALSE | FALSE | FALSE | FALSE |
| ENSG00000134917 | ADAMTS8 | 11 | 130274820 | 130298888 | protein_coding | −2.089972555 | 5.44E−34 | FALSE | FALSE | FALSE | FALSE | FALSE | FALSE |
| ENSG00000126895 | AVPR2 | X | 153167985 | 153172620 | protein_coding | −2.095319306 | 9.97E−31 | FALSE | FALSE | FALSE | FALSE | FALSE | FALSE |
| ENSG00000149294 | NCAM1 | 11 | 112831997 | 113149158 | protein_coding | −2.100693498 | 1.25E−29 | FALSE | FALSE | FALSE | FALSE | TRUE | TRUE |
| ENSG00000119508 | NR4A3 | 9 | 102584137 | 102629173 | protein_coding | −2.103588731 | 9.79E−40 | FALSE | FALSE | FALSE | FALSE | FALSE | FALSE |
| ENSG00000083067 | TRPM3 | 9 | 73143979 | 74061820 | protein_coding | −2.104391917 | 5.65E−31 | FALSE | FALSE | FALSE | FALSE | FALSE | FALSE |
| ENSG00000198848 | CES1 | 16 | 55836763 | 55867249 | protein_coding | −2.106435591 | 7.44E−30 | FALSE | FALSE | FALSE | FALSE | FALSE | FALSE |
| ENSG00000166292 | TMEM100 | 17 | 53796988 | 53809482 | protein_coding | −2.111914306 | 1.10E−26 | FALSE | FALSE | FALSE | FALSE | FALSE | FALSE |
| ENSG00000138823 | MTTP | 4 | 100484918 | 100545156 | protein_coding | −2.116259132 | 4.96E−20 | FALSE | FALSE | FALSE | FALSE | FALSE | FALSE |
| ENSG00000261625 | | 11 | 68768233 | 68769516 | sense_overlapping | −2.120098771 | 1.66E−31 | FALSE | FALSE | FALSE | FALSE | FALSE | FALSE |
| ENSG00000080493 | SLC4A4 | 4 | 72053003 | 72437804 | protein_coding | −2.122355429 | 9.35E−29 | FALSE | FALSE | FALSE | FALSE | FALSE | TRUE |
| ENSG00000147257 | GPC3 | X | 132669773 | 133119922 | protein_coding | −2.127126046 | 1.67E−21 | FALSE | FALSE | FALSE | FALSE | FALSE | FALSE |
| ENSG00000169715 | MT1E | 16 | 56659387 | 56661024 | protein_coding | −2.127885633 | 2.91E−35 | FALSE | FALSE | FALSE | FALSE | FALSE | FALSE |
| ENSG00000226237 | | 9 | 89563614 | 89616948 | lincRNA | −2.129919861 | 2.27E−49 | FALSE | FALSE | FALSE | FALSE | FALSE | FALSE |
| ENSG00000182851 | GPIHBP1 | 8 | 144295068 | 144299044 | protein_coding | −2.132797117 | 7.52E−35 | FALSE | FALSE | FALSE | FALSE | FALSE | FALSE |
| ENSG00000108924 | HLF | 17 | 53342373 | 53402426 | protein_coding | −2.132942686 | 2.07E−43 | FALSE | FALSE | FALSE | FALSE | FALSE | FALSE |
| ENSG00000100448 | CTSG | 14 | 25042728 | 25045466 | protein_coding | −2.139496378 | 7.20E−24 | FALSE | FALSE | FALSE | FALSE | FALSE | FALSE |
| ENSG00000138722 | MMRN1 | 4 | 90800683 | 90875780 | protein_coding | −2.146712126 | 6.95E−29 | FALSE | FALSE | FALSE | FALSE | FALSE | FALSE |
| ENSG00000111339 | ART4 | 12 | 14978503 | 14996429 | protein_coding | −2.151831107 | 7.25E−34 | FALSE | FALSE | FALSE | FALSE | FALSE | FALSE |
| ENSG00000164128 | NPY1R | 4 | 164245113 | 164265984 | protein_coding | −2.156688955 | 8.00E−42 | FALSE | FALSE | FALSE | FALSE | FALSE | TRUE |
| ENSG00000127083 | OMD | 9 | 95176527 | 95186743 | protein_coding | −2.158237522 | 2.99E−27 | FALSE | FALSE | FALSE | FALSE | FALSE | FALSE |
| ENSG00000140538 | NTRK3 | 15 | 88418230 | 88799999 | protein_coding | −2.162655725 | 7.80E−37 | FALSE | FALSE | FALSE | FALSE | FALSE | FALSE |
| ENSG00000175287 | PHYHD1 | 9 | 131683174 | 131704320 | protein_coding | −2.163614153 | 3.43E−43 | FALSE | FALSE | FALSE | FALSE | FALSE | FALSE |
| ENSG00000050030 | KIAA2022 | X | 73952684 | 74145282 | protein_coding | −2.170930741 | 3.35E−27 | FALSE | FALSE | FALSE | FALSE | FALSE | FALSE |
| ENSG00000109906 | ZBTB16 | 11 | 113930315 | 114121398 | protein_coding | −2.172161704 | 1.97E−32 | FALSE | FALSE | FALSE | FALSE | FALSE | FALSE |
| ENSG00000164488 | DACT2 | 6 | 168693510 | 168720434 | protein_coding | −2.173127051 | 4.30E−19 | FALSE | FALSE | FALSE | FALSE | FALSE | FALSE |
| ENSG00000112782 | CLIC5 | 6 | 45868045 | 46048132 | protein_coding | −2.174870515 | 3.58E−27 | FALSE | FALSE | FALSE | FALSE | FALSE | TRUE |
| ENSG00000171714 | ANO5 | 11 | 22214722 | 22304903 | protein_coding | −2.175690983 | 2.40E−33 | FALSE | FALSE | FALSE | FALSE | FALSE | FALSE |
| ENSG00000112319 | EYA4 | 6 | 133561736 | 133853258 | protein_coding | −2.185116408 | 3.33E−24 | FALSE | FALSE | FALSE | FALSE | FALSE | FALSE |
| ENSG00000148053 | NTRK2 | 9 | 87283466 | 87638505 | protein_coding | −2.185320339 | 8.01E−29 | FALSE | FALSE | FALSE | FALSE | FALSE | TRUE |
| ENSG00000166405 | RIC3 | 11 | 8127597 | 8190602 | protein_coding | −2.187039487 | 7.88E−39 | FALSE | FALSE | FALSE | FALSE | TRUE | TRUE |
| ENSG00000188783 | PRELP | 1 | 203444956 | 203460480 | protein_coding | −2.195202442 | 9.59E−44 | FALSE | FALSE | FALSE | FALSE | FALSE | FALSE |
| ENSG00000172935 | MRGPRF | 11 | 68771863 | 68780877 | protein_coding | −2.197124466 | 5.26E−45 | FALSE | FALSE | FALSE | FALSE | FALSE | FALSE |
| ENSG00000136872 | ALDOB | 9 | 104182860 | 104198105 | protein_coding | −2.202880344 | 6.59E−16 | FALSE | FALSE | FALSE | FALSE | FALSE | TRUE |
| ENSG00000141469 | SLC14A1 | 18 | 43304092 | 43332485 | protein_coding | −2.202996386 | 5.71E−38 | FALSE | FALSE | FALSE | FALSE | FALSE | FALSE |
| ENSG00000258498 | DIO30S | 14 | 102018558 | 102026768 | lincRNA | −2.203604965 | 5.67E−31 | FALSE | FALSE | TRUE | TRUE | FALSE | TRUE |
| ENSG00000187957 | DNER | 2 | 230222345 | 230579274 | protein_coding | −2.20446187 | 9.16E−18 | FALSE | FALSE | FALSE | FALSE | FALSE | FALSE |
| ENSG00000241684 | ADAMTS9-AS: | 3 | 64670585 | 64997143 | antisense | −2.216710641 | 4.81E−41 | FALSE | FALSE | FALSE | FALSE | FALSE | TRUE |
| ENSG00000197705 | KLHL14 | 18 | 30252634 | 30353025 | protein_coding | −2.217057571 | 3.99E−32 | FALSE | FALSE | FALSE | FALSE | FALSE | TRUE |
| ENSG00000244476 | ERVFRD-1 | 6 | 11102722 | 11111965 | protein_coding | −2.217677535 | 4.86E−36 | FALSE | FALSE | FALSE | FALSE | FALSE | FALSE |
| ENSG00000133878 | DUSP26 | 8 | 33448856 | 33457624 | protein_coding | −2.220173157 | 8.71E−31 | FALSE | FALSE | FALSE | FALSE | FALSE | FALSE |
| ENSG00000198963 | RORB | 9 | 77112281 | 77308093 | protein_coding | −2.220413603 | 2.08E−30 | FALSE | FALSE | FALSE | FALSE | FALSE | FALSE |
| ENSG00000165269 | AQP7 | 9 | 33384765 | 33402643 | protein_coding | −2.224805893 | 7.98E−38 | FALSE | FALSE | FALSE | FALSE | FALSE | FALSE |
| ENSG00000170743 | SYT9 | 11 | 7260009 | 7490273 | protein_coding | −2.228348896 | 5.93E−22 | FALSE | FALSE | FALSE | FALSE | FALSE | FALSE |
| ENSG00000205362 | MT1A | 16 | 56672578 | 56673999 | protein_coding | −2.237925869 | 2.54E−32 | FALSE | FALSE | FALSE | FALSE | FALSE | FALSE |
| ENSG00000162896 | PIGR | 1 | 207101863 | 207119811 | protein_coding | −2.241558108 | 1.13E−18 | FALSE | FALSE | FALSE | FALSE | FALSE | TRUE |
| ENSG00000183844 | FAM3B | 21 | 42676139 | 42729358 | protein_coding | −2.25173389 | 1.72E−19 | FALSE | FALSE | FALSE | FALSE | FALSE | FALSE |
| ENSG00000167236 | CCL23 | 17 | 34340096 | 34345005 | protein_coding | −2.254269227 | 8.11E−38 | FALSE | FALSE | FALSE | FALSE | FALSE | FALSE |
| ENSG00000198300 | PEG3 | 19 | 57321445 | 57352096 | protein_coding | −2.257521089 | 8.27E−55 | FALSE | FALSE | FALSE | FALSE | FALSE | FALSE |
| ENSG00000180509 | KCNE1 | 21 | 35818988 | 35884573 | protein_coding | −2.263323729 | 8.09E−33 | FALSE | FALSE | FALSE | TRUE | TRUE | FALSE |
| ENSG00000161798 | AQP5 | 12 | 50355653 | 50359464 | protein_coding | −2.263510307 | 2.87E−13 | FALSE | FALSE | FALSE | FALSE | FALSE | TRUE |
| ENSG00000122824 | NUDT10 | X | 51075083 | 51080377 | protein_coding | −2.265006859 | 1.43E−33 | FALSE | FALSE | FALSE | FALSE | FALSE | FALSE |
| ENSG00000187889 | C1orf168 | 1 | 57184477 | 57285369 | protein_coding | −2.265408562 | 1.85E−26 | FALSE | FALSE | FALSE | FALSE | FALSE | FALSE |
| ENSG00000133636 | NTS | 12 | 86268073 | 86276770 | protein_coding | −2.267326254 | 9.65E−15 | FALSE | FALSE | FALSE | FALSE | FALSE | FALSE |
| ENSG00000166856 | GPR182 | 12 | 57388230 | 57390468 | protein_coding | −2.293971073 | 8.03E−37 | FALSE | FALSE | FALSE | TRUE | TRUE | FALSE |
| ENSG00000070808 | CAMK2A | 5 | 149599054 | 149669854 | protein_coding | −2.305057424 | 9.33E−24 | FALSE | FALSE | FALSE | FALSE | FALSE | FALSE |
| ENSG00000241186 | TDGF1 | 3 | 46616045 | 46668033 | protein_coding | −2.305094075 | 2.47E−17 | FALSE | FALSE | FALSE | FALSE | FALSE | TRUE |
| ENSG00000151892 | GFRA1 | 10 | 117816444 | 118032979 | protein_coding | −2.305850753 | 2.42E−36 | FALSE | FALSE | FALSE | FALSE | FALSE | TRUE |
| ENSG00000164120 | HPGD | 4 | 175411328 | 175444305 | protein_coding | −2.311887003 | 4.77E−35 | FALSE | FALSE | FALSE | FALSE | FALSE | FALSE |
| ENSG00000149451 | ADAM33 | 20 | 3648612 | 3662893 | protein_coding | −2.315602428 | 2.34E−47 | FALSE | TRUE | TRUE | TRUE | TRUE | TRUE |
| ENSG00000185985 | SLITRK2 | X | 144899350 | 144907360 | protein_coding | −2.32296863 | 8.59E−28 | FALSE | FALSE | FALSE | FALSE | FALSE | FALSE |
| ENSG00000189056 | RELN | 7 | 103112231 | 103629963 | protein_coding | −2.324724708 | 2.75E−29 | FALSE | FALSE | FALSE | FALSE | FALSE | FALSE |
| ENSG00000124440 | HIF3A | 19 | 46800303 | 46846690 | protein_coding | −2.328061993 | 4.58E−41 | FALSE | FALSE | FALSE | TRUE | FALSE | TRUE |
| ENSG00000248290 | TNXA | 6 | 31976391 | 31980249 | pseudogene | −2.334032304 | 8.95E−24 | FALSE | FALSE | FALSE | FALSE | FALSE | TRUE |
| ENSG00000244734 | HBB | 11 | 5246694 | 5250625 | protein_coding | −2.339863104 | 1.19E−42 | FALSE | FALSE | FALSE | TRUE | FALSE | FALSE |
| ENSG00000154258 | ABCA9 | 17 | 66970629 | 67057205 | protein_coding | −2.34250724 | 2.60E−54 | FALSE | TRUE | TRUE | TRUE | FALSE | TRUE |
| ENSG00000173641 | HSPB7 | 1 | 16340523 | 16346089 | protein_coding | −2.345642708 | 3.30E−34 | FALSE | FALSE | FALSE | TRUE | TRUE | TRUE |
| ENSG00000172260 | NEGR1 | 1 | 71861623 | 72748417 | protein_coding | −2.349702287 | 9.04E−46 | FALSE | FALSE | FALSE | FALSE | FALSE | FALSE |
| ENSG00000004799 | PDK4 | 7 | 95212811 | 95225803 | protein_coding | −2.362799607 | 1.75E−48 | FALSE | FALSE | FALSE | FALSE | FALSE | FALSE |
| ENSG00000142973 | CYP4B1 | 1 | 47223510 | 47285085 | protein_coding | −2.36326079 | 1.64E−25 | FALSE | FALSE | FALSE | FALSE | FALSE | TRUE |
| ENSG00000166183 | ASPG | 14 | 104552016 | 104579098 | protein_coding | −2.366201044 | 5.91E−24 | FALSE | FALSE | FALSE | FALSE | TRUE | FALSE |
| ENSG00000007062 | PROM1 | 4 | 15964699 | 16086001 | protein_coding | −2.366639259 | 1.51E−22 | FALSE | FALSE | FALSE | FALSE | FALSE | FALSE |
| ENSG00000137077 | CCL21 | 9 | 34709002 | 34710121 | protein_coding | −2.367419805 | 9.56E−29 | FALSE | FALSE | FALSE | FALSE | FALSE | FALSE |
| ENSG00000154330 | PGM5 | 9 | 70943224 | 71145977 | protein_coding | −2.369006064 | 3.09E−52 | FALSE | FALSE | FALSE | FALSE | FALSE | FALSE |
| ENSG00000158104 | HPD | 12 | 122277433 | 122301502 | protein_coding | −2.369999218 | 1.73E−29 | FALSE | FALSE | FALSE | FALSE | FALSE | FALSE |
| ENSG00000198576 | ARC | 8 | 143692405 | 143696833 | protein_coding | −2.380995802 | 3.65E−34 | FALSE | FALSE | FALSE | FALSE | FALSE | FALSE |
| ENSG00000102678 | FGF9 | 13 | 22245522 | 22278637 | protein_coding | −2.382927069 | 5.60E−22 | FALSE | FALSE | FALSE | FALSE | FALSE | FALSE |
| ENSG00000019102 | VSIG2 | 11 | 124617368 | 124622134 | protein_coding | −2.390724951 | 3.23E−27 | FALSE | FALSE | FALSE | FALSE | TRUE | TRUE |
| ENSG00000094963 | FMO2 | 1 | 171154347 | 171181822 | protein_coding | −2.392082656 | 7.97E−38 | FALSE | FALSE | FALSE | TRUE | FALSE | TRUE |
| ENSG00000065325 | GLP2R | 17 | 9725523 | 9795419 | protein_coding | −2.411233073 | 3.20E−20 | FALSE | FALSE | FALSE | FALSE | FALSE | FALSE |
| ENSG00000134020 | PEBP4 | 8 | 22570769 | 22857513 | protein_coding | −2.418029808 | 6.62E−25 | FALSE | FALSE | FALSE | FALSE | FALSE | TRUE |
| ENSG00000048540 | LMO3 | 12 | 16701307 | 16763528 | protein_coding | −2.427343364 | 1.73E−32 | FALSE | FALSE | FALSE | FALSE | FALSE | TRUE |
| ENSG00000182132 | KCNIP1 | 5 | 169780491 | 170163636 | protein_coding | −2.427834805 | 1.84E−31 | FALSE | FALSE | FALSE | FALSE | FALSE | FALSE |
| ENSG00000165495 | PKNOX2 | 11 | 125034583 | 125303285 | protein_coding | −2.434875501 | 5.49E−56 | FALSE | FALSE | FALSE | FALSE | FALSE | FALSE |
| ENSG00000164946 | FREM1 | 9 | 14734664 | 14910993 | protein_coding | −2.436959335 | 2.82E−38 | FALSE | FALSE | FALSE | FALSE | FALSE | FALSE |
| ENSG00000165125 | TRPV6 | 7 | 142568956 | 142583507 | protein_coding | −2.445639418 | 1.43E−27 | FALSE | FALSE | FALSE | FALSE | TRUE | TRUE |
| ENSG00000206172 | HBA1 | 16 | 226679 | 227521 | protein_coding | −2.448435242 | 5.57E−32 | FALSE | FALSE | FALSE | TRUE | FALSE | FALSE |
| ENSG00000167741 | GGT6 | 17 | 4460222 | 4464113 | protein_coding | −2.448676899 | 6.24E−12 | FALSE | FALSE | FALSE | FALSE | FALSE | TRUE |
| ENSG00000107317 | PTGDS | 9 | 139871956 | 139879887 | protein_coding | −2.45000787 | 2.84E−41 | FALSE | FALSE | FALSE | FALSE | FALSE | FALSE |
| ENSG00000127324 | TSPAN8 | 12 | 71518865 | 71835678 | protein_coding | −2.450601863 | 7.40E−22 | FALSE | FALSE | FALSE | FALSE | FALSE | TRUE |
| ENSG00000138207 | RBP4 | 10 | 95351444 | 95361501 | protein_coding | −2.459062844 | 4.12E−24 | FALSE | FALSE | FALSE | FALSE | FALSE | TRUE |
| ENSG00000168477 | TNXB | 6 | 32008931 | 32083111 | protein_coding | −2.47204173 | 2.77E−50 | FALSE | FALSE | FALSE | TRUE | TRUE | TRUE |
| ENSG00000114200 | BCHE | 3 | 165490692 | 165555260 | protein_coding | −2.475483732 | 4.21E−34 | FALSE | FALSE | FALSE | FALSE | FALSE | FALSE |
| ENSG00000165966 | PDZRN4 | 12 | 41582250 | 41968392 | protein_coding | −2.482527148 | 4.96E−31 | FALSE | FALSE | FALSE | FALSE | FALSE | FALSE |
| ENSG00000039537 | C6 | 5 | 41142336 | 41261540 | protein_coding | −2.485298355 | 5.18E−25 | FALSE | FALSE | FALSE | FALSE | FALSE | FALSE |
| ENSG00000115602 | IL1RL1 | 2 | 102927962 | 102968497 | protein_coding | −2.495492343 | 1.77E−33 | FALSE | FALSE | FALSE | FALSE | TRUE | FALSE |
| ENSG00000197565 | COL4A6 | X | 107386780 | 107682727 | protein_coding | −2.498733654 | 7.15E−31 | FALSE | FALSE | FALSE | FALSE | FALSE | TRUE |
| ENSG00000077274 | CAPN6 | X | 110488331 | 110513751 | protein_coding | −2.503155284 | 6.53E−28 | FALSE | FALSE | FALSE | FALSE | FALSE | FALSE |
| ENSG00000144891 | AGTR1 | 3 | 148415571 | 148460795 | protein_coding | −2.513200991 | 4.19E−43 | FALSE | FALSE | FALSE | FALSE | FALSE | FALSE |
| ENSG00000160808 | MYL3 | 3 | 46899362 | 46923659 | protein_coding | −2.514194772 | 3.17E−35 | FALSE | FALSE | TRUE | TRUE | TRUE | TRUE |
| ENSG00000189320 | FAM180A | 7 | 135413096 | 135433594 | protein_coding | −2.528429748 | 1.14E−42 | FALSE | FALSE | FALSE | FALSE | FALSE | FALSE |
| ENSG00000184908 | CLCNKB | 1 | 16370272 | 16383803 | protein_coding | −2.53130351 | 1.78E−22 | FALSE | FALSE | FALSE | FALSE | FALSE | FALSE |
| ENSG00000135298 | BAI3 | 6 | 69345259 | 70099403 | protein_coding | −2.532606818 | 4.83E−49 | FALSE | FALSE | FALSE | FALSE | FALSE | FALSE |
| ENSG00000188488 | SERPINA5 | 14 | 95027779 | 95059457 | protein_coding | −2.546150364 | 5.41E−29 | FALSE | FALSE | FALSE | FALSE | FALSE | TRUE |
| ENSG00000259417 | | 15 | 80487826 | 80544555 | lincRNA | −2.549653894 | 4.23E−36 | FALSE | FALSE | FALSE | FALSE | FALSE | FALSE |
| ENSG00000163710 | PCOLCE2 | 3 | 142534764 | 142608045 | protein_coding | −2.550740467 | 2.43E−35 | FALSE | FALSE | FALSE | FALSE | FALSE | FALSE |
| ENSG00000145362 | ANK2 | 4 | 113739265 | 114304896 | protein_coding | −2.553702696 | 3.61E−66 | FALSE | FALSE | FALSE | FALSE | FALSE | FALSE |
| ENSG00000153822 | KCNJ16 | 17 | 68049570 | 68131749 | protein_coding | −2.554138056 | 2.01E−23 | FALSE | FALSE | FALSE | FALSE | FALSE | TRUE |
| ENSG00000167588 | GPD1 | 12 | 50497602 | 50505102 | protein_coding | −2.564798254 | 9.13E−34 | FALSE | FALSE | FALSE | FALSE | FALSE | FALSE |
| ENSG00000118094 | TREH | 11 | 118528026 | 118550399 | protein_coding | −2.571886177 | 4.46E−37 | FALSE | FALSE | FALSE | FALSE | FALSE | FALSE |
| ENSG00000198010 | DLGAP2 | 8 | 1449532 | 1656642 | protein_coding | −2.575534619 | 9.04E−44 | FALSE | FALSE | FALSE | FALSE | FALSE | FALSE |
| ENSG00000175785 | PRIMA1 | 14 | 94184644 | 94254827 | protein_coding | −2.591853755 | 3.79E−28 | FALSE | FALSE | FALSE | FALSE | FALSE | TRUE |
| ENSG00000172005 | MAL | 2 | 95691422 | 95719737 | protein_coding | −2.598046339 | 1.14E−35 | FALSE | FALSE | FALSE | TRUE | TRUE | FALSE |
| ENSG00000018625 | ATP1A2 | 1 | 160085549 | 160113381 | protein_coding | −2.601121047 | 1.15E−32 | FALSE | FALSE | FALSE | FALSE | TRUE | TRUE |
| ENSG00000168702 | LRP1B | 2 | 140988992 | 142889270 | protein_coding | −2.602966655 | 9.13E−27 | FALSE | FALSE | FALSE | FALSE | FALSE | FALSE |
| ENSG00000170381 | SEMA3E | 7 | 82993222 | 83278326 | protein_coding | −2.608929492 | 2.50E−29 | FALSE | FALSE | FALSE | FALSE | FALSE | FALSE |
| ENSG00000164303 | ENPP6 | 4 | 185009859 | 185142383 | protein_coding | −2.6201741 | 5.27E−39 | FALSE | FALSE | FALSE | FALSE | FALSE | FALSE |
| ENSG00000165186 | PTCHD1 | X | 23352133 | 23422489 | protein_coding | −2.621494289 | 2.34E−33 | FALSE | FALSE | FALSE | FALSE | FALSE | FALSE |
| ENSG00000011465 | DCN | 12 | 91539025 | 91576900 | protein_coding | −2.62931207 | 9.86E−50 | FALSE | FALSE | FALSE | FALSE | TRUE | FALSE |
| ENSG00000178343 | SHISA3 | 4 | 42399856 | 42404504 | protein_coding | −2.649717837 | 4.81E−38 | FALSE | FALSE | FALSE | FALSE | FALSE | FALSE |
| ENSG00000095713 | CRTAC1 | 10 | 99624757 | 99790585 | protein_coding | −2.651913549 | 2.29E−31 | FALSE | FALSE | FALSE | FALSE | FALSE | FALSE |
| ENSG00000075035 | WSCD2 | 12 | 108523248 | 108644314 | protein_coding | −2.661003891 | 6.12E−36 | FALSE | FALSE | FALSE | FALSE | FALSE | FALSE |
| ENSG00000163827 | LRRC2 | 3 | 46556913 | 46621589 | protein_coding | −2.673440712 | 7.67E−50 | FALSE | FALSE | FALSE | FALSE | FALSE | FALSE |
| ENSG00000165197 | FIGF | X | 15363713 | 15402498 | protein_coding | −2.67393976 | 6.81E−42 | FALSE | FALSE | TRUE | TRUE | TRUE | TRUE |
| ENSG00000266964 | FXYD1 | 19 | 35629712 | 35634013 | protein_coding | −2.716968889 | 5.67E−57 | FALSE | FALSE | FALSE | FALSE | FALSE | FALSE |
| ENSG00000166482 | MFAP4 | 17 | 19286755 | 19290553 | protein_coding | −2.725318402 | 2.35E−63 | FALSE | FALSE | FALSE | FALSE | FALSE | FALSE |
| ENSG00000241158 | ADAMTS9-AS | 3 | 64547014 | 64573878 | antisense | −2.735112145 | 3.62E−35 | FALSE | FALSE | FALSE | FALSE | FALSE | TRUE |
| ENSG00000163815 | CLEC3B | 3 | 45043040 | 45077563 | protein_coding | −2.739573255 | 2.44E−65 | FALSE | FALSE | FALSE | FALSE | FALSE | FALSE |
| ENSG00000004776 | HSPB6 | 19 | 36245469 | 36248980 | protein_coding | −2.741795886 | 8.58E−47 | FALSE | FALSE | FALSE | FALSE | FALSE | FALSE |
| ENSG00000138356 | AOX1 | 2 | 201450591 | 201541787 | protein_coding | −2.749945154 | 8.39E−56 | FALSE | FALSE | FALSE | FALSE | FALSE | FALSE |
| ENSG00000165072 | MAMDC2 | 9 | 72658497 | 72841886 | protein_coding | −2.754300692 | 7.89E−69 | FALSE | FALSE | FALSE | TRUE | TRUE | FALSE |
| ENSG00000168309 | FAM107A | 3 | 58549844 | 58613337 | protein_coding | −2.773680953 | 4.64E−74 | FALSE | FALSE | FALSE | FALSE | FALSE | TRUE |
| ENSG00000182836 | PLCXD3 | 5 | 41307056 | 41510730 | protein_coding | −2.793456655 | 2.64E−52 | FALSE | FALSE | FALSE | FALSE | FALSE | FALSE |
| ENSG00000125740 | FOSB | 19 | 45971253 | 45978437 | protein_coding | −2.815904606 | 1.17E−53 | FALSE | FALSE | FALSE | FALSE | FALSE | FALSE |
| ENSG00000129596 | CD01 | 5 | 115140430 | 115152651 | protein_coding | −2.82882973 | 1.42E−61 | FALSE | FALSE | FALSE | FALSE | FALSE | FALSE |
| ENSG00000120907 | ADRA1A | 8 | 26605667 | 26724790 | protein_coding | −2.856635809 | 1.31E−44 | FALSE | FALSE | FALSE | FALSE | FALSE | FALSE |
| ENSG00000134201 | GSTM5 | 1 | 110254864 | 110318050 | protein_coding | −2.860846294 | 2.58E−94 | FALSE | FALSE | FALSE | FALSE | FALSE | FALSE |
| ENSG00000107623 | GDF10 | 10 | 48425815 | 48438976 | protein_coding | −2.863496162 | 7.15E−35 | FALSE | FALSE | FALSE | FALSE | FALSE | FALSE |
| ENSG00000177133 | LINC00982 | 1 | 2976179 | 2985001 | antisense | −2.865083966 | 5.02E−37 | FALSE | FALSE | FALSE | FALSE | FALSE | FALSE |
| ENSG00000133800 | LYVE1 | 11 | 10578513 | 10633236 | protein_coding | −2.87538698 | 1.66E−80 | FALSE | FALSE | FALSE | TRUE | TRUE | FALSE |
| ENSG00000163145 | C1QTNF7 | 4 | 15341442 | 15447790 | protein_coding | −2.875515934 | 6.45E−86 | FALSE | FALSE | FALSE | FALSE | FALSE | FALSE |
| ENSG00000152580 | IGSF10 | 3 | 151143172 | 151176497 | protein_coding | −2.887733177 | 3.98E−64 | FALSE | FALSE | FALSE | FALSE | FALSE | FALSE |
| ENSG00000149575 | SCN2B | 11 | 118032666 | 118047388 | protein_coding | −2.888590417 | 7.25E−49 | FALSE | FALSE | FALSE | FALSE | FALSE | FALSE |
| ENSG00000164161 | HHIP | 4 | 145567173 | 145666423 | protein_coding | −2.909250942 | 8.65E−45 | FALSE | FALSE | FALSE | FALSE | FALSE | FALSE |
| ENSG00000178568 | ERBB4 | 2 | 212240446 | 213403565 | protein_coding | −2.914060615 | 1.13E−40 | FALSE | FALSE | FALSE | FALSE | FALSE | TRUE |
| ENSG00000146151 | HMGCLL1 | 6 | 55299167 | 55444012 | protein_coding | −2.936373406 | 5.38E−63 | FALSE | FALSE | FALSE | FALSE | FALSE | FALSE |
| ENSG00000160801 | PTH1R | 3 | 46919236 | 46945287 | protein_coding | −2.940045094 | 1.19E−67 | FALSE | FALSE | FALSE | FALSE | FALSE | TRUE |
| ENSG00000108381 | ASPA | 17 | 3375668 | 3406713 | protein_coding | −2.945025001 | 1.31E−78 | FALSE | FALSE | FALSE | FALSE | FALSE | TRUE |
| ENSG00000183287 | CCBE1 | 18 | 57098172 | 57364612 | protein_coding | −2.990542681 | 6.49E−50 | FALSE | FALSE | FALSE | FALSE | TRUE | FALSE |
| ENSG00000164188 | RANBP3L | 5 | 36248536 | 36302216 | protein_coding | −3.009095994 | 8.79E−53 | FALSE | FALSE | FALSE | FALSE | FALSE | TRUE |
| ENSG00000184226 | PCDH9 | 13 | 66876967 | 67804468 | protein_coding | −3.023330173 | 8.13E−71 | FALSE | FALSE | FALSE | FALSE | FALSE | FALSE |
| ENSG00000171885 | AQP4 | 18 | 24432002 | 24445782 | protein_coding | −3.02850321 | 1.11E−43 | FALSE | FALSE | FALSE | FALSE | FALSE | TRUE |
| ENSG00000122121 | XPNPEP2 | X | 128872950 | 128903514 | protein_coding | −3.036884153 | 3.76E−37 | FALSE | FALSE | FALSE | FALSE | FALSE | FALSE |
| ENSG00000205358 | MT1H | 16 | 56703726 | 56705041 | protein_coding | −3.042177411 | 1.61E−26 | FALSE | FALSE | FALSE | FALSE | FALSE | FALSE |
| ENSG00000173376 | NDNF | 4 | 121956768 | 121994176 | protein_coding | −3.056712466 | 2.80E−52 | FALSE | FALSE | FALSE | FALSE | FALSE | TRUE |
| ENSG00000125144 | MT1G | 16 | 56700643 | 56701977 | protein_coding | −3.067912406 | 2.37E−36 | FALSE | FALSE | FALSE | FALSE | FALSE | FALSE |
| ENSG00000107295 | SH3GL2 | 9 | 17579080 | 17797127 | protein_coding | −3.079187785 | 6.90E−43 | FALSE | FALSE | FALSE | FALSE | FALSE | FALSE |
| ENSG00000124253 | PCK1 | 20 | 56136136 | 56141513 | protein_coding | −3.095732933 | 2.03E−38 | FALSE | FALSE | FALSE | FALSE | TRUE | TRUE |
| ENSG00000163687 | DNASE1L3 | 3 | 58177984 | 58200424 | protein_coding | −3.118832531 | 6.21E−82 | FALSE | FALSE | FALSE | TRUE | TRUE | FALSE |
| ENSG00000116194 | ANGPTL1 | 1 | 178818840 | 178840187 | protein_coding | −3.126400407 | 8.44E−79 | FALSE | FALSE | FALSE | FALSE | FALSE | FALSE |
| ENSG00000180875 | GREM2 | 1 | 240652873 | 240775449 | protein_coding | −3.149274865 | 4.81E−55 | FALSE | FALSE | FALSE | FALSE | FALSE | FALSE |
| ENSG00000141338 | ABCA8 | 17 | 66863433 | 66951533 | protein_coding | −3.158002266 | 2.86E−73 | FALSE | FALSE | FALSE | TRUE | FALSE | TRUE |
| ENSG00000133169 | BEX1 | X | 102317579 | 102319168 | protein_coding | −3.182967538 | 2.45E−49 | FALSE | FALSE | FALSE | FALSE | FALSE | FALSE |
| ENSG00000108018 | SORCS1 | 10 | 108333421 | 108924292 | protein_coding | −3.195900046 | 1.11E−45 | FALSE | FALSE | FALSE | FALSE | FALSE | FALSE |
| ENSG00000168447 | SCNN1B | 16 | 23289552 | 23392620 | protein_coding | −3.197357361 | 1.64E−46 | FALSE | FALSE | FALSE | TRUE | TRUE | TRUE |
| ENSG00000153446 | C16orf89 | 16 | 5094123 | 5116111 | protein_coding | −3.203815421 | 1.82E−53 | FALSE | FALSE | FALSE | FALSE | FALSE | FALSE |
| ENSG00000118526 | TCF21 | 6 | 134210276 | 134216691 | protein_coding | −3.210028446 | 5.71E−79 | FALSE | FALSE | FALSE | TRUE | TRUE | TRUE |
| ENSG00000135447 | PPP1R1A | 12 | 54969171 | 54982443 | protein_coding | −3.237128528 | 3.62E−49 | FALSE | FALSE | FALSE | FALSE | FALSE | FALSE |
| ENSG00000205364 | MT1M | 16 | 56666145 | 56667898 | protein_coding | −3.253892812 | 1.00E−78 | FALSE | FALSE | FALSE | FALSE | FALSE | FALSE |
| ENSG00000111404 | RERGL | 12 | 18233803 | 18473041 | protein_coding | −3.254120002 | 3.39E−67 | FALSE | FALSE | FALSE | FALSE | FALSE | FALSE |
| ENSG00000171243 | SOSTDC1 | 7 | 16501106 | 16570205 | protein_coding | −3.271979651 | 9.37E−47 | FALSE | FALSE | FALSE | FALSE | FALSE | FALSE |
| ENSG00000205038 | PKHD1L1 | 8 | 110374706 | 110542559 | protein_coding | −3.298601884 | 1.07E−72 | FALSE | FALSE | TRUE | TRUE | TRUE | TRUE |
| ENSG00000179399 | GPC5 | 13 | 92050929 | 93519490 | protein_coding | −3.329372428 | 4.25E−42 | FALSE | FALSE | FALSE | FALSE | FALSE | FALSE |
| ENSG00000175161 | CADM2 | 3 | 85008132 | 86123579 | protein_coding | −3.334204823 | 1.71E−54 | FALSE | FALSE | FALSE | TRUE | TRUE | TRUE |
| ENSG00000018236 | CNTN1 | 12 | 41086244 | 41466220 | protein_coding | −3.342743859 | 1.52E−50 | FALSE | FALSE | FALSE | FALSE | TRUE | FALSE |
| ENSG00000089250 | NOS1 | 12 | 117645947 | 117889975 | protein_coding | −3.424618383 | 3.00E−56 | FALSE | FALSE | FALSE | FALSE | FALSE | FALSE |
| ENSG00000134240 | HMGCS2 | 1 | 120290619 | 120311528 | protein_coding | −3.475683864 | 5.80E−39 | FALSE | FALSE | FALSE | FALSE | FALSE | TRUE |
| ENSG00000106809 | OGN | 9 | 95146249 | 95166978 | protein_coding | −3.494127558 | 1.80E−65 | FALSE | FALSE | FALSE | FALSE | FALSE | FALSE |
| ENSG00000141639 | MAPK4 | 18 | 48086448 | 48258194 | protein_coding | −3.497077015 | 2.17E−56 | FALSE | FALSE | FALSE | FALSE | FALSE | FALSE |
| ENSG00000145708 | CRHBP | 5 | 76248538 | 76276983 | protein_coding | −3.519924064 | 1.06E−76 | FALSE | FALSE | FALSE | TRUE | TRUE | FALSE |
| ENSG00000143196 | DPT | 1 | 168664697 | 168698502 | protein_coding | −3.564957615 | 1.22E−80 | FALSE | FALSE | TRUE | TRUE | TRUE | FALSE |
| ENSG00000122756 | CNTFR | 9 | 34551430 | 34590121 | protein_coding | −3.651868034 | 1.16E−70 | FALSE | FALSE | FALSE | FALSE | FALSE | FALSE |
| ENSG00000175084 | DES | 2 | 220283099 | 220291461 | protein_coding | −3.674222802 | 4.12E−63 | FALSE | TRUE | TRUE | TRUE | TRUE | TRUE |
| ENSG00000123560 | PLP1 | X | 103028647 | 103047548 | protein_coding | −3.691475989 | 5.61E−66 | FALSE | FALSE | FALSE | TRUE | TRUE | TRUE |
| ENSG00000150625 | GPM6A | 4 | 176554085 | 176923815 | protein_coding | −3.760184324 | 7.23E−88 | FALSE | FALSE | FALSE | FALSE | FALSE | TRUE |
| ENSG00000119147 | C2orf40 | 2 | 106679702 | 106694615 | protein_coding | −3.762214512 | 1.02E−86 | FALSE | FALSE | FALSE | FALSE | FALSE | TRUE |
| ENSG00000161649 | CD300LG | 17 | 41924516 | 41940997 | protein_coding | −3.776289687 | 5.68E−73 | FALSE | FALSE | FALSE | FALSE | TRUE | FALSE |
| ENSG00000112175 | BMP5 | 6 | 55618443 | 55740362 | protein_coding | −3.793505321 | 5.58E−65 | FALSE | FALSE | FALSE | FALSE | FALSE | TRUE |
| ENSG00000112936 | C7 | 5 | 40909354 | 40983041 | protein_coding | −3.83120439 | 1.07E−83 | FALSE | FALSE | FALSE | FALSE | FALSE | FALSE |
| ENSG00000248144 | ADH1C | 4 | 100257649 | 100274184 | polymorphic_pse [symbol] | −3.898991102 | 3.44E−73 | FALSE | FALSE | FALSE | FALSE | FALSE | FALSE |
| ENSG00000167434 | CA4 | 17 | 58227297 | 58248260 | protein_coding | −4.023885596 | 6.44E−62 | FALSE | FALSE | FALSE | FALSE | TRUE | FALSE |
| ENSG00000130226 | DPP6 | 7 | 153584182 | 154685995 | protein_coding | −4.051300954 | 1.00E−63 | FALSE | FALSE | FALSE | FALSE | FALSE | TRUE |
| ENSG00000101938 | CHRDL1 | X | 109917084 | 110039286 | protein_coding | −4.05550851 | 2.49E−82 | FALSE | FALSE | FALSE | TRUE | FALSE | FALSE |
| ENSG00000136546 | SCN7A | 2 | 167260083 | 167350757 | protein_coding | −4.115503952 | 1.54E−81 | FALSE | FALSE | FALSE | FALSE | FALSE | TRUE |
| ENSG00000104332 | SFRP1 | 8 | 41119481 | 41167016 | protein_coding | −4.214776907 | 7.30E−90 | FALSE | FALSE | FALSE | FALSE | FALSE | FALSE |
| ENSG00000184905 | TCEAL2 | X | 101380660 | 101382683 | protein_coding | −4.224038674 | 4.08E−94 | FALSE | FALSE | FALSE | FALSE | FALSE | FALSE |
| ENSG00000168079 | SCARA5 | 8 | 27727399 | 27850244 | protein_coding | −4.309036451 | 4.43E−92 | FALSE | TRUE | TRUE | TRUE | TRUE | TRUE |
| ENSG00000164530 | PI16 | 6 | 36922209 | 36932613 | protein_coding | −4.782962491 | 1.28E−108 | FALSE | FALSE | FALSE | TRUE | FALSE | FALSE |
| ENSG00000196616 | ADH1B | 4 | 100226121 | 100242558 | protein_coding | −4.852169378 | 1.28E−120 | FALSE | FALSE | TRUE | TRUE | FALSE | TRUE |

[symbol]

indicates data missing or illegible when filed

TABLE C1
Recurcive feature elimination (RFE) analysis for tumor
status and carcinogenesis prediction. Cross-Validation
of 10 fold and repeated 10 times. The number of
genes selected is based on Accuracy and Kappa
and the standard deviation. The training set consist
of 15,507 samples (including the paired samples
used for the differential gene expression).

| Genes | Accuracy | Kappa | AccuracySD | KappaSD | Selected |
|---|---|---|---|---|---|
| 10 | 0.9835 | 0.9669 | 0.003460 | 0.006919 | |
| 20 | 0.9903 | 0.9806 | 0.002066 | 0.004132 | |
| 30 | 0.9915 | 0.983 | 0.001842 | 0.003684 | * |
| 40 | 0.9920 | 0.984 | 0.002146 | 0.004292 | |
| 50 | 0.9925 | 0.985 | 0.001965 | 0.003931 | |
| 60 | 0.9925 | 0.9851 | 0.002004 | 0.004007 | |
| 70 | 0.9928 | 0.9857 | 0.001921 | 0.003842 | |
| 80 | 0.9930 | 0.9859 | 0.001926 | 0.003852 | |
| 90 | 0.9930 | 0.9859 | 0.001868 | 0.003736 | |
| 100 | 0.9930 | 0.986 | 0.001784 | 0.003568 | ** |
| 150 | 0.9933 | 0.9865 | 0.001981 | 0.003961 | |
| 200 | 0.9933 | 0.9867 | 0.002110 | 0.004219 | |
| 250 | 0.9933 | 0.9866 | 0.001866 | 0.003731 | |
| 300 | 0.9932 | 0.9865 | 0.002006 | 0.004011 | |
| 350 | 0.9933 | 0.9866 | 0.001995 | 0.00399 | |
| 400 | 0.9933 | 0.9867 | 0.002020 | 0.004039 | |
| 450 | 0.9932 | 0.9865 | 0.001984 | 0.003968 | |
| 500 | 0.9933 | 0.9865 | 0.002061 | 0.004122 | |
| 1885 | 0.9929 | 0.9859 | 0.002106 | 0.004211 | |

** best overall set of predictors,
* best trade-off for parcimony and standard deviation estimated during internal cross-validation.

TABLE C2
Recurcive feature elimination
(RFE) analysis for tumor status and

| Variables | Accuracy | Kappa | AccuracySD | KappaSD |
|---|---|---|---|---|
| 1 | 0.782894 | 0.5662112 | 0.04420337 | 0.08904987 |
| 2 | 0.8773641 | 0.7545603 | 0.01618023 | 0.03224393 |
| 3 | 0.9226158 | 0.8449416 | 0.01379973 | 0.02763054 |
| 4 | 0.9445131 | 0.8888129 | 0.01630438 | 0.03268565 |
| 5 | 0.9580167 | 0.9158706 | 0.01533969 | 0.03075046 |
| 6 | 0.9689446 | 0.9377726 | 0.01260673 | 0.02527323 |
| 7 | 0.9761667 | 0.9522476 | 0.00747566 | 0.01498254 |
| 8 | 0.9790988 | 0.9581236 | 0.00648797 | 0.01300442 |
| 9 | 0.9818224 | 0.9635835 | 0.004971 | 0.00996342 |
| 10 | 0.9838803 | 0.9677071 | 0.00429528 | 0.00860723 |
| 20 | 0.9897513 | 0.9794687 | 0.00256263 | 0.0051358 |
| 30 | 0.9915334 | 0.9830391 | 0.0027168 | 0.00544384 |
| 40 | 0.9919705 | 0.9839148 | 0.00267566 | 0.00536167 |
| 50 | 0.9924749 | 0.9849256 | 0.00256568 | 0.0051412 |
| 60 | 0.9926901 | 0.9853568 | 0.00242074 | 0.0048507 |
| 70 | 0.9929927 | 0.9859633 | 0.00233738 | 0.00468339 |
| 80 | 0.9931743 | 0.9863271 | 0.00222578 | 0.00445978 |
| 90 | 0.9933827 | 0.9867449 | 0.00218497 | 0.00437789 |
| 100 | 0.9934231 | 0.9868256 | 0.00224258 | 0.00449354 |
| 110 | 0.9935374 | 0.9870548 | 0.00216651 | 0.00434078 |
| 120 | 0.9934365 | 0.9868528 | 0.00209637 | 0.00420023 |
| 130 | 0.9936181 | 0.9872165 | 0.00203746 | 0.00408238 |
| 140 | 0.9935845 | 0.9871492 | 0.00201473 | 0.00403674 |
| 150 | 0.9936181 | 0.9872166 | 0.00206414 | 0.00413555 |
| 200 | 0.9936248 | 0.9872302 | 0.00197132 | 0.00394962 |
| 250 | 0.9935643 | 0.9871091 | 0.00214171 | 0.00429089 |
| 300 | 0.9936786 | 0.9873379 | 0.00204526 | 0.00409792 |
| 350 | 0.9936315 | 0.9872436 | 0.00199666 | 0.0040005 |
| 400 | 0.9936584 | 0.9872976 | 0.0020636 | 0.00413443 |
| 450 | 0.9936046 | 0.9871898 | 0.00215933 | 0.00432629 |
| 500 | 0.9935105 | 0.9870012 | 0.00201463 | 0.00403655 |
| 1878 | 0.9928784 | 0.985735 | 0.00201383 | 0.00403488 |

TABLE D
Comparison of 8 different predictive models for cancer diagnosis with 30 genes. All models were
trained with the same training set of 15,507 samples, were set with the same seeds and the same
repeated leave-group out cross-validation parameters (K = 10 with 10 repeats and 70% of training
set used as training). Models were tested on an external data set of 5,484 samples.

| Models | Accuracy | Precision | Recall | F1-Score | ROC | Selected |
|---|---|---|---|---|---|---|
| bagCART | 0.990 | 0.991 | 0.991 | 0.991 | 0.989 | |
| CART | 0.907 | 0.905 | 0.936 | 0.920 | 0.902 | |
| Deepboost | 0.991 | 0.993 | 0.990 | 0.992 | 0.991 | |
| GBM | 0.993 | 0.993 | 0.994 | 0.994 | 0.993 | |
| KNN | 0.961 | 0.979 | 0.953 | 0.966 | 0.963 | |
| LDA | 0.960 | 0.979 | 0.951 | 0.965 | 0.962 | |
| RF | 0.992 | 0.993 | 0.994 | 0.993 | 0.992 | * |
| SVM | 0.922 | 0.992 | 0.870 | 0.927 | 0.931 | |

Accuracy: measure of the overall precision of the model.
ROC: area under the Receiver Operating Characteristic curve.
Precision: the ratio of correctly predicted positive observations to the total predicted positive observations.
Recall: the ratio of correctly predicted positive observations to the all observations in actual class.
F1 Score: for binary classification measures the accuracy of the test and represents the harmonic average of the precision and recall.
bagCART: Bootstrap Aggregation of a CART algorithm.
CART: Classification and Regression Tree.
GBM: Gradient Boosting Machines.
KNN: K-Nearest Neighbour.
LDA: Linear Discriminant Analysis.
RF: Random Forest.
SVM: Support Vector Machine.
CART: The final value used for the model was cp = 0.05093446.
Deepboost: The final values used for the model were num_iter = 100, tree_depth = 3, beta = 0.00390625, lambda = 0.015625 and loss_type = I.
GBM: The final values used for the model were n.trees = 150, interaction.depth = 3, shrinkage = 0.1 and n.minobsinnode = 10.
KNN: The final value used for the model was k = 5.
RF: The final value used for the model was mtry = 16.
SVM: The final values used for the model were sigma = 0.07547102 and C = 1.

TABLE E
30 biomarkers and their importance in the RF-RKFCV.

| # | Ensembl gene id | Symbol | Chr | Start | End | Gene Biotype | Varlmp |
|---|---|---|---|---|---|---|---|
| 1 | ENSG00000154258 | ABCA9 | 17 | 66970629 | 67057205 | protein_coding | 0.922786743 |
| 2 | ENSG00000272789 | | 2 | 128383572 | 128384423 | antisense | 0.921527652 |
| 3 | ENSG00000154263 | ABCA10 | 17 | 67143355 | 67240987 | protein_coding | 0.919024182 |
| 4 | ENSG00000149451 | ADAM33 | 20 | 3648612 | 3662893 | protein_coding | 0.908624654 |
| 5 | ENSG00000254726 | MEX3A | 1 | 156041804 | 156051789 | protein_coding | 0.905491946 |
| 6 | ENSG00000144230 | GPR17 | 2 | 128403439 | 128410213 | protein_coding | 0.903839862 |
| 7 | ENSG00000124766 | SOX4 | 6 | 21593972 | 21598847 | protein_coding | 0.901898707 |
| 8 | ENSG00000092529 | CAPN3 | 15 | 42640301 | 42704516 | protein_coding | 0.8970102 |
| 9 | ENSG00000213070 | HMGB3P6 | 1 | 164326004 | 164326601 | pseudogene | 0.894045042 |
| 10 | ENSG00000258498 | DIO3OS | 14 | 102018558 | 102026768 | lincRNA | 0.881428011 |
| 11 | ENSG00000168079 | SCARA5 | 8 | 27727399 | 27850244 | protein_coding | 0.875628559 |
| 12 | ENSG00000196616 | ADH1B | 4 | 100226121 | 100242558 | protein_coding | 0.874248356 |
| 13 | ENSG00000165197 | FIGF | X | 15363713 | 15402498 | protein_coding | 0.872691225 |
| 14 | ENSG00000160808 | MYL3 | 3 | 46899362 | 46923659 | protein_coding | 0.871392146 |
| 15 | ENSG00000167676 | PLIN4 | 19 | 4502204 | 4517716 | protein_coding | 0.867277212 |
| 16 | ENSG00000099953 | MMP11 | 22 | 24110413 | 24126503 | protein_coding | 0.861015807 |
| 17 | ENSG00000164283 | ESM1 | 5 | 54273692 | 54318499 | protein_coding | 0.854274959 |
| 18 | ENSG00000175084 | DES | 2 | 220283099 | 220291461 | protein_coding | 0.840015278 |
| 19 | ENSG00000267336 | | 18 | 12912843 | 12913905 | \|pseudogene | 0.836779313 |
| 20 | ENSG00000255353 | | 11 | 107047012 | 107048256 | pseudogene | 0.836287231 |
| 21 | ENSG00000205038 | PKHD1L1 | 8 | 110374706 | 110542559 | protein_coding | 0.817821799 |
| 22 | ENSG00000111405 | ENDOU | 12 | 48103517 | 48119350 | protein_coding | 0.806619343 |
| 23 | ENSG00000143196 | DPT | 1 | 168664697 | 168698502 | protein_coding | 0.781947537 |
| 24 | ENSG00000123500 | COL10A1 | 6 | 116440086 | 116479910 | protein_coding | 0.779552275 |
| 25 | ENSG00000114854 | TNNC1 | 3 | 52485118 | 52488086 | protein_coding | 0.758064648 |
| 26 | ENSG00000120913 | PDLIM2 | 8 | 22435792 | 22455538 | protein_coding | 0.756858659 |
| 27 | ENSG00000188257 | PLA2G2A | 1 | 20301925 | 20306932 | protein_coding | 0.736299782 |
| 28 | ENSG00000136826 | KLF4 | 9 | 110247133 | 110252763 | protein_coding | 0.716191476 |
| 29 | ENSG00000187730 | GABRD | 1 | 1950780 | 1962192 | protein_coding | 0.666355092 |
| 30 | ENSG00000180871 | CXCR2 | 2 | 218990012 | 219001976 | protein_coding | 0.596828723 |

TABLE F
100 biomarkers and their importance in the RF-RKFCV.

| # | Ensembl gene id | Symbol | Chr | Start | End | Gene Biotype | Varlmp |
|---|---|---|---|---|---|---|---|
| 1 | ENSG00000237649 | KIFC1 | 6 | 33359313 | 33377701 | protein_coding | 0.92253848 |
| 2 | ENSG00000154258 | ABCA9 | 17 | 66970629 | 67057205 | protein_coding | 0.92235668 |
| 3 | ENSG00000189057 | FAM111B | 11 | 58874658 | 58894883 | protein_coding | 0.9222245 |
| 4 | ENSG00000104889 | RNASEH2A | 19 | 12917394 | 12924452 | protein_coding | 0.92131625 |
| 5 | ENSG00000272789 | | 2 | 128383572 | 128384423 | antisense | 0.92130984 |
| 6 | ENSG00000154263 | ABCA10 | 17 | 67143355 | 67240987 | protein_coding | 0.91895394 |
| 7 | ENSG00000149451 | ADAM33 | 20 | 3648612 | 3662893 | protein_coding | 0.90864061 |
| 8 | ENSG00000254726 | MEX3A | 1 | 156041804 | 156051789 | protein_coding | 0.90548257 |
| 9 | ENSG00000144230 | GPR17 | 2 | 128403439 | 128410213 | protein_coding | 0.90365705 |
| 10 | ENSG00000124766 | SOX4 | 6 | 21593972 | 21598847 | protein_coding | 0.9020606 |
| 11 | ENSG00000124440 | HIF3A | 19 | 46800303 | 46846690 | protein_coding | 0.90034714 |
| 12 | ENSG00000068976 | PYGM | 11 | 64513861 | 64527769 | protein_coding | 0.8982835 |
| 13 | ENSG00000092529 | CAPN3 | 15 | 42640301 | 42704516 | protein_coding | 0.89707637 |
| 14 | ENSG00000213070 | HMGB3P6 | 1 | 164326004 | 164326601 | pseudogene | 0.89419323 |
| 15 | ENSG00000079462 | PAFAH1B3 | 19 | 42801185 | 42807698 | protein_coding | 0.89308495 |
| 16 | ENSG00000141338 | ABCA8 | 17 | 66863433 | 66951533 | protein_coding | 0.89252208 |
| 17 | ENSG00000228168 | HNRNPA1P21 | 3 | 39376470 | 39377430 | pseudogene | 0.89149291 |
| 18 | ENSG00000068831 | RASGRP2 | 11 | 64494383 | 64512928 | protein_coding | 0.88486805 |
| 19 | ENSG00000258498 | DIO3OS | 14 | 102018558 | 102026768 | lincRNA | 0.88135848 |
| 20 | ENSG00000168477 | TNXB | 6 | 32008931 | 32083111 | protein_coding | 0.87951291 |
| 21 | ENSG00000214548 | MEG3 | 14 | 101245747 | 101327368 | lincRNA | 0.87737133 |
| 22 | ENSG00000173641 | HSPB7 | 1 | 16340523 | 16346089 | protein_coding | 0.8767794 |
| 23 | ENSG00000168079 | SCARA5 | 8 | 27727399 | 27850244 | protein_coding | 0.87499063 |
| 24 | ENSG00000196616 | ADH1B | 4 | 100226121 | 100242558 | protein_coding | 0.87401244 |
| 25 | ENSG00000133800 | LYVE1 | 11 | 10578513 | 10633236 | protein_coding | 0.87394333 |
| 26 | ENSG00000165197 | FIGF | X | 15363713 | 15402498 | protein_coding | 0.87265633 |
| 27 | ENSG00000160808 | MYL3 | 3 | 46899362 | 46923659 | protein_coding | 0.87134776 |
| 28 | ENSG00000128482 | RNF112 | 17 | 19314438 | 19320589 | protein_coding | 0.87007032 |
| 29 | ENSG00000167676 | PLIN4 | 19 | 4502204 | 4517716 | protein_coding | 0.86724607 |
| 30 | ENSG00000249669 | MIR143HG | 5 | 148786252 | 148808241 | lincRNA | 0.86508147 |
| 31 | ENSG00000110492 | MDK | 11 | 46402306 | 46405375 | protein_coding | 0.86157602 |
| 32 | ENSG00000099953 | MMP11 | 22 | 24110413 | 24126503 | protein_coding | 0.86099042 |
| 33 | ENSG00000164530 | PI16 | 6 | 36922209 | 36932613 | protein_coding | 0.86085793 |
| 34 | ENSG00000164283 | ESM1 | 5 | 54273692 | 54318499 | protein_coding | 0.85492718 |
| 35 | ENSG00000123560 | PLP1 | X | 103028647 | 103047548 | protein_coding | 0.85461171 |
| 36 | ENSG00000042062 | FAM65C | 20 | 49202645 | 49308065 | protein_coding | 0.85118018 |
| 37 | ENSG00000077522 | ACTN2 | 1 | 236849754 | 236927931 | protein_coding | 0.850411 |
| 38 | ENSG00000165795 | NDRG2 | 14 | 21484922 | 21539031 | protein_coding | 0.84788564 |
| 39 | ENSG00000175084 | DES | 2 | 220283099 | 220291461 | protein_coding | 0.83988689 |
| 40 | ENSG00000267336 | | 18 | 12912843 | 12913905 | pseudogene | 0.83749479 |
| 41 | ENSG00000213886 | UBD | 6 | 29523292 | 29527702 | protein_coding | 0.8368889 |
| 42 | ENSG00000255353 | | 11 | 107047012 | 107048256 | pseudogene | 0.8364889 |
| 43 | ENSG00000244306 | | 14 | 19854098 | 19925348 | lincRNA | 0.82993567 |
| 44 | ENSG00000214456 | PLIN5 | 19 | 4522543 | 4535236 | protein_coding | 0.82883818 |
| 45 | ENSG00000095970 | TREM2 | 6 | 41126244 | 41130924 | protein_coding | 0.82051627 |
| 46 | ENSG00000198624 | CCDC69 | 5 | 150560613 | 150603706 | protein_coding | 0.81962949 |
| 47 | ENSG00000205038 | PKHD1L1 | 8 | 110374706 | 110542559 | protein_coding | 0.81784243 |
| 48 | ENSG00000147889 | CDKN2A | 9 | 21967751 | 21995300 | protein_coding | 0.81739459 |
| 49 | ENSG00000101938 | CHRDL1 | X | 109917084 | 110039286 | protein_coding | 0.81682533 |
| 50 | ENSG00000260920 | | 1 | 40929991 | 40932436 | sense_overlapping | 0.81498748 |
| 51 | ENSG00000197766 | CFD | 19 | 859453 | 863453 | protein_coding | 0.81005367 |
| 52 | ENSG00000165072 | MAMDC2 | 9 | 72658497 | 72841886 | protein_coding | 0.80743179 |
| 53 | ENSG00000111405 | ENDOU | 12 | 48103517 | 48119350 | protein_coding | 0.80639571 |
| 54 | ENSG00000225210 | | 14 | 19650018 | 19718563 | lincRNA | 0.79511751 |
| 55 | ENSG00000175161 | CADM2 | 3 | 85008132 | 86123579 | protein_coding | 0.79339118 |
| 56 | ENSG00000198483 | ANKRD35 | 1 | 145549230 | 145568526 | protein_coding | 0.78609445 |
| 57 | ENSG00000206195 | | 22 | 16147979 | 16193004 | processed_transcr | 0.78184565 |
| 58 | ENSG00000143196 | DPT | 1 | 168664697 | 168698502 | protein_coding | 0.78119654 |
| 59 | ENSG00000188536 | HBA2 | 16 | 222846 | 223709 | protein_coding | 0.77989421 |
| 60 | ENSG00000123500 | COL10A1 | 6 | 116440086 | 116479910 | protein_coding | 0.77976652 |
| 61 | ENSG00000244734 | HBB | 11 | 5246694 | 5250625 | protein_coding | 0.77932183 |
| 62 | ENSG00000094963 | FMO2 | 1 | 171154347 | 171181822 | protein_coding | 0.77867095 |
| 63 | ENSG00000186594 | MIR22HG | 17 | 1614805 | 1620468 | lincRNA | 0.77644635 |
| 64 | ENSG00000228314 | CYP4F29P | 21 | 15215454 | 15220685 | pseudogene | 0.77594469 |
| 65 | ENSG00000162849 | KIF26B | 1 | 245318287 | 245872733 | protein_coding | 0.77060638 |
| 66 | ENSG00000108405 | P2RX1 | 17 | 3799886 | 3819794 | protein_coding | 0.76901874 |
| 67 | ENSG00000174938 | SEZ6L2 | 16 | 29882480 | 29910868 | protein_coding | 0.76764459 |
| 68 | ENSG00000206172 | HBA1 | 16 | 226679 | 227521 | protein_coding | 0.76681579 |
| 69 | ENSG00000114854 | TNNC1 | 3 | 52485118 | 52488086 | protein_coding | 0.75713287 |
| 70 | ENSG00000120913 | PDLIM2 | 8 | 22435792 | 22455538 | protein_coding | 0.75611494 |
| 71 | ENSG00000136011 | STAB2 | 12 | 103981051 | 104160505 | protein_coding | 0.75305358 |
| 72 | ENSG00000137878 | GCOM1 | 15 | 57884106 | 58006943 | protein_coding | 0.75029862 |
| 73 | ENSG00000169684 | CHRNA5 | 15 | 78857862 | 78887611 | protein_coding | 0.7456192 |
| 74 | ENSG00000166856 | GPR182 | 12 | 57388230 | 57390468 | protein_coding | 0.73865679 |
| 75 | ENSG00000145708 | CRHBP | 5 | 76248538 | 76276983 | protein_coding | 0.73770998 |
| 76 | ENSG00000144655 | CSRNP1 | 3 | 39183346 | 39196053 | protein_coding | 0.73622609 |
| 77 | ENSG00000188257 | PLA2G2A | 1 | 20301925 | 20306932 | protein_coding | 0.73609136 |
| 78 | ENSG00000136826 | KLF4 | 9 | 110247133 | 110252763 | protein_coding | 0.71578597 |
| 79 | ENSG00000180509 | KCNE1 | 21 | 35818988 | 35884573 | protein_coding | 0.71190138 |
| 80 | ENSG00000272405 | | 1 | 156611458 | 156614679 | antisense | 0.70505704 |
| 81 | ENSG00000234380 | | 21 | 36118054 | 36157183 | antisense | 0.70401033 |
| 82 | ENSG00000126759 | CFP | X | 47483612 | 47489704 | protein_coding | 0.70379379 |
| 83 | ENSG00000266010 | GATA6-AS1 | 18 | 19746859 | 19748929 | lincRNA | 0.68801768 |
| 84 | ENSG00000106278 | PTPRZ1 | 7 | 121513143 | 121702090 | protein_coding | 0.68590978 |
| 85 | ENSG00000172005 | MAL | 2 | 95691422 | 95719737 | protein_coding | 0.67495788 |
| 86 | ENSG00000118526 | TCF21 | 6 | 134210276 | 134216691 | protein_coding | 0.66652729 |
| 87 | ENSG00000169760 | NLGN1 | 3 | 173114074 | 174004434 | protein_coding | 0.66642064 |
| 88 | ENSG00000187730 | GABRD | 1 | 1950780 | 1962192 | protein_coding | 0.66605713 |
| 89 | ENSG00000153246 | PLA2R1 | 2 | 160788519 | 160919121 | protein_coding | 0.66452896 |
| 90 | ENSG00000089199 | CHGB | 20 | 5892076 | 5906007 | protein_coding | 0.62922778 |
| 91 | ENSG00000229953 | | 1 | 156616299 | 156631216 | antisense | 0.61794996 |
| 92 | ENSG00000086991 | NOX4 | 11 | 89057524 | 89322779 | protein_coding | 0.60678194 |
| 93 | ENSG00000163687 | DNASE1L3 | 3 | 58177984 | 58200424 | protein_coding | 0.60166601 |
| 94 | ENSG00000187583 | PLEKHN1 | 1 | 901877 | 911245 | protein_coding | 0.60101097 |
| 95 | ENSG00000180871 | CXCR2 | 2 | 218990012 | 219001976 | protein_coding | 0.59616565 |
| 96 | ENSG00000168447 | SCNN1B | 16 | 23289552 | 23392620 | protein_coding | 0.59612716 |
| 97 | ENSG00000172478 | C2orf54 | 2 | 241825465 | 241836306 | protein_coding | 0.59226002 |
| 98 | ENSG00000180785 | OR51E1 | 11 | 4664650 | 4676718 | protein_coding | 0.59173014 |
| 99 | ENSG00000140678 | ITGAX | 16 | 31366455 | 31394318 | protein_coding | 0.56243688 |
| 100 | ENSG00000138696 | BMPR1B | 4 | 95679119 | 96079599 | protein_coding | 0.5244039 |

TABLE G
Random Forest RKFCV model with 30 genes performance on external validation data sets. The model ran wi [symbol]
the following parameters: Kfold = 10 with 10 repeats with split of 75% of training set at each fold. The final para

[symbol]

| | | Total | True positive | False | False | True negative |
|---|---|---|---|---|---|---|
| Tissue | Accession | Samples | (Tumour) | negative | positive | (Normal) |
| 50 tissue types | E-MTAB-2706 | 899 | 669 | 2 | 30 | 198 |
| | E-MTAB-2836 | | | | | |
| | E-MTAB-3708 | | | | | |
| | E-MTAB-3716 | | | | | |
| | E-MTAB-513 | | | | | |
| Breast | SRP019936 | 300 | 208 | 8 | 7 | 77 |
| | SRP023262 | | | | | |
| | SRP027589 | | | | | |
| | SRP030401 | | | | | |
| | SRP032789 | | | | | |
| | SRP042620 | | | | | |
| Liver | SRP001558 | 58 | 26 | 3 | 3 | 26 |
| | SRP002272 | | | | | |
| | SRP007359 | | | | | |
| | SRP007412 | | | | | |
| | SRP007461 | | | | | |
| | SRP009123 | | | | | |
| | SRP010670 | | | | | |
| | SRP039694 | | | | | |
| Lung | SRP006575 | 184 | 132 | 10 | 8 | 34 |
| | SRP007461 | | | | | |
| | SRP009266 | | | | | |
| | SRP009408 | | | | | |
| | SRP010166 | | | | | |
| | SRP012656 | | | | | |
| | SRP013935 | | | | | |
| | SRP013981 | | | | | |
| | SRP017019 | | | | | |
| | SRP019807 | | | | | |
| | SRP026620 | | | | | |
| | SRP031698 | | | | | |
| | SRP033095 | | | | | |
| Ovarian | E-MTAB-2836 | 105 | 93 | 0 | 1 | 11 |
| | E-MTAB-513 | | | | | |
| | E-MTAB-2706 | | | | | |
| | OVBM | | | | | |
| Total | | 1546 | 1128 | 23 | 49 | 346 |

RNAseq data were gathered from unstandardized sampling and preservation procedures (FFPE or flash frozen, etc.), libra [symbol] [symbol]
indicates data missing or illegible when filed

TABLE G
Random [symbol] th the following parameter on a training set
n = 15,507 biopsies with the follo [symbol] meters
used for the model were: mtry = 10 and 501 trees.

| | F1 | | | |
|---|---|---|---|---|
| Tissue | Score | Precision | Recall | Comments |
| 50 tissue | 0.98 | 0.96 | 1.00 | Include cancer cell lines; low |
| types | | | | grade gliomas; normal tissue |
| | | | | from 122 individuals |
| Breast | 0.97 | 0.97 | 0.97 | |
| Liver | 0.90 | 0.90 | 0.90 | Include hemangioma and |
| | | | | HCV/HBV samples |
| Lung | 0.94 | 0.94 | 0.93 | Include benign, Idiopathic |
| | | | | pulmonary fibrosis, and FFPE |
| | | | | preserved samples |
| Ovarian | 1.00 | 0.99 | 1.00 | Include cell lines; fallopian and |
| | | | | ovarian tissues; seven ovarian |
| | | | | cancer types samples |
| Total | 0.97 | 0.96 | 0.98 | |

RNAseq data were [symbol] ry preparation, sequencing technology and annotation, potentially affe [symbol] [symbol]
indicates data missing or illegible when filed

TABLE H
450 Biomarkers used for the cancar types modelling and their importance in the RF-RKFCV modelling.

| # | Ensembl gene id | Symbol | Chr | Start | End | Gene Biotype | Varlmp |
|---|---|---|---|---|---|---|---|
| 1 | ENSG00000188488 | SERPINA5 | 14 | 95027779 | 95059457 | protein_coding | 0.996846089 |
| 2 | ENSG00000160862 | AZGP1 | 7 | 99564343 | 99573780 | protein_coding | 0.996236289 |
| 3 | ENSG00000188959 | C9orf152 | 9 | 112952328 | 112970469 | protein_coding | 0.991486845 |
| 4 | ENSG00000134258 | VTCN1 | 1 | 117686209 | 117753556 | protein_coding | 0.991280737 |
| 5 | ENSG00000148795 | CYP17A1 | 10 | 104590288 | 104597290 | protein_coding | 0.989592094 |
| 6 | ENSG00000144057 | ST6GAL2 | 2 | 107418056 | 107503564 | protein_coding | 0.985841279 |
| 7 | ENSG00000148488 | ST8SIA6 | 10 | 17360382 | 17496329 | protein_coding | 0.984981002 |
| 8 | ENSG00000114790 | ARHGEF26 | 3 | 153838792 | 153975616 | protein_coding | 0.984622554 |
| 9 | ENSG00000114812 | VIPR1 | 3 | 42530791 | 42579059 | protein_coding | 0.984532941 |
| 10 | ENSG00000203499 | FAM83H-AS1 | 8 | 144816310 | 144828507 | lincRNA | 0.984102803 |
| 11 | ENSG00000115468 | EFHD1 | 2 | 233470767 | 233547491 | protein_coding | 0.984013191 |
| 12 | ENSG00000124766 | SOX4 | 6 | 21593972 | 21598847 | protein_coding | 0.982292637 |
| 13 | ENSG00000123500 | COL10A1 | 6 | 116440086 | 116479910 | protein_coding | 0.981136641 |
| 14 | ENSG00000165125 | TRPV6 | 7 | 142568956 | 142583507 | protein_coding | 0.981038067 |
| 15 | ENSG00000243069 | ARHGEF26-AS1 | 3 | 153742190 | 153839121 | processed_transcrip | 0.980410782 |
| 16 | ENSG00000168032 | ENTPD3 | 3 | 40428647 | 40470110 | protein_coding | 0.979920101 |
| 17 | ENSG00000184012 | TMPRSS2 | 21 | 42836478 | 42903043 | protein_coding | 0.979209979 |
| 18 | ENSG00000107485 | GATA3 | 10 | 8095567 | 8117161 | protein_coding | 0.977166822 |
| 19 | ENSG00000142494 | SLC47A1 | 17 | 19398698 | 19482347 | protein_coding | 0.976608495 |
| 20 | ENSG00000143416 | SELENBP1 | 1 | 151336778 | 151345209 | protein_coding | 0.975804717 |
| 21 | ENSG00000196338 | NLGN3 | X | 70364681 | 70391051 | protein_coding | 0.973331422 |
| 22 | ENSG00000165272 | AQP3 | 9 | 33441152 | 33447609 | protein_coding | 0.972823802 |
| 23 | ENSG00000135373 | EHF | 11 | 34642640 | 34682604 | protein_coding | 0.97280271 |
| 24 | ENSG00000151715 | TMEM45B | 11 | 129685714 | 129729898 | protein_coding | 0.972666106 |
| 25 | ENSG00000266010 | GATA6-AS1 | 18 | 19746859 | 19748929 | lincRNA | 0.970356298 |
| 26 | ENSG00000145284 | SCD5 | 4 | 83550692 | 83720010 | protein_coding | 0.970300673 |
| 27 | ENSG00000140254 | DUOXA1 | 15 | 45409569 | 45422136 | protein_coding | 0.969985282 |
| 28 | ENSG00000109072 | VTN | 17 | 26691290 | 26700110 | protein_coding | 0.969101728 |
| 29 | ENSG00000109072 | SEBOX | 17 | 26691290 | 26700110 | protein_coding | 0.969101728 |
| 30 | ENSG00000153292 | GPR110 | 6 | 46965440 | 47010099 | protein_coding | 0.969039108 |
| 31 | ENSG00000157765 | SLC34A2 | 4 | 25656923 | 25680370 | protein_coding | 0.965804 |
| 32 | ENSG00000268388 | FENDRR | 16 | 86508135 | 86542705 | lincRNA | 0.964833894 |
| 33 | ENSG00000130413 | STK33 | 11 | 8413418 | 8615836 | protein_coding | 0.964334361 |
| 34 | ENSG00000136872 | ALDOB | 9 | 104182860 | 104198105 | protein_coding | 0.962864721 |
| 35 | ENSG00000144668 | ITGA9 | 3 | 37493606 | 37865005 | protein_coding | 0.96062868 |
| 36 | ENSG00000105523 | FAM83E | 19 | 49104067 | 49118111 | protein_coding | 0.958294501 |
| 37 | ENSG00000197580 | BCO2 | 11 | 112046190 | 112095422 | protein_coding | 0.955229766 |
| 38 | ENSG00000138615 | CILP | 15 | 65488337 | 65503826 | protein_coding | 0.954943007 |
| 39 | ENSG00000157551 | KCNJ15 | 21 | 39529128 | 39679279 | protein_coding | 0.953111859 |
| 40 | ENSG00000129151 | BBOX1 | 11 | 27062272 | 27149356 | protein_coding | 0.952792315 |
| 41 | ENSG00000197565 | COL4A6 | X | 107386780 | 107682727 | protein_coding | 0.952743902 |
| 42 | ENSG00000168679 | SLC16A4 | 1 | 110905470 | 110933704 | protein_coding | 0.952577246 |
| 43 | ENSG00000029153 | ARNTL2 | 12 | 27485787 | 27576241 | protein_coding | 0.951850294 |
| 44 | ENSG00000117707 | PROX1 | 1 | 214156524 | 214214595 | protein_coding | 0.949905383 |
| 45 | ENSG00000164694 | FNDC1 | 6 | 159590429 | 159693141 | protein_coding | 0.94892107 |
| 46 | ENSG00000198624 | CCDC69 | 5 | 150560613 | 150603706 | protein_coding | 0.948419242 |
| 47 | ENSG00000162840 | MT2P1 | 4 | 69242041 | 69242226 | pseudogene | 0.947899491 |
| 48 | ENSG00000180769 | WDFY3-AS2 | 4 | 85887538 | 85932430 | processed_transcrip | 0.94775021 |
| 49 | ENSG00000178568 | ERBB4 | 2 | 212240446 | 213403565 | protein_coding | 0.94759481 |
| 50 | ENSG00000169218 | RSPO1 | 1 | 38076951 | 38100595 | protein_coding | 0.947343896 |
| 51 | ENSG00000144452 | ABCA12 | 2 | 215796266 | 216003151 | protein_coding | 0.946949602 |
| 52 | ENSG00000160868 | CYP3A4 | 7 | 99354604 | 99381888 | protein_coding | 0.945390351 |
| 53 | ENSG00000253953 | PCDHGB4 | 5 | 140767452 | 140892546 | protein_coding | 0.944386039 |
| 54 | ENSG00000149294 | NCAM1 | 11 | 112831997 | 113149158 | protein_coding | 0.94302459 |
| 55 | ENSG00000186529 | CYP4F3 | 19 | 15751707 | 15773635 | protein_coding | 0.942782801 |
| 56 | ENSG00000254122 | PCDHGB7 | 5 | 140797427 | 140892546 | protein_coding | 0.942703953 |
| 57 | ENSG00000254245 | PCDHGA3 | 5 | 140723601 | 140892546 | protein_coding | 0.940653911 |
| 58 | ENSG00000107242 | PIP5K1B | 9 | 71320575 | 71624092 | protein_coding | 0.940285955 |
| 59 | ENSG00000081853 | PCDHGA2 | 5 | 140718539 | 140892546 | protein_coding | 0.940180824 |
| 60 | ENSG00000091831 | ESR1 | 6 | 151977826 | 152450754 | protein_coding | 0.938992042 |
| 61 | ENSG00000232699 | BDH2P1 | 6 | 99622620 | 99623357 | pseudogene | 0.937809162 |
| 62 | ENSG00000236155 | | 1 | 26536232 | 26556331 | pseudogene | 0.93757617 |
| 63 | ENSG00000179023 | KLHDC7A | 1 | 18807424 | 18812478 | protein_coding | 0.936420173 |
| 64 | ENSG00000095637 | SORBS1 | 10 | 97071528 | 97321171 | protein_coding | 0.93565847 |
| 65 | ENSG00000248290 | TNXA | 6 | 31976391 | 31980249 | pseudogene | 0.935029437 |
| 66 | ENSG00000205795 | CYS1 | 2 | 10196907 | 10221071 | protein_coding | 0.934869883 |
| 67 | ENSG00000182795 | C1orf116 | 1 | 207191866 | 207206101 | protein_coding | 0.934819176 |
| 68 | ENSG00000254726 | MEX3A | 1 | 156041804 | 156051789 | protein_coding | 0.934744426 |
| 69 | ENSG00000142973 | CYP4B1 | 1 | 47223510 | 47285085 | protein_coding | 0.932033221 |
| 70 | ENSG00000213886 | UBD | 6 | 29523292 | 29527702 | protein_coding | 0.931993333 |
| 71 | ENSG00000134962 | KLB | 4 | 39408473 | 39453156 | protein_coding | 0.931554233 |
| 72 | ENSG00000179094 | PER1 | 17 | 8043790 | 8059824 | protein_coding | 0.930891103 |
| 73 | ENSG00000046653 | GPM6B | X | 13789150 | 13956757 | protein_coding | 0.930693957 |
| 74 | ENSG00000176595 | KBTBD11 | 8 | 1922044 | 1955102 | protein_coding | 0.928984441 |
| 75 | ENSG00000168477 | TNXB | 6 | 32008931 | 32083111 | protein_coding | 0.928511354 |
| 76 | ENSG00000118526 | TCF21 | 6 | 134210276 | 134216691 | protein_coding | 0.927763639 |
| 77 | ENSG00000175785 | PRIMA1 | 14 | 94184644 | 94254827 | protein_coding | 0.927378307 |
| 78 | ENSG00000227036 | LINC00511 | 17 | 70319264 | 70636611 | lincRNA | 0.926356182 |
| 79 | ENSG00000009765 | IYD | 6 | 150690028 | 150727105 | protein_coding | 0.926338806 |
| 80 | ENSG00000163075 | | 2 | 120302008 | 120419827 | protein_coding | 0.926303616 |
| 81 | ENSG00000184454 | NCMAP | 1 | 24882602 | 24935819 | protein_coding | 0.926034124 |
| 82 | ENSG00000126562 | WNK4 | 17 | 40932696 | 40948954 | protein_coding | 0.924779554 |
| 83 | ENSG00000019102 | VSIG2 | 11 | 124617368 | 124622134 | protein_coding | 0.924253574 |
| 84 | ENSG00000258818 | RNASE4 | 14 | 21152259 | 21168761 | protein_coding | 0.924134347 |
| 85 | ENSG00000175084 | DES | 2 | 220283099 | 220291461 | protein_coding | 0.91873423 |
| 86 | ENSG00000166405 | RIC3 | 11 | 8127597 | 8190602 | protein_coding | 0.9186291 |
| 87 | ENSG00000148053 | NTRK2 | 9 | 87283466 | 87638505 | protein_coding | 0.917072908 |
| 88 | ENSG00000151892 | GFRA1 | 10 | 117816444 | 118032979 | protein_coding | 0.915648075 |
| 89 | ENSG00000183010 | PYCR1 | 17 | 79890260 | 79900288 | protein_coding | 0.91339881 |
| 90 | ENSG00000129654 | FOXJ1 | 17 | 74132414 | 74137380 | protein_coding | 0.913267452 |
| 91 | ENSG00000167741 | GGT6 | 17 | 4460222 | 4464113 | protein_coding | 0.912111018 |
| 92 | ENSG00000215270 | | 22 | 16122720 | 16123768 | pseudogene | 0.909545487 |
| 93 | ENSG00000129244 | ATP1B2 | 17 | 7549945 | 7561086 | protein_coding | 0.908957107 |
| 94 | ENSG00000166819 | PLIN1 | 15 | 90207596 | 90222658 | protein_coding | 0.908631443 |
| 95 | ENSG00000103241 | FOXF1 | 16 | 86544133 | 86548076 | protein_coding | 0.906486543 |
| 96 | ENSG00000010379 | SLC6A13 | 12 | 329789 | 372039 | protein_coding | 0.906480751 |
| 97 | ENSG00000133110 | POSTN | 13 | 38136720 | 38172981 | protein_coding | 0.90544125 |
| 98 | ENSG00000162733 | DDR2 | 1 | 162601163 | 162757190 | protein_coding | 0.904857023 |
| 99 | ENSG00000162896 | PIGR | 1 | 207101863 | 207119811 | protein_coding | 0.904437594 |
| 100 | ENSG00000091262 | ABCC6 | 16 | 16242785 | 16317379 | protein_coding | 0.904278806 |
| 101 | ENSG00000168447 | SCNN1B | 16 | 23289552 | 23392620 | protein_coding | 0.903805719 |
| 102 | ENSG00000168481 | LG13 | 8 | 22004338 | 22014597 | protein_coding | 0.902681196 |
| 103 | ENSG00000114378 | HYAL1 | 3 | 50337320 | 50349812 | protein_coding | 0.898666571 |
| 104 | ENSG00000167676 | PLIN4 | 19 | 4502204 | 4517716 | protein_coding | 0.898076114 |
| 105 | ENSG00000172425 | TTC36 | 11 | 118398187 | 118401912 | protein_coding | 0.897860062 |
| 106 | ENSG00000265415 | | 17 | 57280038 | 57281190 | antisense | 0.897760723 |
| 107 | ENSG00000164283 | ESM1 | 5 | 54273692 | 54318499 | protein_coding | 0.897340311 |
| 108 | ENSG00000140279 | DUOX2 | 15 | 45384848 | 45406542 | protein_coding | 0.896341463 |
| 109 | ENSG00000169562 | GJB1 | X | 70435044 | 70445366 | protein_coding | 0.89563768 |
| 110 | ENSG00000150551 | LYPD1 | 2 | 133402426 | 133429152 | protein_coding | 0.894974769 |
| 111 | ENSG00000068489 | PRR11 | 17 | 57232860 | 57282066 | protein_coding | 0.893345248 |
| 112 | ENSG00000141338 | ABCA8 | 17 | 66863433 | 66951533 | protein_coding | 0.892241379 |
| 113 | ENSG00000188338 | SLC38A3 | 3 | 50242679 | 50258411 | processed_transcrip | 0.892053194 |
| 114 | ENSG00000164344 | KLKB1 | 4 | 187130133 | 187179625 | protein_coding | 0.891925988 |
| 115 | ENSG00000117394 | SLC2A1 | 1 | 43391052 | 43424530 | protein_coding | 0.891820858 |
| 116 | ENSG00000198643 | FAM3D | 3 | 58619673 | 58652575 | protein_coding | 0.890506728 |
| 117 | ENSG00000064655 | EYA2 | 20 | 45523263 | 45817492 | protein_coding | 0.888253638 |
| 118 | ENSG00000131771 | PPP1R1B | 17 | 37782993 | 37792879 | protein_coding | 0.888253638 |
| 119 | ENSG00000113739 | STC2 | 5 | 172741716 | 172756506 | protein_coding | 0.88775181 |
| 120 | ENSG00000170927 | PKHD1 | 6 | 51480098 | 51952423 | protein_coding | 0.887510513 |
| 121 | ENSG00000064205 | WISP2 | 20 | 43343485 | 43357150 | protein_coding | 0.886722035 |
| 122 | ENSG00000225342 | | 12 | 40579811 | 40617605 | antisense | 0.885907275 |
| 123 | ENSG00000134240 | HMGCS2 | 1 | 120290619 | 120311528 | protein_coding | 0.885670732 |
| 124 | ENSG00000182481 | KPNA2 | 17 | 66031635 | 66042958 | protein_coding | 0.88503995 |
| 125 | ENSG00000144354 | CDCA7 | 2 | 174219548 | 174233725 | protein_coding | 0.88362069 |
| 126 | ENSG00000167900 | TK1 | 17 | 76170160 | 76183314 | protein_coding | 0.883252733 |
| 127 | ENSG00000112175 | BMP5 | 6 | 55618443 | 55740362 | protein_coding | 0.883042473 |
| 128 | ENSG00000189058 | APOD | 3 | 195295573 | 195311076 | protein_coding | 0.880403613 |
| 129 | ENSG00000099953 | MMP11 | 22 | 24110413 | 24126503 | protein_coding | 0.879883863 |
| 130 | ENSG00000180871 | CXCR2 | 2 | 218990012 | 219001976 | protein_coding | 0.879835997 |
| 131 | ENSG00000110195 | FOLR1 | 11 | 71900602 | 71907345 | protein_coding | 0.877303032 |
| 132 | ENSG00000169245 | CXCL10 | 4 | 76942273 | 76944650 | protein_coding | 0.877159653 |
| 133 | ENSG00000158258 | CLSTN2 | 3 | 139654027 | 140296239 | protein_coding | 0.876998351 |
| 134 | ENSG00000169862 | CTNND2 | 5 | 10971952 | 11904155 | protein_coding | 0.87595885 |
| 135 | ENSG00000086696 | HSD17B2 | 16 | 82068609 | 82132139 | protein_coding | 0.875499369 |
| 136 | ENSG00000188257 | PLA2G2A | 1 | 20301925 | 20306932 | protein_coding | 0.872792262 |
| 137 | ENSG00000185630 | PBX1 | 1 | 164524821 | 164868533 | protein_coding | 0.870707578 |
| 138 | ENSG00000236882 | C5orf27 | 5 | 95187936 | 95195837 | protein_coding | 0.87003259 |
| 139 | ENSG00000223573 | TINCR | 19 | 5558178 | 5568045 | lincRNA | 0.870006308 |
| 140 | ENSG00000064270 | ATP2C2 | 16 | 84402133 | 84497793 | protein_coding | 0.86999068 |
| 141 | ENSG00000080709 | KCNN2 | 5 | 113696642 | 113832337 | protein_coding | 0.868534483 |
| 142 | ENSG00000125675 | GRIA3 | X | 122318006 | 122624766 | protein_coding | 0.868324222 |
| 143 | ENSG00000175161 | CADM2 | 3 | 85008132 | 86123579 | protein_coding | 0.867893756 |
| 144 | ENSG00000112981 | NME5 | 5 | 137450866 | 137475132 | protein_coding | 0.86758831 |
| 145 | ENSG00000186642 | PDE2A | 11 | 72287185 | 72385635 | protein_coding | 0.867378049 |
| 146 | ENSG00000136546 | SCN7A | 2 | 167260083 | 167350757 | protein_coding | 0.866908022 |
| 147 | ENSG00000131747 | TOP2A | 17 | 38544768 | 38574202 | protein_coding | 0.866642136 |
| 148 | ENSG00000082175 | PGR | 11 | 100900355 | 101001255 | protein_coding | 0.865511985 |
| 149 | ENSG00000106927 | AMBP | 9 | 116822407 | 116840752 | protein_coding | 0.864730447 |
| 150 | ENSG00000151388 | ADAMTS12 | 5 | 33523640 | 33892297 | protein_coding | 0.86357445 |
| 151 | ENSG00000165480 | SKA3 | 13 | 21727734 | 21750741 | protein_coding | 0.863540791 |
| 152 | ENSG00000214145 | LINC00887 | 3 | 194014254 | 194030592 | lincRNA | 0.861333053 |
| 153 | ENSG00000005187 | ACSM3 | 16 | 20621565 | 20808903 | protein_coding | 0.860832318 |
| 154 | ENSG00000125968 | ID1 | 20 | 30193086 | 30194318 | protein_coding | 0.860071489 |
| 155 | ENSG00000256663 | | 12 | 20704524 | 20705946 | pseudogene | 0.857390664 |
| 156 | ENSG00000232855 | | 21 | 29811667 | 30047170 | lincRNA | 0.856970143 |
| 157 | ENSG00000197776 | KLHDC1 | 14 | 50159823 | 50219870 | protein_coding | 0.854079058 |
| 158 | ENSG00000127249 | ATP13A4 | 3 | 193119866 | 193310900 | protein_coding | 0.85296437 |
| 159 | ENSG00000169248 | CXCL11 | 4 | 76954835 | 76962568 | protein_coding | 0.851978636 |
| 160 | ENSG00000123975 | CKS2 | 9 | 91926113 | 91931618 | protein_coding | 0.849612875 |
| 161 | ENSG00000170382 | LRRN2 | 1 | 204586298 | 204654861 | protein_coding | 0.846834899 |
| 162 | ENSG00000169302 | STK32A | 5 | 146614526 | 146767415 | protein_coding | 0.845902932 |
| 163 | ENSG00000237649 | KIFC1 | 6 | 33359313 | 33377701 | protein_coding | 0.845668629 |
| 164 | ENSG00000104738 | MCM4 | 8 | 48872745 | 48890720 | protein_coding | 0.845037847 |
| 165 | ENSG00000162989 | KCNJ3 | 2 | 155554811 | 155714863 | protein_coding | 0.843098072 |
| 166 | ENSG00000144847 | IGSF11 | 3 | 118619404 | 118864915 | protein_coding | 0.842999498 |
| 167 | ENSG00000165490 | C11orf82 | 11 | 82611017 | 82669319 | protein_coding | 0.840832632 |
| 168 | ENSG00000171848 | RRM2 | 2 | 10262455 | 10271545 | protein_coding | 0.83878259 |
| 169 | ENSG00000149451 | ADAM33 | 20 | 3648612 | 3662893 | protein_coding | 0.838124597 |
| 170 | ENSG00000220563 | PKMP3 | 6 | 86369610 | 86370324 | pseudogene | 0.836469722 |
| 171 | ENSG00000241186 | TDGF1 | 3 | 46616045 | 46668033 | protein_coding | 0.836128049 |
| 172 | ENSG00000132437 | DDC | 7 | 50526134 | 50633154 | protein_coding | 0.836101766 |
| 173 | ENSG00000158246 | FAM46B | 1 | 27331511 | 27339327 | protein_coding | 0.834217507 |
| 174 | ENSG00000149557 | FEZ1 | 11 | 125315646 | 125366213 | protein_coding | 0.833554377 |
| 175 | ENSG00000168309 | FAM107A | 3 | 58549844 | 58613337 | protein_coding | 0.833526072 |
| 176 | ENSG00000094963 | FMO2 | 1 | 171154347 | 171181822 | protein_coding | 0.831869668 |
| 177 | ENSG00000186204 | CYP4F12 | 19 | 15783567 | 15807984 | protein_coding | 0.830845248 |
| 178 | ENSG00000197757 | HOXC6 | 12 | 54384408 | 54424607 | protein_coding | 0.830582422 |
| 179 | ENSG00000171747 | LGALS4 | 19 | 39292311 | 39304004 | protein_coding | 0.829583684 |
| 180 | ENSG00000124440 | HIF3A | 19 | 46800303 | 46846690 | protein_coding | 0.82930676 |
| 181 | ENSG00000126778 | SIX1 | 14 | 61110133 | 61124977 | protein_coding | 0.829110597 |
| 182 | ENSG00000092850 | TEKT2 | 1 | 36549676 | 36553876 | protein_coding | 0.829055846 |
| 183 | ENSG00000188906 | LRRK2 | 12 | 40590546 | 40763087 | protein_coding | 0.828900336 |
| 184 | ENSG00000090889 | KIF4A | X | 69509879 | 69640682 | protein_coding | 0.827849033 |
| 185 | ENSG00000079101 | CLUL1 | 18 | 596988 | 650334 | protein_coding | 0.827801276 |
| 186 | ENSG00000053438 | NNAT | 20 | 36149617 | 36152092 | protein_coding | 0.825220774 |
| 187 | ENSG00000169679 | BUB1 | 2 | 111395275 | 111435691 | protein_coding | 0.825168209 |
| 188 | ENSG00000126787 | DLGAP5 | 14 | 55614830 | 55658396 | protein_coding | 0.823906644 |
| 189 | ENSG00000164128 | NPY1R | 4 | 164245113 | 164265984 | protein_coding | 0.823854079 |
| 190 | ENSG00000171509 | RXFP1 | 4 | 159236463 | 159574524 | protein_coding | 0.823320668 |
| 191 | ENSG00000186871 | ERCC6L | X | 71424510 | 71458897 | protein_coding | 0.822382254 |
| 192 | ENSG00000091513 | TF | 3 | 133464800 | 133497850 | protein_coding | 0.822352857 |
| 193 | ENSG00000172367 | PDZD3 | 11 | 119056166 | 119060932 | protein_coding | 0.82193545 |
| 194 | ENSG00000117650 | NEK2 | 1 | 211836114 | 211848960 | protein_coding | 0.821804037 |
| 195 | ENSG00000189057 | FAM111B | 11 | 58874658 | 58894883 | protein_coding | 0.820069386 |
| 196 | ENSG00000144218 | AFF3 | 2 | 100162323 | 100759201 | protein_coding | 0.819198509 |
| 197 | ENSG00000101144 | BMP7 | 20 | 55743804 | 55841685 | protein_coding | 0.818912952 |
| 198 | ENSG00000256612 | CYP2B7P | 19 | 41430124 | 41456565 | pseudogene | 0.818311348 |
| 199 | ENSG00000043591 | ADRB1 | 10 | 115803806 | 115806667 | protein_coding | 0.817914214 |
| 200 | ENSG00000228168 | HNRNPA1P21 | 3 | 39376470 | 39377430 | pseudogene | 0.817861648 |
| 201 | ENSG00000134376 | CRB1 | 1 | 197170592 | 197447585 | protein_coding | 0.817809083 |
| 202 | ENSG00000137807 | KIF23 | 15 | 69706585 | 69740764 | protein_coding | 0.816547519 |
| 203 | ENSG00000148204 | CRB2 | 9 | 126118449 | 126142603 | protein_coding | 0.815811606 |
| 204 | ENSG00000073111 | MCM2 | 3 | 127317066 | 127341276 | protein_coding | 0.81544365 |
| 205 | ENSG00000137812 | CASC5 | 15 | 40886218 | 40956540 | protein_coding | 0.81544365 |
| 206 | ENSG00000198478 | SH3BGRL2 | 6 | 80341000 | 80413372 | protein_coding | 0.815391085 |
| 207 | ENSG00000172955 | ADH6 | 4 | 100123795 | 100140694 | protein_coding | 0.815128259 |
| 208 | ENSG00000101003 | GINS1 | 20 | 25388363 | 25433264 | protein_coding | 0.814760303 |
| 209 | ENSG00000196177 | ACADSB | 10 | 124768495 | 124817827 | protein_coding | 0.814444912 |
| 210 | ENSG00000156970 | BUB1B | 15 | 40453224 | 40513337 | protein_coding | 0.814339781 |
| 211 | ENSG00000114854 | TNNC1 | 3 | 52485118 | 52488086 | protein_coding | 0.813393608 |
| 212 | ENSG00000169418 | NPR1 | 1 | 153651113 | 153666468 | protein_coding | 0.811553827 |
| 213 | ENSG00000114346 | ECT2 | 3 | 172468472 | 172539264 | protein_coding | 0.810416517 |
| 214 | ENSG00000196090 | PTPRT | 20 | 40701392 | 41818610 | protein_coding | 0.809126102 |
| 215 | ENSG00000165304 | MELK | 9 | 36572859 | 36677678 | protein_coding | 0.808347351 |
| 216 | ENSG00000241684 | ADAMTS9-AS2 | 3 | 64670585 | 64997143 | antisense | 0.808189655 |
| 217 | ENSG00000149948 | HMGA2 | 12 | 66217911 | 66360075 | protein_coding | 0.807531006 |
| 218 | ENSG00000138180 | CEP55 | 10 | 95256389 | 95288849 | protein_coding | 0.807033221 |
| 219 | ENSG00000145934 | TENM2 | 5 | 166711804 | 167691162 | protein_coding | 0.806061366 |
| 220 | ENSG00000169252 | ADRB2 | 5 | 148206156 | 148208196 | protein_coding | 0.804983179 |
| 221 | ENSG00000188039 | NWD1 | 19 | 16830787 | 16928774 | protein_coding | 0.804914331 |
| 222 | ENSG00000108187 | PBLD | 10 | 70042417 | 70092806 | protein_coding | 0.804194701 |
| 223 | ENSG00000118137 | APOA1 | 11 | 116706467 | 116708666 | protein_coding | 0.802889096 |
| 224 | ENSG00000157168 | NRG1 | 8 | 31496902 | 32622548 | protein_coding | 0.802010897 |
| 225 | ENSG00000131386 | GALNT15 | 3 | 16216156 | 16273499 | protein_coding | 0.801303616 |
| 226 | ENSG00000173641 | HSPB7 | 1 | 16340523 | 16346089 | protein_coding | 0.801096853 |
| 227 | ENSG00000175063 | UBE2C | 20 | 44441215 | 44445596 | protein_coding | 0.800620269 |
| 228 | ENSG00000136231 | IGF2BP3 | 7 | 23349828 | 23510086 | protein_coding | 0.799913972 |
| 229 | ENSG00000173930 | SLCO4C1 | 5 | 101569690 | 101632253 | protein_coding | 0.79975267 |
| 230 | ENSG00000111341 | MGP | 12 | 15034115 | 15038860 | protein_coding | 0.798444333 |
| 231 | ENSG00000185633 | NDUFA4L2 | 12 | 57628686 | 57634498 | protein_coding | 0.798085884 |
| 232 | ENSG00000146122 | DAAM2 | 6 | 39760142 | 39872648 | protein_coding | 0.797571489 |
| 233 | ENSG00000214548 | MEG3 | 14 | 101245747 | 101327368 | lincRNA | 0.794154752 |
| 234 | ENSG00000165197 | FIGF | X | 15363713 | 15402498 | protein_coding | 0.792840622 |
| 235 | ENSG00000177807 | KCNJ10 | 1 | 160007257 | 160040038 | protein_coding | 0.792709155 |
| 236 | ENSG00000071539 | TRIP13 | 5 | 892758 | 919472 | protein_coding | 0.791316232 |
| 237 | ENSG00000112984 | KIF20A | 5 | 137514408 | 137523404 | protein_coding | 0.791105971 |
| 238 | ENSG00000024526 | DEPDC1 | 1 | 68939835 | 68962904 | protein_coding | 0.790317494 |
| 239 | ENSG00000117724 | CENPF | 1 | 214776538 | 214837931 | protein_coding | 0.789844407 |
| 240 | ENSG00000184661 | CDCA2 | 8 | 25316513 | 25365436 | protein_coding | 0.789318755 |
| 241 | ENSG00000144485 | HES6 | 2 | 239146908 | 239149303 | protein_coding | 0.787619184 |
| 242 | ENSG00000102547 | CAB39L | 13 | 49882786 | 50018262 | protein_coding | 0.78663345 |
| 243 | ENSG00000115163 | CENPA | 2 | 26987157 | 27023935 | protein_coding | 0.786480235 |
| 244 | ENSG00000102384 | CENPI | X | 100353178 | 100418670 | protein_coding | 0.785376367 |
| 245 | ENSG00000088325 | TPX2 | 20 | 30327074 | 30389608 | protein_coding | 0.783957107 |
| 246 | ENSG00000115361 | ACADL | 2 | 211052663 | 211090215 | protein_coding | 0.78390028 |
| 247 | ENSG00000161798 | AQP5 | 12 | 50355653 | 50359464 | protein_coding | 0.783550792 |
| 248 | ENSG00000196136 | SERPINA3 | 14 | 95078714 | 95090392 | protein_coding | 0.783371568 |
| 249 | ENSG00000172201 | ID4 | 6 | 19837617 | 19840915 | protein_coding | 0.782695542 |
| 250 | ENSG00000204385 | SLC44A4 | 6 | 31830969 | 31846823 | protein_coding | 0.781435945 |
| 251 | ENSG00000174371 | EXO1 | 1 | 242011269 | 242058450 | protein_coding | 0.78054037 |
| 252 | ENSG00000183549 | ACSM5 | 16 | 20420856 | 20452658 | protein_coding | 0.779173675 |
| 253 | ENSG00000138769 | CDKL2 | 4 | 76503215 | 76555900 | protein_coding | 0.778873037 |
| 254 | ENSG00000121211 | MND1 | 4 | 154265801 | 154336270 | protein_coding | 0.776811958 |
| 255 | ENSG00000001626 | CFTR | 7 | 117105838 | 117356025 | protein_coding | 0.775270629 |
| 256 | ENSG00000166803 | KIAA0101 | 15 | 64657193 | 64679886 | protein_coding | 0.773654331 |
| 257 | ENSG00000143228 | NUF2 | 1 | 163236366 | 163325554 | protein_coding | 0.773286375 |
| 258 | ENSG00000235997 | | 2 | 30569525 | 30575297 | lincRNA | 0.772806294 |
| 259 | ENSG00000153902 | LG14 | 19 | 35615417 | 35633355 | protein_coding | 0.772760723 |
| 260 | ENSG00000151224 | MAT1A | 10 | 82031576 | 82049440 | protein_coding | 0.772734441 |
| 261 | ENSG00000198554 | WDHD1 | 14 | 55405668 | 55493823 | protein_coding | 0.772024811 |
| 262 | ENSG00000180785 | OR51E1 | 11 | 4664650 | 4676718 | protein_coding | 0.771686142 |
| 263 | ENSG00000198650 | TAT | 16 | 71599563 | 71611033 | protein_coding | 0.771686142 |
| 264 | ENSG00000261616 | | 15 | 99679522 | 99685575 | antisense | 0.771341463 |
| 265 | ENSG00000169031 | COL4A3 | 2 | 228029281 | 228179508 | protein_coding | 0.770185029 |
| 266 | ENSG00000134020 | PEBP4 | 8 | 22570769 | 22857513 | protein_coding | 0.76897603 |
| 267 | ENSG00000205018 | | 16 | 89006197 | 89017932 | protein_coding | 0.768634357 |
| 268 | ENSG00000100162 | CENPM | 22 | 42334725 | 42343168 | protein_coding | 0.768240118 |
| 269 | ENSG00000138160 | KIF11 | 10 | 94353043 | 94415150 | protein_coding | 0.767767031 |
| 270 | ENSG00000164188 | RANBP3L | 5 | 36248536 | 36302216 | protein_coding | 0.766137511 |
| 271 | ENSG00000254528 | | 11 | 117704434 | 117709657 | antisense | 0.76450799 |
| 272 | ENSG00000160801 | PTH1R | 3 | 46919236 | 46945287 | protein_coding | 0.763404121 |
| 273 | ENSG00000119147 | C2orf40 | 2 | 106679702 | 106694615 | protein_coding | 0.759480966 |
| 274 | ENSG00000253293 | HOXA10 | 7 | 27210210 | 27219880 | protein_coding | 0.75921213 |
| 275 | ENSG00000145020 | AMT | 3 | 49454211 | 49460186 | protein_coding | 0.757779647 |
| 276 | ENSG00000122952 | ZWINT | 10 | 58116989 | 58121036 | protein_coding | 0.757674516 |
| 277 | ENSG00000163535 | SGOL2 | 2 | 201374731 | 201448505 | protein_coding | 0.757569386 |
| 278 | ENSG00000149554 | CHEK1 | 11 | 125495036 | 125546150 | protein_coding | 0.756044996 |
| 279 | ENSG00000153446 | C16orf89 | 16 | 5094123 | 5116111 | protein_coding | 0.755806868 |
| 280 | ENSG00000139737 | SLAIN1 | 13 | 78272023 | 78338377 | protein_coding | 0.755520109 |
| 281 | ENSG00000100307 | CBX7 | 22 | 39516172 | 39548679 | protein_coding | 0.75436291 |
| 282 | ENSG00000198932 | GPRASP1 | X | 101906294 | 101914011 | protein_coding | 0.753416737 |
| 283 | ENSG00000143502 | SUSD4 | 1 | 223394161 | 223537544 | protein_coding | 0.752025235 |
| 284 | ENSG00000112782 | CLIC5 | 6 | 45868045 | 46048132 | protein_coding | 0.751469636 |
| 285 | ENSG00000161800 | RACGAP1 | 12 | 50370706 | 50426919 | protein_coding | 0.750683347 |
| 286 | ENSG00000050628 | PTGER3 | 1 | 71318036 | 71513491 | protein_coding | 0.749534017 |
| 287 | ENSG00000146006 | LRRTM2 | 5 | 138204612 | 138211057 | protein_coding | 0.748485555 |
| 288 | ENSG00000239474 | KLHL41 | 2 | 170366212 | 170382772 | protein_coding | 0.74801061 |
| 289 | ENSG00000187741 | FANCA | 16 | 89803957 | 89883065 | protein_coding | 0.746162742 |
| 290 | ENSG00000225308 | ASS1P11 | 7 | 21259832 | 21261047 | pseudogene | 0.745143021 |
| 291 | ENSG00000124713 | GNMT | 6 | 42928496 | 42931618 | protein_coding | 0.743692178 |
| 292 | ENSG00000217791 | ASS1P9 | 5 | 53154996 | 53156231 | pseudogene | 0.743583769 |
| 293 | ENSG00000175906 | ARL4D | 17 | 41476327 | 41478505 | protein_coding | 0.741694701 |
| 294 | ENSG00000136002 | ARHGEF4 | 2 | 131594489 | 131804836 | protein_coding | 0.73974979 |
| 295 | ENSG00000171320 | ESCO2 | 8 | 27629466 | 27670157 | protein_coding | 0.739119008 |
| 296 | ENSG00000112742 | TTK | 6 | 80713604 | 80752244 | protein_coding | 0.737910008 |
| 297 | ENSG00000106804 | C5 | 9 | 123714616 | 123812554 | protein_coding | 0.73680614 |
| 298 | ENSG00000132837 | DMGDH | 5 | 78293438 | 78531861 | protein_coding | 0.735375296 |
| 299 | ENSG00000162391 | FAM151A | 1 | 55074855 | 55089229 | protein_coding | 0.735018923 |
| 300 | ENSG00000127324 | TSPAN8 | 12 | 71518865 | 71835678 | protein_coding | 0.733995268 |
| 301 | ENSG00000121621 | KIF18A | 11 | 28042167 | 28129855 | protein_coding | 0.731286796 |
| 302 | ENSG00000162461 | SLC25A34 | 1 | 16062900 | 16067891 | protein_coding | 0.730267403 |
| 303 | ENSG00000255353 | | 11 | 107047012 | 107048256 | pseudogene | 0.729998566 |
| 304 | ENSG00000229953 | | 1 | 156616299 | 156631216 | antisense | 0.729640118 |
| 305 | ENSG00000139734 | DIAPH3 | 13 | 60239717 | 60738121 | protein_coding | 0.729499579 |
| 306 | ENSG00000111247 | RAD51AP1 | 12 | 4647950 | 4669214 | protein_coding | 0.727291842 |
| 307 | ENSG00000131831 | RAI2 | X | 17818169 | 17879457 | protein_coding | 0.725872582 |
| 308 | ENSG00000168490 | PHYHIP | 8 | 22077222 | 22089854 | protein_coding | 0.723940784 |
| 309 | ENSG00000119547 | ONECUT2 | 18 | 55102917 | 55158529 | protein_coding | 0.723510646 |
| 310 | ENSG00000171433 | GLOD5 | X | 48620154 | 48632064 | protein_coding | 0.72110904 |
| 311 | ENSG00000170312 | CDK1 | 10 | 62538089 | 62554610 | protein_coding | 0.720353238 |
| 312 | ENSG00000171885 | AQP4 | 18 | 24432002 | 24445782 | protein_coding | 0.719504982 |
| 313 | ENSG00000188368 | PRR19 | 19 | 42806250 | 42814973 | protein_coding | 0.71804037 |
| 314 | ENSG00000106327 | TFR2 | 7 | 100218039 | 100240402 | protein_coding | 0.716718044 |
| 315 | ENSG00000138696 | BMPR1B | 4 | 95679119 | 96079599 | protein_coding | 0.715051258 |
| 316 | ENSG00000137960 | GIPC2 | 1 | 78445226 | 78604133 | protein_coding | 0.714925801 |
| 317 | ENSG00000165795 | NDRG2 | 14 | 21484922 | 21539031 | protein_coding | 0.712470428 |
| 318 | ENSG00000118514 | ALDH8A1 | 6 | 135238528 | 135271260 | protein_coding | 0.711732548 |
| 319 | ENSG00000197766 | CFD | 19 | 859453 | 863453 | protein_coding | 0.710365854 |
| 320 | ENSG00000145692 | BHMT | 5 | 78407602 | 78428108 | protein_coding | 0.710301814 |
| 321 | ENSG00000136457 | CHAD | 17 | 48541857 | 48546327 | protein_coding | 0.709746218 |
| 322 | ENSG00000220517 | ASS1P1 | 6 | 25023475 | 25024711 | pseudogene | 0.707183311 |
| 323 | ENSG00000138207 | RBP4 | 10 | 95351444 | 95361501 | protein_coding | 0.706860707 |
| 324 | ENSG00000146411 | SLC2A12 | 6 | 134309835 | 134373774 | protein_coding | 0.705122231 |
| 325 | ENSG00000079462 | PAFAH1B3 | 19 | 42801185 | 42807698 | protein_coding | 0.704531119 |
| 326 | ENSG00000111249 | CUX2 | 12 | 111471828 | 111788358 | protein_coding | 0.7034196 |
| 327 | ENSG00000214456 | PLIN5 | 19 | 4522543 | 4535236 | protein_coding | 0.702533642 |
| 328 | ENSG00000180638 | SLC47A2 | 17 | 19581601 | 19622292 | protein_coding | 0.702254642 |
| 329 | ENSG00000137070 | IL11RA | 9 | 34650699 | 34661889 | protein_coding | 0.700115643 |
| 330 | ENSG00000153822 | KCNJ16 | 17 | 68049570 | 68131749 | protein_coding | 0.696041842 |
| 331 | ENSG00000172159 | FRMD3 | 9 | 85857905 | 86153461 | protein_coding | 0.69480656 |
| 332 | ENSG00000205038 | PKHD1L1 | 8 | 110374706 | 110542559 | protein_coding | 0.694736182 |
| 333 | ENSG00000185739 | SRL | 16 | 4239375 | 4292081 | protein_coding | 0.694596299 |
| 334 | ENSG00000168079 | SCARA5 | 8 | 27727399 | 27850244 | protein_coding | 0.691442389 |
| 335 | ENSG00000169760 | NLGN1 | 3 | 173114074 | 174004434 | protein_coding | 0.68902439 |
| 336 | ENSG00000143476 | DTL | 1 | 212208919 | 212280742 | protein_coding | 0.68771955 |
| 337 | ENSG00000258498 | DIO3OS | 14 | 102018558 | 102026768 | lincRNA | 0.687000631 |
| 338 | ENSG00000087586 | AURKA | 20 | 54944445 | 54967393 | protein_coding | 0.684135828 |
| 339 | ENSG00000104889 | RNASEH2A | 19 | 12917394 | 12924452 | protein_coding | 0.682557889 |
| 340 | ENSG00000150625 | GPM6A | 4 | 176554085 | 176923815 | protein_coding | 0.681402439 |
| 341 | ENSG00000108381 | ASPA | 17 | 3375668 | 3406713 | protein_coding | 0.680351135 |
| 342 | ENSG00000120334 | CENPL | 1 | 173768688 | 173793858 | protein_coding | 0.680030827 |
| 343 | ENSG00000116147 | TNR | 1 | 175284330 | 175712906 | protein_coding | 0.679251201 |
| 344 | ENSG00000105852 | PON3 | 7 | 94989256 | 95025680 | protein_coding | 0.678955481 |
| 345 | ENSG00000175643 | RMI2 | 16 | 11343476 | 11445619 | protein_coding | 0.67791598 |
| 346 | ENSG00000271474 | | 4 | 96470280 | 96473608 | antisense | 0.675304878 |
| 347 | ENSG00000068976 | PYGM | 11 | 64513861 | 64527769 | protein_coding | 0.675155925 |
| 348 | ENSG00000161594 | KLHL10 | 17 | 39991937 | 40004636 | protein_coding | 0.675042052 |
| 349 | ENSG00000213070 | HMGB3P6 | 1 | 164326004 | 164326601 | pseudogene | 0.672098402 |
| 350 | ENSG00000242600 | MBL1P | 10 | 81664654 | 81710092 | pseudogene | 0.670496093 |
| 351 | ENSG00000099937 | SERPIND1 | 22 | 21128167 | 21142008 | protein_coding | 0.669102187 |
| 352 | ENSG00000130208 | APOC1 | 19 | 45417504 | 45422606 | protein_coding | 0.667998318 |
| 353 | ENSG00000078549 | ADCYAP1R1 | 7 | 31092076 | 31151089 | protein_coding | 0.667525231 |
| 354 | ENSG00000131153 | GINS2 | 16 | 85709804 | 85723679 | protein_coding | 0.667157275 |
| 355 | ENSG00000077152 | UBE2T | 1 | 202300785 | 202311108 | protein_coding | 0.665895711 |
| 356 | ENSG00000092529 | CAPN3 | 15 | 42640301 | 42704516 | protein_coding | 0.66524482 |
| 357 | ENSG00000080293 | SCTR | 2 | 120197419 | 120282070 | protein_coding | 0.664294932 |
| 358 | ENSG00000108405 | P2RX1 | 17 | 3799886 | 3819794 | protein_coding | 0.663845669 |
| 359 | ENSG00000213366 | GSTM2 | 1 | 110210644 | 110252171 | protein_coding | 0.662717758 |
| 360 | ENSG00000131781 | FM05 | 1 | 146646930 | 146714700 | protein_coding | 0.661714101 |
| 361 | ENSG00000084674 | APOB | 2 | 21224301 | 21266945 | protein_coding | 0.661355653 |
| 362 | ENSG00000134057 | CCNB1 | 5 | 68462837 | 68474072 | protein_coding | 0.659167368 |
| 363 | ENSG00000117834 | SLC5A9 | 1 | 48688357 | 48714316 | protein_coding | 0.657012195 |
| 364 | ENSG00000110492 | MDK | 11 | 46402306 | 46405375 | protein_coding | 0.654541632 |
| 365 | ENSG00000164106 | SCRG1 | 4 | 174305852 | 174327531 | protein_coding | 0.653899921 |
| 366 | ENSG00000226252 | | 1 | 47691469 | 47696422 | antisense | 0.652754415 |
| 367 | ENSG00000163239 | TDRD10 | 1 | 154474695 | 154520623 | protein_coding | 0.648312658 |
| 368 | ENSG00000087494 | PTHLH | 12 | 28111017 | 28125638 | protein_coding | 0.64681455 |
| 369 | ENSG00000124253 | PCK1 | 20 | 56136136 | 56141513 | protein_coding | 0.64529016 |
| 370 | ENSG00000163032 | VSNL1 | 2 | 17720393 | 17838285 | protein_coding | 0.64490286 |
| 371 | ENSG00000178826 | TMEM139 | 7 | 142977050 | 142985141 | protein_coding | 0.642608789 |
| 372 | ENSG00000272789 | | 2 | 128383572 | 128384423 | antisense | 0.640296468 |
| 373 | ENSG00000111452 | GPR133 | 12 | 131438452 | 131626014 | protein_coding | 0.640027959 |
| 374 | ENSG00000211445 | GPX3 | 5 | 150400124 | 150408554 | protein_coding | 0.63966951 |
| 375 | ENSG00000162367 | TAL1 | 1 | 47681962 | 47697892 | protein_coding | 0.639560555 |
| 376 | ENSG00000174502 | SLC26A9 | 1 | 205882176 | 205912588 | protein_coding | 0.639297729 |
| 377 | ENSG00000173376 | NDNF | 4 | 121956768 | 121994176 | protein_coding | 0.638898846 |
| 378 | ENSG00000164109 | MAD2L1 | 4 | 120976763 | 120988229 | protein_coding | 0.6379836 |
| 379 | ENSG00000137878 | GCOM1 | 15 | 57884106 | 58006943 | protein_coding | 0.637178292 |
| 380 | ENSG00000169116 | PARM1 | 4 | 75858305 | 75975325 | protein_coding | 0.63708868 |
| 381 | ENSG00000168824 | | 4 | 4349867 | 4420785 | protein_coding | 0.635780343 |
| 382 | ENSG00000160808 | MYL3 | 3 | 46899362 | 46923659 | protein_coding | 0.63525021 |
| 383 | ENSG00000109794 | FAM149A | 4 | 187025573 | 187093821 | protein_coding | 0.634669152 |
| 384 | ENSG00000132840 | BHMT2 | 5 | 78365540 | 78385289 | protein_coding | 0.634409167 |
| 385 | ENSG00000100024 | UPB1 | 22 | 24863206 | 24924358 | protein_coding | 0.633199513 |
| 386 | ENSG00000143257 | NR113 | 1 | 161199456 | 161208092 | protein_coding | 0.631658183 |
| 387 | ENSG00000128482 | RNF112 | 17 | 19314438 | 19320589 | protein_coding | 0.631228045 |
| 388 | ENSG00000144230 | GPR17 | 2 | 128403439 | 128410213 | protein_coding | 0.630959209 |
| 389 | ENSG00000213760 | ATP6V1G2 | 6 | 31512239 | 31516204 | protein_coding | 0.630624474 |
| 390 | ENSG00000175556 | LONRF3 | X | 118108581 | 118152318 | protein_coding | 0.627733171 |
| 391 | ENSG00000105011 | ASF1B | 19 | 14230321 | 14247768 | protein_coding | 0.623317914 |
| 392 | ENSG00000249669 | MIR143HG | 5 | 148786252 | 148808241 | lincRNA | 0.619040791 |
| 393 | ENSG00000133116 | KL | 13 | 33590207 | 33640282 | protein_coding | 0.618413506 |
| 394 | ENSG00000171056 | SOX7 | 8 | 10581278 | 10697357 | protein_coding | 0.617903701 |
| 395 | ENSG00000124701 | APOBEC2 | 6 | 41021043 | 41032250 | protein_coding | 0.617768299 |
| 396 | ENSG00000095970 | TREM2 | 6 | 41126244 | 41130924 | protein_coding | 0.617640875 |
| 397 | ENSG00000109846 | CRYAB | 11 | 111779289 | 111794446 | protein_coding | 0.616782565 |
| 398 | ENSG00000184434 | LRRC19 | 9 | 26993134 | 27005691 | protein_coding | 0.614434727 |
| 399 | ENSG00000241978 | AKAP2 | 9 | 112542769 | 112934792 | protein_coding | 0.613807441 |
| 400 | ENSG00000196616 | ADH1B | 4 | 100226121 | 100242558 | protein_coding | 0.613015139 |
| 401 | ENSG00000123560 | PLP1 | X | 103028647 | 103047548 | protein_coding | 0.612594617 |
| 402 | ENSG00000151655 | ITIH2 | 10 | 7745232 | 7791483 | protein_coding | 0.612212345 |
| 403 | ENSG00000122367 | LDB3 | 10 | 88428206 | 88495825 | protein_coding | 0.609989963 |
| 404 | ENSG00000117601 | SERPINC1 | 1 | 173872947 | 173886516 | protein_coding | 0.609792817 |
| 405 | ENSG00000180525 | PRR26 | 10 | 695888 | 711109 | protein_coding | 0.607588358 |
| 406 | ENSG00000106278 | PTPRZ1 | 7 | 121513143 | 121702090 | protein_coding | 0.606477167 |
| 407 | ENSG00000067840 | PDZD4 | X | 153067621 | 153096020 | protein_coding | 0.605498318 |
| 408 | ENSG00000164736 | SOX17 | 8 | 55370495 | 55373448 | protein_coding | 0.605445753 |
| 409 | ENSG00000080493 | SLC4A4 | 4 | 72053003 | 72437804 | protein_coding | 0.604972666 |
| 410 | ENSG00000267336 | | 18 | 12912843 | 12913905 | pseudogene | 0.604972666 |
| 411 | ENSG00000197705 | KLHL14 | 18 | 30252634 | 30353025 | protein_coding | 0.603385547 |
| 412 | ENSG00000018625 | ATP1A2 | 1 | 160085549 | 160113381 | protein_coding | 0.602623844 |
| 413 | ENSG00000126838 | PZP | 12 | 9301436 | 9360966 | protein_coding | 0.599003513 |
| 414 | ENSG00000077522 | ACTN2 | 1 | 236849754 | 236927931 | protein_coding | 0.597613541 |
| 415 | ENSG00000147576 | ADHFE1 | 8 | 67342420 | 67383836 | protein_coding | 0.597336727 |
| 416 | ENSG00000130226 | DPP6 | 7 | 153584182 | 154685995 | protein_coding | 0.595931455 |
| 417 | ENSG00000154258 | ABCA9 | 17 | 66970629 | 67057205 | protein_coding | 0.590329056 |
| 418 | ENSG00000183807 | FAM162B | 6 | 117073363 | 117086886 | protein_coding | 0.590239444 |
| 419 | ENSG00000091622 | PITPNM3 | 17 | 6354584 | 6459814 | protein_coding | 0.588160442 |
| 420 | ENSG00000154263 | ABCA10 | 17 | 67143355 | 67240987 | protein_coding | 0.585891505 |
| 421 | ENSG00000104888 | SLC17A7 | 19 | 49932658 | 49945617 | protein_coding | 0.585681245 |
| 422 | ENSG00000152779 | SLC16A12 | 10 | 91190051 | 91316398 | protein_coding | 0.584217507 |
| 423 | ENSG00000115252 | PDE1A | 2 | 183004763 | 183387919 | protein_coding | 0.581791421 |
| 424 | ENSG00000173597 | SULT1B1 | 4 | 70586880 | 70653679 | protein_coding | 0.581672521 |
| 425 | ENSG00000147606 | SLC26A7 | 8 | 92221722 | 92410378 | protein_coding | 0.581475375 |
| 426 | ENSG00000168078 | PBK | 8 | 27667137 | 27695612 | protein_coding | 0.579772744 |
| 427 | ENSG00000203709 | C1orf132 | 1 | 207974863 | 208042495 | processed_transcrip | 0.579636249 |
| 428 | ENSG00000079337 | RAPGEF3 | 12 | 48128455 | 48164823 | protein_coding | 0.576044878 |
| 429 | ENSG00000025423 | HSD17B6 | 12 | 57145945 | 57181574 | protein_coding | 0.575919421 |
| 430 | ENSG00000137731 | FXYD2 | 11 | 117671559 | 117699413 | protein_coding | 0.575168209 |
| 431 | ENSG00000158220 | ESYT3 | 3 | 138153428 | 138200528 | protein_coding | 0.574038057 |
| 432 | ENSG00000048540 | LMO3 | 12 | 16701307 | 16763528 | protein_coding | 0.573065601 |
| 433 | ENSG00000146469 | VIP | 6 | 153071933 | 153080900 | protein_coding | 0.572971181 |
| 434 | ENSG00000241158 | ADAMTS9-AS1 | 3 | 64547014 | 64573878 | antisense | 0.570647603 |
| 435 | ENSG00000042062 | FAM65C | 20 | 49202645 | 49308065 | protein_coding | 0.567335997 |
| 436 | ENSG00000068831 | RASGRP2 | 11 | 64494383 | 64512928 | protein_coding | 0.563499176 |
| 437 | ENSG00000169994 | MYO7B | 2 | 128293378 | 128395304 | protein_coding | 0.563130782 |
| 438 | ENSG00000214491 | SEC14L6 | 22 | 30918786 | 30942669 | protein_coding | 0.557145674 |
| 439 | ENSG00000162407 | PPAP2B | 1 | 56960419 | 57110974 | protein_coding | 0.555989677 |
| 440 | ENSG00000101542 | CDH20 | 18 | 59000815 | 59223006 | protein_coding | 0.53795971 |
| 441 | ENSG00000171227 | TMEM37 | 2 | 120187477 | 120196096 | protein_coding | 0.516610597 |
| 442 | ENSG00000171766 | GATM | 15 | 45653322 | 45694525 | protein_coding | 0.514718251 |
| 443 | ENSG00000187699 | C2orf88 | 2 | 190744335 | 191068210 | protein_coding | 0.511511775 |

TABLE I
10 biomarkers and their importance in the RF-RKFCV.
Genes are rank based on their performance in the machine-learning model

| # | Ensembl gene id | Symbol | Gene Description | VarImp |
|---|---|---|---|---|
| 1 | ENSG00000154258 | ABCA9 | ATP binding cassette subfamily A member 9 | 1 |
| | | | [Source: HGNC Symbol; Acc: HGNC: 39] | |
| 2 | ENSG00000092529 | CAPN3 | calpain 3 [Source: HGNC Symbol; Acc: HGNC: 1480] | 0.35868747 |
| 3 | ENSG00000099953 | MMP11 | matrix metallopeptidase 11 | 0.26953528 |
| | | | [Source: HGNC Symbol; Acc: HGNC: 7157] | |
| 4 | ENSG00000267336 | EIF4A2P1 | eukaryotic translation initiation factor 4A2 pseudogene 1 | 0.16884656 |
| | | | [Source: HGNC Symbol; Acc: HGNC: 37929] | |
| 5 | ENSG00000175084 | DES | desmin [Source: HGNC Symbol; Acc: HGNC: 2770] | 0.16060116 |
| 6 | ENSG00000254726 | MEX3A | mex-3 RNA binding family member A | 0.14055028 |
| | | | [Source: HGNC Symbol; Acc: HGNC: 33482] | |
| 7 | ENSG00000154263 | ABCA10 | ATP binding cassette subfamily A member 10 | 0.10305968 |
| | | | [Source: HGNC Symbol; Acc: HGNC: 30] | |
| 8 | ENSG00000168079 | SCARA5 | scavenger receptor class A member 5 | 0.0714065 |
| | | | [Source: HGNC Symbol; Acc: HGNC: 28701] | |
| 9 | ENSG00000149451 | ADAM33 | ADAM metallopeptidase domain 33 | 0.05927563 |
| | | | [Source: HGNC Symbol; Acc: HGNC: 15478] | |
| 10 | ENSG00000164283 | ESM1 | endothelial cell specific molecule 1 | 0.04453125 |
| | | | [Source: HGNC Symbol; Acc: HGNC: 3466] | |

TABLE J
150 biomarkers from to 1:1 mammalian orhtologus (Human, Mouse, Dog and Tasmanian Devil) and their importance
in the RF-RKFCV.

| # | Ensembl gene id | Symbol | Chr | Start | End | Gene Biotype | Varlmp |
|---|---|---|---|---|---|---|---|
| 1 | ENSG00000149451 | ADAM33 | 20 | 3648612 | 3662893 | protein_coding | 1 |
| 2 | ENSG00000144230 | GPR17 | 2 | 128403439 | 128410213 | protein_coding | 0.91560762 |
| 3 | ENSG00000124766 | SOX4 | 6 | 21593972 | 21598847 | protein_coding | 0.87720641 |
| 4 | ENSG00000092529 | CAPN3 | 15 | 42640301 | 42704516 | protein_coding | 0.79759359 |
| 5 | ENSG00000254726 | MEX3A | 1 | 156041804 | 156051789 | protein_coding | 0.70186072 |
| 6 | ENSG00000166803 | KIAA0101 | 15 | 64657193 | 64679886 | protein_coding | 0.58685649 |
| 7 | ENSG00000104889 | RNASEH2A | 19 | 12917394 | 12924452 | protein_coding | 0.51565931 |
| 8 | ENSG00000131153 | GINS2 | 16 | 85709804 | 85723679 | protein_coding | 0.42265272 |
| 9 | ENSG00000165480 | SKA3 | 13 | 21727734 | 21750741 | protein_coding | 0.37026968 |
| 10 | ENSG00000143476 | DTL | 1 | 212208919 | 212280742 | protein_coding | 0.32718263 |
| 11 | ENSG00000160808 | MYL3 | 3 | 46899362 | 46923659 | protein_coding | 0.32002338 |
| 12 | ENSG00000131747 | TOP2A | 17 | 38544768 | 38574202 | protein_coding | 0.31108945 |
| 13 | ENSG00000175084 | DES | 2 | 220283099 | 220291461 | protein_coding | 0.30064751 |
| 14 | ENSG00000128482 | RNF112 | 17 | 19314438 | 19320589 | protein_coding | 0.2873905 |
| 15 | ENSG00000099953 | MMP11 | 22 | 24110413 | 24126503 | protein_coding | 0.26799585 |
| 16 | ENSG00000167676 | PLIN4 | 19 | 4502204 | 4517716 | protein_coding | 0.21045811 |
| 17 | ENSG00000153902 | LGI4 | 19 | 35615417 | 35633355 | protein_coding | 0.20592297 |
| 18 | ENSG00000168079 | SCARA5 | 8 | 27727399 | 27850244 | protein_coding | 0.20582616 |
| 19 | ENSG00000149554 | CHEK1 | 11 | 125495036 | 125546150 | protein_coding | 0.20509689 |
| 20 | ENSG00000068976 | PYGM | 11 | 64513861 | 64527769 | protein_coding | 0.19462437 |
| 21 | ENSG00000165197 | FIGF | X | 15363713 | 15402498 | protein_coding | 0.19216667 |
| 22 | ENSG00000168496 | FEN1 | 11 | 61560109 | 61564716 | protein_coding | 0.1724678 |
| 23 | ENSG00000122367 | LDB3 | 10 | 88428206 | 88495825 | protein_coding | 0.16052007 |
| 24 | ENSG00000123560 | PLP1 | X | 103028647 | 103047548 | protein_coding | 0.14192236 |
| 25 | ENSG00000068489 | PRR11 | 17 | 57232860 | 57282066 | protein_coding | 0.13431417 |
| 26 | ENSG00000205038 | PKHD1L1 | 8 | 110374706 | 110542559 | protein_coding | 0.12494792 |
| 27 | ENSG00000165795 | NDRG2 | 14 | 21484922 | 21539031 | protein_coding | 0.11592328 |
| 28 | ENSG00000018625 | ATP1A2 | 1 | 160085549 | 160113381 | protein_coding | 0.10353594 |
| 29 | ENSG00000114854 | TNNC1 | 3 | 52485118 | 52488086 | protein_coding | 0.10269071 |
| 30 | ENSG00000095970 | TREM2 | 6 | 41126244 | 41130924 | protein_coding | 0.10220395 |
| 31 | ENSG00000079462 | PAFAH1B3 | 19 | 42801185 | 42807698 | protein_coding | 0.10016085 |
| 32 | ENSG00000197766 | CFD | 19 | 859453 | 863453 | protein_coding | 0.09884555 |
| 33 | ENSG00000168477 | TNXB | 6 | 32008931 | 32083111 | protein_coding | 0.09752753 |
| 34 | ENSG00000068831 | RASGRP2 | 11 | 64494383 | 64512928 | protein_coding | 0.09175784 |
| 35 | ENSG00000133800 | LYVE1 | 11 | 10578513 | 10633236 | protein_coding | 0.09091349 |
| 36 | ENSG00000143196 | DPT | 1 | 168664697 | 168698502 | protein_coding | 0.08217266 |
| 37 | ENSG00000173641 | HSPB7 | 1 | 16340523 | 16346089 | protein_coding | 0.07686728 |
| 38 | ENSG00000148795 | CYP17A1 | 10 | 104590288 | 104597290 | protein_coding | 0.07087703 |
| 39 | ENSG00000162461 | SLC25A34 | 1 | 16062900 | 16067891 | protein_coding | 0.06767641 |
| 40 | ENSG00000042062 | FAM65C | 20 | 49202645 | 49308065 | protein_coding | 0.06354495 |
| 41 | ENSG00000146122 | DAAM2 | 6 | 39760142 | 39872648 | protein_coding | 0.06303238 |
| 42 | ENSG00000175161 | CADM2 | 3 | 85008132 | 86123579 | protein_coding | 0.06203265 |
| 43 | ENSG00000197580 | BCO2 | 11 | 112046190 | 112095422 | protein_coding | 0.05833392 |
| 44 | ENSG00000180509 | KCNE1 | 21 | 35818988 | 35884573 | protein_coding | 0.05590818 |
| 45 | ENSG00000077522 | ACTN2 | 1 | 236849754 | 236927931 | protein_coding | 0.05392317 |
| 46 | ENSG00000120913 | PDLIM2 | 8 | 22435792 | 22455538 | protein_coding | 0.05067719 |
| 47 | ENSG00000126759 | CFP | X | 47483612 | 47489704 | protein_coding | 0.049834 |
| 48 | ENSG00000179094 | PER1 | 17 | 8043790 | 8059824 | protein_coding | 0.04891431 |
| 49 | ENSG00000106278 | PTPRZ1 | 7 | 121513143 | 121702090 | protein_coding | 0.0462895 |
| 50 | ENSG00000140678 | ITGAX | 16 | 31366455 | 31394318 | protein_coding | 0.0458302 |
| 51 | ENSG00000011465 | DCN | 12 | 91539025 | 91576900 | protein_coding | 0.04429449 |
| 52 | ENSG00000187730 | GABRD | 1 | 1950780 | 1962192 | protein_coding | 0.04429266 |
| 53 | ENSG00000198624 | CCDC69 | 5 | 150560613 | 150603706 | protein_coding | 0.04428925 |
| 54 | ENSG00000157152 | SYN2 | 3 | 12045876 | 12232900 | processed_transcrip | 0.04301054 |
| 55 | ENSG00000165072 | MAMDC2 | 9 | 72658497 | 72841886 | protein_coding | 0.04293754 |
| 56 | ENSG00000108405 | P2RX1 | 17 | 3799886 | 3819794 | protein_coding | 0.04291264 |
| 57 | ENSG00000163431 | LMOD1 | 1 | 201865580 | 201915715 | protein_coding | 0.04180652 |
| 58 | ENSG00000166405 | RIC3 | 11 | 8127597 | 8190602 | protein_coding | 0.04006344 |
| 59 | ENSG00000166819 | PLIN1 | 15 | 90207596 | 90222658 | protein_coding | 0.03960025 |
| 60 | ENSG00000167434 | CA4 | 17 | 58227297 | 58248260 | protein_coding | 0.03832742 |
| 61 | ENSG00000124253 | PCK1 | 20 | 56136136 | 56141513 | protein_coding | 0.03786443 |
| 62 | ENSG00000164932 | CTHRC1 | 8 | 104383743 | 104395225 | protein_coding | 0.0355614 |
| 63 | ENSG00000118526 | TCF21 | 6 | 134210276 | 134216691 | protein_coding | 0.03526908 |
| 64 | ENSG00000169258 | GPRIN1 | 5 | 176022803 | 176037134 | protein_coding | 0.03453332 |
| 65 | ENSG00000196569 | LAMA2 | 6 | 129204342 | 129837714 | protein_coding | 0.03449663 |
| 66 | ENSG00000141744 | PNMT | 17 | 37824234 | 37826728 | protein_coding | 0.03413458 |
| 67 | ENSG00000136011 | STAB2 | 12 | 103981051 | 104160505 | protein_coding | 0.03343702 |
| 68 | ENSG00000145020 | AMT | 3 | 49454211 | 49460186 | protein_coding | 0.03321589 |
| 69 | ENSG00000077157 | PPP1R12B | 1 | 202317827 | 202561834 | protein_coding | 0.03321273 |
| 70 | ENSG00000069702 | TGFBR3 | 1 | 92145902 | 92371892 | protein_coding | 0.03309275 |
| 71 | ENSG00000118194 | TNNT2 | 1 | 201328136 | 201346890 | protein_coding | 0.03214898 |
| 72 | ENSG00000117834 | SLC5A9 | 1 | 48688357 | 48714316 | protein_coding | 0.03209673 |
| 73 | ENSG00000118785 | SPP1 | 4 | 88896819 | 88904562 | protein_coding | 0.03208906 |
| 74 | ENSG00000161649 | CD300LG | 17 | 41924516 | 41940997 | protein_coding | 0.03187562 |
| 75 | ENSG00000166856 | GPR182 | 12 | 57388230 | 57390468 | protein_coding | 0.03187371 |
| 76 | ENSG00000174938 | SEZ6L2 | 16 | 29882480 | 29910868 | protein_coding | 0.03113724 |
| 77 | ENSG00000101425 | BPI | 20 | 36888551 | 36965907 | protein_coding | 0.0310241 |
| 78 | ENSG00000136826 | KLF4 | 9 | 110247133 | 110252763 | protein_coding | 0.03082667 |
| 79 | ENSG00000134013 | LOXL2 | 8 | 23154702 | 23282841 | protein_coding | 0.0301798 |
| 80 | ENSG00000019102 | VSIG2 | 11 | 124617368 | 124622134 | protein_coding | 0.02946848 |
| 81 | ENSG00000104888 | SLC17A7 | 19 | 49932658 | 49945617 | protein_coding | 0.02940086 |
| 82 | ENSG00000115602 | IL1RL1 | 2 | 102927962 | 102968497 | protein_coding | 0.02921207 |
| 83 | ENSG00000198483 | ANKRD35 | 1 | 145549230 | 145568526 | protein_coding | 0.02888838 |
| 84 | ENSG00000183287 | CCBE1 | 18 | 57098172 | 57364612 | protein_coding | 0.02860167 |
| 85 | ENSG00000145708 | CRHBP | 5 | 76248538 | 76276983 | protein_coding | 0.02719697 |
| 86 | ENSG00000137878 | GCOM1 | 15 | 57884106 | 58006943 | protein_coding | 0.02524558 |
| 87 | ENSG00000168447 | SCNN1B | 16 | 23289552 | 23392620 | protein_coding | 0.02518725 |
| 88 | ENSG00000137573 | SULF1 | 8 | 70378859 | 70573150 | protein_coding | 0.02494342 |
| 89 | ENSG00000049249 | TNFRSF9 | 1 | 7979907 | 8000926 | protein_coding | 0.02486396 |
| 90 | ENSG00000086991 | NOX4 | 11 | 89057524 | 89322779 | protein_coding | 0.02432495 |
| 91 | ENSG00000169684 | CHRNA5 | 15 | 78857862 | 78887611 | protein_coding | 0.02401424 |
| 92 | ENSG00000134853 | PDGFRA | 4 | 55095264 | 55164414 | protein_coding | 0.02382877 |
| 93 | ENSG00000114812 | VIPR1 | 3 | 42530791 | 42579059 | protein_coding | 0.0235023 |
| 94 | ENSG00000149294 | NCAM1 | 11 | 112831997 | 113149158 | protein_coding | 0.02305201 |
| 95 | ENSG00000163661 | PTX3 | 3 | 157154578 | 157161417 | protein_coding | 0.02246859 |
| 96 | ENSG00000108821 | COL1A1 | 17 | 48260650 | 48278993 | protein_coding | 0.02239369 |
| 97 | ENSG00000153246 | PLA2R1 | 2 | 160788519 | 160919121 | protein_coding | 0.02236067 |
| 98 | ENSG00000064205 | WISP2 | 20 | 43343485 | 43357150 | protein_coding | 0.02223716 |
| 99 | ENSG00000175832 | ETV4 | 17 | 41605212 | 41656988 | protein_coding | 0.02210647 |
| 100 | ENSG00000164512 | ANKRD55 | 5 | 55395507 | 55529186 | protein_coding | 0.02190911 |
| 101 | ENSG00000111405 | ENDOU | 12 | 48103517 | 48119350 | protein_coding | 0.02163363 |
| 102 | ENSG00000203710 | CR1 | 1 | 207669492 | 207813992 | protein_coding | 0.02125239 |
| 103 | ENSG00000138678 | AGPAT9 | 4 | 84457067 | 84527028 | protein_coding | 0.02122404 |
| 104 | ENSG00000180785 | OR51E1 | 11 | 4664650 | 4676718 | protein_coding | 0.0211259 |
| 105 | ENSG00000163687 | DNASE1L3 | 3 | 58177984 | 58200424 | protein_coding | 0.01993235 |
| 106 | ENSG00000119547 | ONECUT2 | 18 | 55102917 | 55158529 | protein_coding | 0.0197463 |
| 107 | ENSG00000162849 | KIF26B | 1 | 245318287 | 245872733 | protein_coding | 0.01971843 |
| 108 | ENSG00000135119 | RNFT2 | 12 | 117176096 | 117291436 | protein_coding | 0.01961622 |
| 109 | ENSG00000144485 | HES6 | 2 | 239146908 | 239149303 | protein_coding | 0.01944706 |
| 110 | ENSG00000102010 | BMX | X | 15482369 | 15574652 | protein_coding | 0.01919883 |
| 111 | ENSG00000018236 | CNTN1 | 12 | 41086244 | 41466220 | protein_coding | 0.0189451 |
| 112 | ENSG00000119121 | TRPM6 | 9 | 77337411 | 77503010 | protein_coding | 0.01889901 |
| 113 | ENSG00000164879 | CA3 | 8 | 86285665 | 86361269 | protein_coding | 0.01863909 |
| 114 | ENSG00000157766 | ACAN | 15 | 89346674 | 89418585 | protein_coding | 0.01855134 |
| 115 | ENSG00000091622 | PITPNM3 | 17 | 6354584 | 6459814 | protein_coding | 0.01841295 |
| 116 | ENSG00000185633 | NDUFA4L2 | 12 | 57628686 | 57634498 | protein_coding | 0.018106 |
| 117 | ENSG00000126778 | SIX1 | 14 | 61110133 | 61124977 | protein_coding | 0.01735917 |
| 118 | ENSG00000174514 | MFSD4 | 1 | 205538013 | 205572046 | protein_coding | 0.01732019 |
| 119 | ENSG00000203747 | FCGR3A | 1 | 161511549 | 161600917 | protein_coding | 0.01660236 |
| 120 | ENSG00000165272 | AQP3 | 9 | 33441152 | 33447609 | protein_coding | 0.01628838 |
| 121 | ENSG00000204385 | SLC44A4 | 6 | 31830969 | 31846823 | protein_coding | 0.01614723 |
| 122 | ENSG00000166183 | ASPG | 14 | 104552016 | 104579098 | protein_coding | 0.01614657 |
| 123 | ENSG00000136457 | CHAD | 17 | 48541857 | 48546327 | protein_coding | 0.01606077 |
| 124 | ENSG00000165125 | TRPV6 | 7 | 142568956 | 142583507 | protein_coding | 0.01583674 |
| 125 | ENSG00000162745 | OLFML2B | 1 | 161952982 | 161993644 | protein_coding | 0.01568089 |
| 126 | ENSG00000172005 | MAL | 2 | 95691422 | 95719737 | protein_coding | 0.01557144 |
| 127 | ENSG00000127249 | ATP13A4 | 3 | 193119866 | 193310900 | protein_coding | 0.01528597 |
| 128 | ENSG00000184012 | TMPRSS2 | 21 | 42836478 | 42903043 | protein_coding | 0.01518453 |
| 129 | ENSG00000213088 | DARC | 1 | 159173097 | 159176290 | protein_coding | 0.01515086 |
| 130 | ENSG00000160161 | CILP2 | 19 | 19649057 | 19657468 | protein_coding | 0.01494213 |
| 131 | ENSG00000146070 | PLA2G7 | 6 | 46671938 | 46703430 | protein_coding | 0.01435515 |
| 132 | ENSG00000148848 | ADAM12 | 10 | 127700950 | 128077024 | protein_coding | 0.01419065 |
| 133 | ENSG00000161640 | SIGLEC11 | 19 | 50452242 | 50464429 | protein_coding | 0.01405047 |
| 134 | ENSG00000171747 | LGALS4 | 19 | 39292311 | 39304004 | protein_coding | 0.01387363 |
| 135 | ENSG00000164694 | FNDC1 | 6 | 159590429 | 159693141 | protein_coding | 0.01323024 |
| 136 | ENSG00000136002 | ARHGEF4 | 2 | 131594489 | 131804836 | protein_coding | 0.01318945 |
| 137 | ENSG00000128016 | ZFP36 | 19 | 39897453 | 39900052 | protein_coding | 0.013168 |
| 138 | ENSG00000135373 | EHF | 11 | 34642640 | 34682604 | protein_coding | 0.01232076 |
| 139 | ENSG00000118596 | SLC16A7 | 12 | 59989848 | 60176395 | protein_coding | 0.01229893 |
| 140 | ENSG00000167244 | IGF2 | 11 | 2150342 | 2170833 | protein_coding | 0.01219069 |
| 141 | ENSG00000123454 | DBH | 9 | 136501482 | 136524466 | protein_coding | 0.01167961 |
| 142 | ENSG00000169245 | CXCL10 | 4 | 76942273 | 76944650 | protein_coding | 0.01076985 |
| 143 | ENSG00000120729 | MYOT | 5 | 137203480 | 137223540 | protein_coding | 0.01072576 |
| 144 | ENSG00000089199 | CHGB | 20 | 5892076 | 5906007 | protein_coding | 0.00976823 |
| 145 | ENSG00000126500 | FLRT1 | 11 | 63870660 | 63886645 | protein_coding | 0.0096213 |
| 146 | ENSG00000140254 | DUOXA1 | 15 | 45409569 | 45422136 | protein_coding | 0.00958637 |
| 147 | ENSG00000187642 | C1orf170 | 1 | 910579 | 917497 | protein_coding | 0.00930035 |
| 148 | ENSG00000198910 | L1CAM | X | 153126969 | 153174677 | protein_coding | 0.00896413 |
| 149 | ENSG00000152092 | ASTN1 | 1 | 176826438 | 177134109 | protein_coding | 0.00857287 |
| 150 | ENSG00000141527 | CARD14 | 17 | 78143791 | 78183130 | protein_coding | 0.00677388 |

TABLE K
Most stable genes across cancer and normal tissues.
Criteria:
1—Not differentially expressed <0.5 fold & expressed with >100 count per million/10 counts VST transformed.
2—Not found on driver list.
3—Stable expression across tissue and/cancers.
4—Not being a prognostic gene.
5—Single gene family orthologous.

| # | ensembl_gene_id | hgnc_symb [symbol] chromosome_name | | start_position | end_position | gene_biotype |
|---|---|---|---|---|---|---|
| 1 | ENSG00000015479 | MATR3 | 5 | 138609441 | 138667360 | protein_coding |
| 2 | ENSG00000172775 | FAM192A | 16 | 57186378 | 57220028 | protein_coding |
| 3 | ENSG00000204256 | BRD2 | 6 | 32936437 | 32949282 | protein_coding |
| 4 | ENSG00000108312 | UBTF | 17 | 42282401 | 42298994 | protein_coding |
| 5 | ENSG00000163166 | IWS1 | 2 | 128193783 | 128284462 | protein_coding |
| 6 | ENSG00000100796 | SMEK1 | 14 | 91923955 | 91976898 | protein_coding |
| 7 | ENSG00000122557 | HERPUD2 | 7 | 35672269 | 35735181 | protein_coding |

| # | ensembl_gene_id | description |
|---|---|---|
| 1 | ENSG00000015479 | matrin 3 [Source: HGNC Symbol; Acc: 6912] |
| 2 | ENSG00000172775 | family with sequence similarity 192, member A [Source: HGNC Symbol; Acc: 29856] |
| 3 | ENSG00000204256 | bromodomain containing 2 [Source: HGNC Symbol; Acc 1103] |
| 4 | ENSG00000108312 | upstream binding transcription factor, RNA polymerase I [Source: HGNC Symbol; Acc: 12511] |
| 5 | ENSG00000163166 | IWS1 homolog (S. cerevisiae) [Source: HGNC Symbol; Acc 25647] |
| 6 | ENSG00000100796 | SMEK homolog 1, suppressor of mek1 (Dictyostelium) [Source: HGNC Symbol; Acc: 20219] |
| 7 | ENSG00000122557 | HERPUD family member 2 [Source: HGNC Symbol; Acc 21915] |

[symbol]

indicates data missing or illegible when filed

1. A method of diagnosing cancerous cells in a patient, the method comprising:
a) providing a sample containing genetic material from patient cells suspected of being cancerous;
b) determining or measuring expression levels in the patient cells of at least 3 of the 1919 genes listed in Table B; and
c) computing a score using a classifier that takes said expression level values as input, the classifier having been previously trained on known cancerous and non-cancerous samples; wherein the score provides a likelihood of a cancerous cell.

2. The method of claim 1, wherein the at least 3 genes are genes found in at least one of Tables E, F, and I.

3. The method of claim 2, wherein the at least 3 genes are genes found in at least two of Tables E, F, and I.

4. The method of claim 3, wherein the at least 3 genes are genes found in all of Tables E, F, and I.

5. The method of claim 1, wherein the at least 3 genes is at least 10 genes.

6. The method of claim 1, wherein the at least 3 genes is at least 30 genes.

7. The method of claim 1, wherein the at least 3 genes is at least 100 genes.

8. The method of claim 2, wherein the at least 3 genes is at least 20, 40, 50, 60, 70, 80, 90, 150, 250, 300, 350, 400, 450, 500 or 1800 genes.

9. The method of claim 1, wherein the at least 3 genes are the 10 genes in Table I.

10. The method of claim 1, wherein the at least 3 genes consists of the 10 genes in Table I.

11. The method of claim 1, wherein the at least 3 genes are the 30 genes in Table E.

12. The method of claim 1, wherein the at least 3 genes consists of 30 the genes in Table E.

13. The method of claim 1, wherein the at least 3 genes are the 100 genes in Table F.

14. The method of claim 1, wherein the at least 3 genes consists of the 100 genes in Table F.

15. The method of claim 1, further comprising determining the tissue of origin of the patient cell by:
d) determining or measuring expression levels in the patient cells of at least 3 genes of the 450 genes listed in Table H; and
e) computing a score using a classifier that takes said expression level values as input, the classifier having been previously trained on known cancerous and non-cancerous samples from known tissues of origin; wherein the score provides a likelihood of the patient cell's tissue of origin.

16. The method of claim 15, wherein the at least 3 genes are the genes with the highest VarImp.

17. The method of claim 16, wherein the at least 3 genes is at least 20, 30, 40, 50, 60, 70, 80, 90, 100, 150, 200, 250, 300, 350, 400, 450, 500 or 1800 genes.

18. The method of claim 1, wherein the cancer is selected from the cancers identified in Table A.

19. The method of claim 1, wherein if there is a low likelihood of cancer, further comprising managing the patient with active surveillance.

20. The method of claim 1, wherein if there is a high likelihood of cancer, further comprising treating the patient with surgery, endocrine therapy, chemotherapy, radiotherapy, hormone therapy, gene therapy, thermal therapy, or ultrasound therapy.

21.-25. (canceled)
